(19) United States
(12) Patent Application Publication
(10) Pub. No.: US 2025/0318428 A1
(43) Pub. Date: Oct. 9, 2025

(54) NOVEL COMPOUND AND ORGANIC LIGHT EMITTING DEVICE COMPRISING THE SAME

(71) Applicant: LG CHEM, LTD., Seoul (KR)

(72) Inventors: Hoon Jun Kim, Daejeon (KR); Min Woo Jung, Daejeon (KR); Miyeon Han, Daejeon (KR); Jungha Lee, Daejeon (KR); Joongsuk Oh, Daejeon (KR); Hye Min Cho, Daejeon (KR); Hojung Lee, Daejeon (KR); Jeonghoe Heo, Daejeon (KR)

(21) Appl. No.: 18/865,994

(22) PCT Filed: Feb. 8, 2024
(86) PCT No.: PCT/KR2024/001891

§ 371 Date: Nov. 14, 2024

(30) Foreign Application Priority Data
Feb. 9, 2023 (KR) 10-2023-0017584

(51) Int. Cl. H10K 85/60; C07B 59/00; C07D 405/14; C07D 409/14; C09K 11/06; H10K 50/11

(52) U.S. Cl. CPC H10K 85/654; C07B 59/002; C07D 405/14; C07D 409/14; C09K 11/06; H10K 85/6572; H10K 85/6574; H10K 85/6576; C09K 2211/1007; C09K 2211/1018; H10K 50/11

(57) ABSTRACT

A compound of Chemical Formula 1:

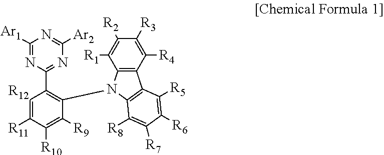

wherein: Ar₁ and Ar₂ are each independently a substituted or unsubstituted C₆₋₆₀ aryl or substituted or unsubstituted C₂₋₆₀ heteroaryl containing at least one heteroatom selected from N, O and S; R₁ to R₈ are each independently deuterium or a substituted or unsubstituted C₆₋₆₀ aryl, with the proviso that at least one of R₁ to R₈ is deuterium; any one of R₉ to R₁₁ is a bond with the following Chemical Formula 2, and the rest are each independently hydrogen or deuterium:

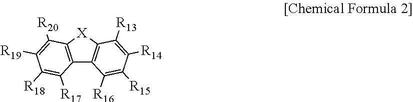

wherein: X is O or S; any one of R₁₃ to R₁₆ is a bond with the Chemical Formula 1, and the rest are each independently hydrogen, deuterium, or a substituted or unsubstituted C₆₋₆₀ aryl; and the other substituents are as defined in the specification; and an organic light-emitting device including the same.

### CROSS-REFERENCE TO RELATED APPLICATION(S)

This application is a National Stage Application of International Application No. PCT/KR2024/001891 filed on Feb. 8, 2024, which claims priority to and the benefit of Korean Patent Application No. 10-2023-0017584 filed on Feb. 9, 2023 in the Korean Intellectual Property Office, the disclosure of which is incorporated herein by reference in its entirety.

### TECHNICAL FIELD

The present disclosure relates to a novel compound and an organic light emitting device comprising the same.

### BACKGROUND ART

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic material used in the organic light emitting device as described above.

### PRIOR ART LITERATURE

### Patent Literature

(Patent Literature 0001) Korean Unexamined Patent Publication No. 10-2000-0051826

### DETAILED DESCRIPTION OF THE INVENTION

### Technical Problem

It is an object of the present disclosure to provide a novel organic light emitting material and an organic light emitting device comprising the same.

### Technical Solution

Provided herein is a compound represented by the following Chemical Formula 1:

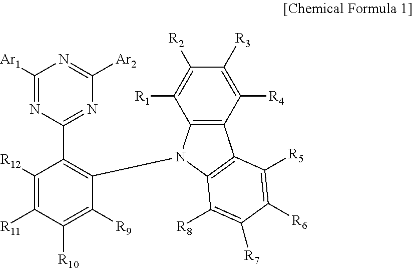

wherein, in Chemical Formula 1,
Ar₁ and Ar₂ are each independently a substituted or unsubstituted C₆₋₆₀ aryl;
or a substituted or unsubstituted C₂₋₆₀ heteroaryl containing at least one heteroatom selected from the group consisting of N, O and S,
R₁ to R₈ are each independently deuterium; or a substituted or unsubstituted C₁₋₆₀ aryl, with the proviso that at least one of R₁ to R₈ is deuterium,
any one of R₉ to R₁ is a bond with the following Chemical Formula 2, and the rest are each independently hydrogen or deuterium,
R₁₂ is hydrogen or deuterium,

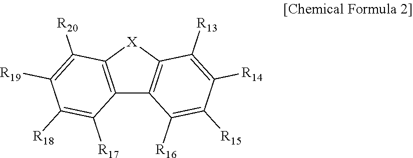

wherein, in Chemical Formula 2,
X is O or S,
any one of R₁₃ to R₁₆ is a bond with the Chemical Formula 1, and the rest are each independently hydrogen; deuterium; or a substituted or unsubstituted C₆₋₆₀ aryl, and
R₁₇ to R₂₀ are each independently hydrogen; deuterium; or a substituted or unsubstituted C₆₋₆₀ aryl.

Also provided herein is an organic light emitting device comprising: a first electrode; a second electrode that is provided opposite to the first electrode; and one or more organic material layers that are provided between the first electrode and the second electrode, wherein at least one layer of the organic material layers comprises the compound represented by Chemical Formula 1.

### Advantageous Effects

The compound represented by Chemical Formula 1 described above can be used as a material of an organic material layer of an organic light emitting device, and can improve the efficiency, achieve low driving voltage and/or improve lifetime characteristics in the organic light emitting device. In particular, the compound represented by Chemical Formula 1 can be used as a hole injection material, hole transport material, hole injection and transport material, electron blocking material, light emitting material, electron transport material, or electron injection material.

### BRIEF DESCRIPTION OF THE DRAWINGS

FIG. 1 shows an example of an organic light emitting device comprising a substrate 1, an anode 2, an organic material layer 3, and a cathode 4.

FIG. 2 shows an example of an organic light emitting device comprising a substrate 1, an anode 2, a hole injection layer 5, a hole transport layer 6, an electron blocking layer 7, a light emitting layer 8, a hole blocking layer 9, an electron transport layer 10, an electron injection layer 11, and a cathode 4.

FIG. 3 shows an example of an organic light emitting device comprising a substrate 1, an anode 2, a hole injection layer 5, a hole transport layer 6, an electron blocking layer 7, a light emitting layer 8, a hole blocking layer 9, an electron injection and transport layer 12, and a cathode 4.

### DETAILED DESCRIPTION OF THE EMBODIMENTS

Hereinafter, embodiments of the present disclosure will be described to help understanding of the disclosed subject matter.

The present disclosure provides the compound represented by Chemical Formula 1.

In the present disclosure, the notation

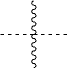

or [symbol] means a bond linked to another substituent group, and “D” means deuterium.

In the present disclosure, the term “substituted or unsubstituted” means being unsubstituted or substituted with one or more substituents selected from the group consisting of deuterium; a halogen group; a cyano group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heterocyclic group containing at least one of N, O and S atoms, or being unsubstituted or substituted with a substituent to which two or more substituents of the above-exemplified substituents are linked. For example, “a substituent in which two or more substituents are linked” may be a biphenylyl group. Namely, a biphenylyl group may be an aryl group, or it may be interpreted as a substituent formed by linking two phenyl groups. In one example, the term “substituted or unsubstituted” may be understood as meaning “being unsubstituted or substituted with one or more substituents selected from the group consisting of deuterium, halogen, cyano, a C₁₋₁₀ alkyl, a C₁₋₁₀ alkoxy and a C₆₋₂₀ aryl”, or “being unsubstituted or substituted with one or more substituents selected from the group consisting of deuterium, halogen, cyano, methyl, ethyl, phenyl and naphthyl”. Further, the term “substituted with one or more substituents” as used herein may be understood as meaning “being substituted with mono to the maximum number of substitutable hydrogens”. Alternatively, the term “substituted with one or more substituents” as used herein may be understood as meaning “being substituted with 1 to 5 substituents”, or “being substituted with one or two substituents”.

In the present disclosure, “linking two or more substituents among the substituents illustrated above” refers to linking hydrogen of any one substituent to another substituent.

In the present disclosure, “when a substituent is not indicated in the chemical formula or compound structure” may mean that hydrogen and deuterium mixedly exist in the chemical formula or compound structure unless deuterium is explicitly excluded, such as “the content of deuterium is 0%,” “the content of hydrogen is 100%,”.

In the present disclosure, the carbon number of a carbonyl group is not particularly limited, but is preferably 1 to 40. Specifically, it may be a substituent group having the following structure, but is not limited thereto.

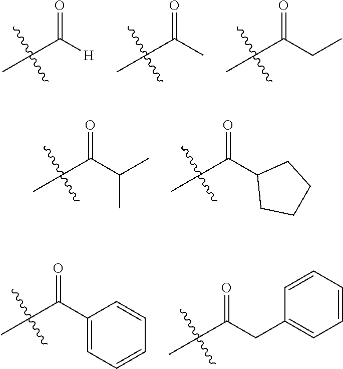

In the present disclosure, an ester group may have a structure in which oxygen of the ester group may be substituted by a straight-chain, branched-chain, or cyclic alkyl group having 1 to 25 carbon atoms, or an aryl group having 6 to 25 carbon atoms. Specifically, the ester group may be a substituent group having the following structural formulas, but is not limited thereto.

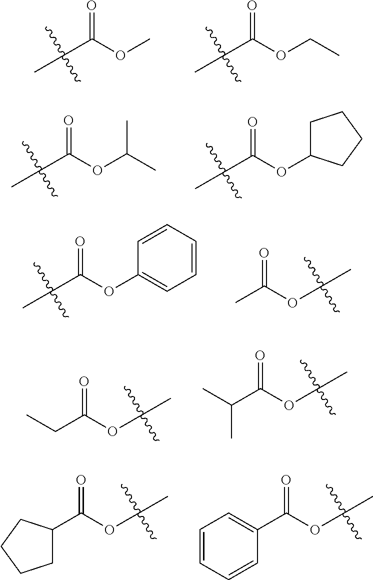

In the present disclosure, the carbon number of an imide group is not particularly limited, but is preferably 1 to 25. Specifically, the imide group may be a substituent group having the following structural formulas, but is not limited thereto.

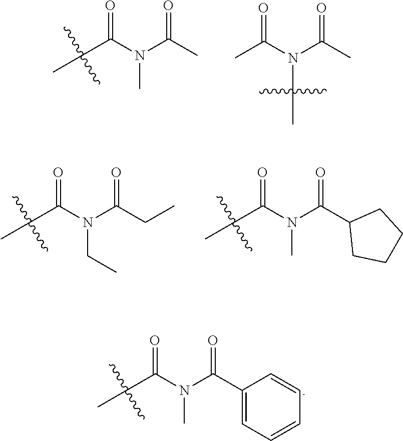

In the present disclosure, a substituted or unsubstituted silyl group means —Si(Z₁)(Z₂)(Z₃), wherein Z₁, Z₂ and Z₃ are each independently hydrogen, deuterium, a substituted or unsubstituted C₁₋₆₀ alkyl, a substituted or unsubstituted C₁₋₆₀ haloalkyl, a substituted or unsubstituted C₂₋₆₀ alkenyl, a substituted or unsubstituted C₂₋₆₀ haloalkenyl, or a substituted or unsubstituted C₆₋₆₀ aryl. According to one embodiment, Z₁, Z₂ and Z₃ may be each independently hydrogen, deuterium, a substituted or unsubstituted C₁₋₁₀ alkyl, a substituted or unsubstituted C₁₋₁₀ haloalkyl, or a substituted or unsubstituted C₆₋₂₀ aryl. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluoro, chloro, bromo, or iodo.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, 1-ethyl-propyl, 1,1-dimethylpropyl, hexyl, n-hexyl, 1-methylpentyl, 2-methylpentyl, 4-methyl-2-pentyl, 3,3-dimethylbutyl, 2-ethylbutyl, heptyl, n-heptyl, isohexyl, 1-methylhexyl, 2-methylhexyl, 3-methylhexyl, 4-methylhexyl, 5-methylhexyl, cyclopentylmethyl, cyclohexylmethyl, octyl, n-octyl, tert-octyl, 1-methylheptyl, 2-ethylhexyl, 2,4,4-trimethyl-1-pentyl, 2,4,4-trimethyl-2-pentyl, 2-propylpentyl, n-nonyl, 2,2-dimethylheptyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, the alicyclic group means a monovalent substituent derived from a saturated or unsaturated hydrocarbon ring compound that contains only carbon as a ring-forming atom, but does not have aromaticity, which is understood to encompass both monocyclic and fused polycyclic compounds. According to one embodiment, the carbon number of the alicyclic group is 3 to 60. According to another embodiment, the carbon number of the alicyclic group is 3 to 30. According to another embodiment, the carbon number of the alicyclic group is 3 to 20. Examples of the alicyclic group include a monocyclic group such as a cycloalkyl group, a bridged hydrocarbon group, a spiro hydrocarbon group, a substituent derived from hydrogenated derivatives of aromatic hydrocarbon compound.

Specifically, examples of the cycloalkyl group include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

Further, examples of the bridged hydrocarbon group include bicyclo[1.1.0]butyl, bicyclo[2.2.1]heptyl, bicyclo[4.2.0]octa-1,3,5-trienyl, adamantyl, decalinyl, and the like, but are not limited thereto.

Further, examples of the spiro hydrocarbon group include spiro[3.4]octyl, spiro[5.5]undecanyl, and the like, but are not limited thereto.

Further, a substituent derived from a hydrogenated derivative of the aromatic hydrocarbon compound means a substituent derived from a monocyclic or polycyclic aromatic hydrocarbon compound in which a part of the compound is hydrogenated. Examples of such a substituent include 1H-indenyl, 2H-indenyl, 4H-indenyl, 2,3-dihydro-1H-indenyl, 1,4-dihydronaphthalenyl, 1,2,3,4-tetrahydronaphthalenyl, 6,7,8,9-tetrahydro-5H-benzo[7]annulenyl, 6,7-dihydro-5H-benzocycloheptenyl, and the like, but are not limited thereto.

In the present disclosure, an aryl group is understood to mean a substituent derived from a monocyclic or fused polycyclic compound containing only carbon as a ring-forming atom and also having aromaticity, and the carbon number thereof is not particularly limited, but is preferably 6 to 60. According to one embodiment, the carbon number of the aryl group is 6 to 30. According to one embodiment, the carbon number of the aryl group is 6 to 20. The aryl group may be a phenyl group, a biphenylyl group, a terphenylyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, or the like, but is not limited thereto.

In the present disclosure, the fluorenyl group may be substituted, and two substituent groups may be linked with each other to form a spiro structure. In the case where the fluorenyl group is substituted,

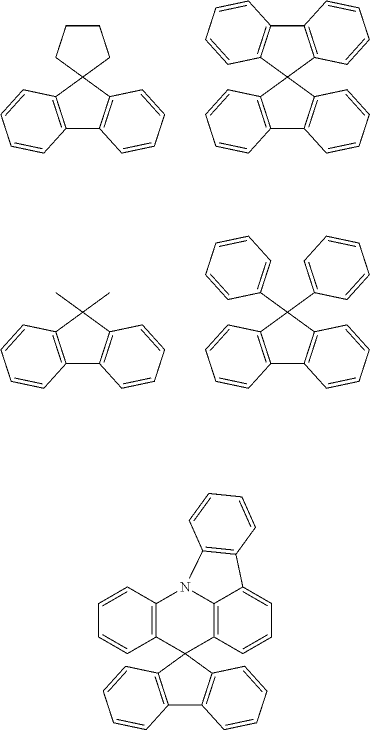

and the like can be formed. However, the structure is not limited thereto.

In the present disclosure, a heterocyclic group means a monovalent substituent derived from a monocyclic or fused polycyclic compound that further contains at least one heteroatom selected from O, N, Si, and S in addition to carbon as a ring-forming atom, and is understood to encompass both substituents with aromaticity and substituents without aromaticity. According to one embodiment, the carbon number of the heterocyclic group is 2 to 60 carbon atoms. According to another embodiment, the carbon number of the heterocyclic group is 2 to 30. According to another embodiment, the carbon number of the heterocyclic group is 2 to 20. Examples of such a heterocyclic group include a heteroaryl group, a substituent derived from a hydrogenated derivative of the heteroaromatic compound, and the like.

Specifically, the heteroaryl group means a substituent derived from a monocyclic or fused polycyclic compound which further contains at least one heteroatom selected from N, O and S in addition to carbon as a ring forming atom, and refers to a substituent having aromaticity. According to one embodiment, the carbon number of the heteroaryl group is 2 to 60. According to another embodiment, the carbon number of the heteroaryl group is 2 to 30. According to another embodiment, the carbon number of the heteroaryl group is 2 to 20. Examples of the heteroaryl group include a thiophenyl group, a furanyl group, a pyrrole group, an imidazolyl group, a thiazolyl group, an oxazolyl group, an oxadiazolyl group, a triazolyl group, a pyridinyl group, a bipyridinyl group, a pyrimidinyl group, a triazinyl group, an acridinyl group, a pyridazinyl group, a pyrazinyl group, a quinolinyl group, a quinazolinyl group, a quinoxalinyl group, a phthalazinyl group, a pyridopyrimidinyl group, a pyridopyrazinyl group, an isoquinolinyl group, an indolyl group, a carbazolyl group, a benzoxazolyl group, a benzoimidazolyl group, a benzothiazolyl group, a benzocarbazolyl group, a benzothiophenyl group, a dibenzothiophenyl group, a benzofuranyl group, a dibenzofuranyl group, a phenanthrolinyl group, an isoxazolyl group, a thiadiazolyl group, a phenothiazinyl group, and the like, but are not limited thereto.

Further, a substituent derived from a hydrogenated derivative of a heteroaromatic compound means a substituent derived from a monocyclic or polycyclic heteroaromatic compound in which a part of the unsaturated bond of the compound is hydrogenated. Examples of such substituents include 1,3-dihydroisobenzofuranyl, 2,3-dihydrobenzofuranyl, 1,3-dihydrobenzo[c]thiophenyl, 2,3-dihydro[b]thiophenyl, and the like, but are not limited thereto.

In the present disclosure, the aryl group in the aralkyl group, the aralkenyl group, the alkylaryl group, the arylamine group and the arylsilyl group is the same as the examples of the aryl group as defined above. In the present disclosure, the alkyl group in the aralkyl group, the alkylaryl group and the alkylamine group is the same as the examples of the alkyl group as defined above. In the present disclosure, the heteroaryl in the heteroarylamine can be applied to the description of the heteroaryl as defined above. In the present disclosure, the alkenyl group in the aralkenyl group is the same as the examples of the alkenyl group as defined above. In the present disclosure, the description of the aryl group as defined above may be applied except that the arylene is a divalent group. In the present disclosure, the description of the heteroaryl as defined above can be applied except that the heteroarylene is a divalent group. In the present disclosure, the description of the aryl group or cycloalkyl group as defined above can be applied except that the hydrocarbon ring is not a monovalent group but formed by combining two substituent groups. In the present disclosure, the description of the heteroaryl as defined above can be applied, except that the heterocycle is not a monovalent group but formed by combining two substituent groups.

In the present disclosure, the term “deuterated or substituted with deuterium” means that at least one of the substitutable hydrogens in a compound, a divalent linking group, or a monovalent substituent has been substituted with deuterium.

Further, the term “unsubstituted or substituted with deuterium” or “substituted or unsubstituted with deuterium” means that “mono to the maximum number of unsubstituted or substitutable hydrogens have been substituted with deuterium.” In one example, the term “phenanthryl unsubstituted or substituted with deuterium” may be understood as meaning “phenanthryl unsubstituted or substituted with 1 to 9 deuterium atoms”, considering that the maximum number of hydrogen that can be substituted with deuterium in the phenanthryl structure is 9.

Further, “deuterated structure” means to include compounds, divalent linking groups, or monovalent substituents of all structures in which at least one hydrogen is substituted with deuterium. As an example, the deuterated structure of phenyl can be understood to refer to monovalent substituents of all structures in which at least one substitutable hydrogen in the phenyl group is substituted with deuterium, as follows.

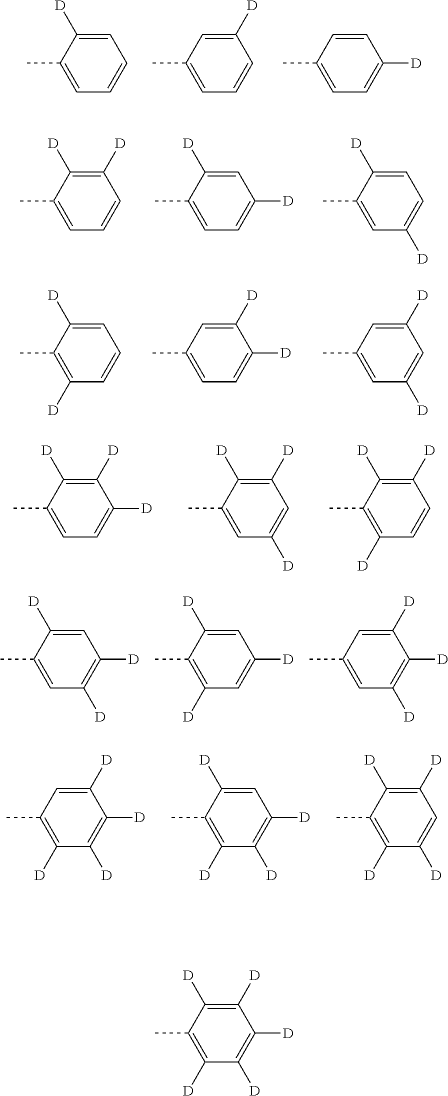

In addition, the “deuterium substitution rate” or “degree of deuteration” of a compound means that the ratio of the number of substituted deuterium atoms to the total number of hydrogen atoms (the sum of the number of hydrogen atoms substitutable with deuterium and the number of substituted deuterium atoms in a compound) that can exist in the compound is calculated as a percentage. Therefore, when the “deuterium substitution rate” or “degree of deuteration” of a compound is “K %”, it means that K % of the hydrogen atoms substitutable with deuterium in the compound are substituted with deuterium.

At this time, the presence or absence of deuterium substitution of a compound can be determined according to a commonly known method using MALDI-TOF MS (Matrix-Assisted Laser Desorption/Ionization Time-of-Flight Mass Spectrometer), a nuclear magnetic resonance spectroscopy (¹H NMR), TLC/MS (Thin-Layer Chromatography/Mass Spectrometry), GO/MS (Gas Chromatography/Mass Spectrometry), or the like.

More specifically, when using MALDI-TOF MS, the “deuterium substitution rate” or “degree of deuteration” may be obtained by determining the number of substituted deuterium in the compound through MALDI-TOF MS analysis, and then calculating the ratio of the number of substituted deuterium to the total number of hydrogen atoms that can exist in the compound as a percentage.

In addition, when analyzing the “deuterium substitution rate” or “degree of deuteration” through TLC/MS (thin-layer chromatography/mass spectrometry), the substitution rate can be calculated based on the maximum value (max. value) of distribution that molecular weights form at the end of the reaction.

Further, when analyzing the “deuterium substitution rate” or “degree of deuteration” using nuclear magnetic resonance (¹H NMR), the deuterium substitution rate or degree of deuteration can be calculated from the integration quantity of total peaks through the integration ratio in ¹H NMR.

On the other hand, the present disclosure provides a compound represented by the Chemical Formula 1.

The compound of Chemical Formula 1 includes a triazine and carbazole bonded at an ortho position, wherein the carbazole includes at least one deuterium. In addition, the compound of Chemical Formula 1 has a structure in which dibenzofuran or dibenzothiophene is bonded to a benzene ring to which triazine and carbazole are bonded, but is bonded to carbon rather than at the ortho position relative to the triazine. As it satisfies this structure, the compound represented by Chemical Formula 1 can be used in an organic light emitting device and exhibit low voltage, high efficiency, and long lifetime characteristics.

More specifically, the present inventors have found that in the case of a compound having a structure in which a carbazolyl group is substituted at the ortho-position of a triazine on the basis of a benzene ring, a steric hindrance is induced and a triazine moiety and a carbazole moiety have a distorted structure, and in such a structure, the stability of the overall molecule increases due to the electron donating properties of the carbazolyl group, while the electron distribution is separated, and the compound has additional CT (charge transfer) properties, which leads to an increase in the voltage and efficiency of devices employing the above compound, and completed the present disclosure.

However, in such a structure, an electron deficiency phenomenon may occur in the carbazolyl group, and therefore, in order to overcome this problem, the compound represented by Chemical Formula 1 has a deuterium or aryl group instead of hydrogen in the carbazolyl group, and also further contains dibenzofuranyl or dibenzothiophenyl in the benzene ring, thereby maximizing the molecular stability and electronic properties of the compound represented by Chemical Formula 1. Therefore, it is possible to further improve the lifetime properties of an organic light emitting device employing the compound represented by Chemical Formula 1.

Preferably, R₁₀ of Chemical Formula 1 is a bond with Chemical Formula 2, and R₉ and R₁₁ are each independently hydrogen or deuterium; or
R₁ of Chemical Formula 1 is a bond with Chemical Formula 2, and R₉ and R₁₀ may each independently be hydrogen or deuterium.

In addition, R₁₃ of Chemical Formula 2 is a bond with Chemical Formula 1, and R₁₄, R₁₅ and R₁₆ are each independently hydrogen; deuterium; or a substituted or unsubstituted C₆₋₆₀ aryl; or
R₁₄ of Chemical Formula 2 is a bond with Chemical Formula 1, and R₁₃, R₁₅, and R₁₆ are each independently hydrogen; deuterium; or a substituted or unsubstituted C₆₋₆₀ aryl; or
R₁₅ of Chemical Formula 2 is a bond with Chemical Formula 1, and R₁₃, R₁₄, and R₁₆ are each independently hydrogen; deuterium; or a substituted or unsubstituted C₆₋₆₀ aryl; or
R₁₆ of Chemical Formula 2 is a bond with Chemical Formula 1, and R₁₃, R₁₄, and R₁₅ are each independently hydrogen; deuterium; or a substituted or unsubstituted C₆₋₆₀ aryl.

Specifically, the Chemical Formula 1 may be represented by any one of the following Chemical Formulas 1-1 to 1-6.

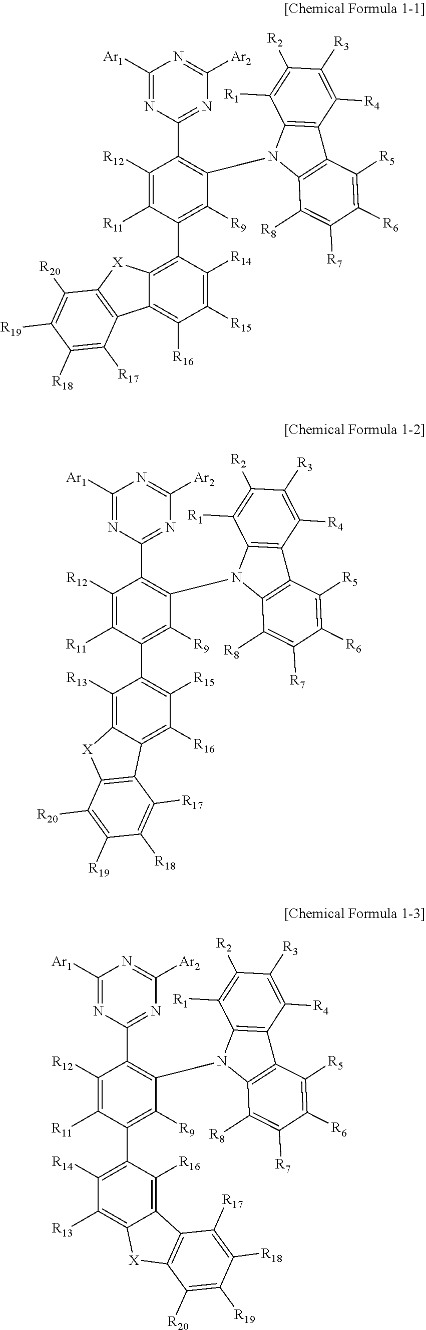

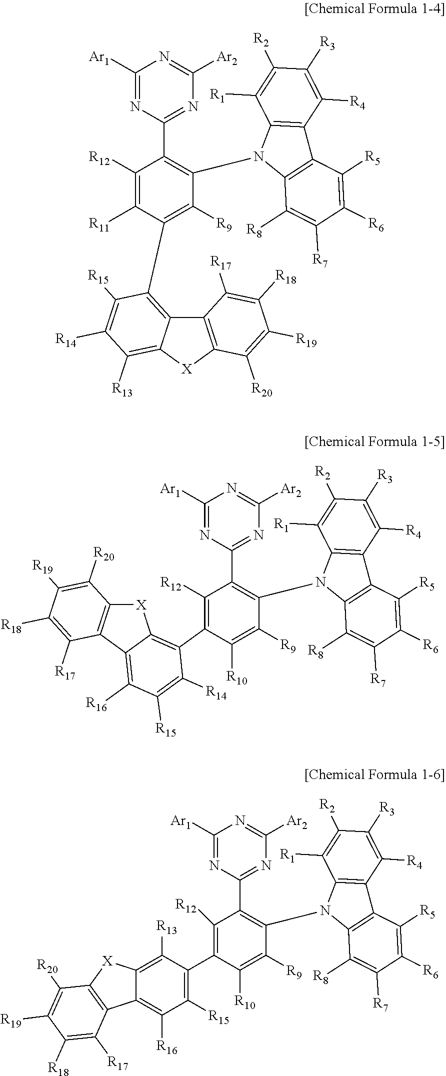

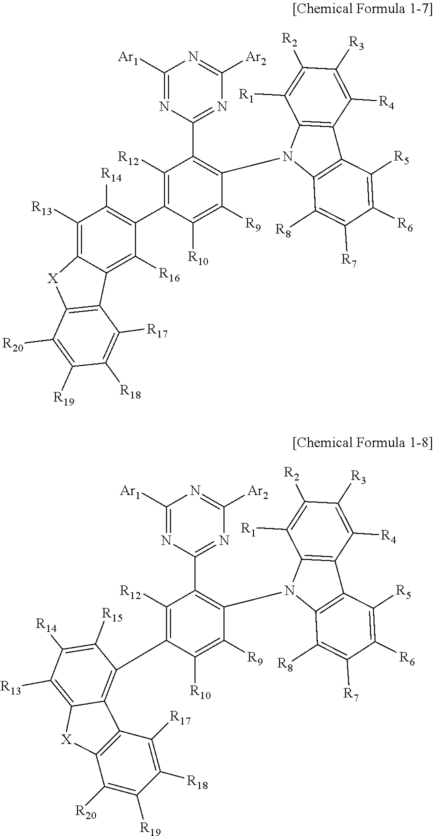

wherein, in Chemical Formulas 1-1 to 1-8,
Ar₁, Ar₂, X, and R₁ to R₂₀ are as defined in Chemical Formulas 1.

Preferably, R₁ to R₈ are each independently deuterium; or a C₆₋₂₀ aryl substituted or unsubstituted with deuterium.

Preferably, R₁ to R₈ are each independently deuterium; or phenyl substituted with at least one deuterium (preferably, phenyl substituted with 5 deuteriums).

More preferably, R₁ to R₈ are each independently deuterium; or phenyl substituted with 5 deuteriums.

Preferably, all of R₁ to R₈ are deuterium; or any one of R₁ to R₈ may be a C₆₋₁₂ aryl substituted or unsubstituted with deuterium, and all the rest may be deuterium.

As an example, all of R₁ to R₈ may be deuterium. Alternatively, any one of R₁ to R₈ may be phenyl substituted with at least one deuterium, and the rest may be deuterium.

Preferably, Ar₁ and Ar₂ are each independently a substituted or unsubstituted C₆₋₂₀ aryl; or a substituted or unsubstituted C₂₋₂₀ heteroaryl containing at least one heteroatom selected from the group consisting of N, O, and S.

Preferably, Ar₁ and Ar₂ are each independently phenyl; phenyl substituted with at least one deuterium; biphenylyl; or biphenylyl substituted with at least one deuterium.

Preferably, Ar₁ and Ar₂ are each independently phenyl; or phenyl substituted with at least one deuterium (preferably, phenyl substituted with 5 deuteriums).

Preferably, Ar₁ and Ar₂ are each independently phenyl; or phenyl substituted with 1 to 5 deuteriums.

Preferably, all the rest of R₉ to R₁ not bonding with Chemical Formula 2 are hydrogen.

Preferably, R₁₂ is hydrogen.

Preferably, the rest of R₁₃ to R₁₆ not bonding with Chemical Formula 1 and R₁₇ to R₂₀ are each independently hydrogen; deuterium; or a substituted or unsubstituted C₆₋₂₀ aryl.

Preferably, the rest of R₁₃ to R₁₆ not bonding with Chemical Formula 1 and R₁₇ to R₂₀ are each independently hydrogen; deuterium; phenyl; or phenyl substituted with at least one deuterium.

Preferably, all of the rest of R₁₃ to R₁₆ not bonding with Chemical Formula 1 and R₁₇ to R₂₀ are hydrogen; or all are deuterium; or one of the rest of R₁₃ to R₁₆ not bonding with Chemical Formula 1 and R₁₇ to R₂₀ is an unsubstituted C₆₋₂₀ aryl, and all the rest are hydrogen; or one of the rest of R₁₃ to R₁₆ not bonding with Chemical Formula 1 and R₁₇ to R₂₀ is a C₆₋₂₀ aryl in which all substitutable positions are substituted with deuterium, and all the rest are deuterium.

More preferably, all of the rest of R₁₃ to R₁₆ not bonding with Chemical Formula 1 and R₁₇ to R₂₀ are hydrogen; or all are deuterium; or one of the rest of R₁₃ to R₁₆ not bonding with Chemical Formula 1 and R₁₇ to R₂₀ is phenyl, and all the rest are hydrogen; or one of the rest of R₁₃ to R₁₆ not bonding with Chemical Formula 1 and R₁₇ to R₂₀ is phenyl substituted with 5 deuteriums, and all the rest are deuterium.

Representative examples of the compound represented by Chemical Formula 1 are as follows:

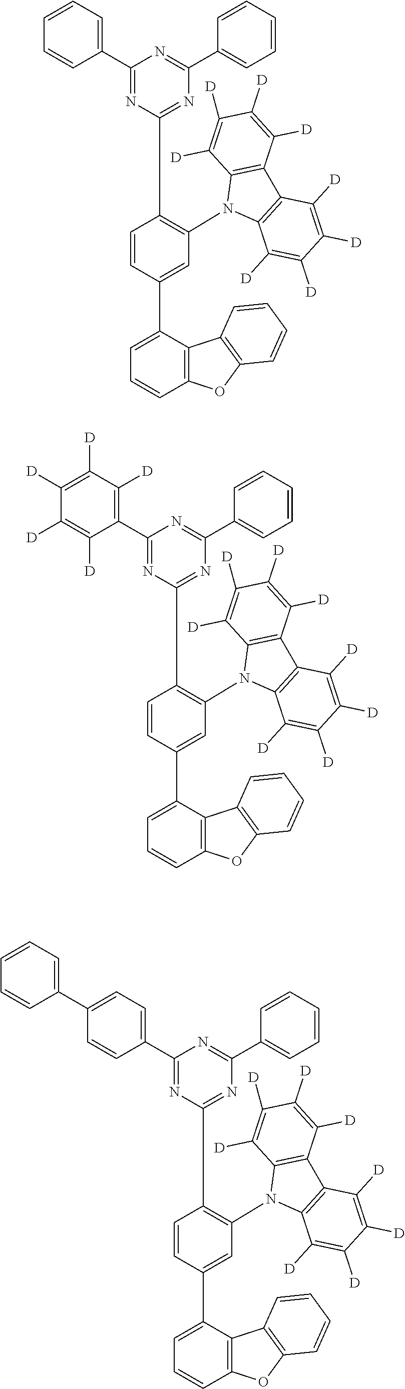 [SMILES: [2H]c1c([2H])c([2H])c2c(c1[2H])c1c([2H])c([2H])c([2H])c([2H])c1n2-c1cc(-c2cccc3oc4ccccc4c23)ccc1-c1nc(-c2ccccc2)nc(-c2ccccc2)n1 ; [2H]c1c([2H])c([2H])c(-c2nc(-c3ccccc3)nc(-c3ccc(-c4cccc5oc6ccccc6c45)cc3-n3c4c([2H])c([2H])c([2H])c([2H])c4c4c([2H])c([2H])c([2H])c([2H])c43)n2)c([2H])c1[2H] ; [2H]c1c([2H])c([2H])c2c(c1[2H])c1c([2H])c([2H])c([2H])c([2H])c1n2-c1cc(-c2cccc3oc4ccccc4c23)ccc1-c1nc(-c2ccccc2)nc(-c2ccc(-c3ccccc3)cc2)n1]

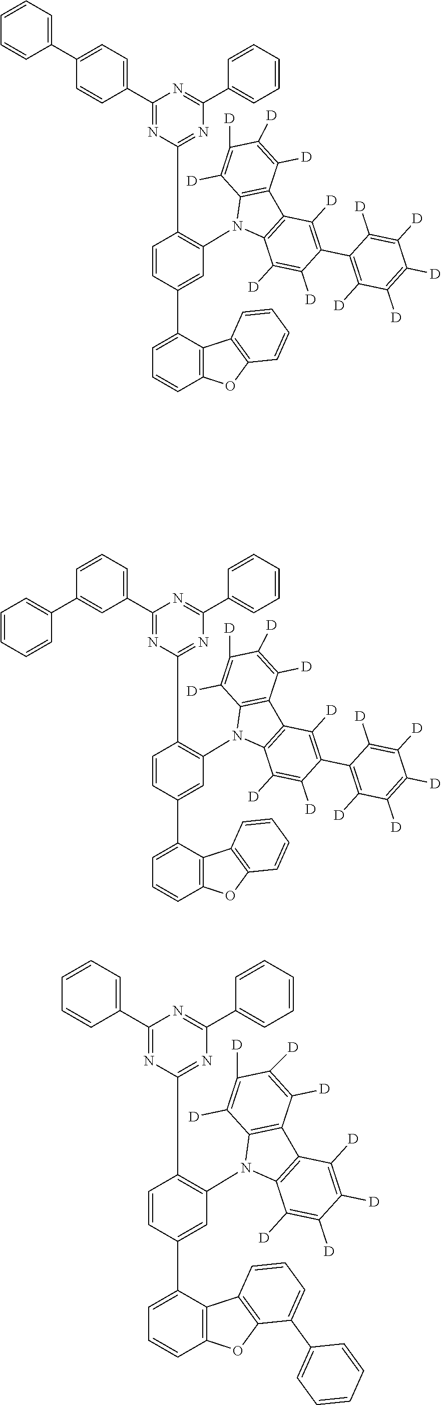 [SMILES: [2H]c1c([2H])c([2H])c(-c2c([2H])c([2H])c3c(c2[2H])c2c([2H])c([2H])c([2H])c([2H])c2n3-c2cc(-c3cccc4oc5ccccc5c34)ccc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2cc(-c3cccc4oc5ccccc5c34)ccc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1cc(-c2cccc3oc4c(-c5ccccc5)cccc4c23)ccc1-c1nc(-c2ccccc2)nc(-c2ccccc2)n1]

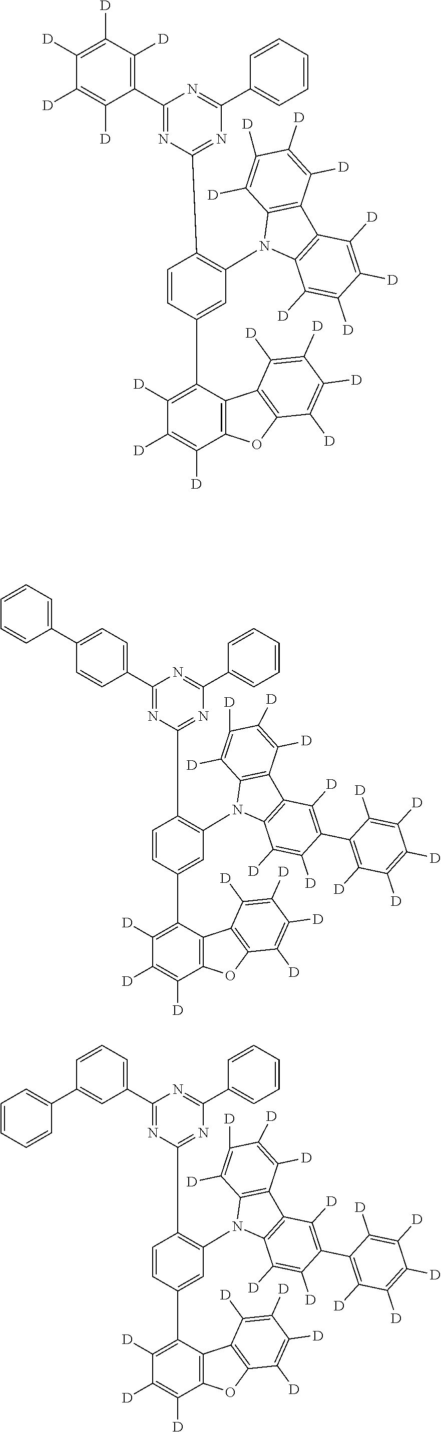

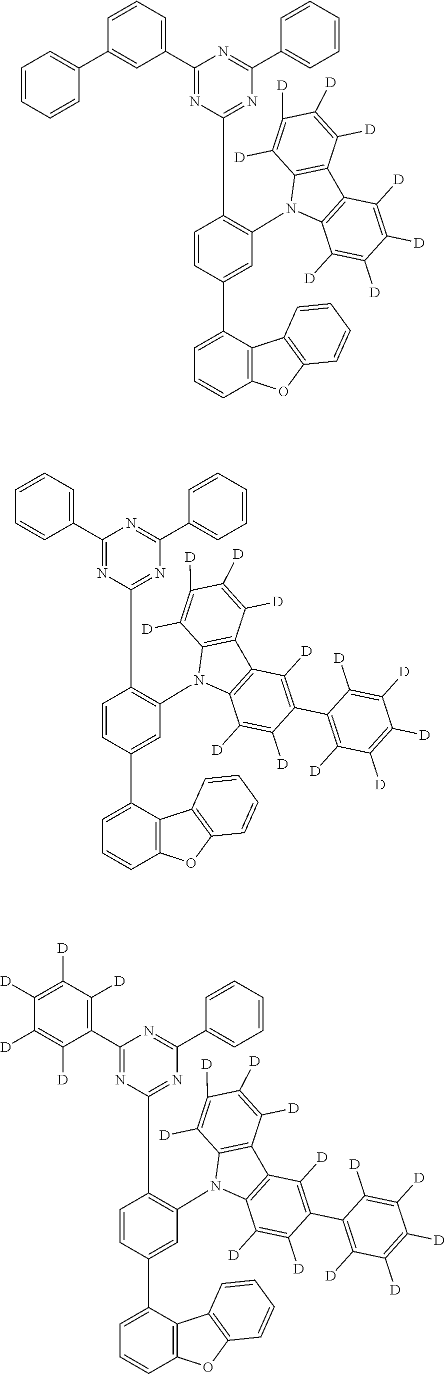 [SMILES: [2H]c1c([2H])c([2H])c2c(c1[2H])c1c([2H])c([2H])c([2H])c([2H])c1n2-c1cc(-c2cccc3oc4ccccc4c23)ccc1-c1nc(-c2ccccc2)nc(-c2cccc(-c3ccccc3)c2)n1 ; [2H]c1c([2H])c([2H])c(-c2c([2H])c([2H])c3c(c2[2H])c2c([2H])c([2H])c([2H])c([2H])c2n3-c2cc(-c3cccc4oc5ccccc5c34)ccc2-c2nc(-c3ccccc3)nc(-c3ccccc3)n2)c([2H])c1[2H] ; [2H]c1c([2H])c([2H])c(-c2nc(-c3ccccc3)nc(-c3ccc(-c4cccc5oc6ccccc6c45)cc3-n3c4c([2H])c([2H])c([2H])c([2H])c4c4c([2H])c(-c5c([2H])c([2H])c([2H])c([2H])c5[2H])c([2H])c([2H])c43)n2)c([2H])c1[2H]]

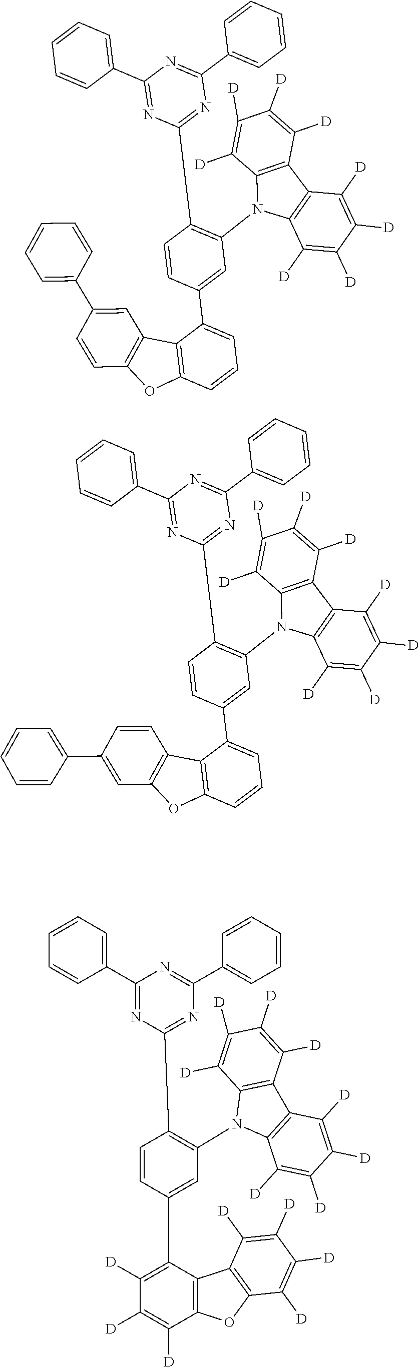 [SMILES: [2H]c1c([2H])c([2H])c2c(c1[2H])c1c([2H])c([2H])c([2H])c([2H])c1n2-c1cc(-c2cccc3oc4ccc(-c5ccccc5)cc4c23)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cc(-c2cccc3oc4cc(-c5ccccc5)ccc4c23)ccc1-c1nc(-c2ccccc2)nc(-c2ccccc2)n1 ; [2H]c1c([2H])c([2H])c2c(oc3c([2H])c([2H])c([2H])c(-c4ccc(-c5nc(-c6ccccc6)nc(-c6ccccc6)n5)c(-n5c6c([2H])c([2H])c([2H])c([2H])c6c6c([2H])c([2H])c([2H])c([2H])c65)c4)c32)c1[2H]]

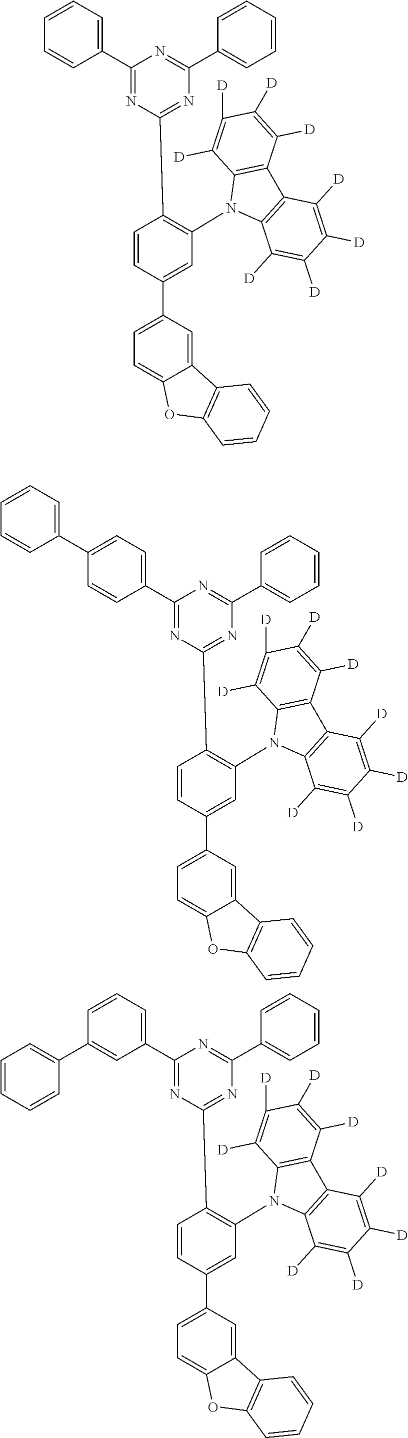

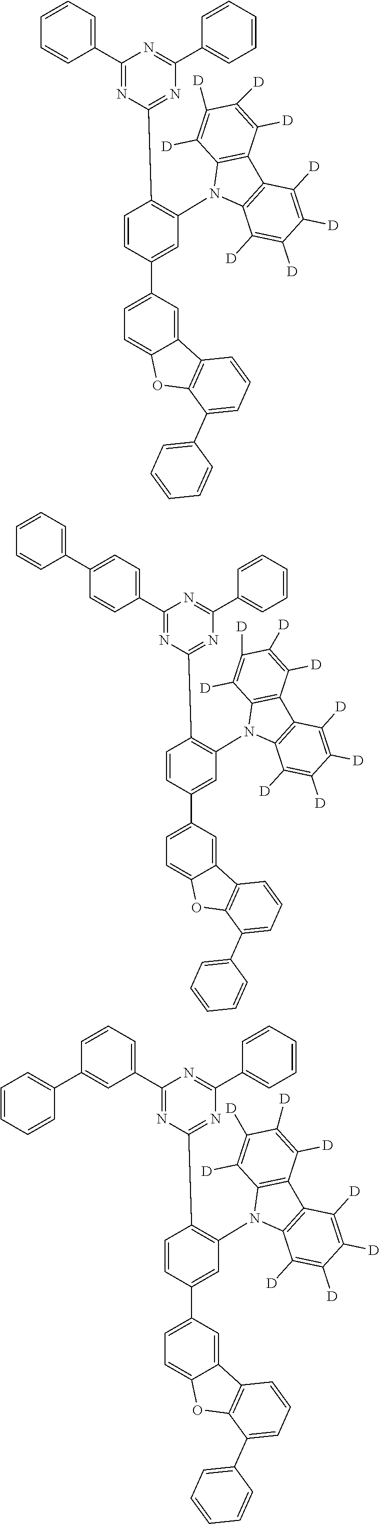

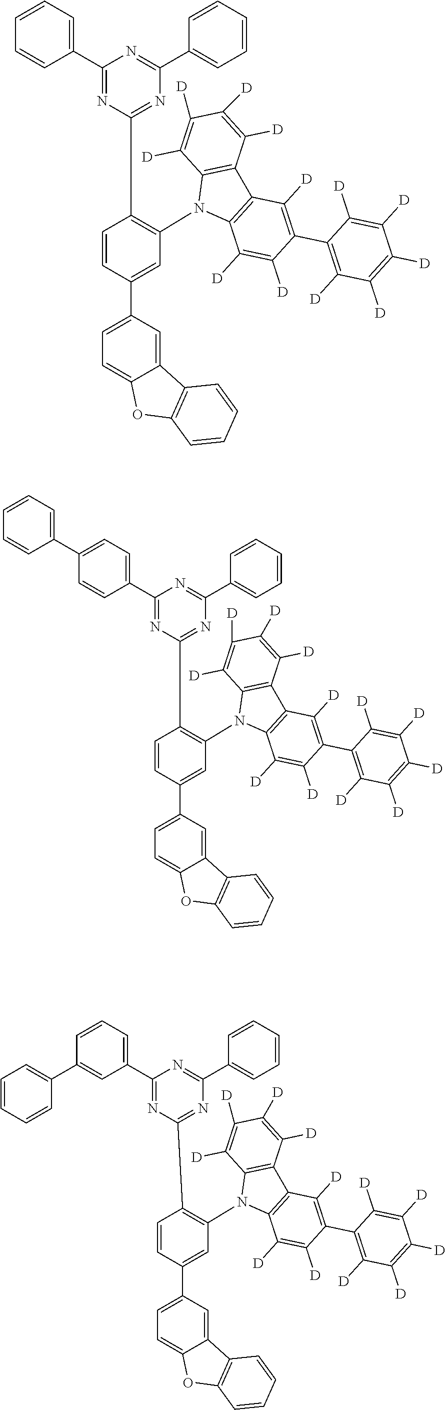

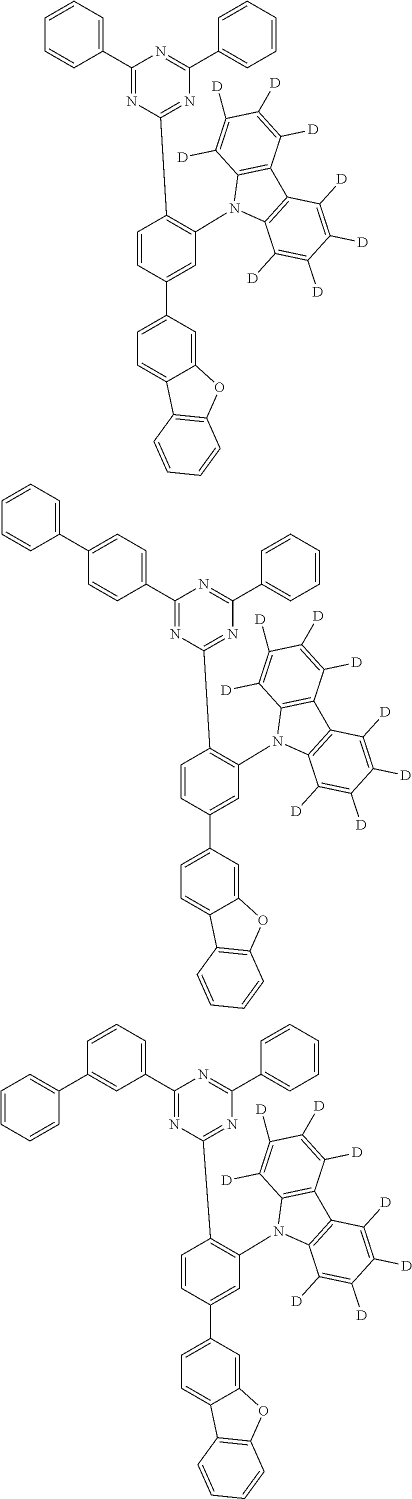

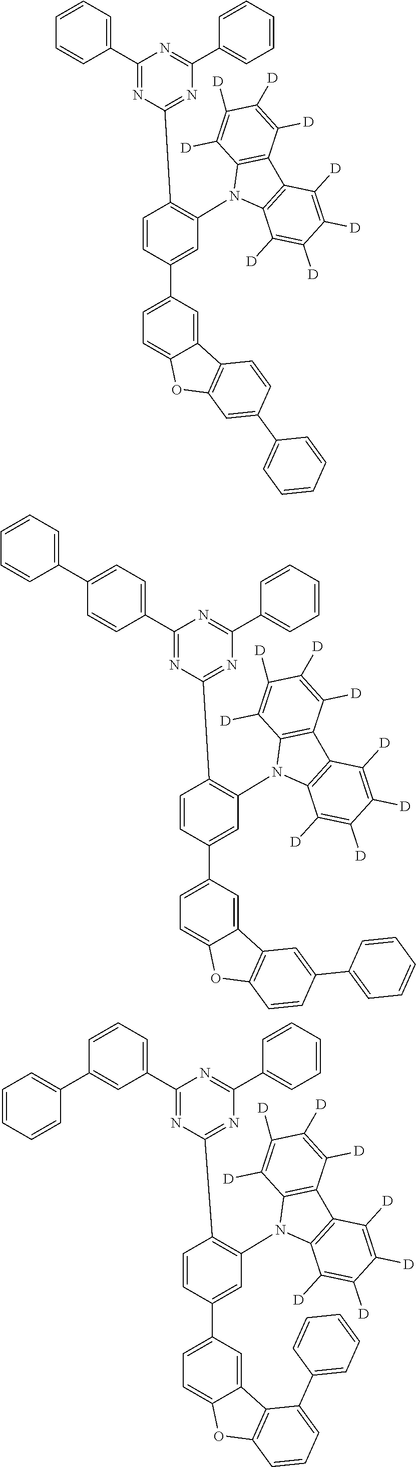

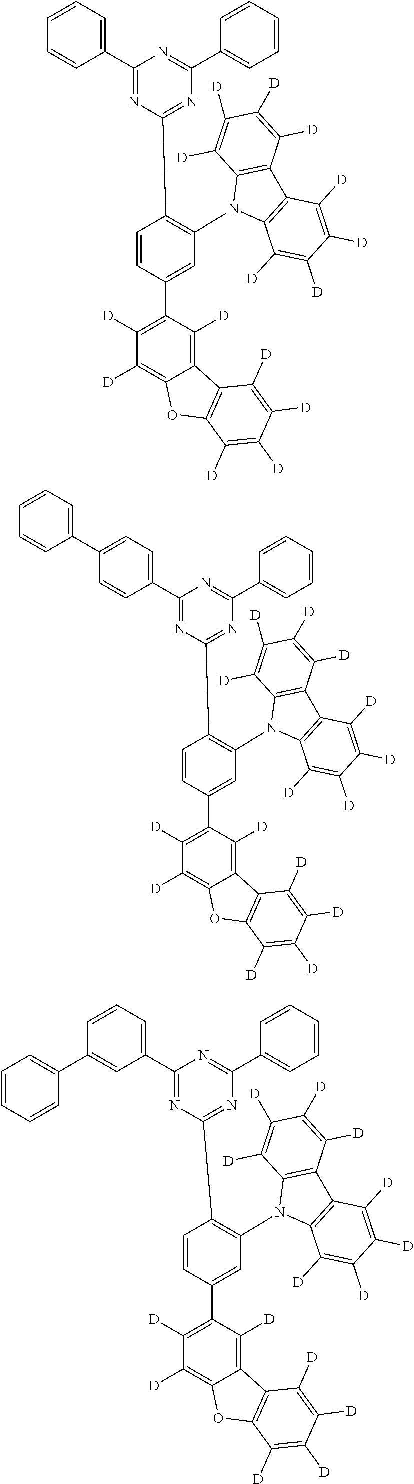

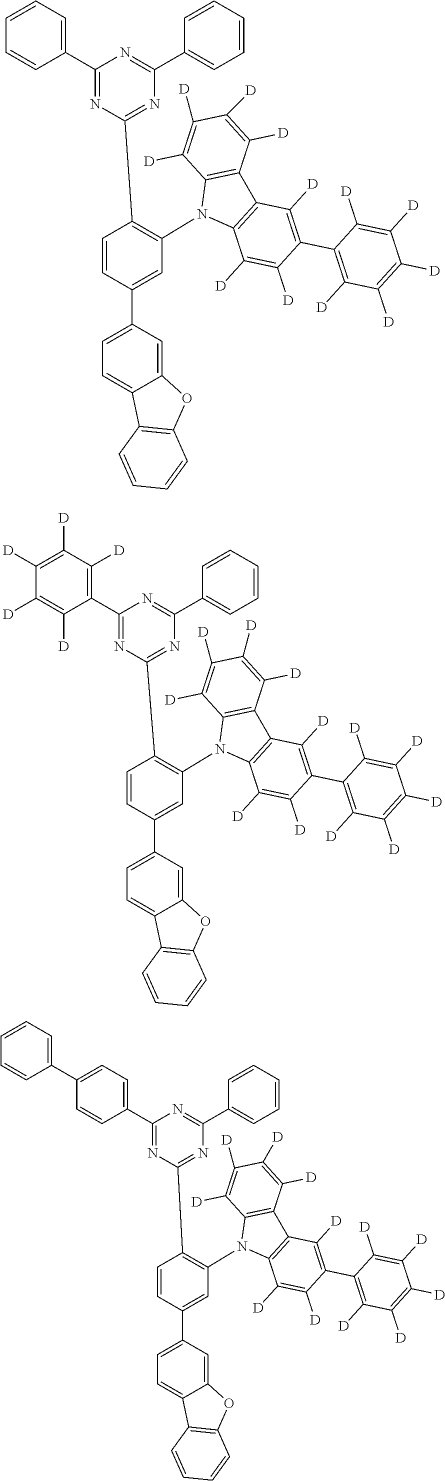

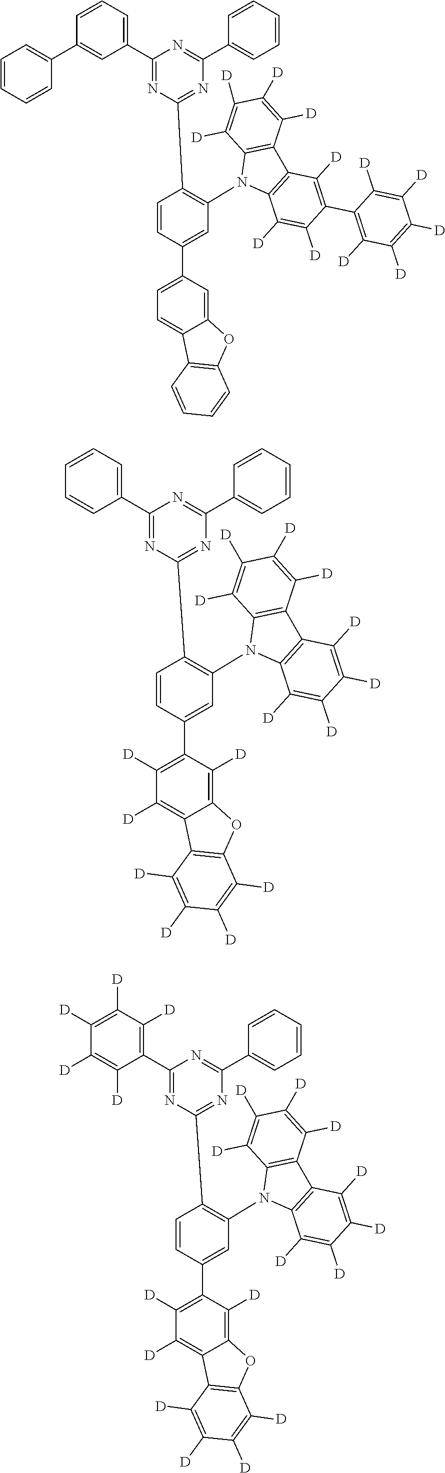

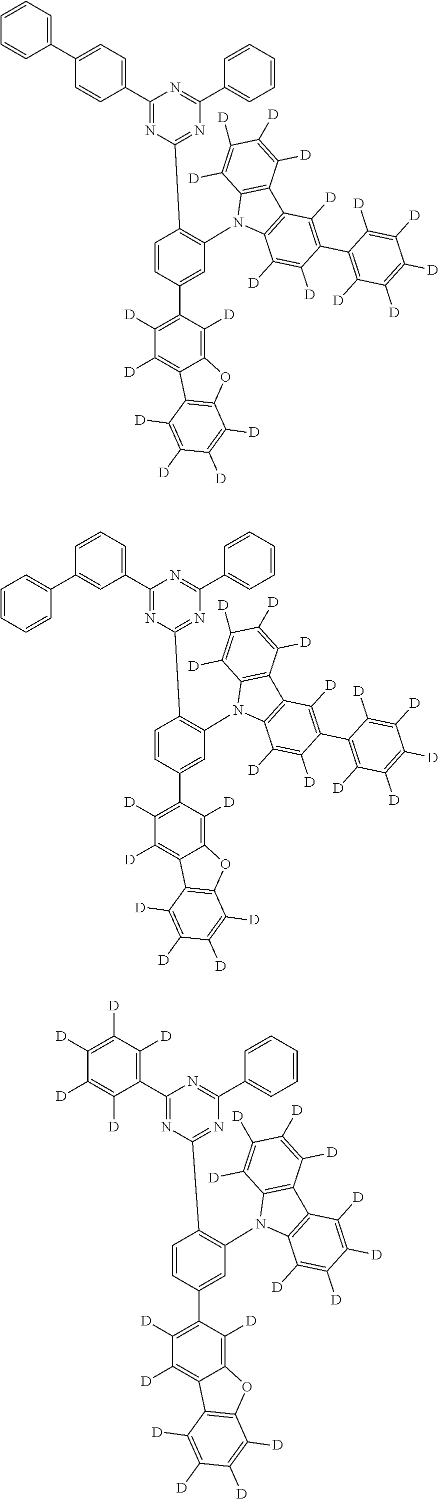

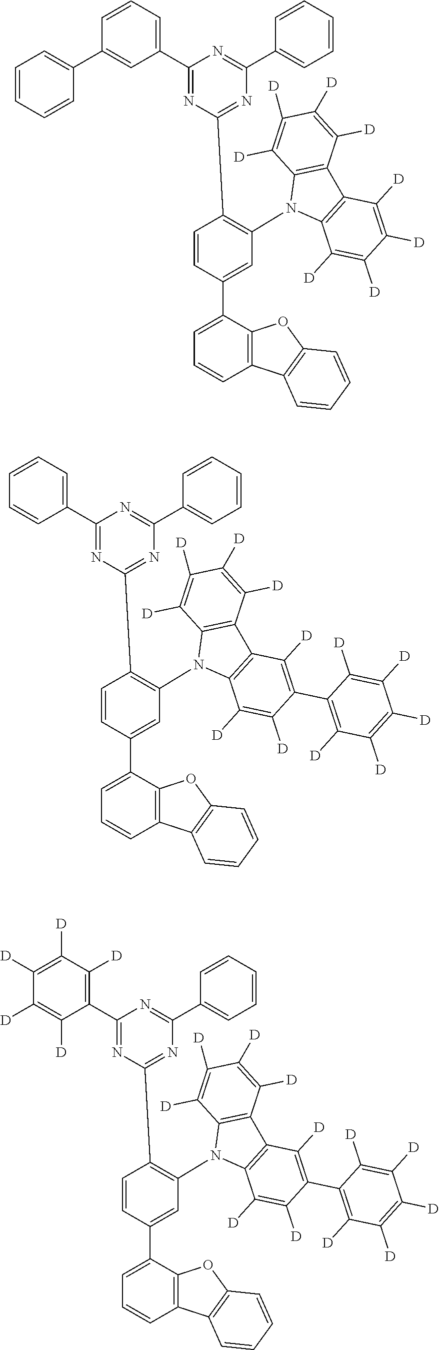 [SMILES: [2H]c1c([2H])c([2H])c2c(c1[2H])c1c([2H])c([2H])c([2H])c([2H])c1n2-c1cc(-c2cccc3c2oc2ccccc23)ccc1-c1nc(-c2ccccc2)nc(-c2cccc(-c3ccccc3)c2)n1 ; [2H]c1c([2H])c([2H])c(-c2c([2H])c([2H])c3c(c2[2H])c2c([2H])c([2H])c([2H])c([2H])c2n3-c2cc(-c3cccc4c3oc3ccccc34)ccc2-c2nc(-c3ccccc3)nc(-c3ccccc3)n2)c([2H])c1[2H] ; [2H]c1c([2H])c([2H])c(-c2nc(-c3ccccc3)nc(-c3ccc(-c4cccc5c4oc4ccccc45)cc3-n3c4c([2H])c([2H])c([2H])c([2H])c4c4c([2H])c(-c5c([2H])c([2H])c([2H])c([2H])c5[2H])c([2H])c([2H])c43)n2)c([2H])c1[2H]]

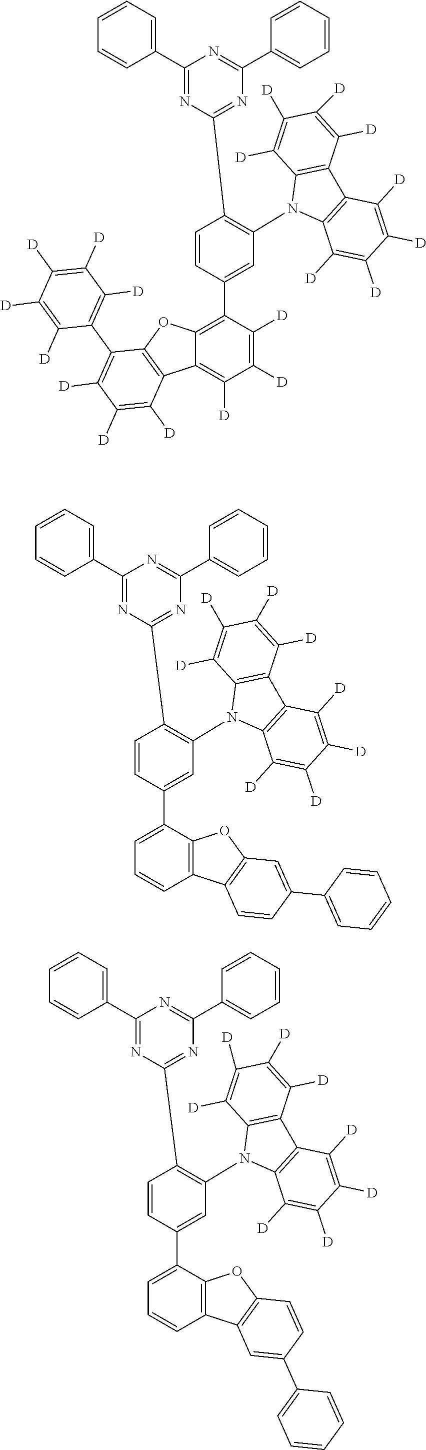

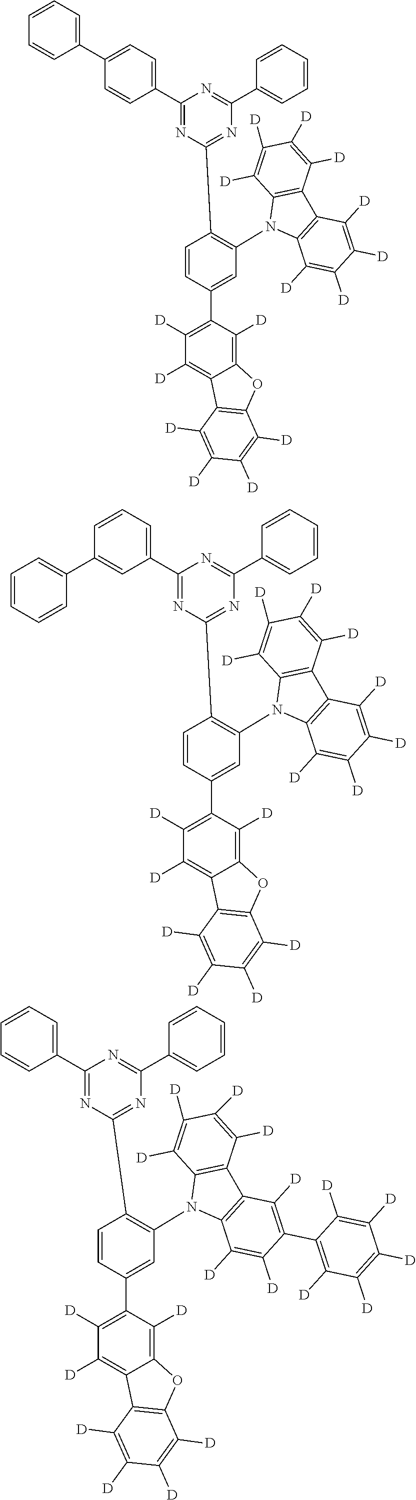

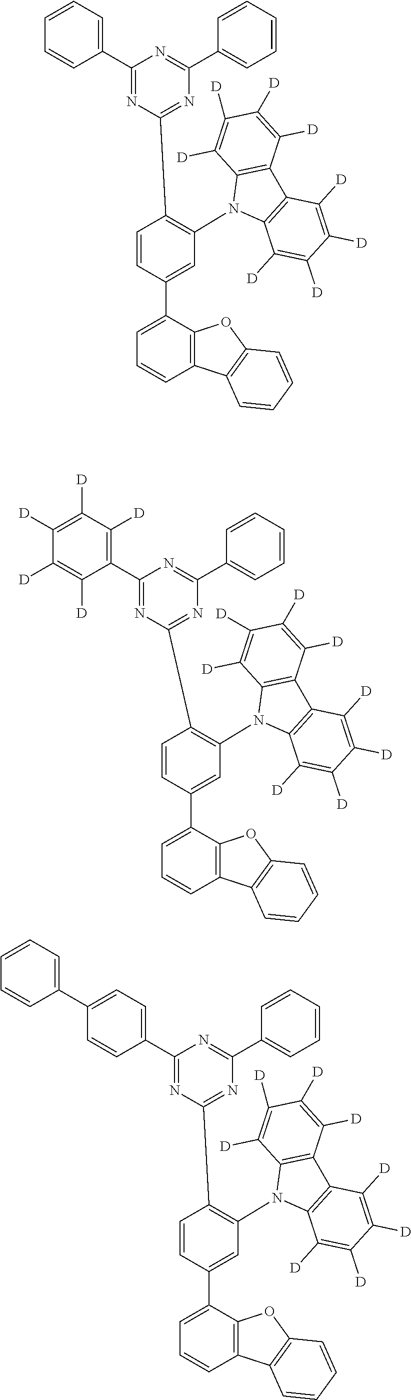 [SMILES: [2H]c1c([2H])c([2H])c2c(c1[2H])c1c([2H])c([2H])c([2H])c([2H])c1n2-c1cc(-c2cccc3c2oc2ccccc23)ccc1-c1nc(-c2ccccc2)nc(-c2ccccc2)n1 ; [2H]c1c([2H])c([2H])c(-c2nc(-c3ccccc3)nc(-c3ccc(-c4cccc5c4oc4ccccc45)cc3-n3c4c([2H])c([2H])c([2H])c([2H])c4c4c([2H])c([2H])c([2H])c([2H])c43)n2)c([2H])c1[2H] ; [2H]c1c([2H])c([2H])c2c(c1[2H])c1c([2H])c([2H])c([2H])c([2H])c1n2-c1cc(-c2cccc3c2oc2ccccc23)ccc1-c1nc(-c2ccccc2)nc(-c2ccc(-c3ccccc3)cc2)n1]

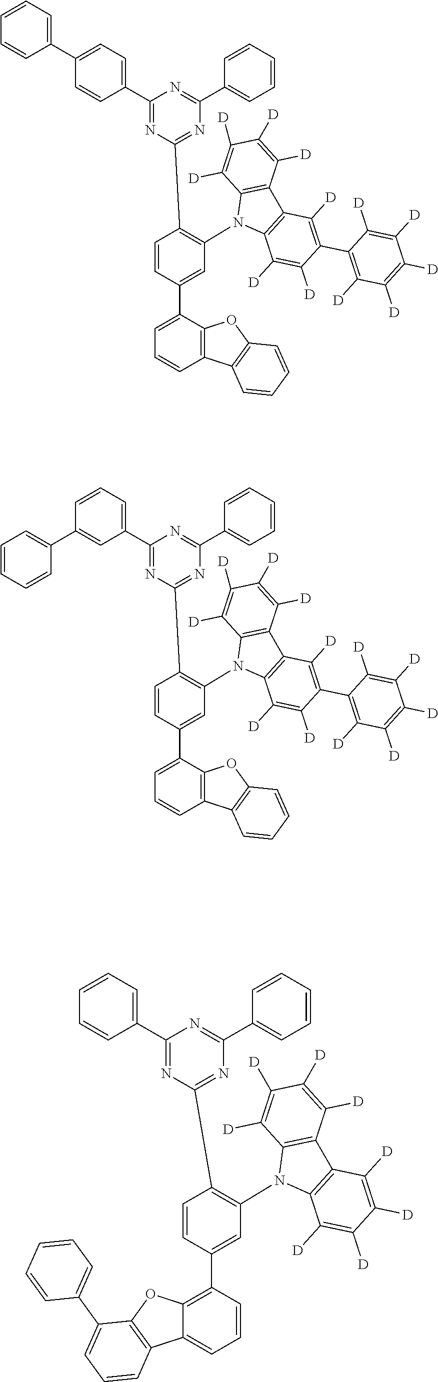 [SMILES: [2H]c1c([2H])c([2H])c(-c2c([2H])c([2H])c3c(c2[2H])c2c([2H])c([2H])c([2H])c([2H])c2n3-c2cc(-c3cccc4c3oc3ccccc34)ccc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2cc(-c3cccc4c3oc3ccccc34)ccc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1cc(-c2cccc3c2oc2c(-c4ccccc4)cccc23)ccc1-c1nc(-c2ccccc2)nc(-c2ccccc2)n1]

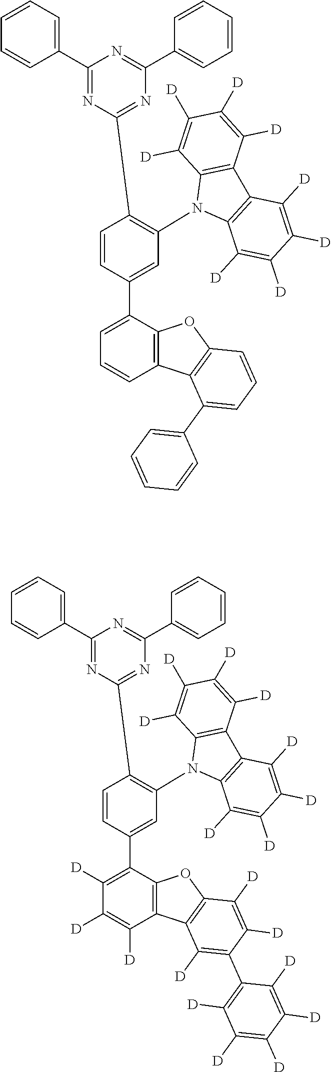 [SMILES: [2H]c1c([2H])c([2H])c2c(c1[2H])c1c([2H])c([2H])c([2H])c([2H])c1n2-c1cc(-c2cccc3c2oc2cccc(-c4ccccc4)c23)ccc1-c1nc(-c2ccccc2)nc(-c2ccccc2)n1 ; [2H]c1c([2H])c([2H])c(-c2c([2H])c([2H])c3oc4c(-c5ccc(-c6nc(-c7ccccc7)nc(-c7ccccc7)n6)c(-n6c7c([2H])c([2H])c([2H])c([2H])c7c7c([2H])c([2H])c([2H])c([2H])c76)c5)c([2H])c([2H])c([2H])c4c3c2[2H])c([2H])c1[2H]]

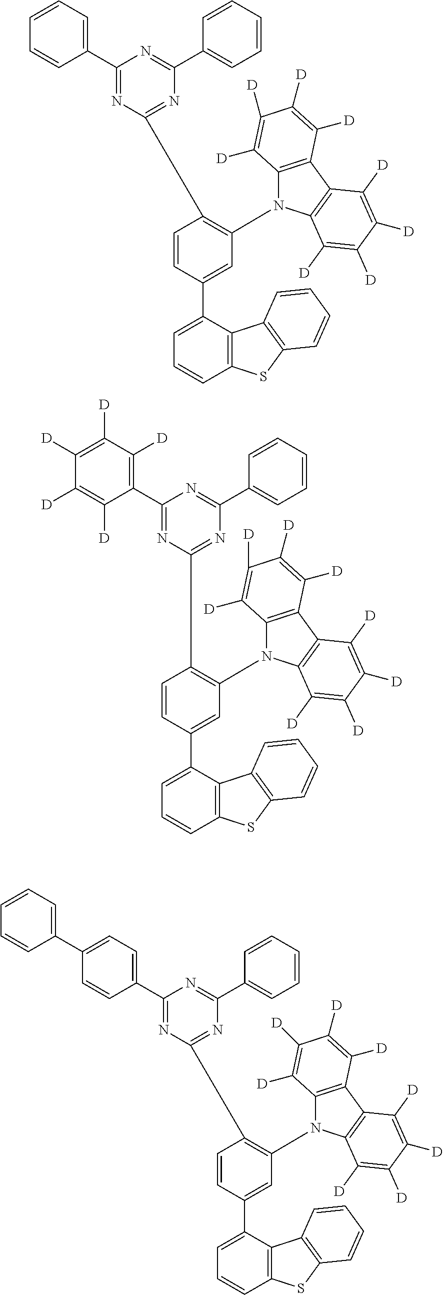 [SMILES: [2H]c1c([2H])c([2H])c2c(c1[2H])c1c([2H])c([2H])c([2H])c([2H])c1n2-c1cc(-c2cccc3sc4ccccc4c23)ccc1-c1nc(-c2ccccc2)nc(-c2ccccc2)n1 ; [2H]c1c([2H])c([2H])c(-c2nc(-c3ccccc3)nc(-c3ccc(-c4cccc5sc6ccccc6c45)cc3-n3c4c([2H])c([2H])c([2H])c([2H])c4c4c([2H])c([2H])c([2H])c([2H])c43)n2)c([2H])c1[2H] ; [2H]c1c([2H])c([2H])c2c(c1[2H])c1c([2H])c([2H])c([2H])c([2H])c1n2-c1cc(-c2cccc3sc4ccccc4c23)ccc1-c1nc(-c2ccccc2)nc(-c2ccc(-c3ccccc3)cc2)n1]

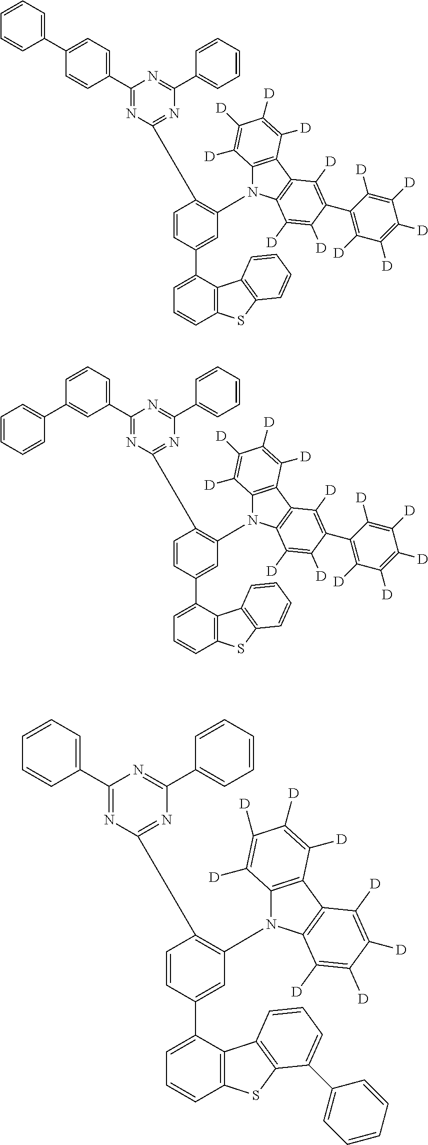 [SMILES: [2H]c1c([2H])c([2H])c(-c2c([2H])c([2H])c3c(c2[2H])c2c([2H])c([2H])c([2H])c([2H])c2n3-c2cc(-c3cccc4sc5ccccc5c34)ccc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2cc(-c3cccc4sc5ccccc5c34)ccc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1cc(-c2cccc3sc4c(-c5ccccc5)cccc4c23)ccc1-c1nc(-c2ccccc2)nc(-c2ccccc2)n1]

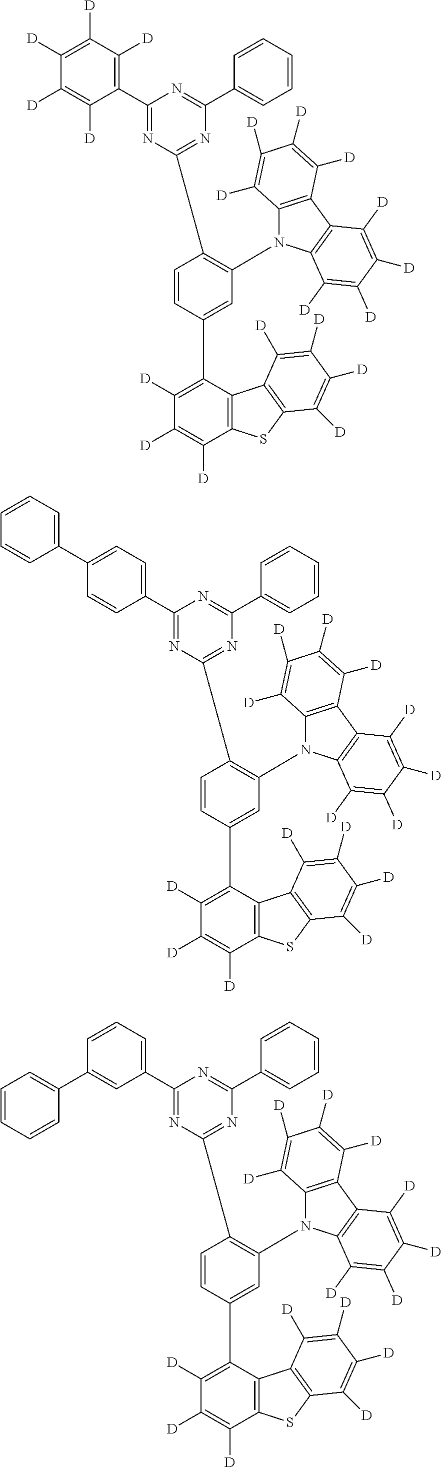

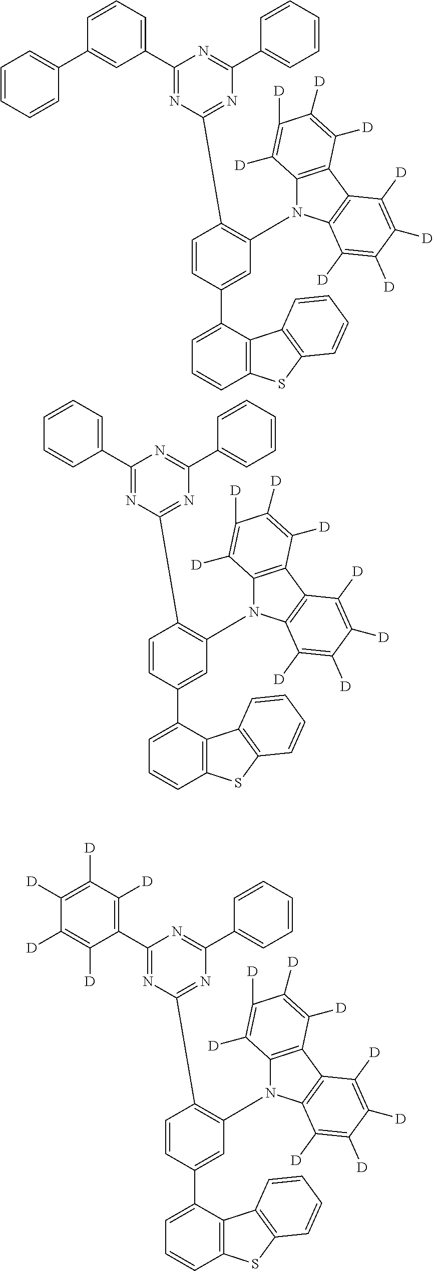

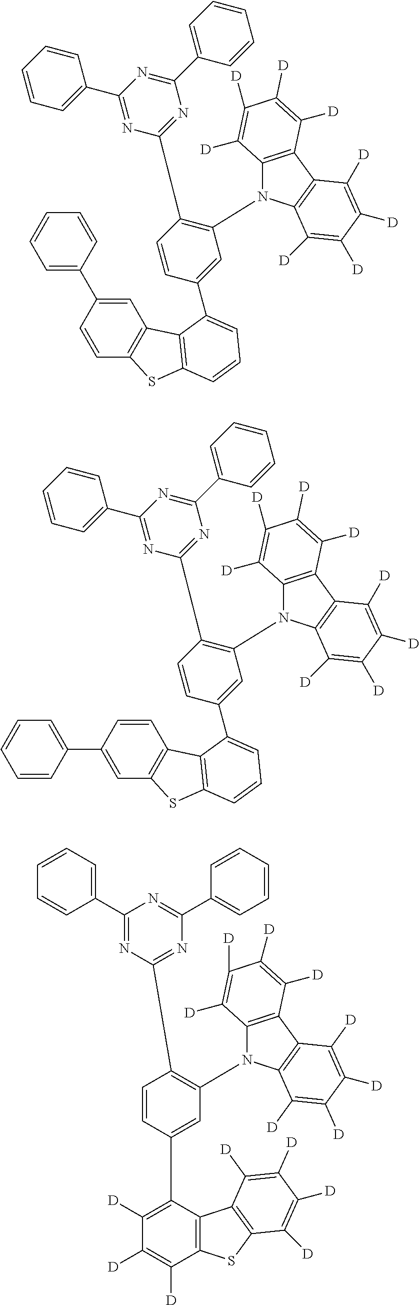

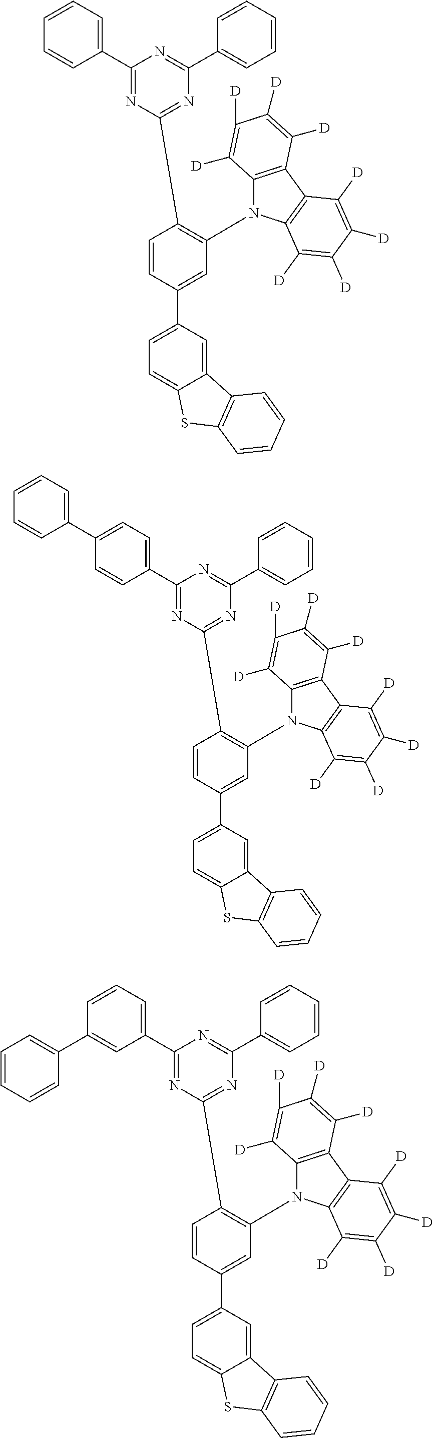

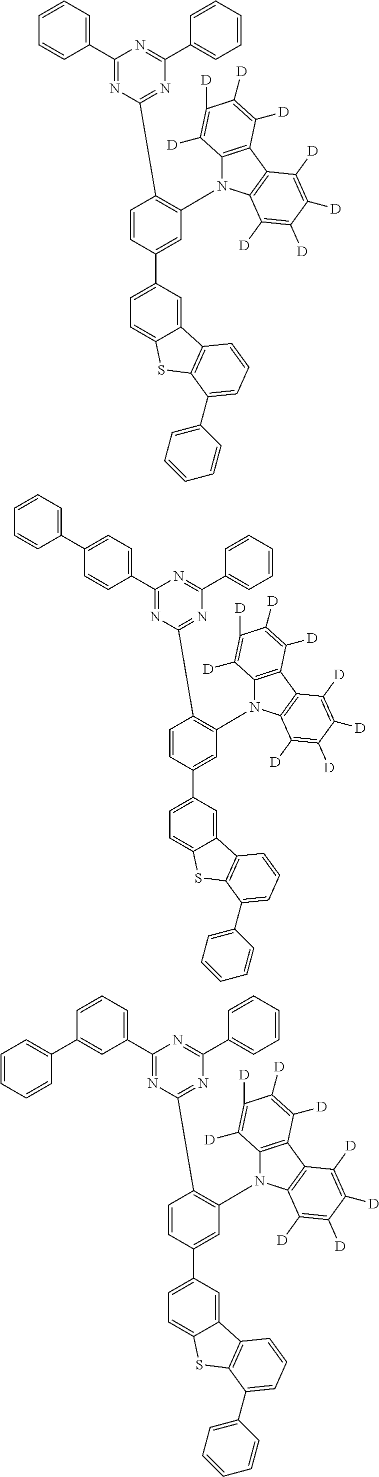

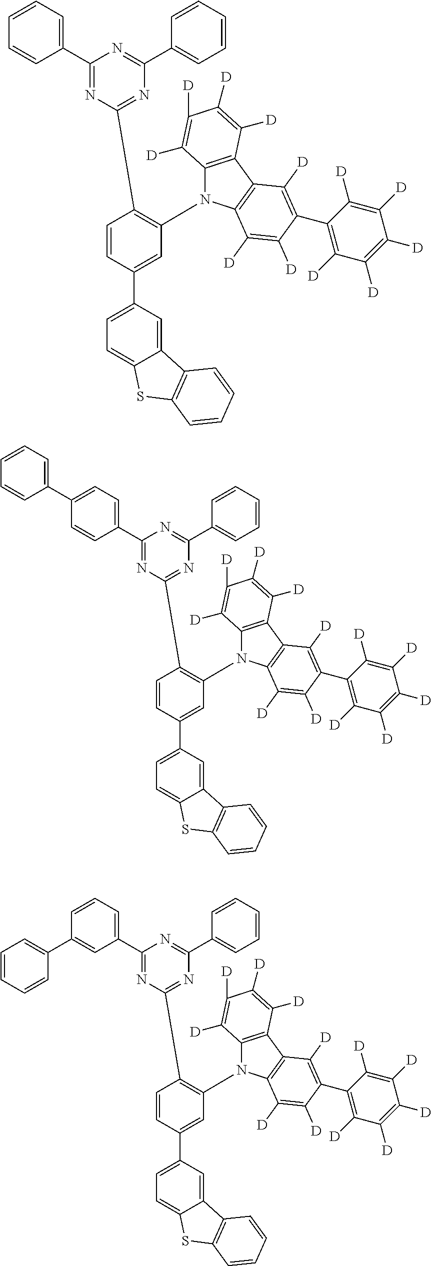

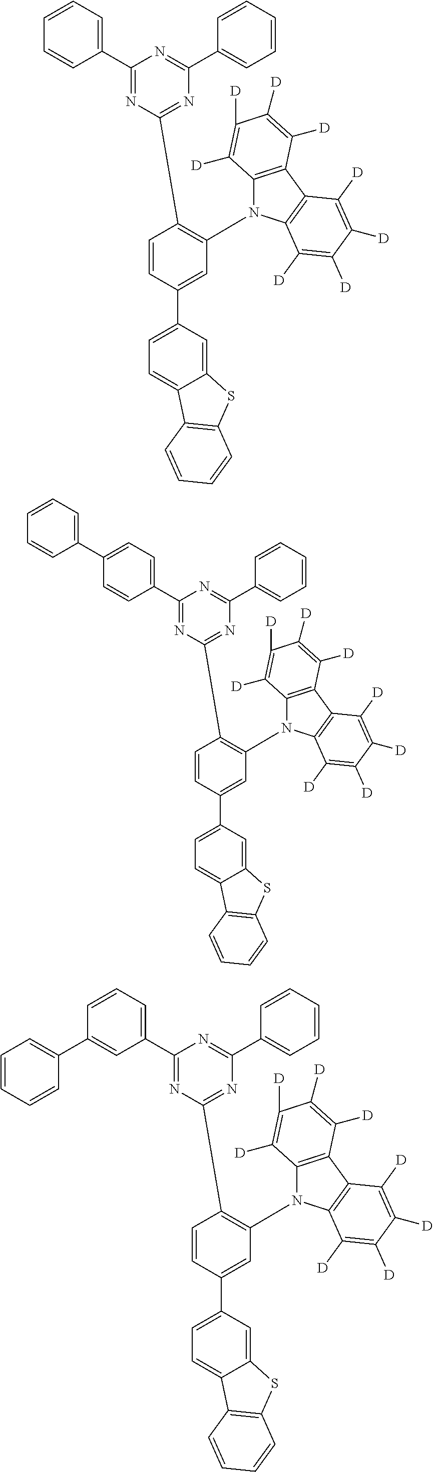

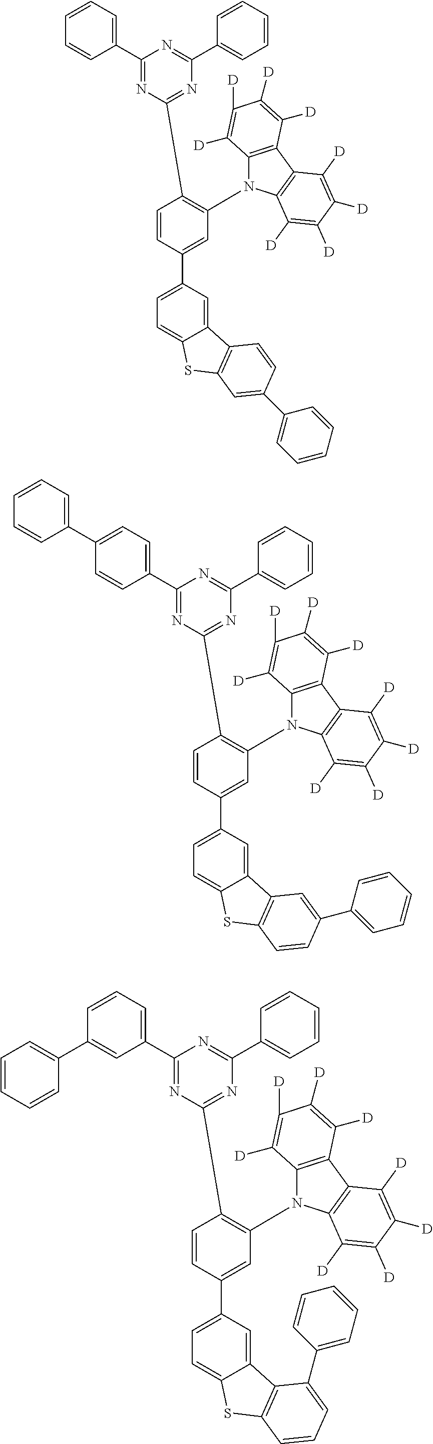

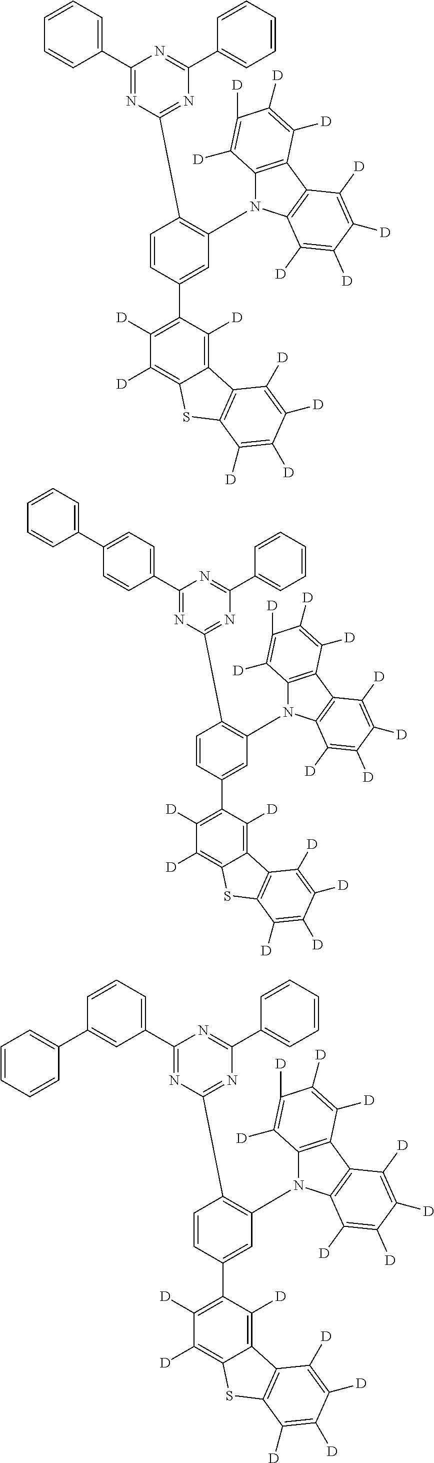

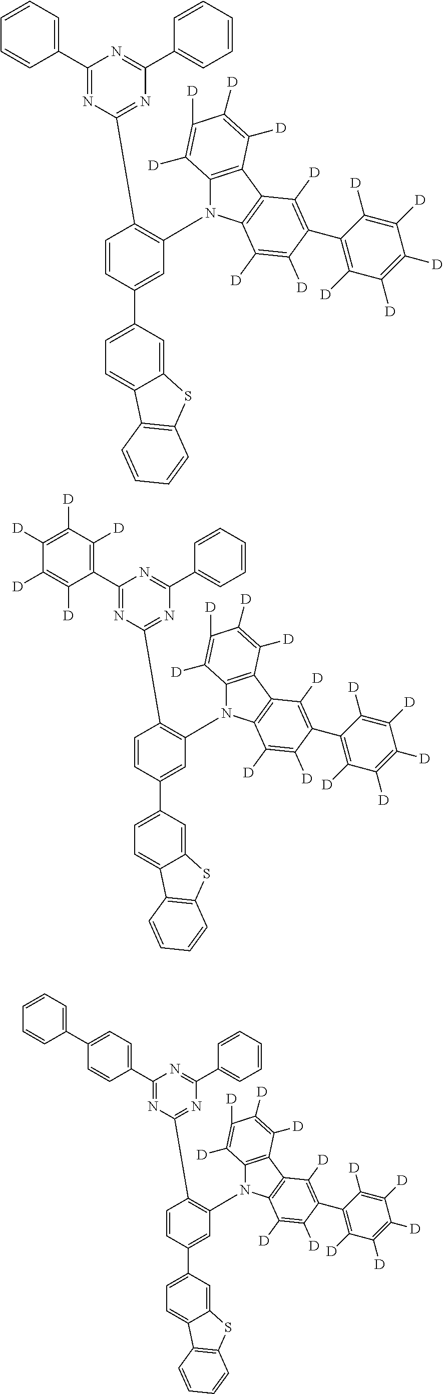

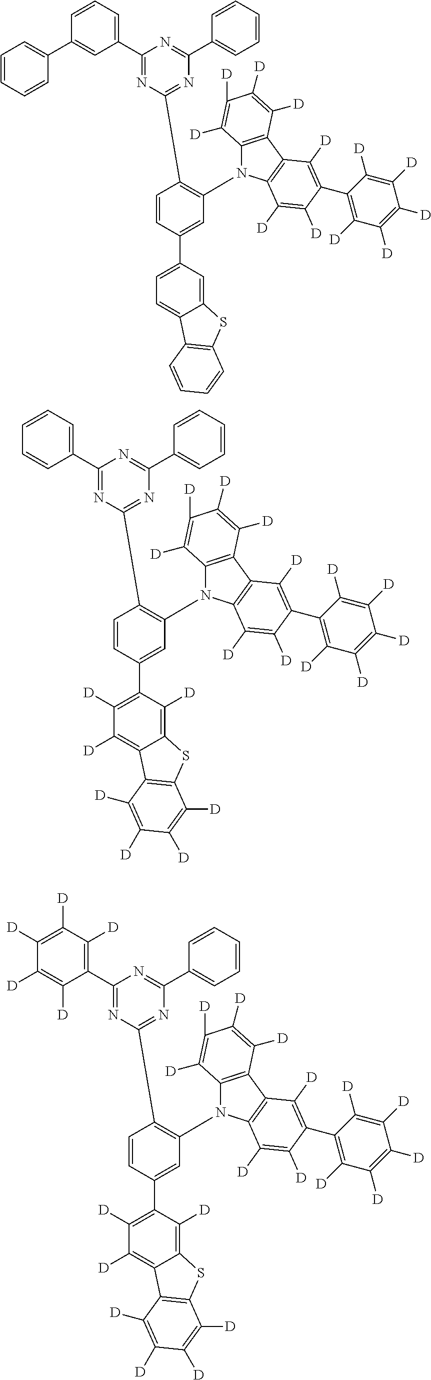

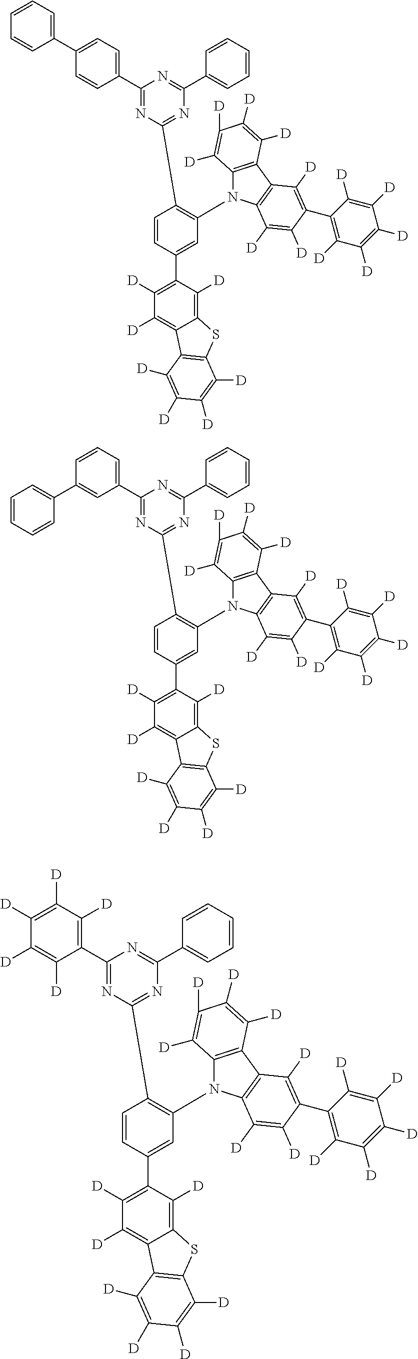

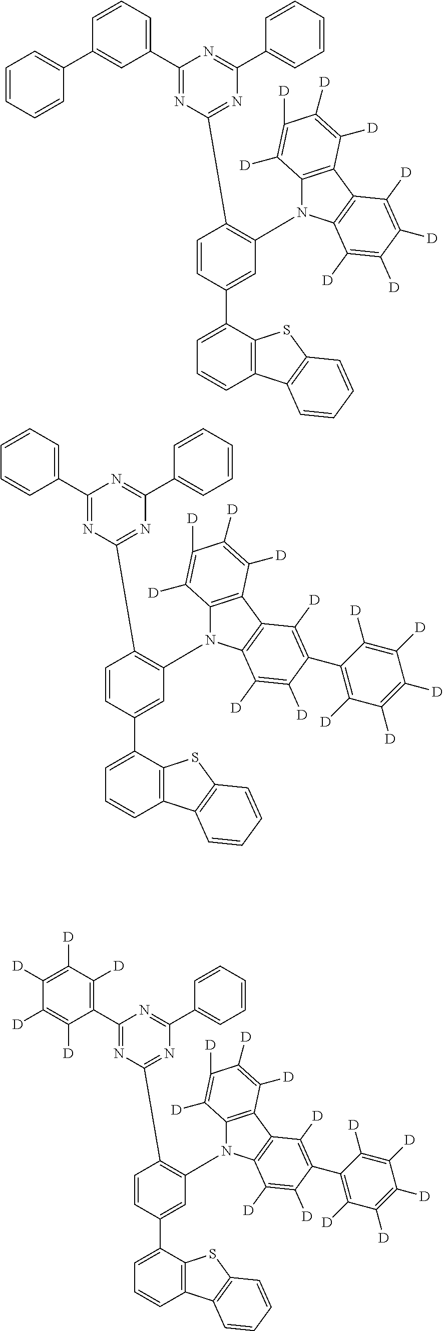

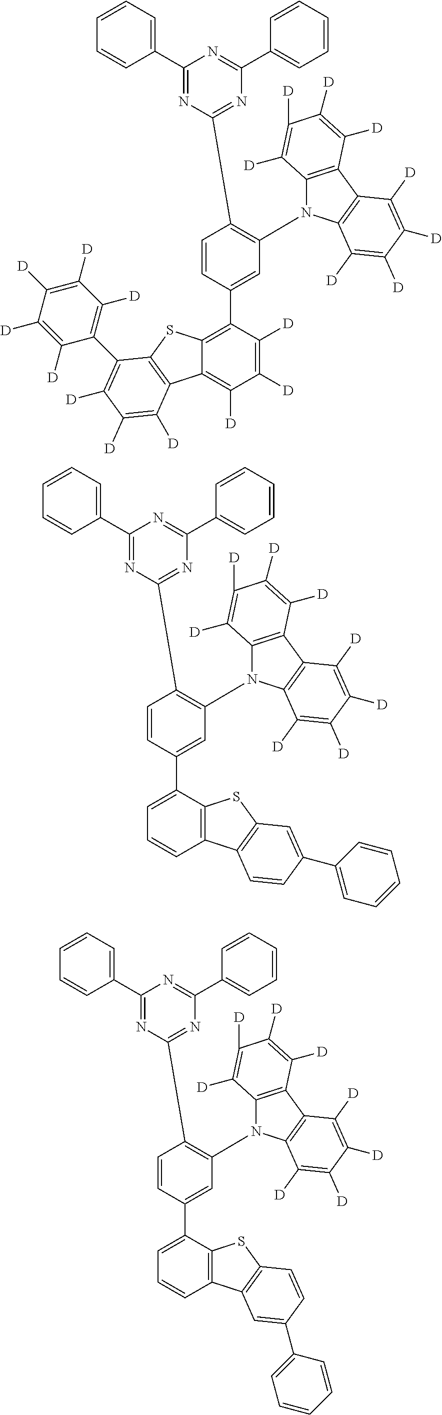

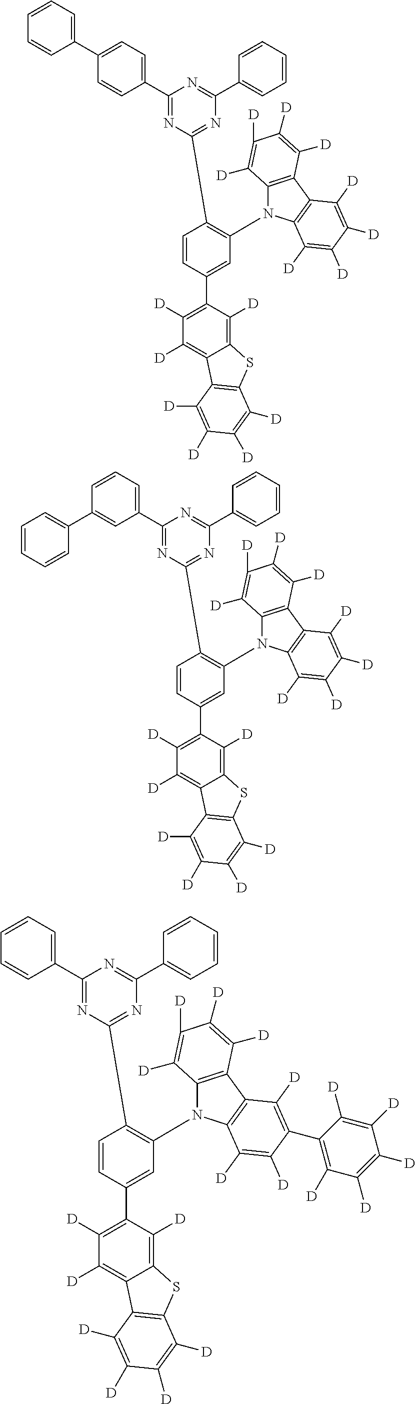

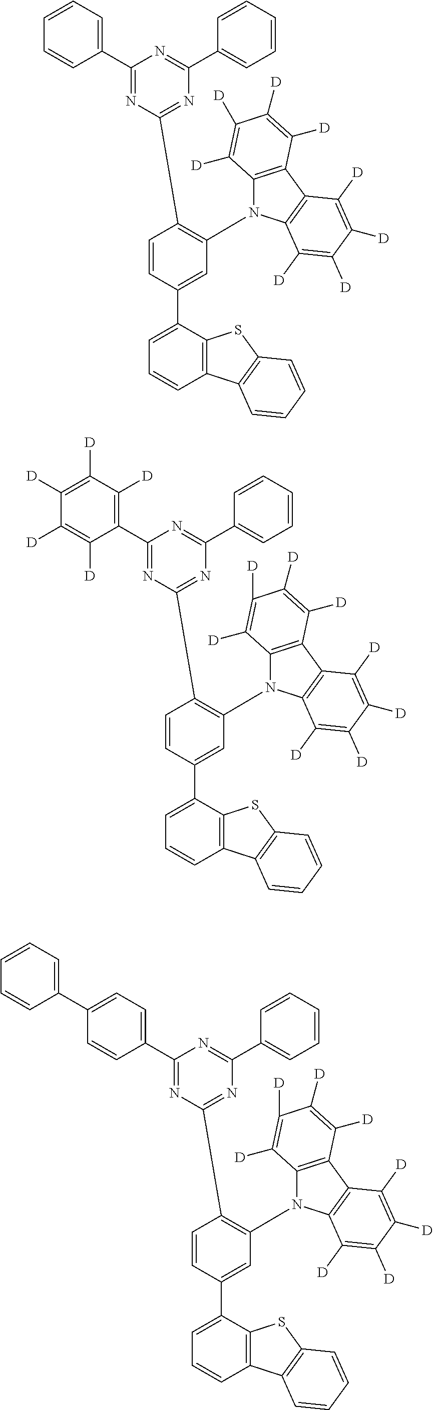 [SMILES: [2H]c1c([2H])c([2H])c2c(c1[2H])c1c([2H])c([2H])c([2H])c([2H])c1n2-c1cc(-c2cccc3c2sc2ccccc23)ccc1-c1nc(-c2ccccc2)nc(-c2ccccc2)n1 ; [2H]c1c([2H])c([2H])c(-c2nc(-c3ccccc3)nc(-c3ccc(-c4cccc5c4sc4ccccc45)cc3-n3c4c([2H])c([2H])c([2H])c([2H])c4c4c([2H])c([2H])c([2H])c([2H])c43)n2)c([2H])c1[2H] ; [2H]c1c([2H])c([2H])c2c(c1[2H])c1c([2H])c([2H])c([2H])c([2H])c1n2-c1cc(-c2cccc3c2sc2ccccc23)ccc1-c1nc(-c2ccccc2)nc(-c2ccc(-c3ccccc3)cc2)n1]

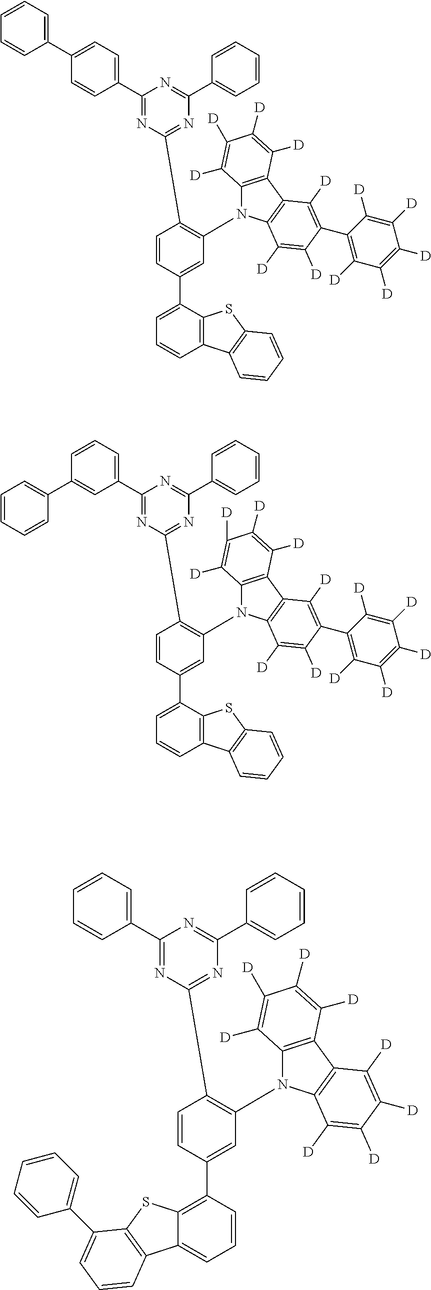 [SMILES: [2H]c1c([2H])c([2H])c(-c2c([2H])c([2H])c3c(c2[2H])c2c([2H])c([2H])c([2H])c([2H])c2n3-c2cc(-c3cccc4c3sc3ccccc34)ccc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2cc(-c3cccc4c3sc3ccccc34)ccc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1cc(-c2cccc3c2sc2c(-c4ccccc4)cccc23)ccc1-c1nc(-c2ccccc2)nc(-c2ccccc2)n1]

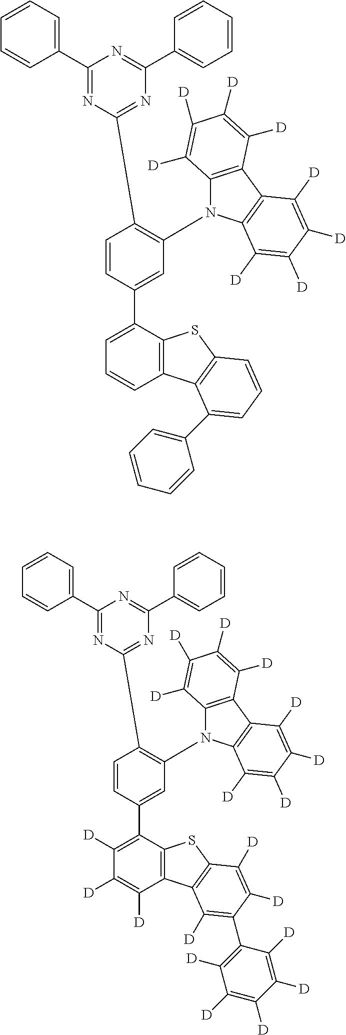 [SMILES: [2H]c1c([2H])c([2H])c2c(c1[2H])c1c([2H])c([2H])c([2H])c([2H])c1n2-c1cc(-c2cccc3c2sc2cccc(-c4ccccc4)c23)ccc1-c1nc(-c2ccccc2)nc(-c2ccccc2)n1 ; [2H]c1c([2H])c([2H])c(-c2c([2H])c([2H])c3sc4c(-c5ccc(-c6nc(-c7ccccc7)nc(-c7ccccc7)n6)c(-n6c7c([2H])c([2H])c([2H])c([2H])c7c7c([2H])c([2H])c([2H])c([2H])c76)c5)c([2H])c([2H])c([2H])c4c3c2[2H])c([2H])c1[2H]]

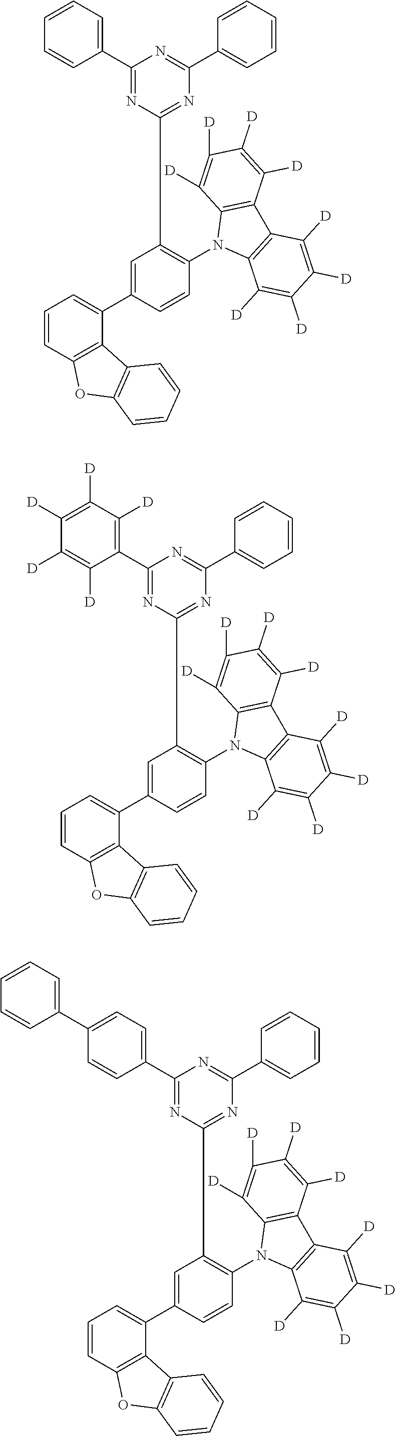 [SMILES: [2H]c1c([2H])c([2H])c2c(c1[2H])c1c([2H])c([2H])c([2H])c([2H])c1n2-c1ccc(-c2cccc3oc4ccccc4c23)cc1-c1nc(-c2ccccc2)nc(-c2ccccc2)n1 ; [2H]c1c([2H])c([2H])c(-c2nc(-c3ccccc3)nc(-c3cc(-c4cccc5oc6ccccc6c45)ccc3-n3c4c([2H])c([2H])c([2H])c([2H])c4c4c([2H])c([2H])c([2H])c([2H])c43)n2)c([2H])c1[2H] ; [2H]c1c([2H])c([2H])c2c(c1[2H])c1c([2H])c([2H])c([2H])c([2H])c1n2-c1ccc(-c2cccc3oc4ccccc4c23)cc1-c1nc(-c2ccccc2)nc(-c2ccc(-c3ccccc3)cc2)n1]

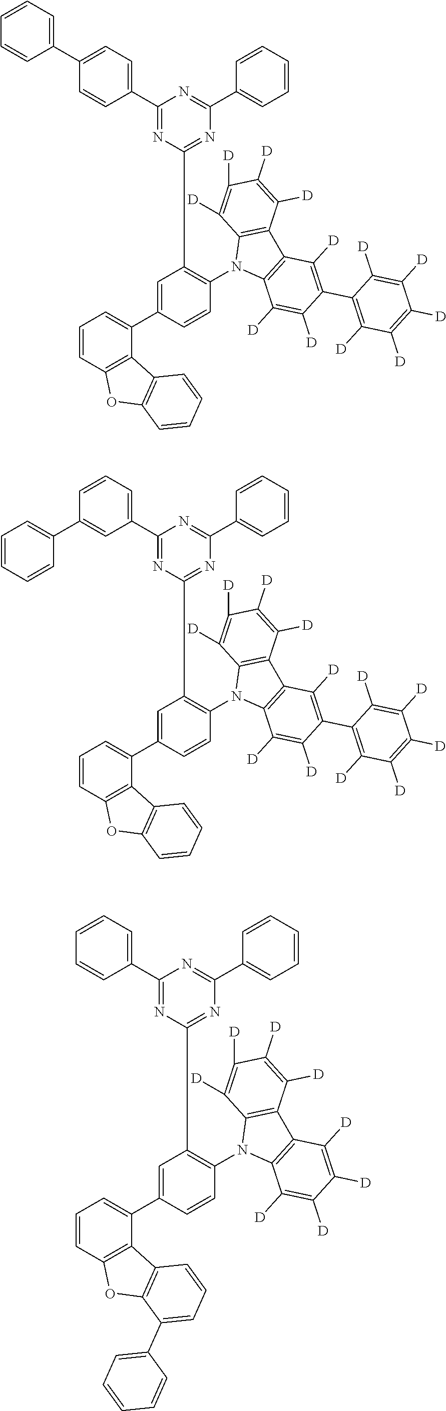 [SMILES: [2H]c1c([2H])c([2H])c(-c2c([2H])c([2H])c3c(c2[2H])c2c([2H])c([2H])c([2H])c([2H])c2n3-c2ccc(-c3cccc4oc5ccccc5c34)cc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2ccc(-c3cccc4oc5ccccc5c34)cc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1ccc(-c2cccc3oc4c(-c5ccccc5)cccc4c23)cc1-c1nc(-c2ccccc2)nc(-c2ccccc2)n1]

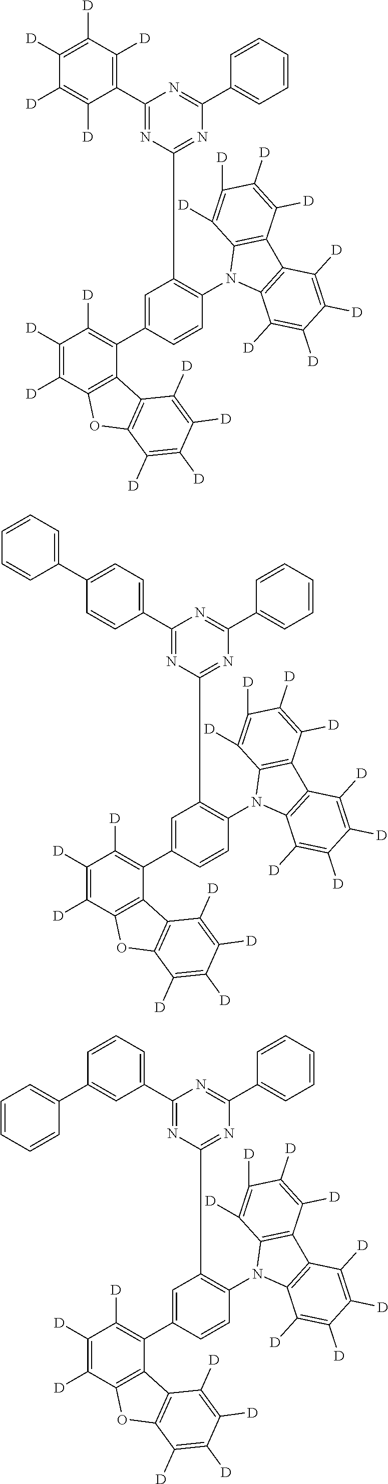

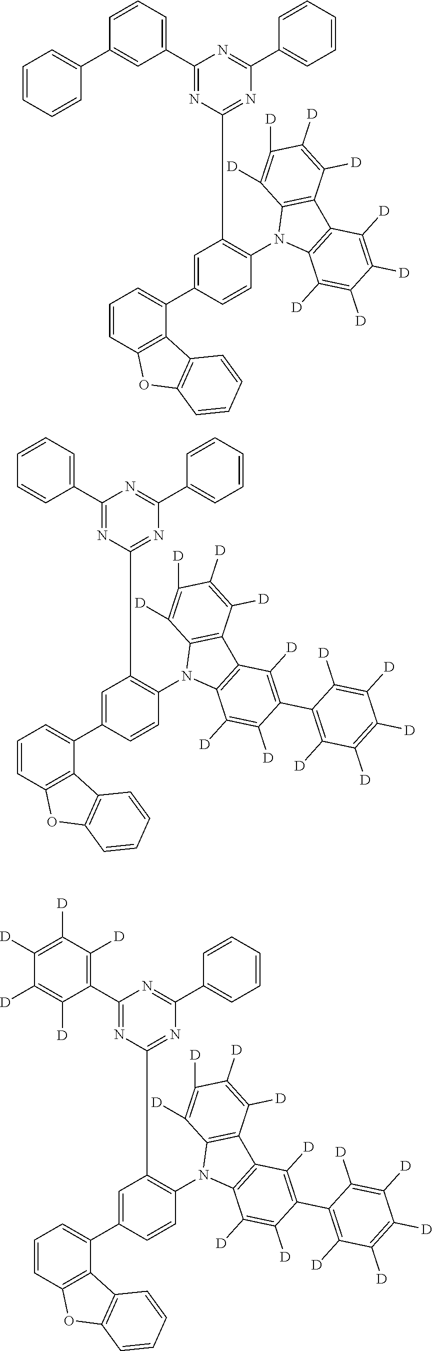

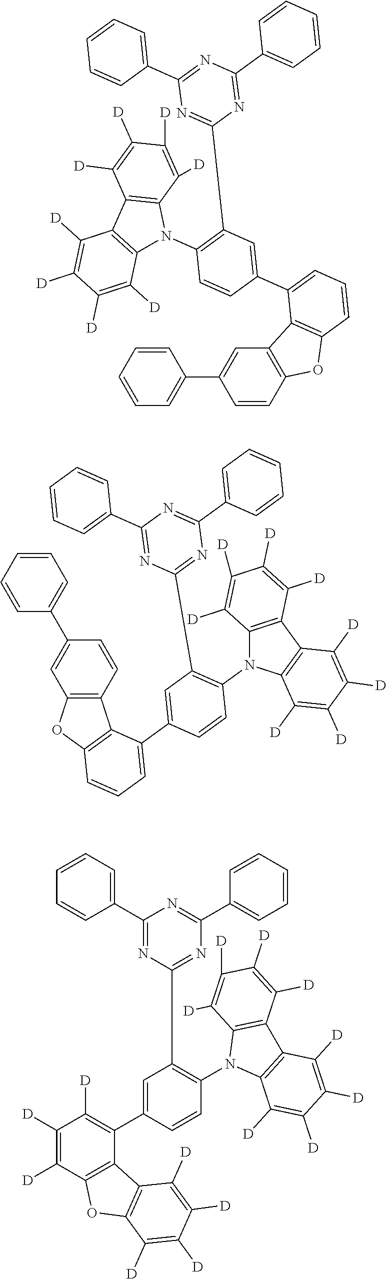

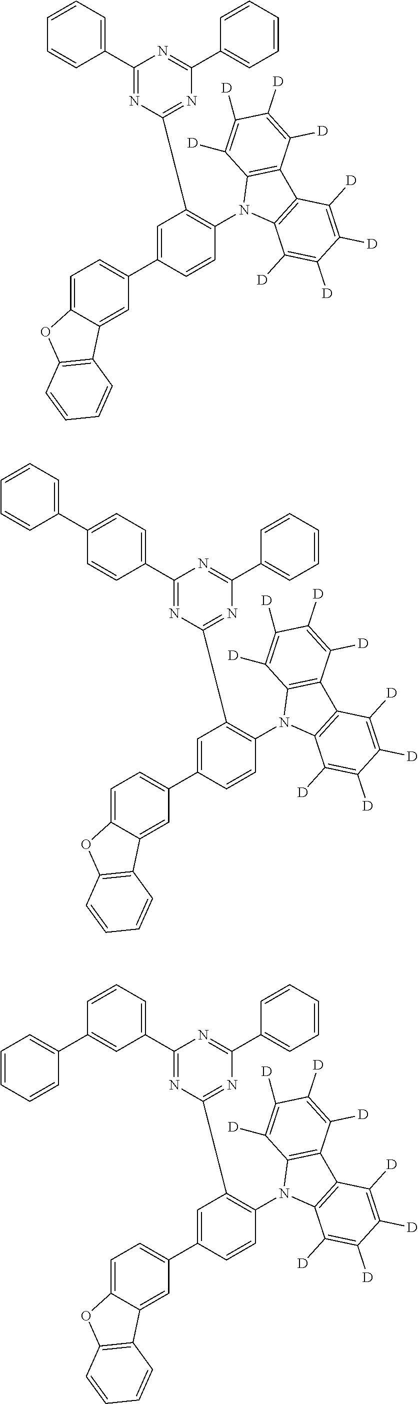 [SMILES: [2H]c1c([2H])c([2H])c2c(c1[2H])c1c([2H])c([2H])c([2H])c([2H])c1n2-c1ccc(-c2ccc3oc4ccccc4c3c2)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc(-c2ccc3oc4ccccc4c3c2)cc1-c1nc(-c2ccccc2)nc(-c2ccc(-c3ccccc3)cc2)n1 ; [2H]c1c([2H])c([2H])c2c(c1[2H])c1c([2H])c([2H])c([2H])c([2H])c1n2-c1ccc(-c2ccc3oc4ccccc4c3c2)cc1-c1nc(-c2ccccc2)nc(-c2cccc(-c3ccccc3)c2)n1]

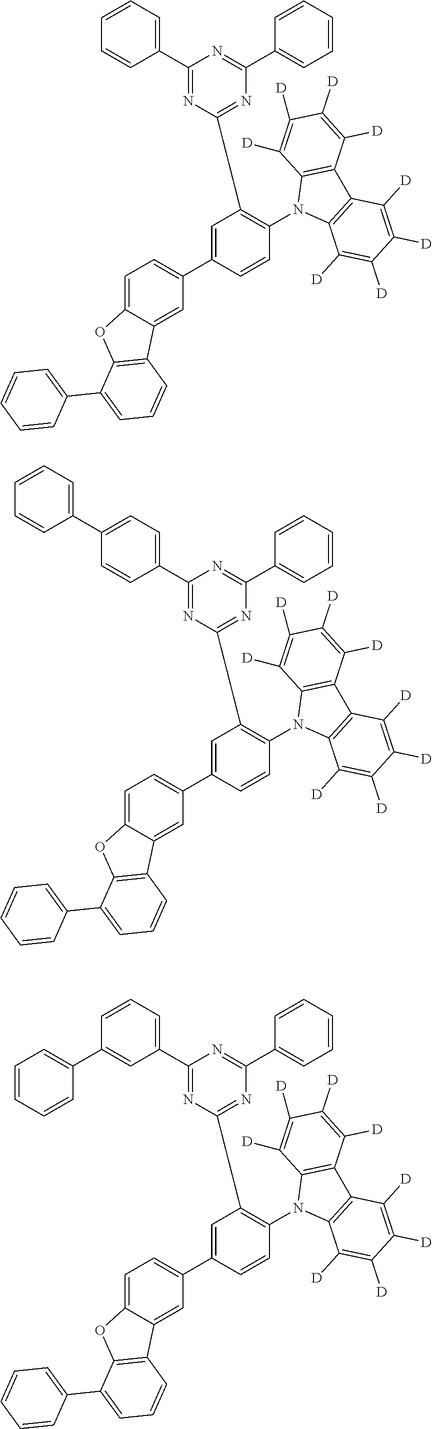

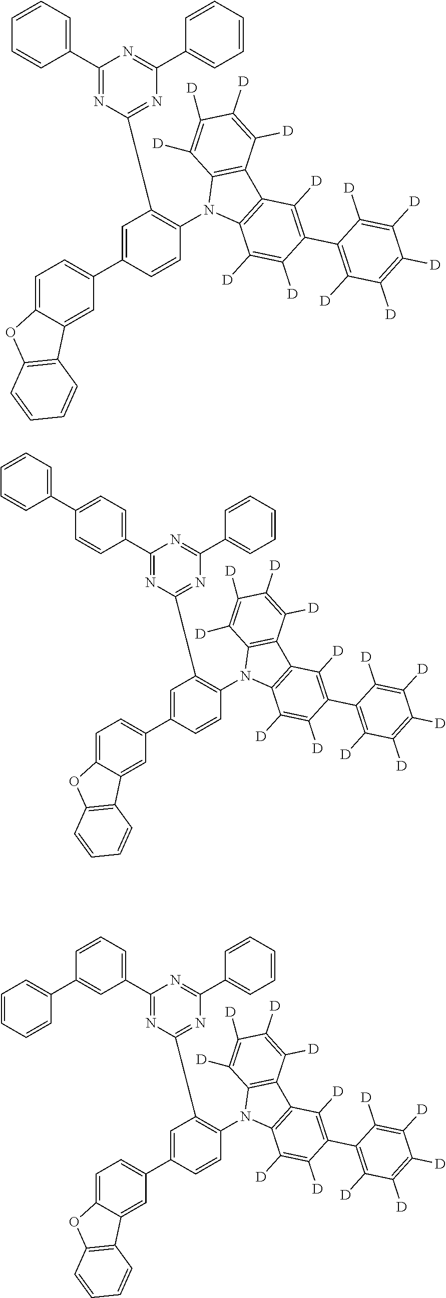

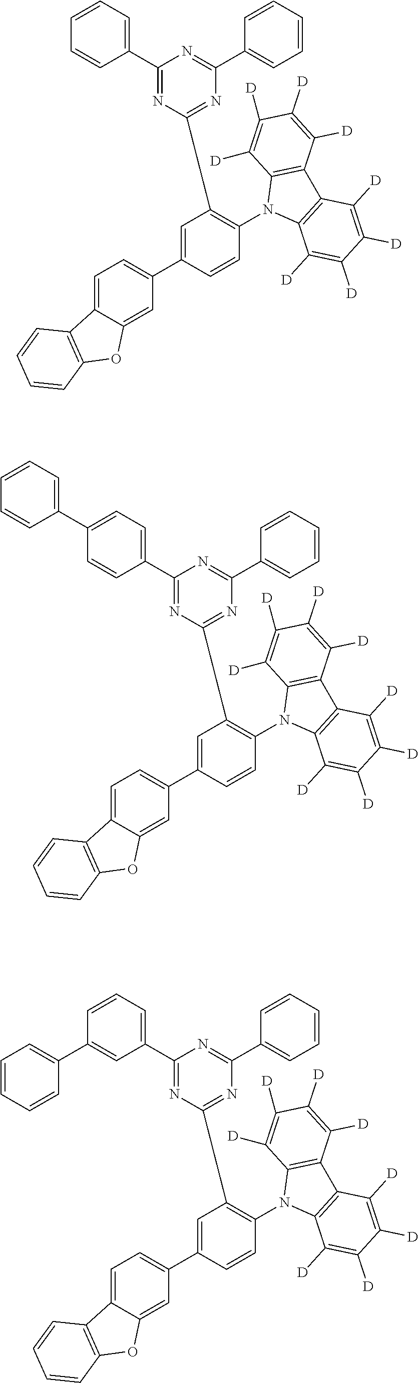 [SMILES: [2H]c1c([2H])c([2H])c2c(c1[2H])c1c([2H])c([2H])c([2H])c([2H])c1n2-c1ccc(-c2ccc3c(c2)oc2ccccc23)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc(-c2ccc3c(c2)oc2ccccc23)cc1-c1nc(-c2ccccc2)nc(-c2ccc(-c3ccccc3)cc2)n1 ; [2H]c1c([2H])c([2H])c2c(c1[2H])c1c([2H])c([2H])c([2H])c([2H])c1n2-c1ccc(-c2ccc3c(c2)oc2ccccc23)cc1-c1nc(-c2ccccc2)nc(-c2cccc(-c3ccccc3)c2)n1]

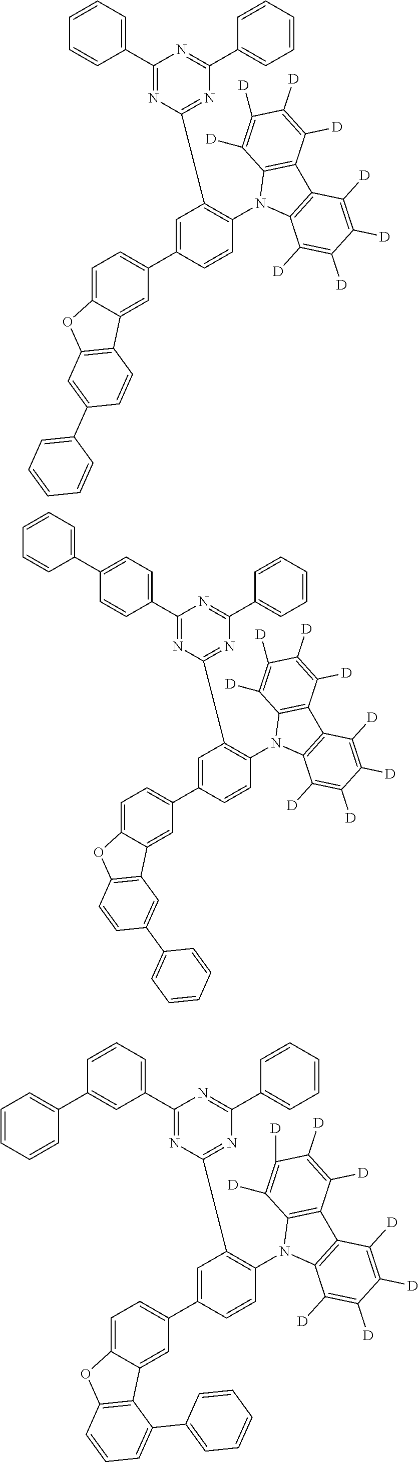

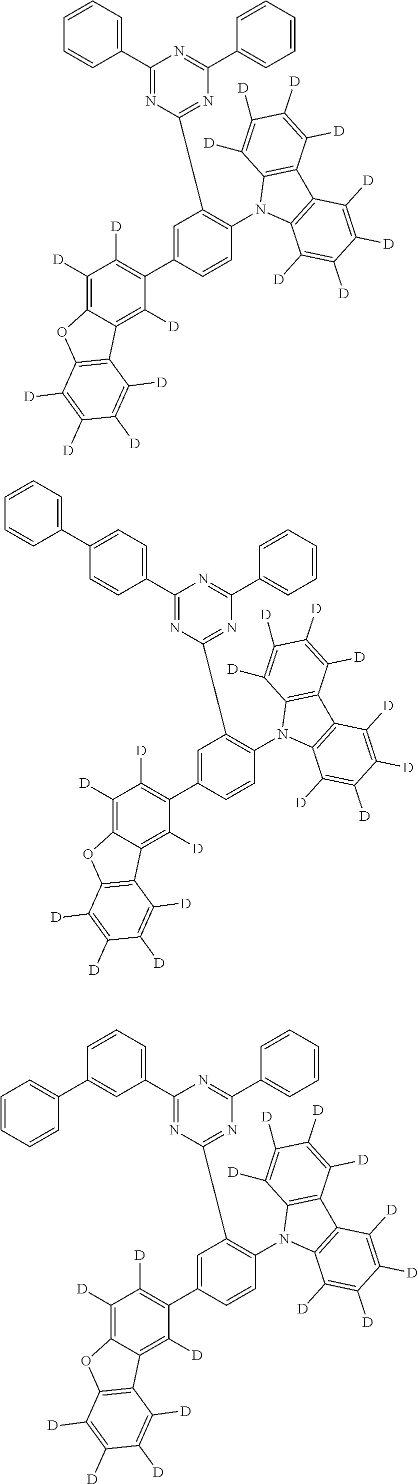

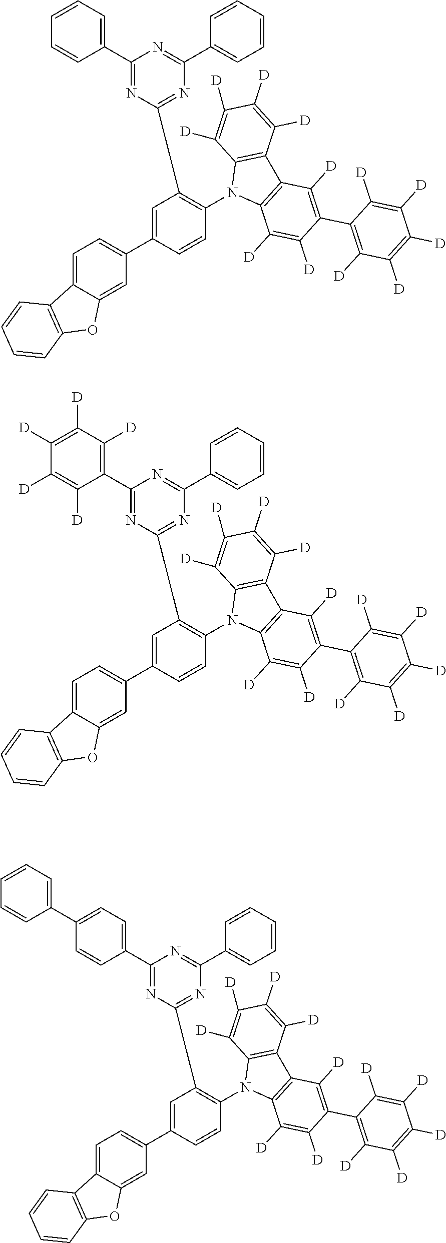

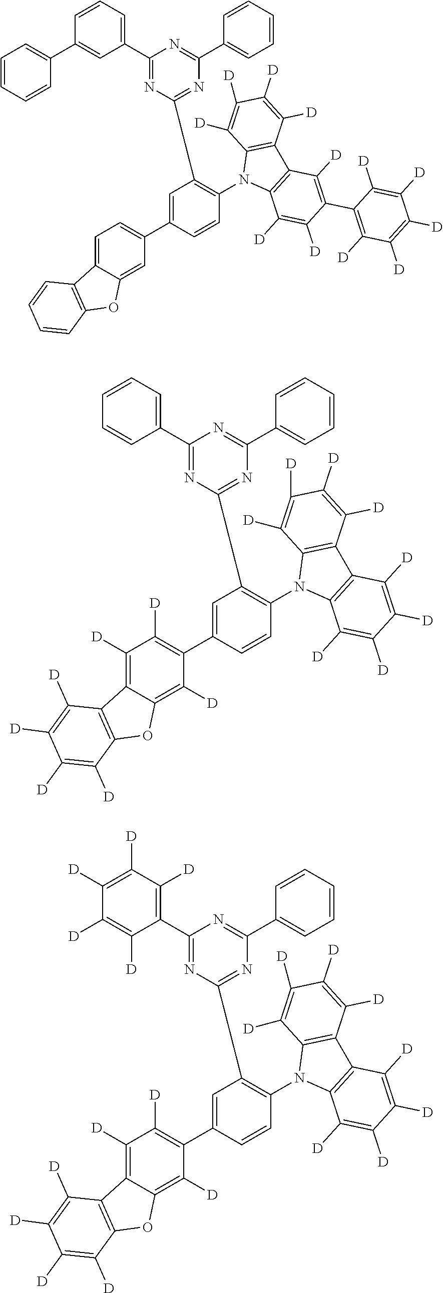

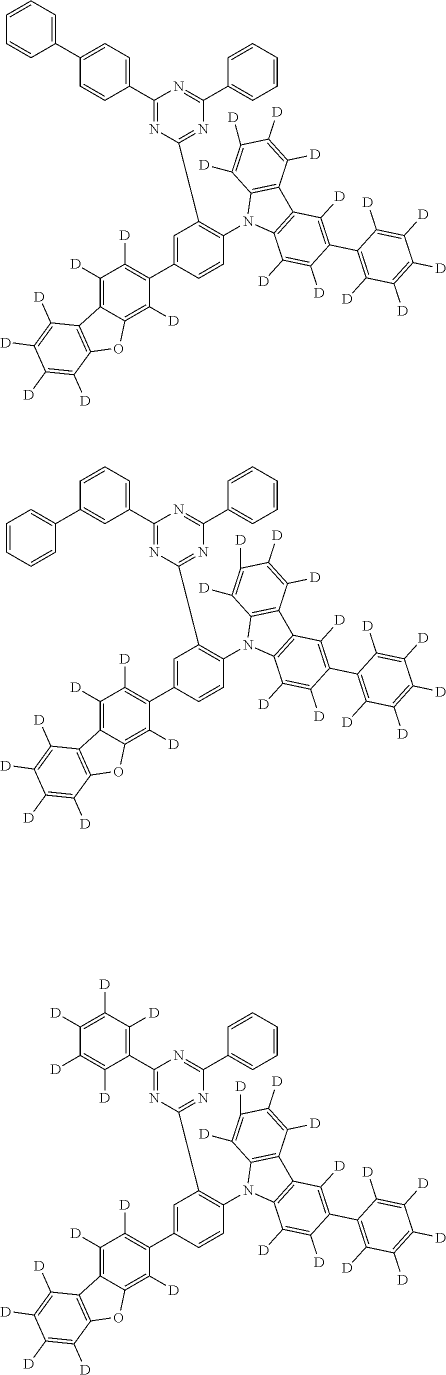

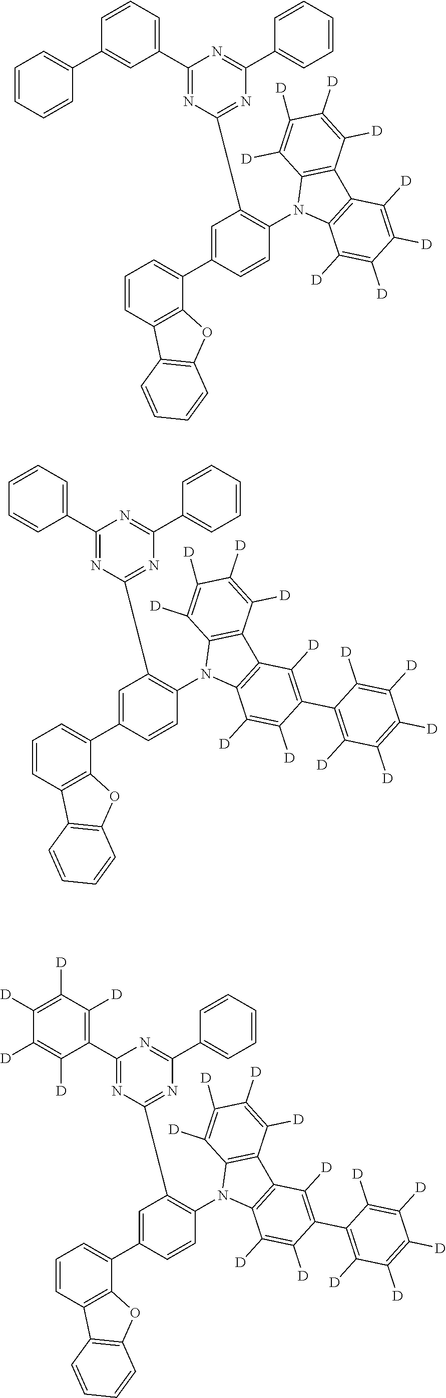 [SMILES: [2H]c1c([2H])c([2H])c2c(c1[2H])c1c([2H])c([2H])c([2H])c([2H])c1n2-c1ccc(-c2cccc3c2oc2ccccc23)cc1-c1nc(-c2ccccc2)nc(-c2cccc(-c3ccccc3)c2)n1 ; [2H]c1c([2H])c([2H])c(-c2c([2H])c([2H])c3c(c2[2H])c2c([2H])c([2H])c([2H])c([2H])c2n3-c2ccc(-c3cccc4c3oc3ccccc34)cc2-c2nc(-c3ccccc3)nc(-c3ccccc3)n2)c([2H])c1[2H] ; [2H]c1c([2H])c([2H])c(-c2nc(-c3ccccc3)nc(-c3cc(-c4cccc5c4oc4ccccc45)ccc3-n3c4c([2H])c([2H])c([2H])c([2H])c4c4c([2H])c(-c5c([2H])c([2H])c([2H])c([2H])c5[2H])c([2H])c([2H])c43)n2)c([2H])c1[2H]]

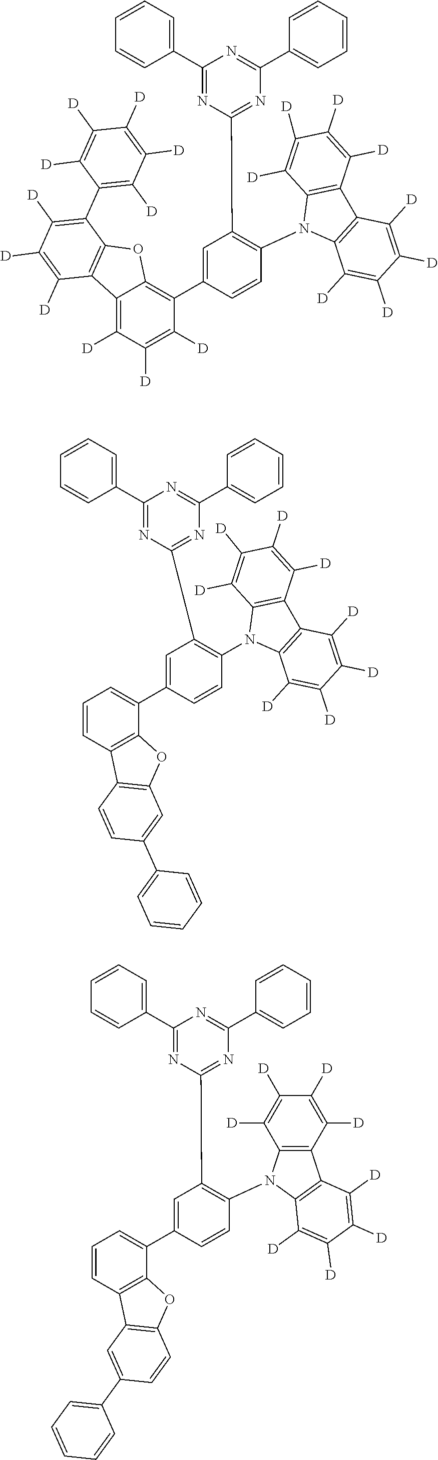 [SMILES: [2H]c1c([2H])c([2H])c(-c2c([2H])c([2H])c([2H])c3c2oc2c(-c4ccc(-n5c6c([2H])c([2H])c([2H])c([2H])c6c6c([2H])c([2H])c([2H])c([2H])c65)c(-c5nc(-c6ccccc6)nc(-c6ccccc6)n5)c4)c([2H])c([2H])c([2H])c23)c([2H])c1[2H] ; [2H]c1c([2H])c([2H])c2c(c1[2H])c1c([2H])c([2H])c([2H])c([2H])c1n2-c1ccc(-c2cccc3c2oc2cc(-c4ccccc4)ccc23)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc(-c2cccc3c2oc2ccc(-c4ccccc4)cc23)cc1-c1nc(-c2ccccc2)nc(-c2ccccc2)n1]

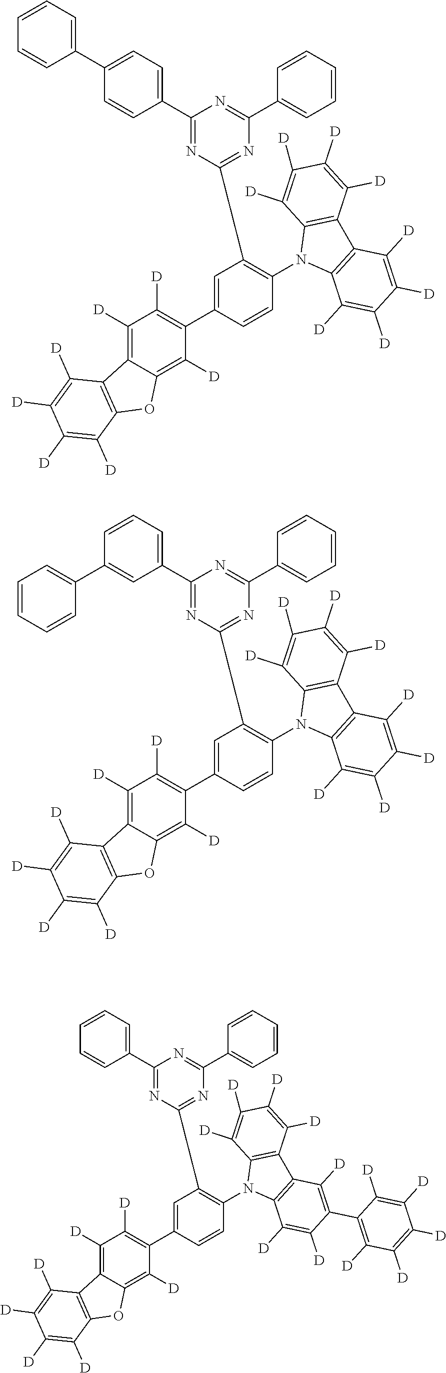

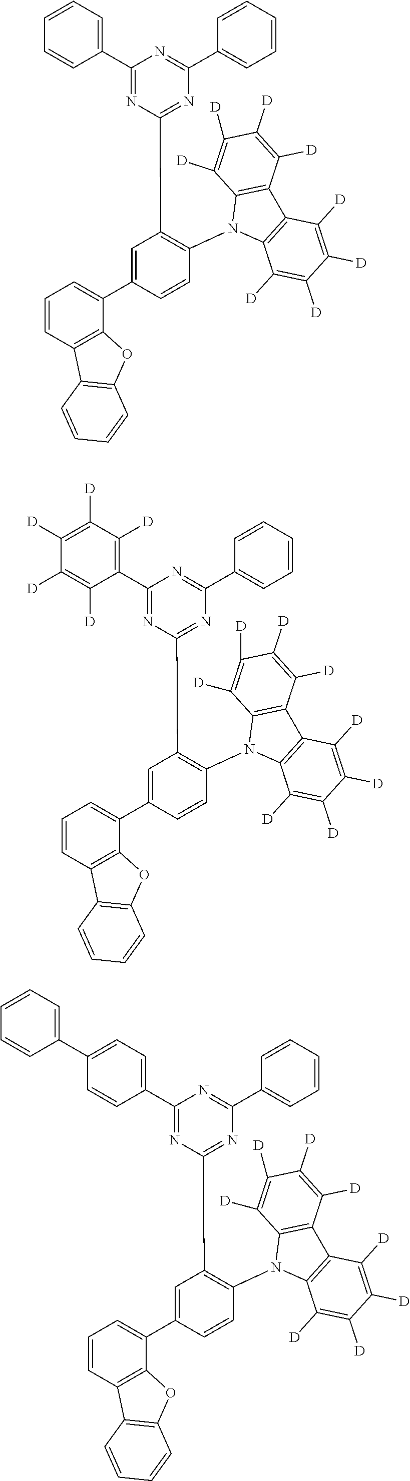 [SMILES: [2H]c1c([2H])c([2H])c2c(c1[2H])c1c([2H])c([2H])c([2H])c([2H])c1n2-c1ccc(-c2cccc3c2oc2ccccc23)cc1-c1nc(-c2ccccc2)nc(-c2ccccc2)n1 ; [2H]c1c([2H])c([2H])c(-c2nc(-c3ccccc3)nc(-c3cc(-c4cccc5c4oc4ccccc45)ccc3-n3c4c([2H])c([2H])c([2H])c([2H])c4c4c([2H])c([2H])c([2H])c([2H])c43)n2)c([2H])c1[2H] ; [2H]c1c([2H])c([2H])c2c(c1[2H])c1c([2H])c([2H])c([2H])c([2H])c1n2-c1ccc(-c2cccc3c2oc2ccccc23)cc1-c1nc(-c2ccccc2)nc(-c2ccc(-c3ccccc3)cc2)n1]

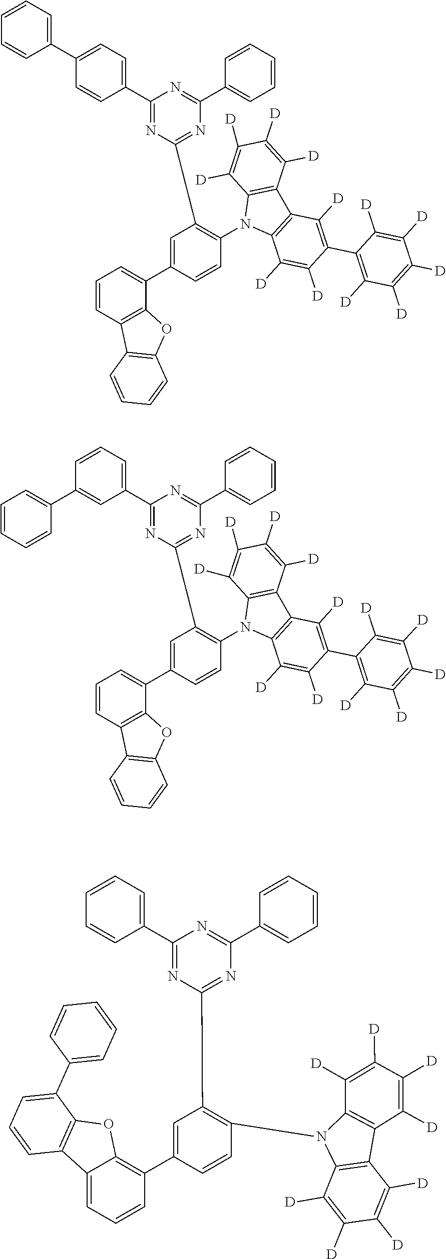 [SMILES: [2H]c1c([2H])c([2H])c(-c2c([2H])c([2H])c3c(c2[2H])c2c([2H])c([2H])c([2H])c([2H])c2n3-c2ccc(-c3cccc4c3oc3ccccc34)cc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2ccc(-c3cccc4c3oc3ccccc34)cc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1ccc(-c2cccc3c2oc2c(-c4ccccc4)cccc23)cc1-c1nc(-c2ccccc2)nc(-c2ccccc2)n1]

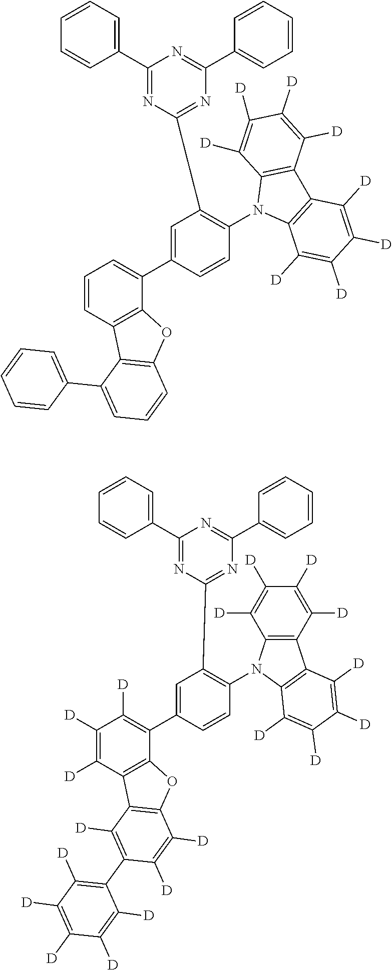 [SMILES: [2H]c1c([2H])c([2H])c2c(c1[2H])c1c([2H])c([2H])c([2H])c([2H])c1n2-c1ccc(-c2cccc3c2oc2cccc(-c4ccccc4)c23)cc1-c1nc(-c2ccccc2)nc(-c2ccccc2)n1 ; [2H]c1c([2H])c([2H])c(-c2c([2H])c([2H])c3oc4c(-c5ccc(-n6c7c([2H])c([2H])c([2H])c([2H])c7c7c([2H])c([2H])c([2H])c([2H])c76)c(-c6nc(-c7ccccc7)nc(-c7ccccc7)n6)c5)c([2H])c([2H])c([2H])c4c3c2[2H])c([2H])c1[2H]]

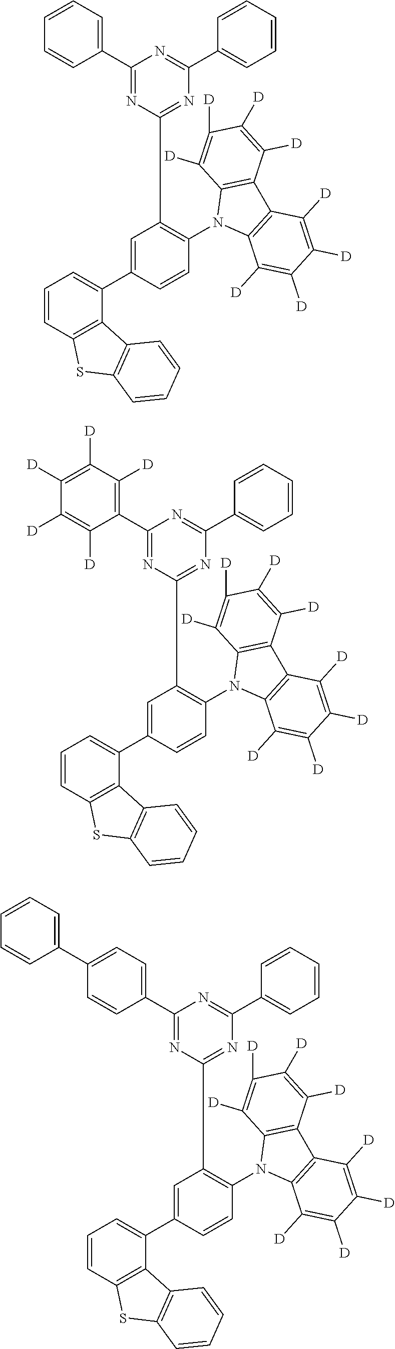 [SMILES: [2H]c1c([2H])c([2H])c2c(c1[2H])c1c([2H])c([2H])c([2H])c([2H])c1n2-c1ccc(-c2cccc3sc4ccccc4c23)cc1-c1nc(-c2ccccc2)nc(-c2ccccc2)n1 ; [2H]c1c([2H])c([2H])c(-c2nc(-c3ccccc3)nc(-c3cc(-c4cccc5sc6ccccc6c45)ccc3-n3c4c([2H])c([2H])c([2H])c([2H])c4c4c([2H])c([2H])c([2H])c([2H])c43)n2)c([2H])c1[2H] ; [2H]c1c([2H])c([2H])c2c(c1[2H])c1c([2H])c([2H])c([2H])c([2H])c1n2-c1ccc(-c2cccc3sc4ccccc4c23)cc1-c1nc(-c2ccccc2)nc(-c2ccc(-c3ccccc3)cc2)n1]

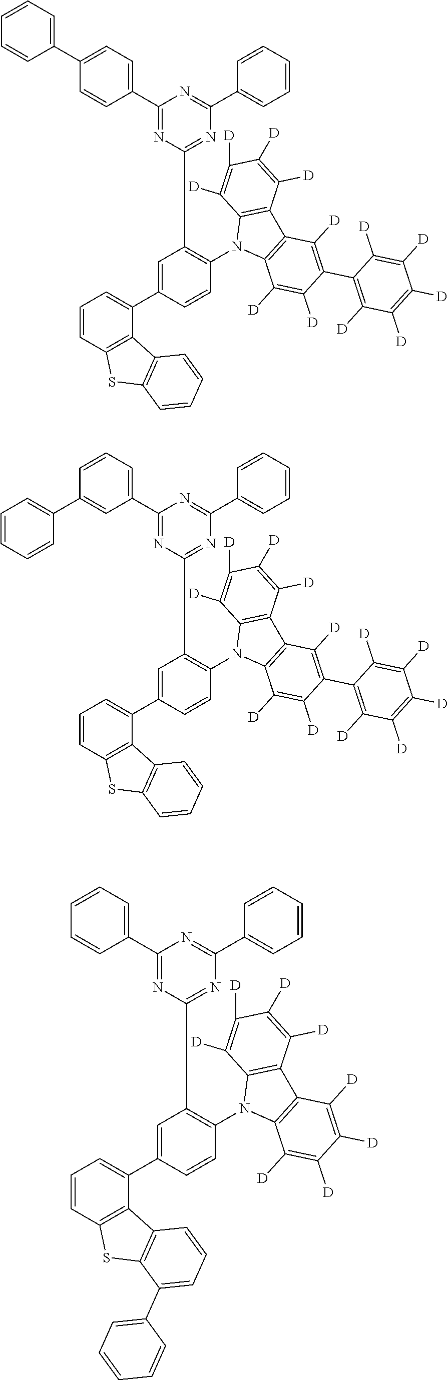 [SMILES: [2H]c1c([2H])c([2H])c(-c2c([2H])c([2H])c3c(c2[2H])c2c([2H])c([2H])c([2H])c([2H])c2n3-c2ccc(-c3cccc4sc5ccccc5c34)cc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2ccc(-c3cccc4sc5ccccc5c34)cc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1ccc(-c2cccc3sc4c(-c5ccccc5)cccc4c23)cc1-c1nc(-c2ccccc2)nc(-c2ccccc2)n1]

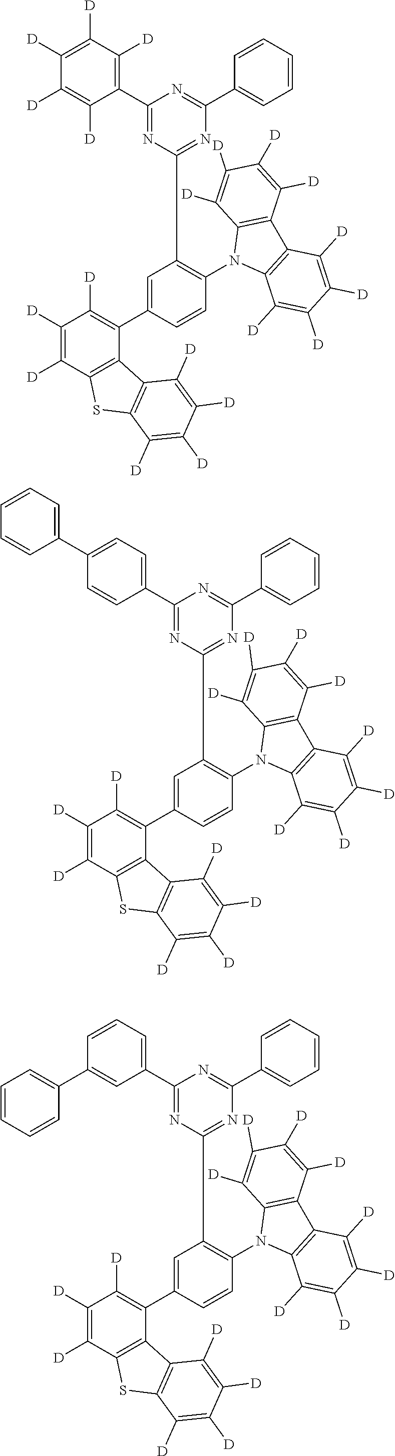

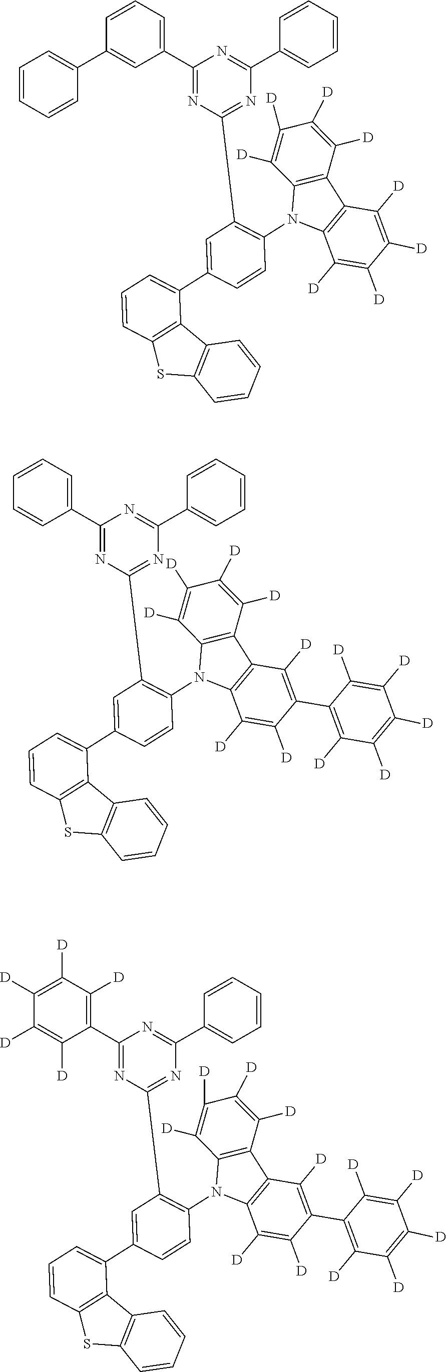 [SMILES: [2H]c1c([2H])c([2H])c2c(c1[2H])c1c([2H])c([2H])c([2H])c([2H])c1n2-c1ccc(-c2cccc3sc4ccccc4c23)cc1-c1nc(-c2ccccc2)nc(-c2cccc(-c3ccccc3)c2)n1 ; [2H]c1c([2H])c([2H])c(-c2c([2H])c([2H])c3c(c2[2H])c2c([2H])c([2H])c([2H])c([2H])c2n3-c2ccc(-c3cccc4sc5ccccc5c34)cc2-c2nc(-c3ccccc3)nc(-c3ccccc3)n2)c([2H])c1[2H] ; [2H]c1c([2H])c([2H])c(-c2nc(-c3ccccc3)nc(-c3cc(-c4cccc5sc6ccccc6c45)ccc3-n3c4c([2H])c([2H])c([2H])c([2H])c4c4c([2H])c(-c5c([2H])c([2H])c([2H])c([2H])c5[2H])c([2H])c([2H])c43)n2)c([2H])c1[2H]]

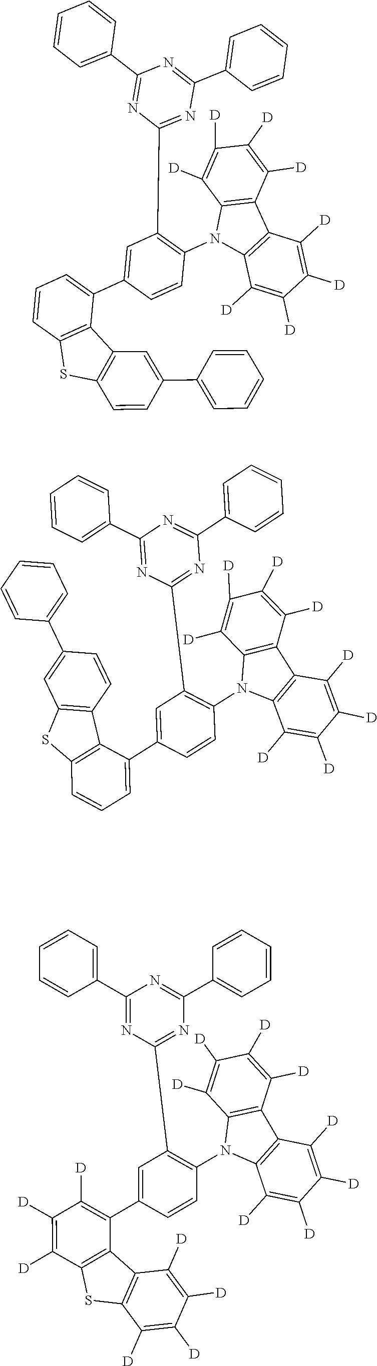 [SMILES: [2H]c1c([2H])c([2H])c2c(c1[2H])c1c([2H])c([2H])c([2H])c([2H])c1n2-c1ccc(-c2cccc3sc4ccc(-c5ccccc5)cc4c23)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc(-c2cccc3sc4cc(-c5ccccc5)ccc4c23)cc1-c1nc(-c2ccccc2)nc(-c2ccccc2)n1 ; [2H]c1c([2H])c([2H])c2c(sc3c([2H])c([2H])c([2H])c(-c4ccc(-n5c6c([2H])c([2H])c([2H])c([2H])c6c6c([2H])c([2H])c([2H])c([2H])c65)c(-c5nc(-c6ccccc6)nc(-c6ccccc6)n5)c4)c32)c1[2H]]

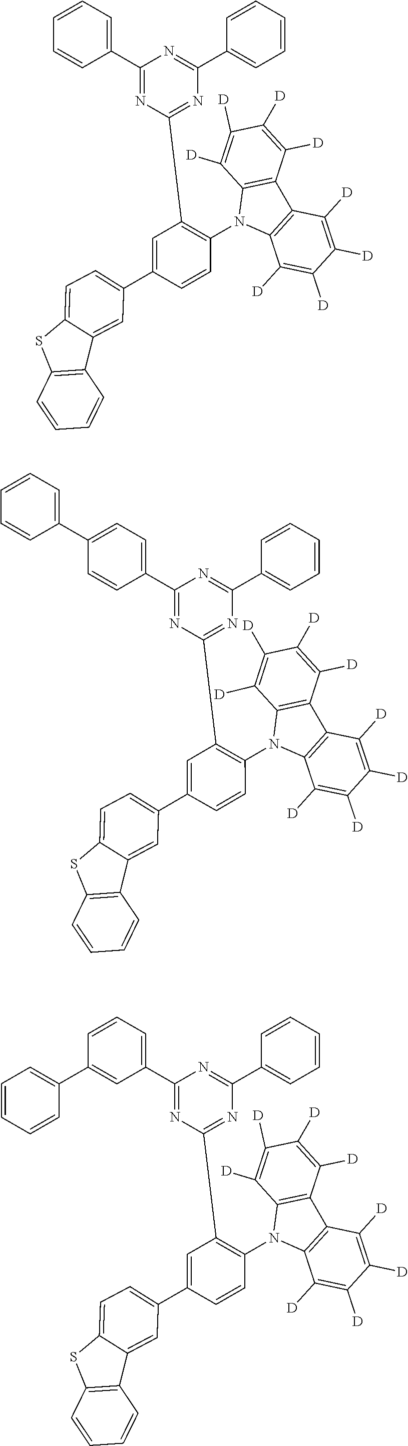 [SMILES: [2H]c1c([2H])c([2H])c2c(c1[2H])c1c([2H])c([2H])c([2H])c([2H])c1n2-c1ccc(-c2ccc3sc4ccccc4c3c2)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc(-c2ccc3sc4ccccc4c3c2)cc1-c1nc(-c2ccccc2)nc(-c2ccc(-c3ccccc3)cc2)n1 ; [2H]c1c([2H])c([2H])c2c(c1[2H])c1c([2H])c([2H])c([2H])c([2H])c1n2-c1ccc(-c2ccc3sc4ccccc4c3c2)cc1-c1nc(-c2ccccc2)nc(-c2cccc(-c3ccccc3)c2)n1]

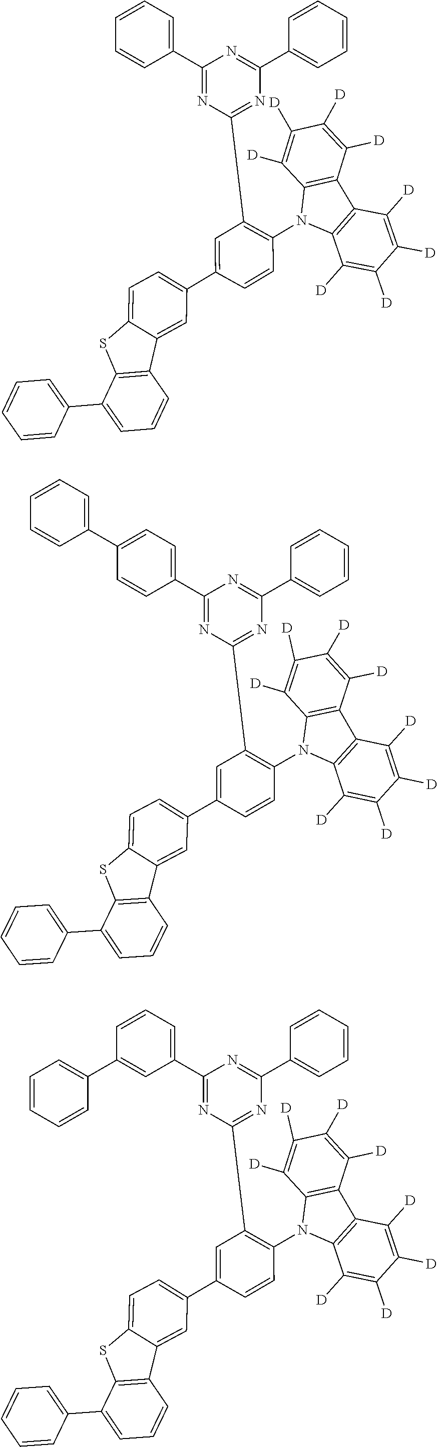

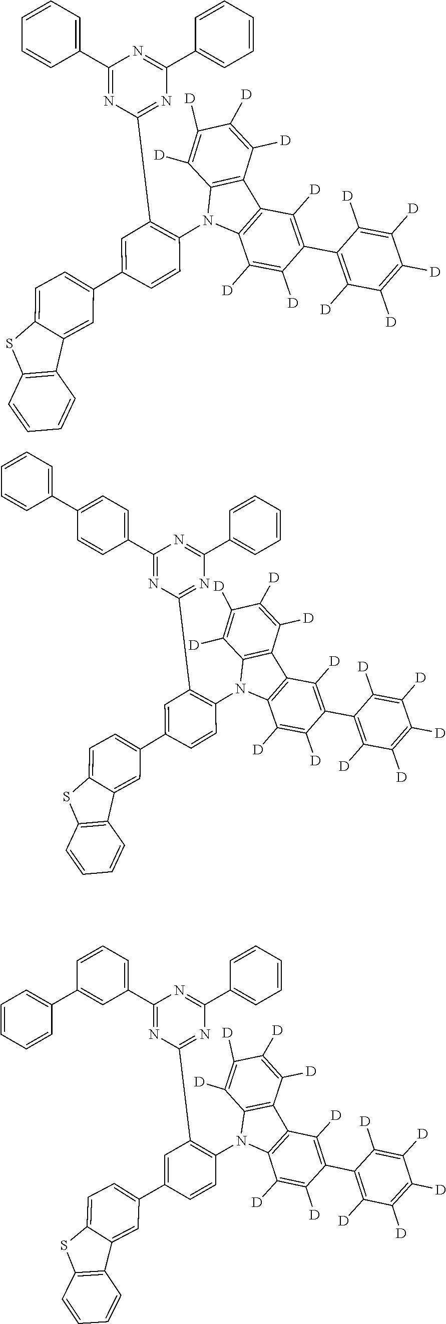

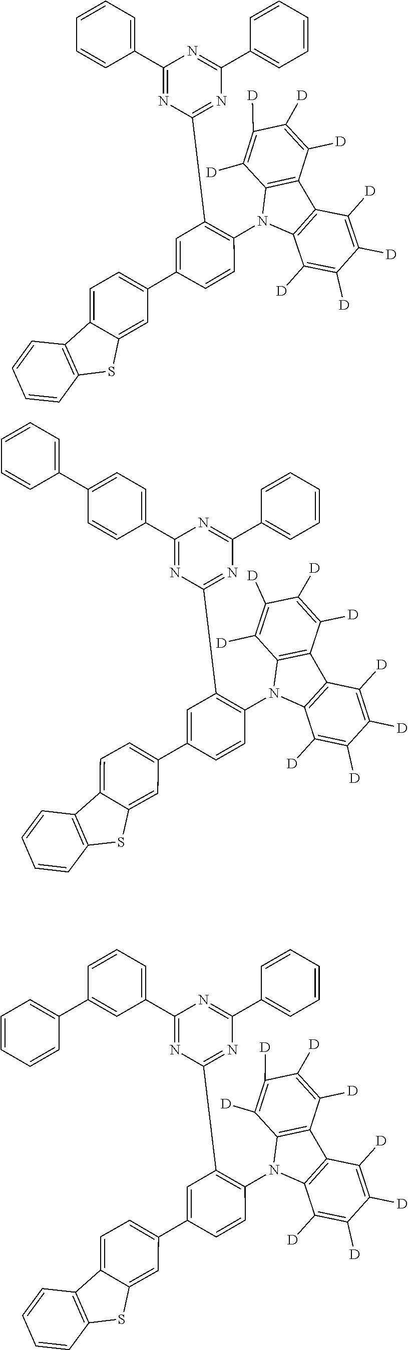

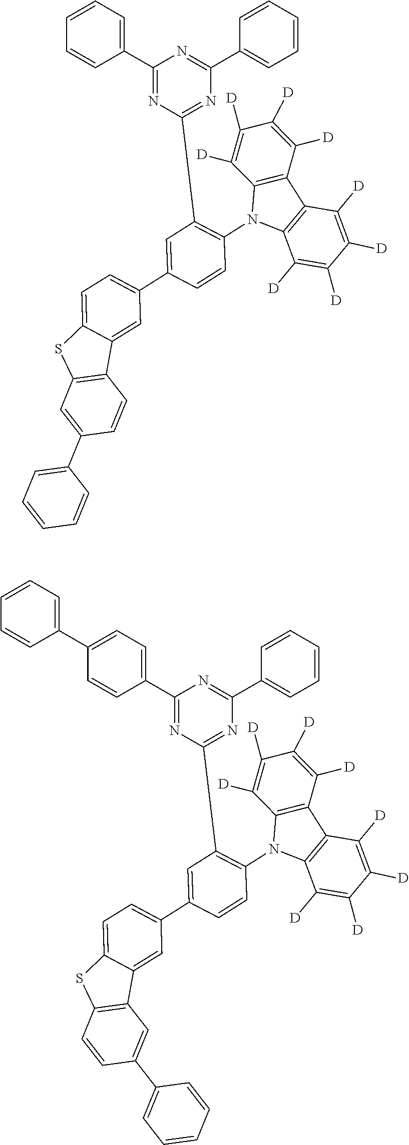 [SMILES: [2H]c1c([2H])c([2H])c2c(c1[2H])c1c([2H])c([2H])c([2H])c([2H])c1n2-c1ccc(-c2ccc3sc4cc(-c5ccccc5)ccc4c3c2)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc(-c2ccc3sc4ccc(-c5ccccc5)cc4c3c2)cc1-c1nc(-c2ccccc2)nc(-c2ccc(-c3ccccc3)cc2)n1]

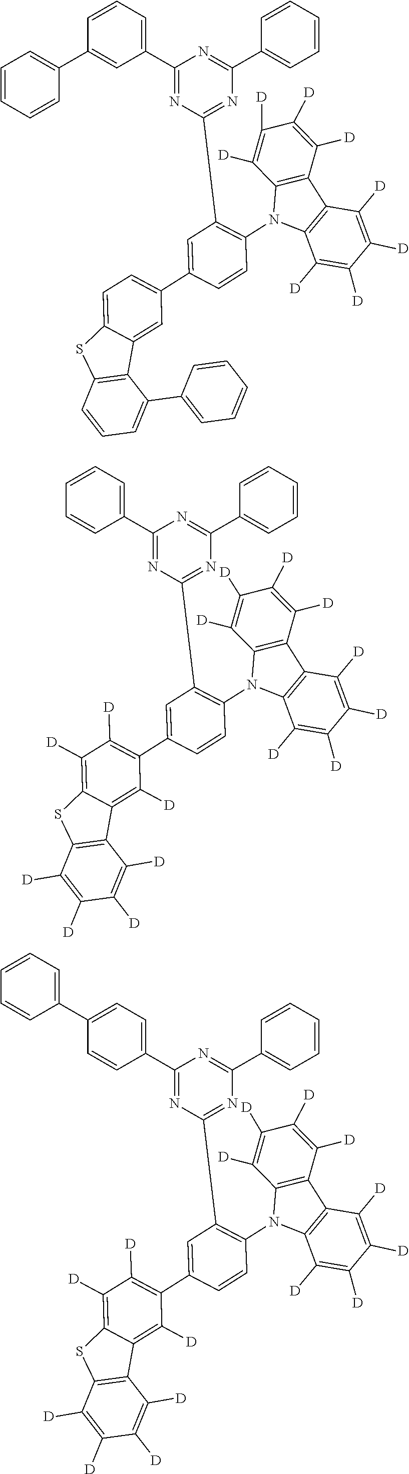

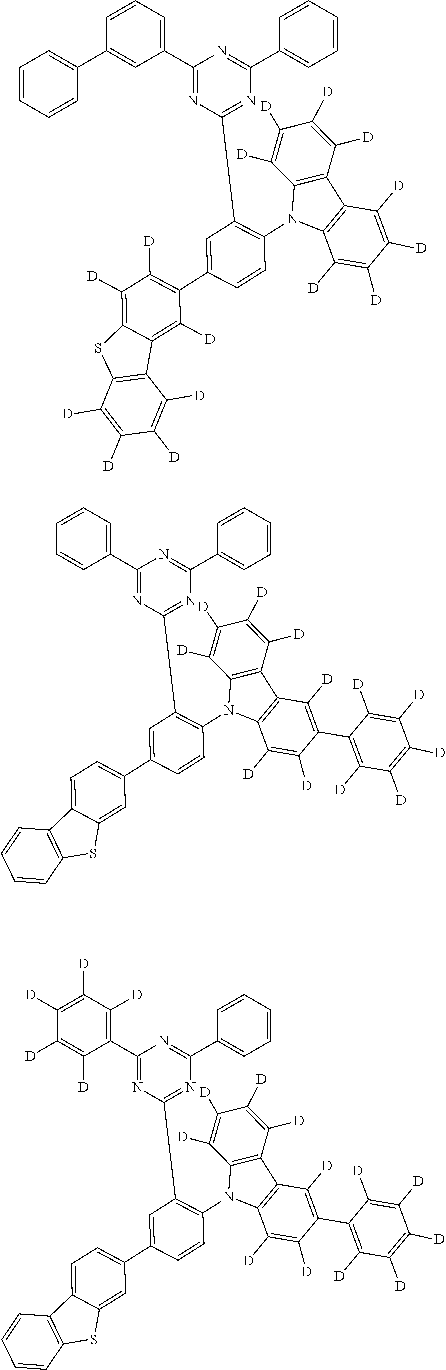

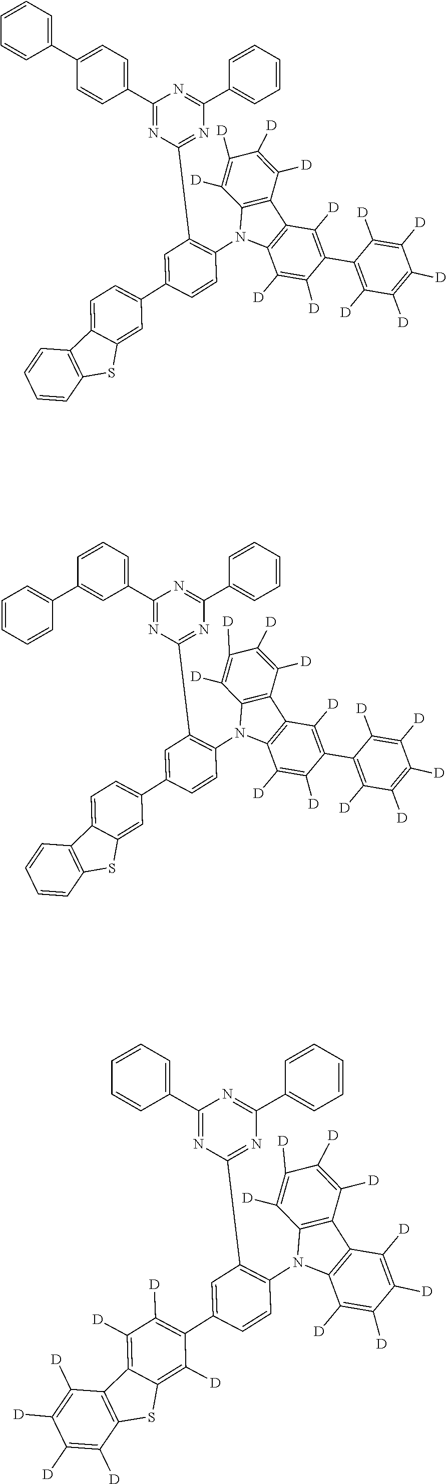

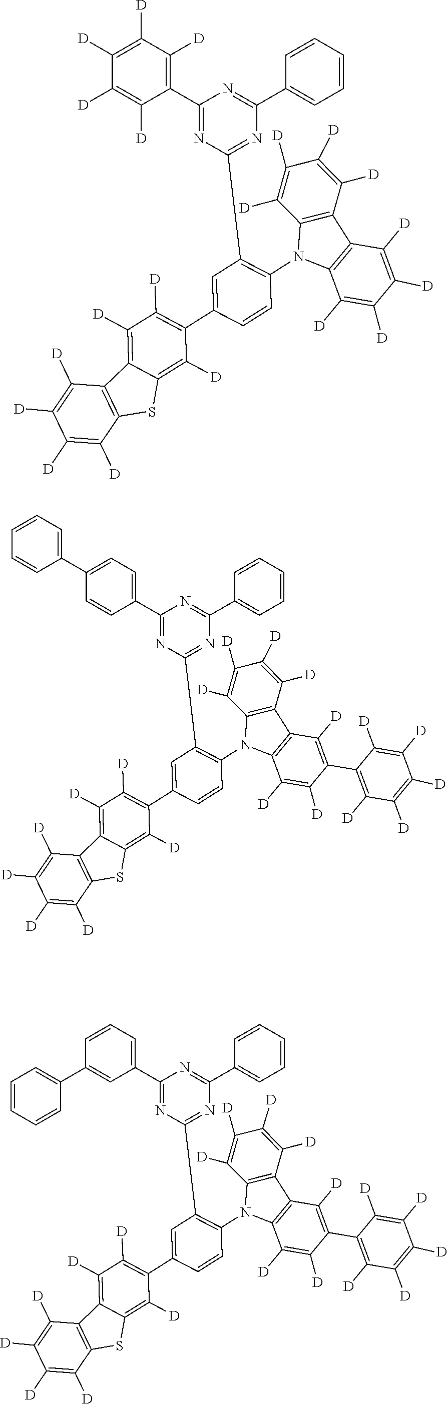

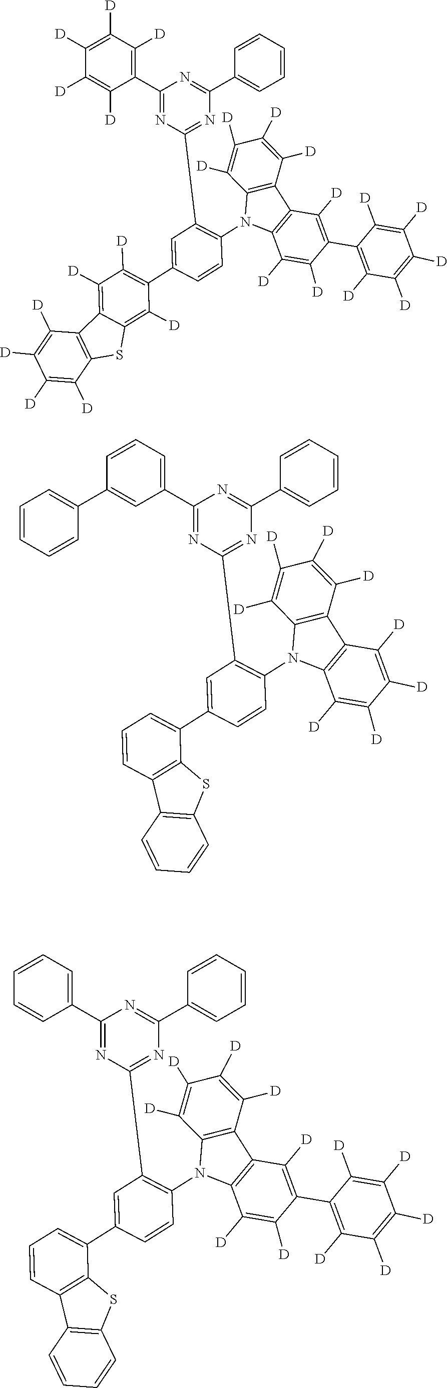

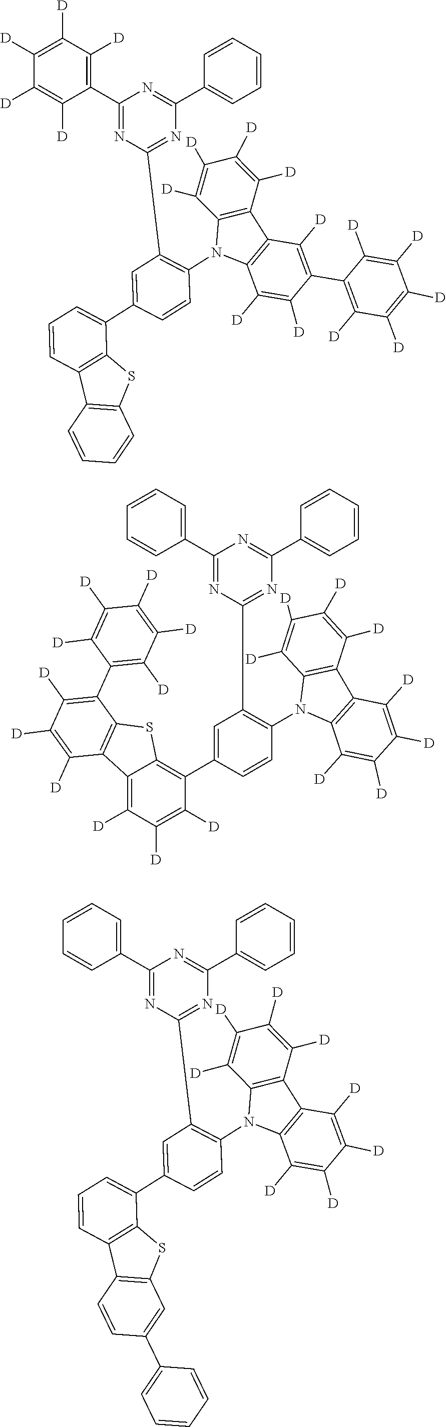

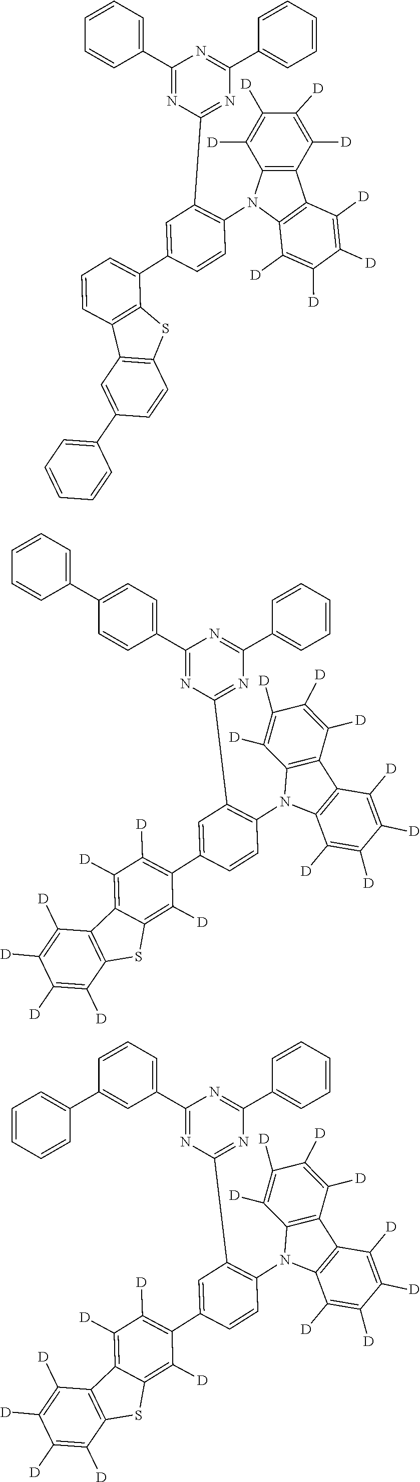

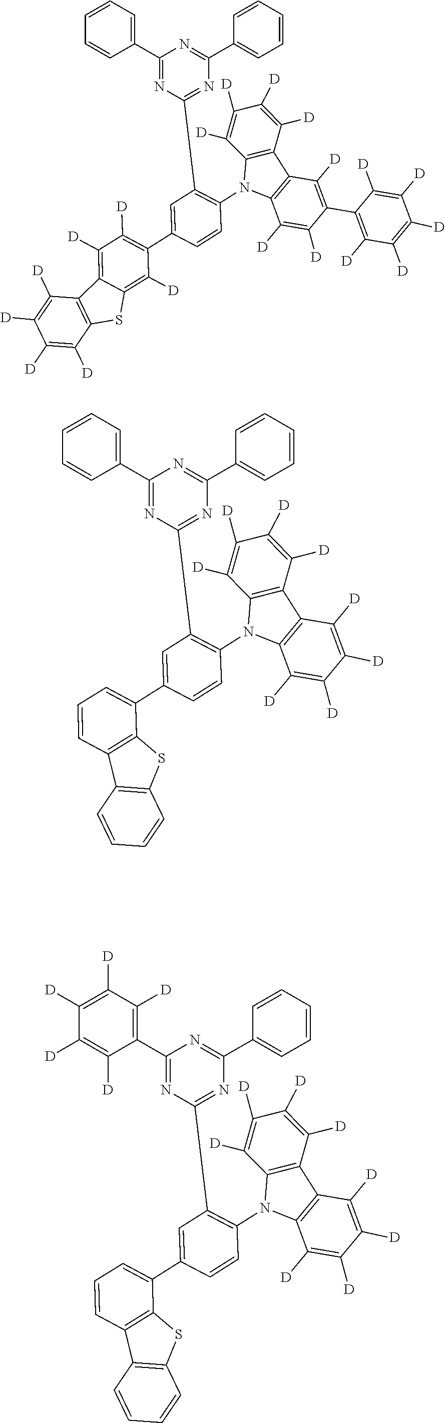

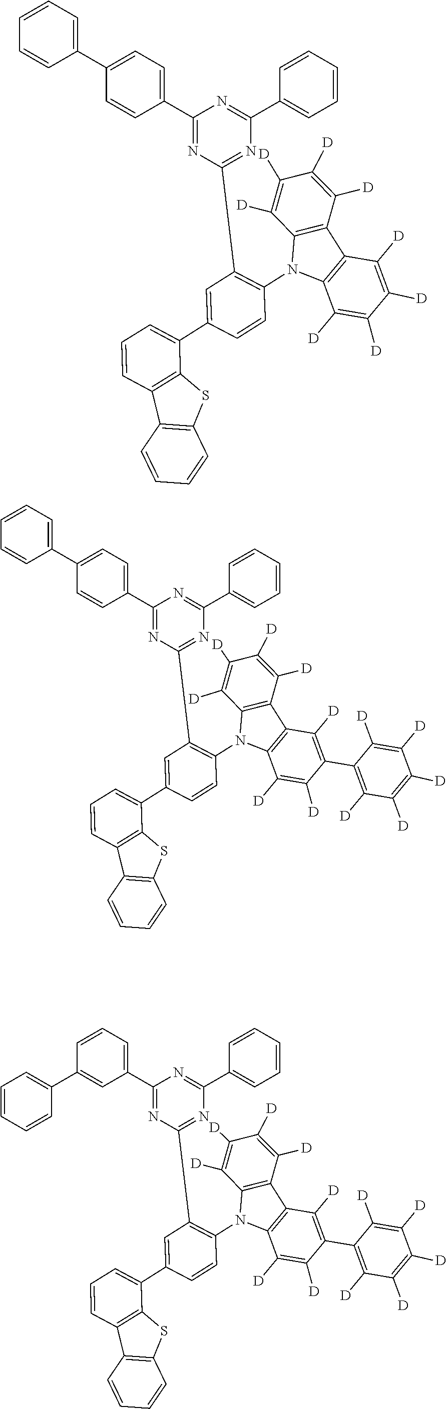 [SMILES: [2H]c1c([2H])c([2H])c2c(c1[2H])c1c([2H])c([2H])c([2H])c([2H])c1n2-c1ccc(-c2cccc3c2sc2ccccc23)cc1-c1nc(-c2ccccc2)nc(-c2ccc(-c3ccccc3)cc2)n1 ; [2H]c1c([2H])c([2H])c(-c2c([2H])c([2H])c3c(c2[2H])c2c([2H])c([2H])c([2H])c([2H])c2n3-c2ccc(-c3cccc4c3sc3ccccc34)cc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2ccc(-c3cccc4c3sc3ccccc34)cc2-c2nc(-c3ccccc3)nc(-c3cccc(-c4ccccc4)c3)n2)c([2H])c1[2H]]

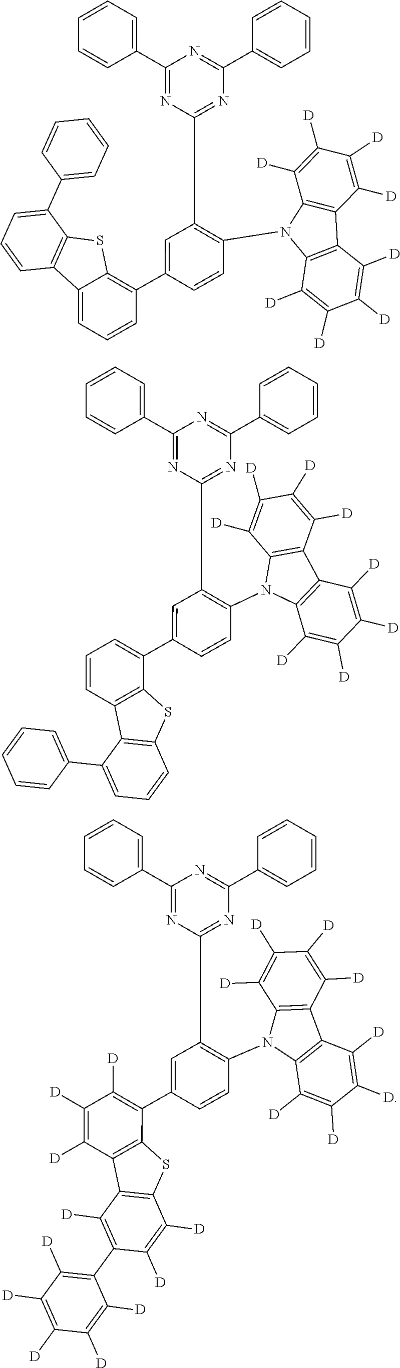

In addition, according to the present disclosure, there is provided a method for preparing the compound represented by Chemical Formula 1.

In one example, the compound represented by Chemical Formula 1 can be prepared by a preparation method as shown in the following Reaction Scheme 1. The Reaction Scheme 1 illustrates a case where R₁₀ of Chemical Formula 1 is a bond with Chemical Formula 2, R₉, R₁₁, and R₁₂ are hydrogen, and all of the rest of R₁₃ to R₁₆ not bonding with Chemical Formula 1 and R₁₇ to R₂₀ are hydrogen:

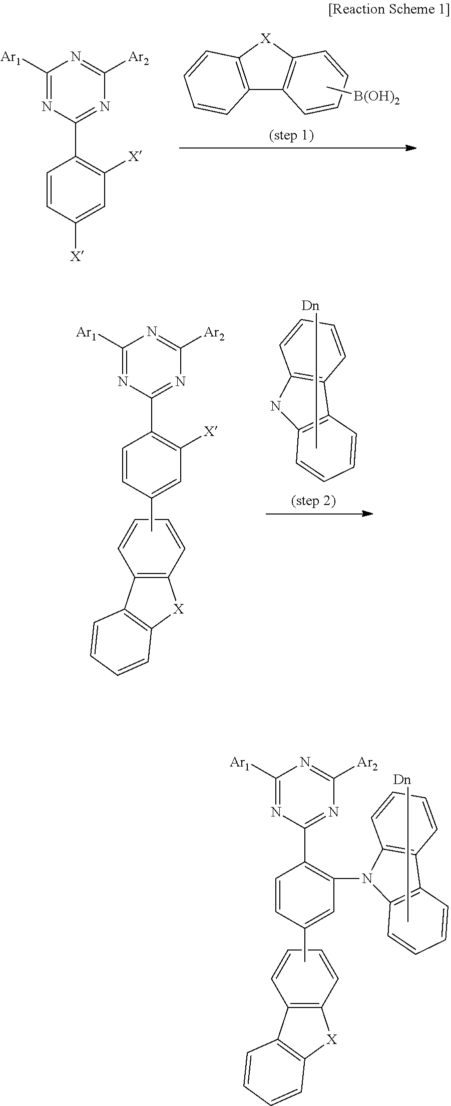

wherein, Ar₁, Ar₂, and X are as defined in Chemical Formulas 1 and 2, D is deuterium, n is an integer from 1 to 8, and X is halogen. Preferably, X is each independently fluoro, chloro, or bromo.

Step 1 of the Reaction Scheme 1 is a Suzuki coupling reaction, which is preferably carried out in the presence of a palladium catalyst and a base, and a reactive group for the Suzuki coupling reaction can be modified as known in the art.

Step 2 of the Reaction Scheme 1 is an amine substitution reaction, which is preferably carried out in the presence of a palladium catalyst and a base, and a reactive group for the Suzuki coupling reaction can be modified as known in the art.

The preparation method can be further embodied in Preparation Examples described hereinafter.

Further, according to the present disclosure, there is provided an organic light emitting device comprising a compound represented by Chemical Formula 1. In one example, the present disclosure provides an organic light emitting device comprising: a first electrode; a second electrode that is provided opposite to the first electrode; and one or more organic material layers that are provided between the first electrode and the second electrode, wherein at least one layer of the organic material layers includes the compound represented by Chemical Formula 1.

The organic material layer of the organic light emitting device of the present disclosure may have a single-layer structure, or it may have a multilayered structure in which two or more organic material layers are stacked. For example, the organic light emitting device of the present disclosure may have a structure comprising a hole injection layer, a hole transport layer, a light emitting layer, an electron transport layer, an electron injection layer and the like as the organic material layer. However, the structure of the organic light emitting device is not limited thereto, and it may include a smaller number of organic layers.

Further, the organic material layer may include a light emitting layer, wherein the organic material layer may include the compound represented by Chemical Formula 1. In particular, the compound according to the present disclosure can be used as a host of a light emitting layer.

Further, the organic material layer may include a hole injection layer, a hole transport layer, or an electron blocking layer, wherein the hole injection layer, hole transport layer, or electron blocking layer includes the compound represented by Chemical Formula 1.

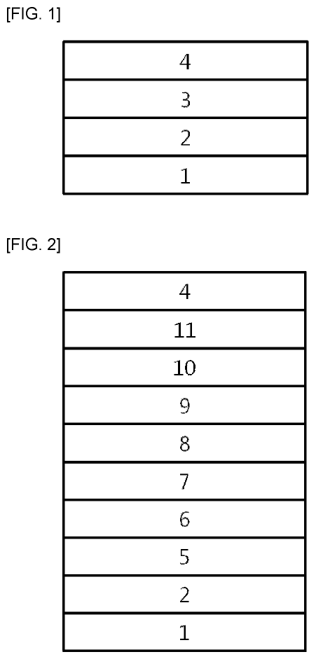

Further, the organic light emitting device according to the present disclosure may be a normal type organic light emitting device in which an anode, one or more organic material layers, and a cathode are sequentially stacked on a substrate. Further, the organic light emitting device according to the present disclosure may be an inverted type organic light emitting device in which a cathode, one or more organic material layers, and an anode are sequentially stacked on a substrate. For example, the structure of the organic light emitting device according to an embodiment of the present disclosure is illustrated in FIGS. 1 to 3.

FIG. 1 shows an example of an organic light emitting device comprising a substrate 1, an anode 2, an organic material layer 3, and a cathode 4. In such a structure, the compound represented by Chemical Formula 1 may be included in the organic material layer.

FIG. 2 shows an example of an organic light emitting device comprising a substrate 1, an anode 2, a hole injection layer 5, a hole transport layer 6, an electron blocking layer 7, a light emitting layer 8, a hole blocking layer 9, an electron transport layer 10, an electron injection layer 11, and a cathode 4. In such a structure, the compound represented by Chemical Formula 1 may be included in at least one layer of the hole injection layer, hole transport layer, electron blocking layer, light emitting layer, hole blocking layer, electron transport layer, and electron injection layer, and for example, it may be included in the light emitting layer.

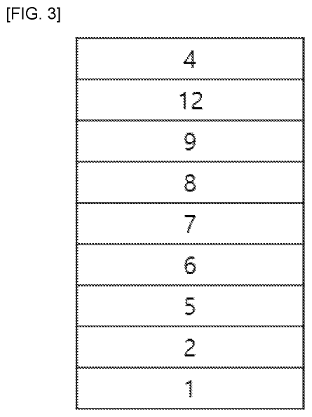

FIG. 3 shows an example of an organic light emitting device comprising a substrate 1, an anode 2, a hole injection layer 5, a hole transport layer 6, an electron blocking layer 7, a light emitting layer 8, a hole blocking layer 9, an electron injection and transport layer 12, and a cathode 4. In such a structure, the compound represented by Chemical Formula 1 may be included in at least one layer of the hole injection layer, hole transport layer, electron blocking layer, light emitting layer, hole blocking layer, and electron injection and transport layer, and for example, it may be included in the light emitting layer.

The organic light emitting device according to the present disclosure may be manufactured by materials and methods known in the art, except that at least one layer of the organic material layers includes the compound represented by Chemical Formula 1. Further, when the organic light emitting device includes a plurality of organic material layers, the organic material layers may be formed of the same material or different materials.

For example, the organic light emitting device according to the present disclosure can be manufactured by sequentially stacking a first electrode, an organic material layer and a second electrode on a substrate. In this case, the organic light emitting device may be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound represented by Chemical Formula 1 can be formed into an organic layer by a solution coating method as well as a vacuum deposition method at the time of manufacturing an organic light emitting device. Wherein, the solution coating method means a spin coating, a dip coating, a doctor blading, an inkjet printing, a screen printing, a spray method, a roll coating, or the like, but is not limited thereto.

In addition to such a method, the organic light emitting device may be manufactured by sequentially depositing a cathode material, an organic material layer and an anode material on a substrate (International Publication WO2003/012890). However, the manufacturing method is not limited thereto.

In one example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrin, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which may receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto.

The electron blocking layer serves to suppress the electrons injected from the cathode from being transmitted toward the anode without being recombined in the light emitting layer, thereby improving the efficiency of the organic light emitting device. The electron blocking layer is preferably a material having a smaller electron affinity than the electron transport layer.

The light emitting material is preferably a material which may receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq₃); a carbazole-based compound; a dimerized styryl compound; BAlq; a 10-hydroxybenzoquinoline-metal compound; a benzoxazole, benzthiazole and benzimidazole-based compound; a poly(p-phenylenevinylene)(PPV)-based polymer; a spiro compound; polyfluorene, rubrene, and the like, but are not limited thereto.

The light emitting layer may include a host material and a dopant material. The host material includes a fused aromatic ring derivative, a heterocycle-containing compound, or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto. In particular, in the present disclosure, the compound represented by Chemical Formula 1 can be used as a host material for the light emitting layer, and in this case, it is possible to obtain low voltage, high efficiency, and/or long lifetime characteristics of the organic light emitting device.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The electron transport layer is a layer that may receive the electrons from the electron injection layer and transport the electrons to the light emitting layer, and an electron transport material is suitably a material which may receive well injection of electrons from a cathode and transfer the electrons to a light emitting layer, and has a large mobility for electrons. Specific examples of the electron transport material include: an Al complex of 8-hydroxyquinoline; a complex including Alq₃; an organic radical compound; a hydroxyflavone-metal complex, and the like, but are not limited thereto. The electron transport layer may be used with any desired cathode material, as used according to a conventional technique. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

According to one embodiment of the present disclosure, an electron transport material and an electron injection material can be deposited simultaneously to produce a single layer of electron injection and transport layer.

The hole blocking layer prevents the holes injected in the anode from being transferred to the electron transport layer without being recombined in the light emitting layer. The hole blocking layer is preferably a material having the large ionization energy.

The organic light emitting device according to the present disclosure may be a bottom emission device, a top emission device, or a double-sided light emitting device depending on the materials used.

In addition, the compound represented by Chemical Formula 1 may be included in an organic solar cell or an organic transistor in addition to an organic light emitting device.

The preparation of the compound represented by Chemical Formula 1 and the organic light emitting device including the same will be specifically described in the following Examples. However, the following Examples are provided for illustrative purposes only, and are not intended to limit the scope of the present disclosure.

### PREPARATION EXAMPLE

### Preparation Example 1: Preparation of Compound GH1

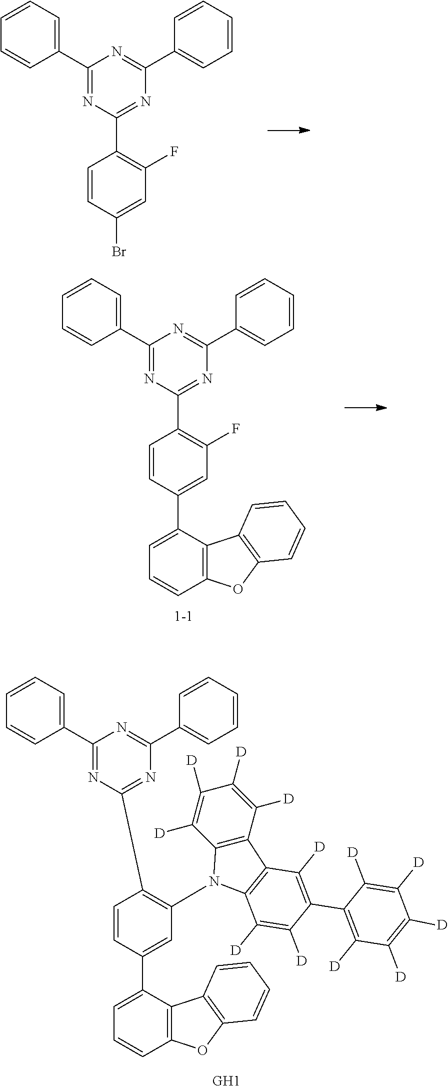

### Preparation Example 1-1: Preparation of Compound 1-1

2-(4-Bromo-2-fluorophenyl)-4,6-diphenyl-1,3,5-triazine (30 g, 73.8 mmol) and dibenzo[b,d]furan-1-ylboronic acid (15.7 g, 73.8 mmol) were added to 600 ml of tetrahydrofuran under a nitrogen atmosphere, and the mixture was stirred and refluxed. Then, potassium carbonate (30.6 g, 221.5 mmol) was dissolved in 31 ml of water, added thereto, sufficiently stirred and then tetrakistriphenyl-phosphinopalladium (2.6 g, 2.2 mmol) was added. After the reaction for 2 hours, the reaction mixture was cooled to room temperature, and then the resulting solid was filtered. The solid was added to and dissolved in 1822 mL of chloroform, washed twice with water, and the organic layer was separated. Anhydrous magnesium sulfate was added thereto, stirred, filtered, and the filtrate was distilled under reduced pressure. The concentrated compound was recrystallized from chloroform and ethyl acetate to prepare Compound 1-1 as a white solid (29.2 g, 80%, MS: [M+H]+=494.5).

### Preparation Example 1-2: Preparation of Compound GH1

1-1 (10 g, 20.3 mmol) and 3-(phenyl-d5)-9H-carbazole-1,2,4,5,6,7,8-d7(5.2 g, 20.3 mmol) were added to 200 ml of DMF under a nitrogen atmosphere, and the mixture was stirred and refluxed. Then, potassium acetate (6 g, 60.8 mmol) was added thereto, and after the reaction for 6 hours, the reaction mixture was cooled to room temperature, and then the resulting solid was filtered. The solid was added to and dissolved in 443 mL of chloroform, washed twice with water, and the organic layer was separated. Anhydrous magnesium sulfate was added thereto, stirred, filtered, and the filtrate was distilled under reduced pressure. The concentrated compound was purified by a silica column using chloroform and ethyl acetate to prepare Compound GH1 as a yellow solid (10.9 g, 74%, MS: [M+H]⁺=729.9).

### Preparation Example 2: Preparation of Compound GH2

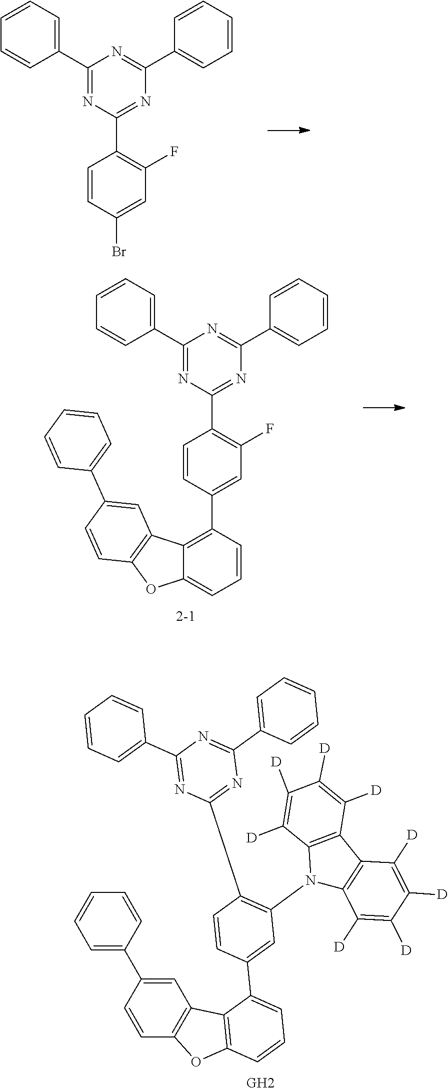

### Preparation Example 2-1: Preparation of Compound 2-1

Compound 2-1(31.1 g, 74%, MS: [M+H]⁺=570.6) was prepared in the same manner as in Preparation Example 1-1, except that (8-phenyldibenzo[b,d]furan-1-yl)boronic acid was used instead of dibenzo[b,d]furan-1-ylboronic acid.

### Preparation Example 2-2: Preparation of Compound GH2

Compound GH2 (8.8 g, 69%, MS: [M+H]⁺=725.9) was prepared in the same manner as in Preparation Example 1-2, except that Compound 2-1 was used instead of Compound 1-1, and 9H-carbazole-1,2,3,4,5,6,7,8-d8 was used instead of 3-(phenyl-d5)-9H-carbazole-1,2,4,5,6,7,8-d7.

### Preparation Example 3: Preparation of Compound GH3

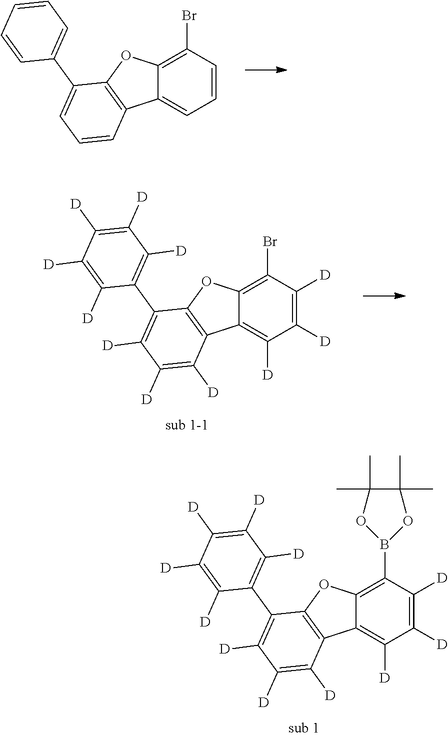 [SMILES: Brc1cccc2c1oc1c(-c3ccccc3)cccc12 ; [2H]c1c([2H])c([2H])c(-c2c([2H])c([2H])c([2H])c3c2oc2c(Br)c([2H])c([2H])c([2H])c23)c([2H])c1[2H] ; [2H]c1c([2H])c([2H])c(-c2c([2H])c([2H])c([2H])c3c2oc2c(B4OC(C)(C)C(C)(C)O4)c([2H])c([2H])c([2H])c23)c([2H])c1[2H]]

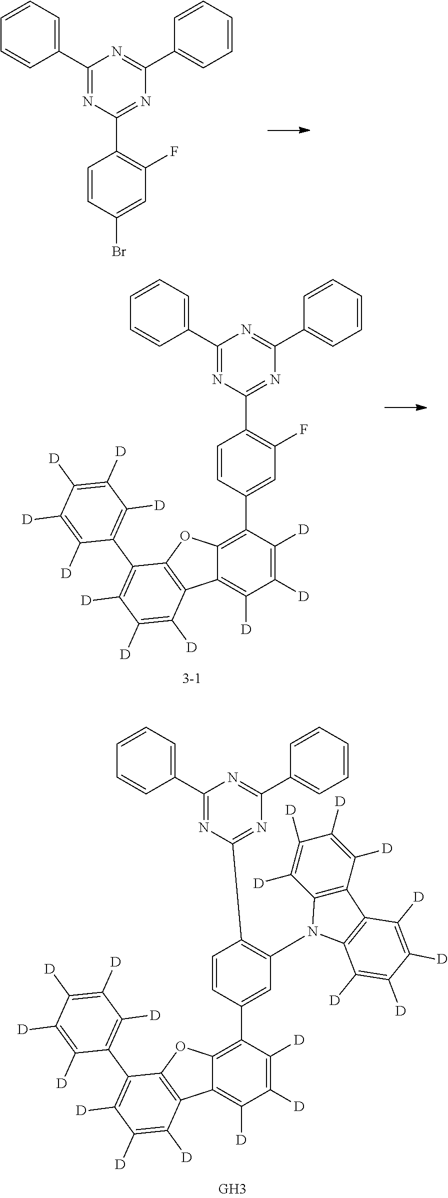 [SMILES: Fc1cc(Br)ccc1-c1nc(-c2ccccc2)nc(-c2ccccc2)n1 ; [2H]c1c([2H])c([2H])c(-c2c([2H])c([2H])c([2H])c3c2oc2c(-c4ccc(-c5nc(-c6ccccc6)nc(-c6ccccc6)n5)c(F)c4)c([2H])c([2H])c([2H])c23)c([2H])c1[2H] ; [2H]c1c([2H])c([2H])c(-c2c([2H])c([2H])c([2H])c3c2oc2c(-c4ccc(-c5nc(-c6ccccc6)nc(-c6ccccc6)n5)c(-n5c6c([2H])c([2H])c([2H])c([2H])c6c6c([2H])c([2H])c([2H])c([2H])c65)c4)c([2H])c([2H])c([2H])c23)c([2H])c1[2H]]

### Preparation Example 3-1: Preparation of Compound Sub 1-1

4-Bromo-6-phenyldibenzo[b,d]furan (50 g, 154.7 mmol) and TfOH (10 ml) were added to C₆D₆ (500 ml) under a nitrogen atmosphere, and stirred at 40° C. for 6 hours. After completion of the reaction, the temperature was lowered to room temperature, D₂O (100 ml) was added thereto, stirred for 30 minutes, and trimethylamine (12 ml) was added dropwise. The reaction solution was transferred to a separatory funnel, and extracted with water and chloroform. The extract was dried over MgSO₄ and recrystallized from ethanol to obtain 44.5 g of Compound sub 1-1. (yield: 86%, MS[M+H]⁺=334)

### Preparation Example 3-2: Preparation of Compound Sub 1

sub1-1 (50 g, 149.7 mmol) and bis(pinacolato)diborone (38.4 g, 164.7 mmol) were added to 1000 ml of Diox under a nitrogen atmosphere, and the mixture was stirred and refluxed. Then, potassium carbonate (43.2 g, 449.1 mmol) was added thereto, sufficiently stirred, and then palladiumdibenzylideneacetonepalladium (2.6 g, 4.5 mmol) and tricyclohexylphosphine (2.5 g, 9 mmol) were added. After the reaction for 5 hours, the reaction mixture was cooled to room temperature, the organic layer was filtered to remove salt, and then the filtered organic layer was distilled. This was added again to 571 mL of chloroform, dissolved and washed twice with water. The organic layer was then separated, anhydrous magnesium sulfate was added thereto, stirred, filtered, and the filtrate was distilled under reduced pressure. The concentrated compound was recrystallized from chloroform and ethanol to prepare Compound sub1 as a gray solid (51.4 g, 90%, MS: [M+H]⁺=382.4)

### Preparation Example 3-3: Preparation of Compound 3-1

Compound 3-1 as a white solid (24.4 g, 57%, MS: [M+H]⁺=581.7) was prepared in the same manner as in Preparation Example 1-1, except that Compound sub1 was used instead of dibenzo[b,d]furan-1-ylboronic acid.

### Preparation Example 3-4: Preparation of Compound GH3

Compound GH3 as a yellow solid (8 g, 63%, MS: [M+H]⁺=737) was prepared in the same manner as in Preparation Example 1-2, except that Compound 3-1 was used instead of Compound 1-1, and 9H-carbazole-1,2,3,4,5,6,7,8-d8 was used instead of 3-(phenyl-d5)-9H-carbazole-1,2,4,5,6,7,8-d7.

### Preparation Example 4: Preparation of Compound GH4

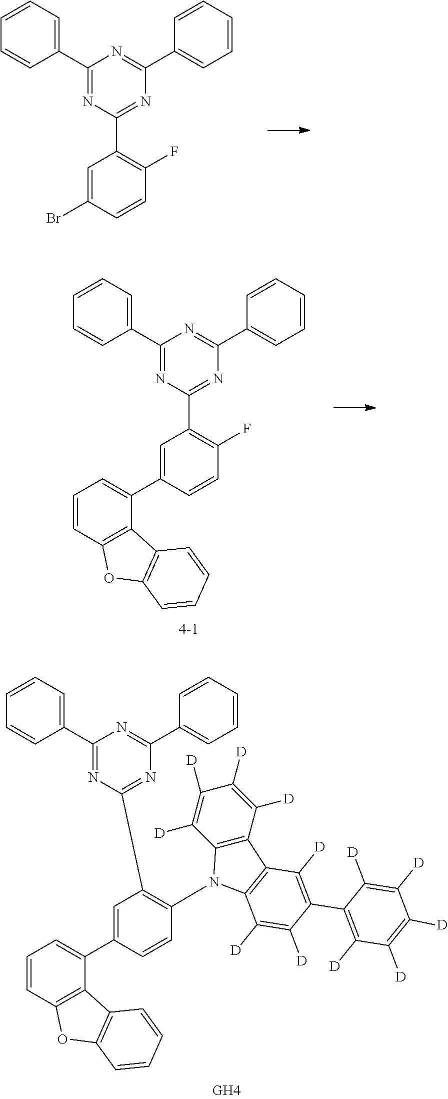 [SMILES: Fc1ccc(Br)cc1-c1nc(-c2ccccc2)nc(-c2ccccc2)n1 ; Fc1ccc(-c2cccc3oc4ccccc4c23)cc1-c1nc(-c2ccccc2)nc(-c2ccccc2)n1 ; [2H]c1c([2H])c([2H])c(-c2c([2H])c([2H])c3c(c2[2H])c2c([2H])c([2H])c([2H])c([2H])c2n3-c2ccc(-c3cccc4oc5ccccc5c34)cc2-c2nc(-c3ccccc3)nc(-c3ccccc3)n2)c([2H])c1[2H]]

### Preparation Example 4-1: Preparation of Compound 4-1

Compound 4-1 as a white solid (26.2 g, 72%, MS: [M+H]⁺=494.5) was prepared in the same manner as in Preparation Example 3-3, except that 2-(5-bromo-2-fluorophenyl)-4,6-diphenyl-1,3,5-triazine was used instead of 2-(4-bromo-2-fluorophenyl)-4,6-diphenyl-1,3,5-triazine.

### Preparation Example 4-2: Preparation of Compound GH4

Compound GH4 as a yellow solid (7.5 g, 51%, MS: [M+H]⁺=729.9) was prepared in the same manner as in Preparation Example 1-2, except that Compound 4-1 was used instead of Compound 1-1.

### Preparation Example 5: Preparation of Compound GH5

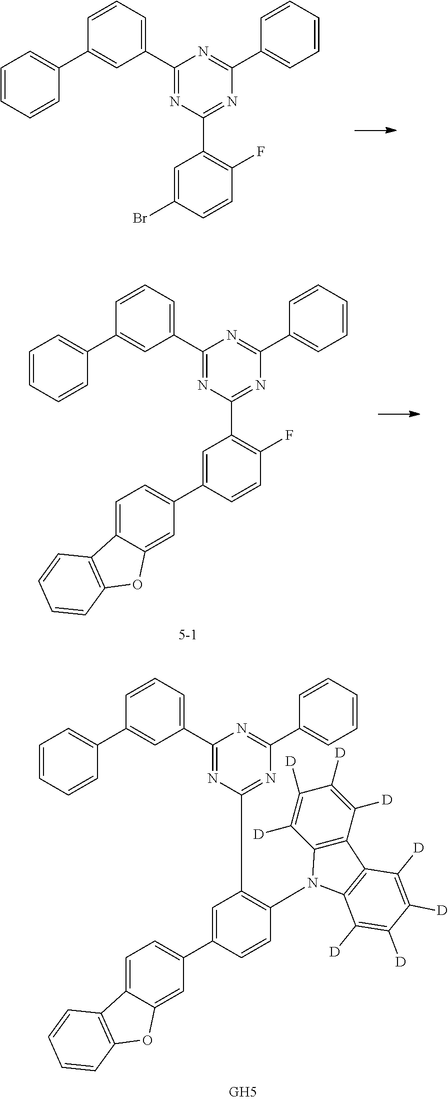 [SMILES: Fc1ccc(Br)cc1-c1nc(-c2ccccc2)nc(-c2cccc(-c3ccccc3)c2)n1 ; Fc1ccc(-c2ccc3c(c2)oc2ccccc23)cc1-c1nc(-c2ccccc2)nc(-c2cccc(-c3ccccc3)c2)n1 ; [2H]c1c([2H])c([2H])c2c(c1[2H])c1c([2H])c([2H])c([2H])c([2H])c1n2-c1ccc(-c2ccc3c(c2)oc2ccccc23)cc1-c1nc(-c2ccccc2)nc(-c2cccc(-c3ccccc3)c2)n1]

### Preparation Example 5-1: Preparation of Compound 5-1

Compound 5-1 as a white solid (20.5 g, 58%, MS: [M+H]⁺=570.6) was prepared in the same manner as in Preparation Example 1-1, except that 2-([1,1′-biphenyl]-3-yl)-4-(5-bromo-2-fluorophenyl)-6-phenyl-1,3,5-triazine was used instead of 2-(4-bromo-2-fluorophenyl)-4,6-diphenyl-1,3,5-triazine, and dibenzo[b,d]furan-3-ylboronic acid was used instead of dibenzo[b,d]furan-1-ylboronic acid.

### Preparation Example 5-2: Preparation of Compound GH5

Compound GH5 as a yellow solid (9.4 g, 74%, MS: [M+H]⁺=725.9) was prepared in the same manner as in Preparation Example 1-2, except that Compound 5-1 was used instead of Compound 1-1, and 9H-carbazole-1,2,3,4,5,6,7,8-d8 was used instead of 3-(phenyl-d5)-9H-carbazole-1,2,4,5,6,7,8-d7.

### Preparation Example 6: Preparation of Compound GH6

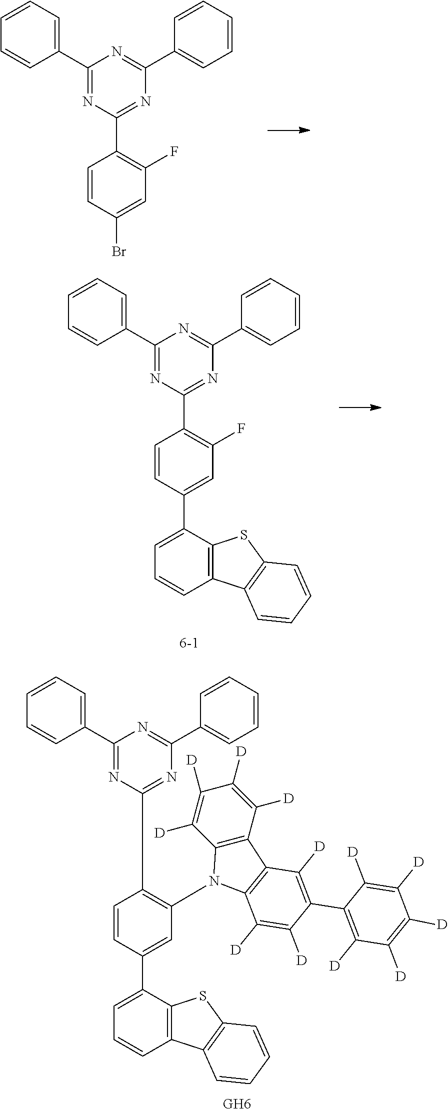

### Preparation Example 6-1: Preparation of Compound 6-1

Compound 6-1 as a white solid (24.8 g, 66%, MS: [M+H]⁺=510.6) was prepared in the same manner as in Preparation Example 1-1, except that dibenzo[b,d]thiophen-4-ylboronic acid was used instead of dibenzo[b,d]furan-1-ylboronic acid.

### Preparation Example 6-2: Preparation of Compound GH6

Compound GH6 as a yellow solid (7 g, 48%, MS: [M+H]⁺=746) was prepared in the same manner as in Preparation Example 1-2, except that Compound 6-1 was used instead of Compound 1-1.

### Preparation Example 7: Preparation of Compound GH7

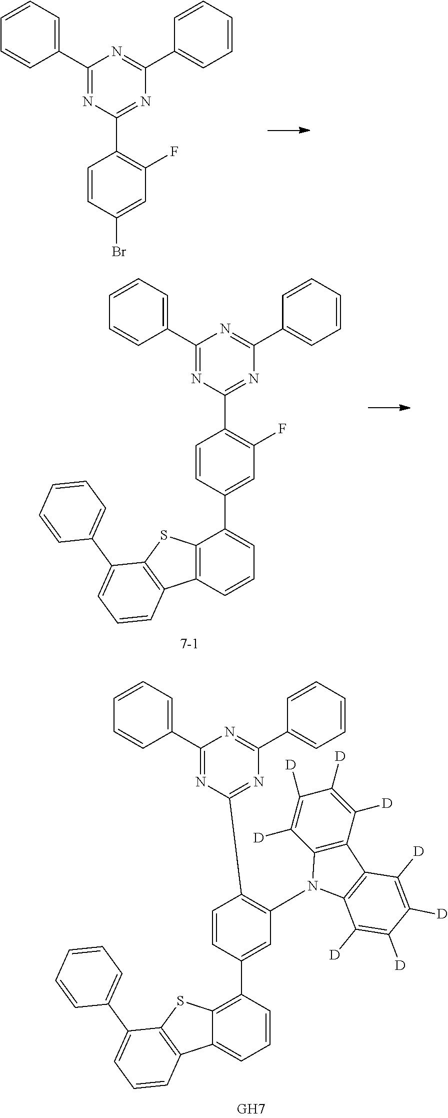

### Preparation Example 7-1: Preparation of Compound 7-1

Compound 7-1 as a white solid (30.3 g, 70%, MS: [M+H]⁺=586.7) was prepared in the same manner as in Preparation Example 1-1, except that (6-phenyldibenzo[b,d]thiophen-4-yl)boronic acid was used instead of dibenzo[b,d]furan-1-ylboronic acid.

### Preparation Example 7-2: Preparation of Compound GH7

Compound GH7 as a yellow solid (6.6 g, 52%, MS: [M+H]⁺=742) was prepared in the same manner as in Preparation Example 1-2, except that Compound 7-1 was used instead of Compound 1-1, and 9H-carbazole-1,2,3,4,5,6,7,8-d8 was used instead of 3-(phenyl-d5)-9H-carbazole-1,2,4,5,6,7,8-d7.

### Preparation Example 8: Preparation of Compound GH8

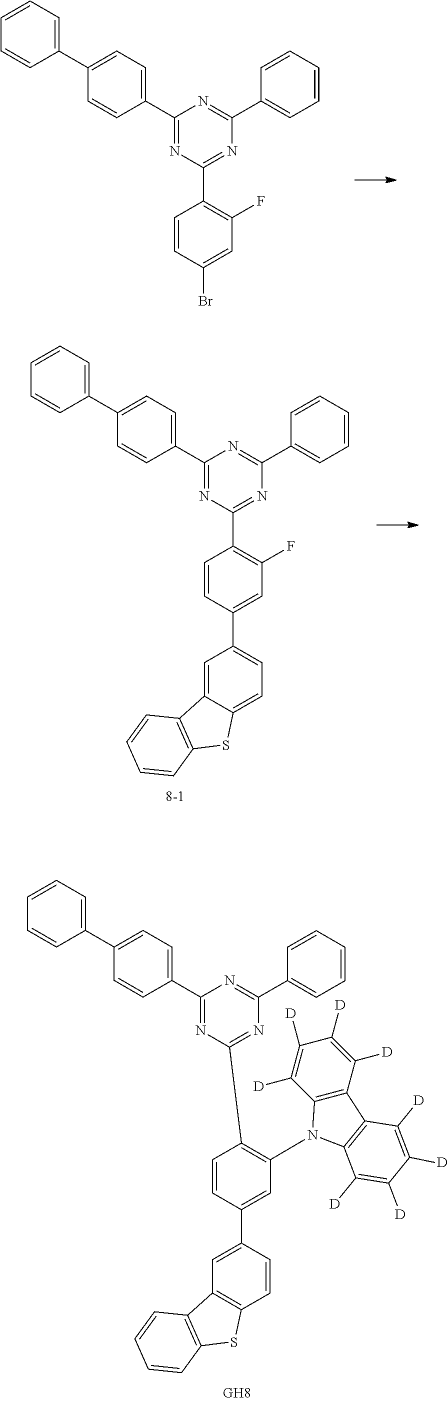 [SMILES: Fc1cc(Br)ccc1-c1nc(-c2ccccc2)nc(-c2ccc(-c3ccccc3)cc2)n1 ; Fc1cc(-c2ccc3sc4ccccc4c3c2)ccc1-c1nc(-c2ccccc2)nc(-c2ccc(-c3ccccc3)cc2)n1 ; [2H]c1c([2H])c([2H])c2c(c1[2H])c1c([2H])c([2H])c([2H])c([2H])c1n2-c1cc(-c2ccc3sc4ccccc4c3c2)ccc1-c1nc(-c2ccccc2)nc(-c2ccc(-c3ccccc3)cc2)n1]

### Preparation Example 8-1: Preparation of Compound 8-1

Compound 8-1 as a white solid (23.3 g, 64%, MS: [M+H]⁺=586.7) was prepared in the same manner as in Preparation Example 1-1, except that 2-([1,1′-biphenyl]-4-yl)-4-(4-bromo-2-fluorophenyl)-6-phenyl-1,3,5-triazine was used instead of 2-(4-bromo-2-fluorophenyl)-4,6-diphenyl-1,3,5-triazine, and dibenzo[b,d]thiophen-2-ylboronic acid was used instead of dibenzo[b,d]furan-1-ylboronic acid.

### Preparation Example 8-2: Preparation of Compound GH8

Compound GH8 as a yellow solid (7.6 g, 60%, MS: [M+H]⁺=742) was prepared in the same manner as in Preparation Example 1-2, except that Compound 8-1 was used instead of Compound 1-1, and 9H-carbazole-1,2,3,4,5,6,7,8-d8 was used instead of 3-(phenyl-d5)-9H-carbazole-1,2,4,5,6,7,8-d7.

### Preparation Example 9: Preparation of Compound GH9

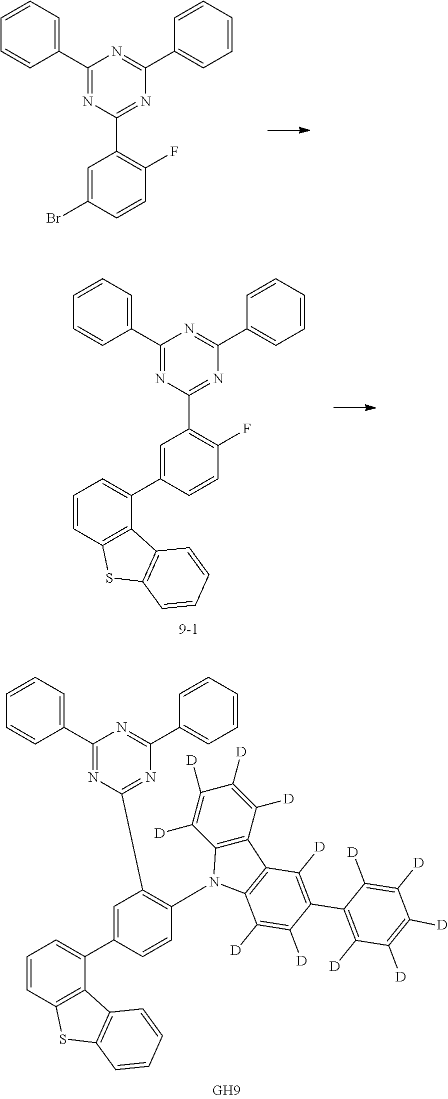 [SMILES: Fc1ccc(Br)cc1-c1nc(-c2ccccc2)nc(-c2ccccc2)n1 ; Fc1ccc(-c2cccc3sc4ccccc4c23)cc1-c1nc(-c2ccccc2)nc(-c2ccccc2)n1 ; [2H]c1c([2H])c([2H])c(-c2c([2H])c([2H])c3c(c2[2H])c2c([2H])c([2H])c([2H])c([2H])c2n3-c2ccc(-c3cccc4sc5ccccc5c34)cc2-c2nc(-c3ccccc3)nc(-c3ccccc3)n2)c([2H])c1[2H]]

### Preparation Example 9-1: Preparation of Compound 9-1

Compound 9-1 as a white solid (28.6 g, 76%, MS: [M+H]⁺=510.6) was prepared in the same manner as in Preparation Example 1-1, except that 2-(5-bromo-2-fluorophenyl)-4,6-diphenyl-1,3,5-triazine was used instead of 2-(4-bromo-2-fluorophenyl)-4,6-diphenyl-1,3,5-triazine, and dibenzo[b,d]thiophen-1-ylboronic acid was used instead of dibenzo[b,d]furan-1-ylboronic acid.

### Preparation Example 9-2: Preparation of Compound GH9

Compound GH9 as a yellow solid (11 g, 75%, MS: [M+H]⁺=746) was prepared in the same manner as in Preparation Example 1-2, except that Compound 9-1 was used instead of Compound 1-1.

### Preparation Example 10: Preparation of Compound GH10

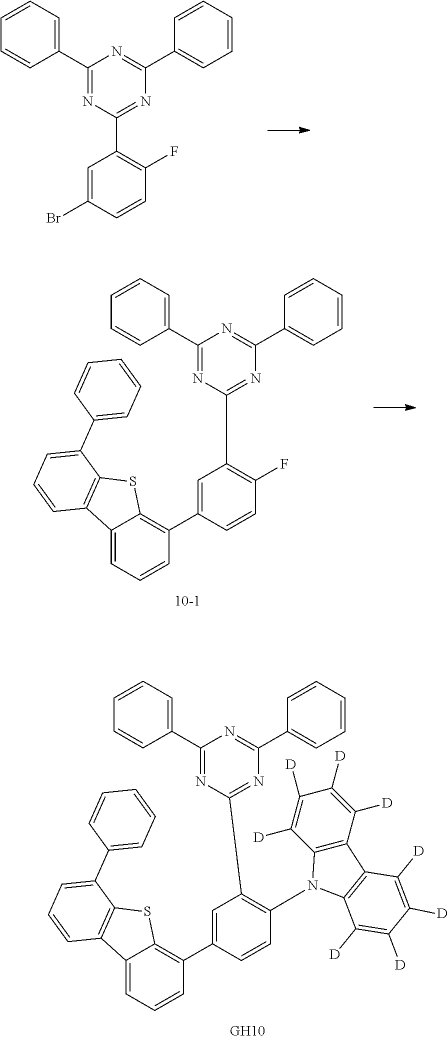 [SMILES: Fc1ccc(Br)cc1-c1nc(-c2ccccc2)nc(-c2ccccc2)n1 ; Fc1ccc(-c2cccc3c2sc2c(-c4ccccc4)cccc23)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc(-c2cccc3c2sc2c(-c4ccccc4)cccc23)cc1-c1nc(-c2ccccc2)nc(-c2ccccc2)n1]

### Preparation Example 10-1: Preparation of Compound 10-1

Compound 10-1 as a white solid (22.9 g, 53%, MS: [M+H]⁺=586.7) was prepared in the same manner as in Preparation Example 1-1, except that 2-(5-bromo-2-fluorophenyl)-4,6-diphenyl-1,3,5-triazine was used instead of 2-(4-bromo-2-fluorophenyl)-4,6-diphenyl-1,3,5-triazine, and (6-phenyldibenzo[b,d]thiophen-4-yl)boronic acid was used instead of dibenzo[b,d]furan-1-ylboronic acid.

### Preparation Example 10-2: Preparation of Compound GH10

Compound GH10 as a yellow solid (8.2 g, 65%, MS: [M+H]⁺=742) was prepared in the same manner as in Preparation Example 1-2, except that Compound 10-1 was used instead of Compound 1-1, and 9H-carbazole-1,2,3,4,5,6,7,8-d8 was used instead of 3-(phenyl-d5)-9H-carbazole-1,2,4,5,6,7,8-d7.

### Examples

### Example 1

A glass substrate on which ITO (indium tin oxide) was coated as a thin film to a thickness of 100 nm was put into distilled water in which a detergent was dissolved, and ultrasonically cleaned. A product manufactured by Fischer Co. was used as the detergent, and as the distilled water, distilled water filtered twice using a filter manufactured by Millipore Co. was used. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. In addition, the substrate was cleaned for 5 minutes using oxygen plasma and then transferred to a vacuum depositor.

On the ITO transparent electrode thus prepared, the following compound HT-A was thermally vacuum-deposited to a thickness of 60 nm to form a hole injection layer.

The following compound HAT were vacuum-deposited on the hole injection layer to form a first hole transport layer with a thickness of 5 nm, and the following compound HT-A was vacuum-deposited on the first hole transport layer to form a second hole transport layer with a thickness of 50 nm.

The following compound HT-B was thermally vacuum-deposited to a thickness of 45 nm on the hole transport layer to form an electron blocking layer.

The previously prepared compound GH1 was mixed with the following compound GH-H at a weight ratio of 1:1, and then vacuum-deposited with the following compound GD at a weight ratio of 90:10 to a thickness of 40 nm on the electron blocking layer to form a light emitting layer.

The following compound ET-A was vacuum-deposited to a thickness of 5 nm on the light emitting layer to form a hole blocking layer.

The following compound ET-B and the following compound LiQ were vacuum-deposited at a weight ratio of 1:1 on the hole blocking layer to form an electron injection and transport layer with a thickness of 35 nm.

Lithium fluoride (LiF) and aluminum were sequentially deposited to have a thickness of 1 nm and 100 nm, respectively, on the electron injection and transport layer to form a cathode, thereby completing the manufacture of an organic light emitting device.

In the above-mentioned processes, the vapor deposition rate of the organic material was maintained at 0.04 nm/sec to 0.09 nm/sec, the deposition rate of lithium fluoride was maintained at 0.03 nm/sec, and the deposition rate of aluminum was maintained at 0.2 nm/sec. The degree of vacuum during the deposition was maintained at 1*10⁻⁷ torr to 5*10⁻⁵ torr.

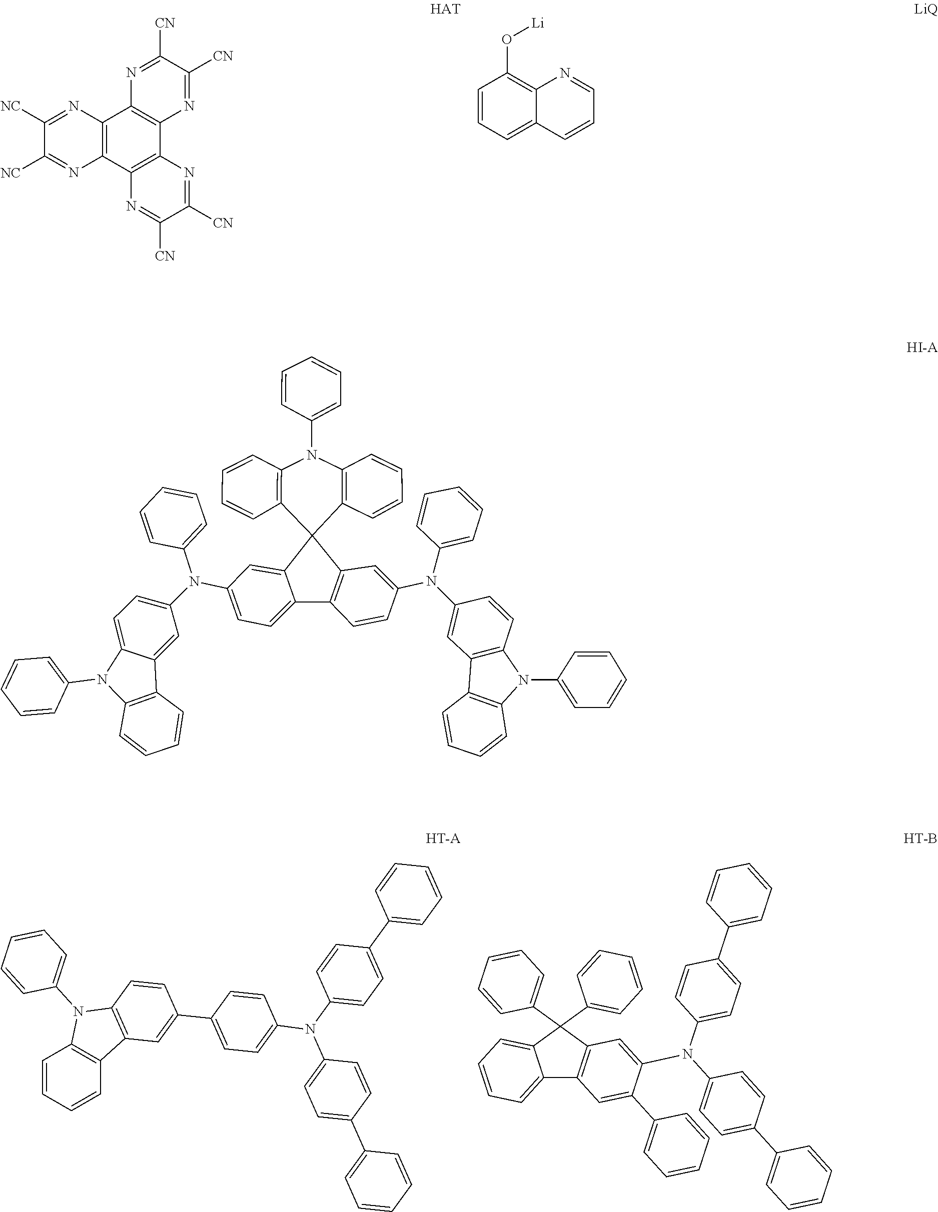

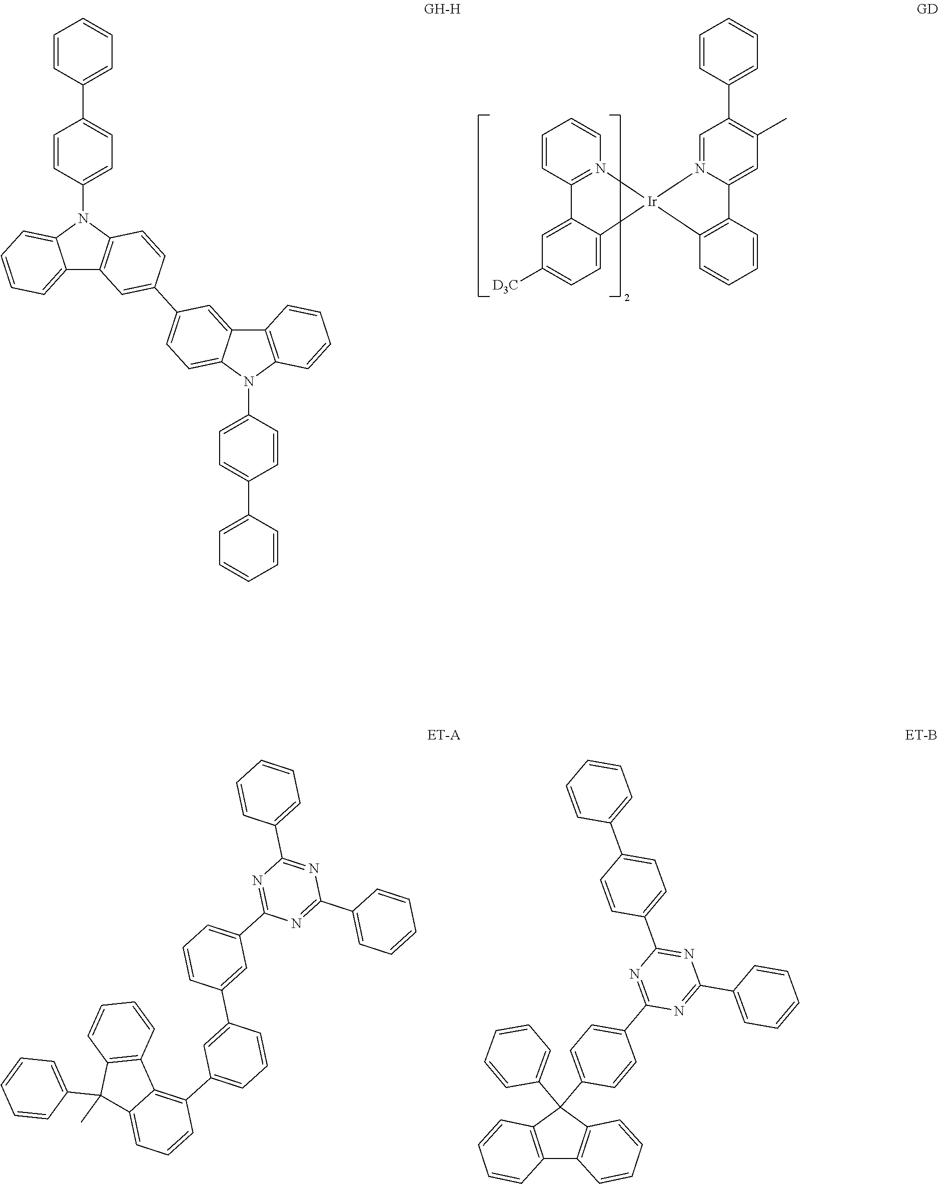

### Examples 2 to 10

The organic light emitting devices of Examples 2 to 10 were manufactured in the same manner as in Example 1, except that in Example 1, the compounds listed in Table 1 below were used instead of GH1. The structures of the compounds used in Examples are summarized as follows.

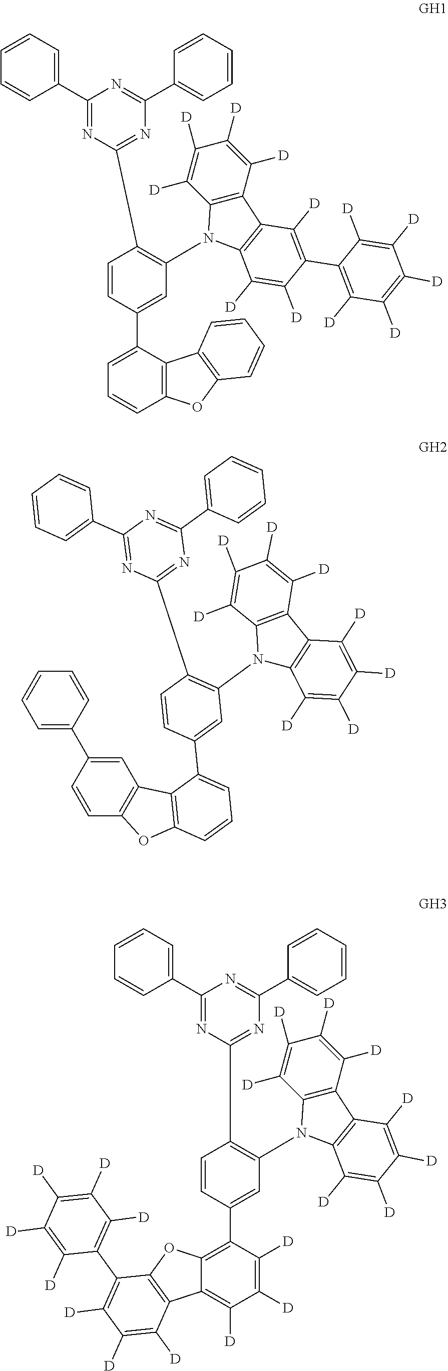

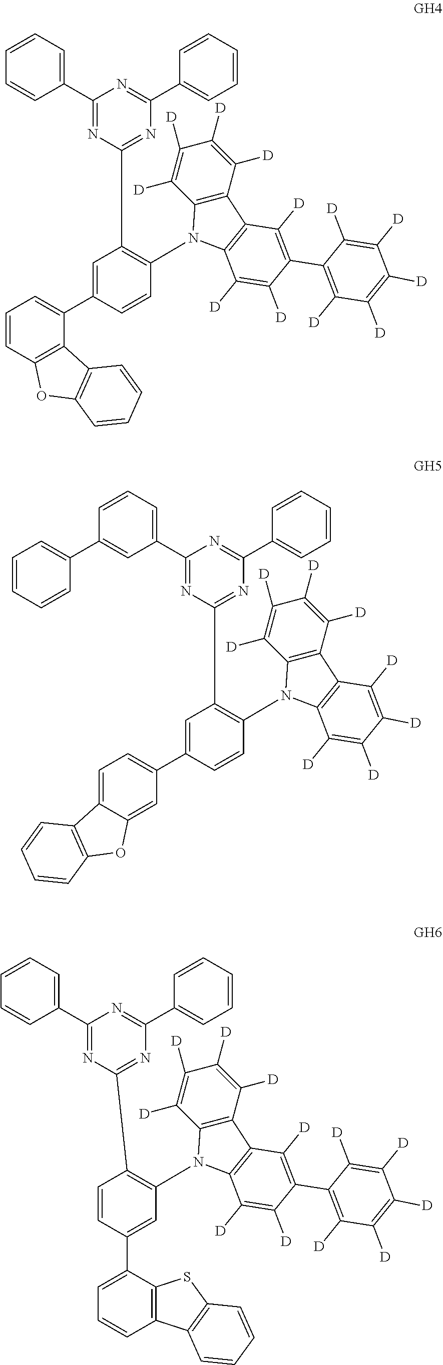 [SMILES: [2H]c1c([2H])c([2H])c(-c2c([2H])c([2H])c3c(c2[2H])c2c([2H])c([2H])c([2H])c([2H])c2n3-c2ccc(-c3cccc4oc5ccccc5c34)cc2-c2nc(-c3ccccc3)nc(-c3ccccc3)n2)c([2H])c1[2H] ; [2H]c1c([2H])c([2H])c2c(c1[2H])c1c([2H])c([2H])c([2H])c([2H])c1n2-c1ccc(-c2ccc3c(c2)oc2ccccc23)cc1-c1nc(-c2ccccc2)nc(-c2cccc(-c3ccccc3)c2)n1 ; [2H]c1c([2H])c([2H])c(-c2c([2H])c([2H])c3c(c2[2H])c2c([2H])c([2H])c([2H])c([2H])c2n3-c2cc(-c3cccc4c3sc3ccccc34)ccc2-c2nc(-c3ccccc3)nc(-c3ccccc3)n2)c([2H])c1[2H]]

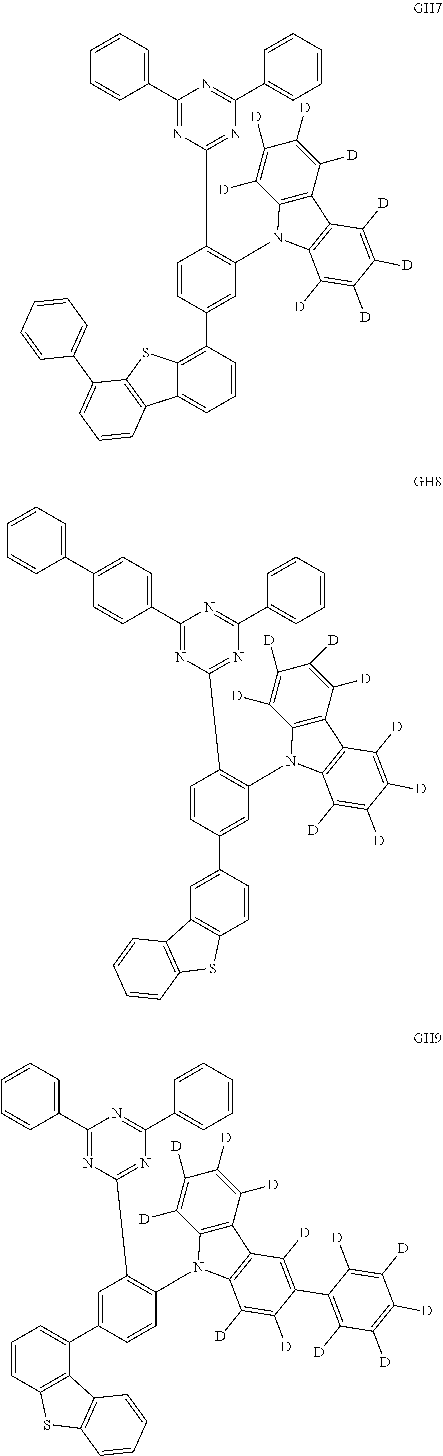 [SMILES: [2H]c1c([2H])c([2H])c2c(c1[2H])c1c([2H])c([2H])c([2H])c([2H])c1n2-c1cc(-c2cccc3c2sc2c(-c4ccccc4)cccc23)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cc(-c2ccc3sc4ccccc4c3c2)ccc1-c1nc(-c2ccccc2)nc(-c2ccc(-c3ccccc3)cc2)n1 ; [2H]c1c([2H])c([2H])c(-c2c([2H])c([2H])c3c(c2[2H])c2c([2H])c([2H])c([2H])c([2H])c2n3-c2ccc(-c3cccc4sc5ccccc5c34)cc2-c2nc(-c3ccccc3)nc(-c3ccccc3)n2)c([2H])c1[2H]]

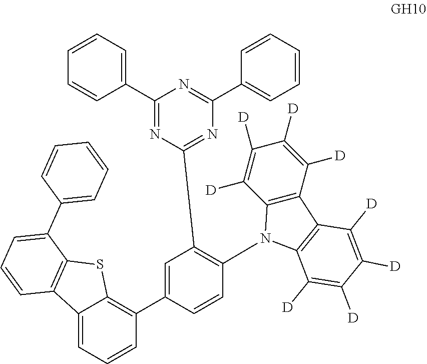 [SMILES: [2H]c1c([2H])c([2H])c2c(c1[2H])c1c([2H])c([2H])c([2H])c([2H])c1n2-c1ccc(-c2cccc3c2sc2c(-c4ccccc4)cccc23)cc1-c1nc(-c2ccccc2)nc(-c2ccccc2)n1]

### Comparative Examples 1 to 5

The organic light emitting devices of Comparative Examples 1 to 5 were manufactured in the same manner as in Example 1, except that in Example 1, the compounds listed in Table 1 below were used instead of GH1. The structures of GH-A to GH-E used in Comparative Examples 1 to 5 are summarized as follows.

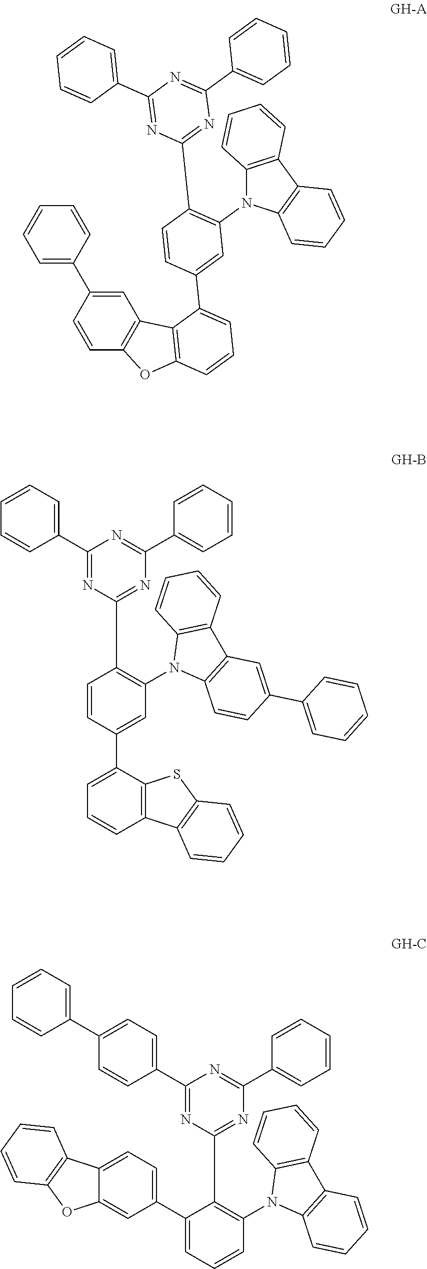 [SMILES: c1ccc(-c2ccc3oc4cccc(-c5ccc(-c6nc(-c7ccccc7)nc(-c7ccccc7)n6)c(-n6c7ccccc7c7ccccc76)c5)c4c3c2)cc1 ; c1ccc(-c2ccc3c(c2)c2ccccc2n3-c2cc(-c3cccc4c3sc3ccccc34)ccc2-c2nc(-c3ccccc3)nc(-c3ccccc3)n2)cc1 ; c1ccc(-c2ccc(-c3nc(-c4ccccc4)nc(-c4c(-c5ccc6c(c5)oc5ccccc56)cccc4-n4c5ccccc5c5ccccc54)n3)cc2)cc1]

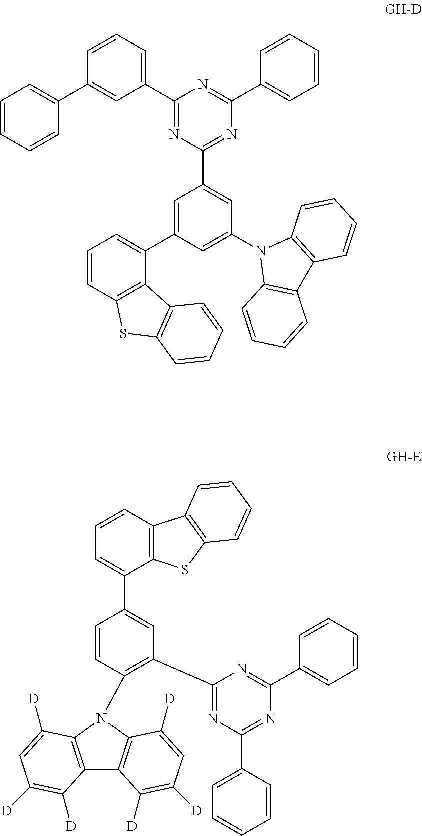 [SMILES: c1ccc(-c2cccc(-c3nc(-c4ccccc4)nc(-c4cc(-c5cccc6sc7ccccc7c56)cc(-n5c6ccccc6c6ccccc65)c4)n3)c2)cc1 ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccccc2)n1]

### Experimental Example

The voltage, efficiency, luminous color, and lifetime (T₉₅) were measured by applying a current to the organic light emitting devices manufactured in Examples 1 to 10 and Comparative Examples 1 to 5, and the results are shown in Table 1 below. At this time, the voltage and efficiency were measured by applying a current density of 10 mA/cm², and T₉₅ means the time (hr) required for the luminance to be reduced to 95% of the initial luminance at a current density of 20 mA/cm².

TABLE 1

| | Light | Voltage | Efficiency | | |
|---|---|---|---|---|---|
| | emitting | (V) | (cd/A) | | T₉₅(hr) |
| | layer | (@10 | (@10 | Luminous | (@20 |
| Category | compound | mA/cm²) | mA/cm²) | color | mA/cm²) |
| Example 1 | GH1 | 3.51 | 90.4 | Green | 160 |
| Example 2 | GH2 | 3.44 | 89.1 | Green | 180 |
| Example 3 | GH3 | 3.55 | 91.7 | Green | 155 |
| Example 4 | GH4 | 3.47 | 90.5 | Green | 160 |
| Example 5 | GH5 | 3.64 | 91.4 | Green | 175 |
| Example 6 | GH6 | 3.48 | 93.2 | Green | 170 |
| Example 7 | GH7 | 3.51 | 93.5 | Green | 180 |
| Example 8 | GH8 | 3.46 | 88.8 | Green | 155 |
| Example 9 | GH9 | 3.49 | 89.1 | Green | 175 |
| Example 10 | GH10 | 3.57 | 92.2 | Green | 175 |
| Comparative | GH-A | 3.46 | 89.3 | Green | 90 |
| Example 1 | | | | | |
| Comparative | GH-B | 3.47 | 93.2 | Green | 95 |
| Example 2 | | | | | |
| Comparative | GH-C | 3.87 | 75.4 | Green | 110 |
| Example 3 | | | | | |
| Comparative | GH-D | 3.75 | 71.8 | Green | 105 |
| Example 4 | | | | | |
| Comparative | GH-E | 3.51 | 88.9 | Green | 130 |
| Example 5 | | | | | |

As shown in Table 1, it can be confirmed that in the case of the organic light emitting devices of Examples manufactured using the compound according to the present disclosure as the host of the light emitting layer exhibits superior performance in terms of voltage, efficiency and/or lifetime as compared to the organic light emitting device of Comparative Examples.

More specifically, it can be seen that the organic light emitting devices of Examples exhibit lower driving voltage, higher efficiency, and significantly improved lifetime properties as compared to the organic light emitting device of Comparative Example 4 using the comparative compound GH-D. Thereby, as a triazine moiety and a carbazole moiety have a distorted structure on the basis of the benzene ring so as to induce steric hindrance, the compound represented by Chemical Formula 1, which exhibits additional CT (charge transfer) properties, can improve device properties, while the comparative compound GH-D does not exhibit CT properties because the carbazolyl group exists at the meta-position of the triazine and thus cannot cause steric hindrance, so it does not contribute to improvement of device properties.

In addition, it can be seen that the organic light emitting devices of Examples exhibit significantly improved lifetime properties as compared to the organic light emitting devices of Comparative Examples 1 and 2 employing the comparative compounds GH-A and GH-B, respectively. This is considered to be because, in the case of comparative compounds GH-A and GH-B, the stability of the molecules is reduced due to an electron deficiency phenomenon in the carbazole moiety. Accordingly, it is confirmed that the compound represented by Chemical Formula 1 has deuterium in the carbazole moiety, unlike the comparative compounds GH-A and GH-B, thereby improving the molecular stability.

Further, in order to compare the effects of the compound represented by Chemical Formula 1 and the compound in which dibenzofuranyl or dibenzothiophenyl is located at the R₁₂ position, the characteristics of the organic light emitting device of Comparative Example 3 employing the comparative compound GH-C were confirmed. As a result, it can be seen that the organic light emitting device of Comparative Example 3 exhibits inferior characteristics in terms of voltage, efficiency, and lifetime compared to the organic light emitting device of Examples. This is considered to be because when all dibenzofuranyl-triazine-carbazole moieties form a distorted structure, the effect of improving molecular stability and electronic properties shown by dibenzofuranyl/dibenzothiophenyl in the compound represented by Chemical Formula 1 was not exhibited.

In addition, in the case of a structure in which a carbazolyl group exists at the ortho-position of the triazine in the benzene ring and a dibenzofuranyl/dibenzothiophenyl group exists at the para-position of the carbazolyl group, the electron donating properties of the dibenzofuranyl/dibenzothiophenyl group directly affect the carbazolyl group, so that an electron deficiency phenomenon of the carbazolyl group may become more severe. Therefore, in the case of a compound having this structure and in which only a part of the carbazolyl group is substituted with deuterium, the increase in lifetime properties of a device employing the compound may not be large due to the instability of the hydrogen atom existing in the carbazolyl group. This can be confirmed by comparing the lifetime data of the organic light emitting devices of Examples and the lifetime data of the organic light emitting device employing the comparative compound GH-E.

[Description of Symbols]

| | |
|---|---|
| 1: substrate | 2: anode |
| 3: organic material layer | 4: cathode |
| 5: hole injection layer | 6: first hole transport layer |
| 7: electron blocking layer | 8: light emitting layer |
| 9: hole blocking layer | 10: electron transport layer |
| 11: electron injection layer | 12: electron injection and transport layer |

1. A compound represented by the following Chemical Formula 1: 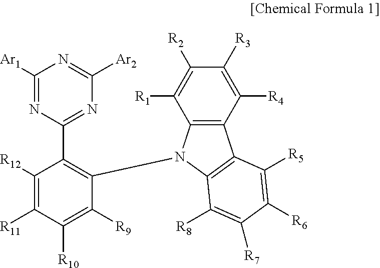
wherein, in Chemical Formula 1,
Ar₁ and Ar₂ are each independently a substituted or unsubstituted C₆₋₆₀ aryl; or a substituted or unsubstituted C₂₋₆₀ heteroaryl containing at least one heteroatom selected from the group consisting of N, O and S,
R₁ to R₈ are each independently deuterium; or a substituted or unsubstituted C₆₋₆₀ aryl, with the proviso that at least one of R₁ to R₈ is deuterium,
any one of R₉ to R₁ is a bond with the following Chemical Formula 2, and the rest are each independently hydrogen or deuterium,
R₁₂ is hydrogen or deuterium, 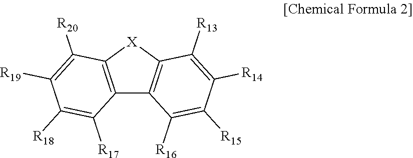
wherein, in Chemical Formula 2,
X is O or S,
any one of R₁₃ to R₁₆ is a bond with the Chemical Formula 1, and the rest are each independently hydrogen; deuterium; or a substituted or unsubstituted C₆₋₆₀ aryl, and
R₁₇ to R₂₀ are each independently hydrogen; deuterium; or a substituted or unsubstituted C₆₋₆₀ aryl.

2. The compound as claimed in claim 1, wherein:
the Chemical Formula 1 is represented by any one of the following Chemical Formulas 1-1 to 1-6: 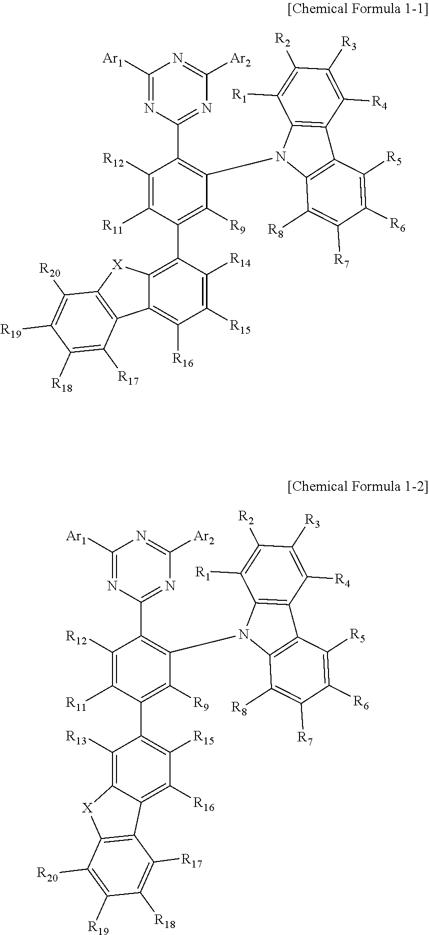 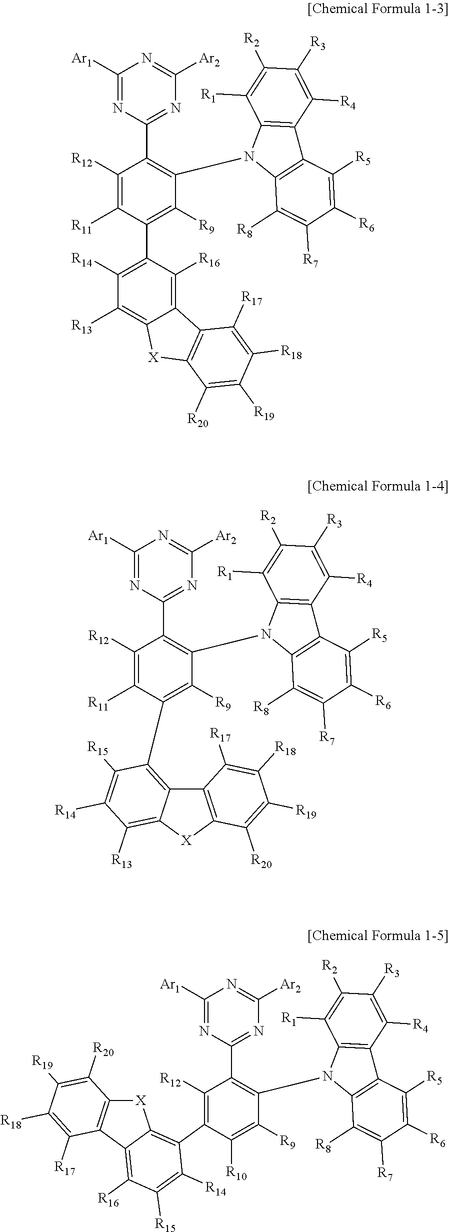 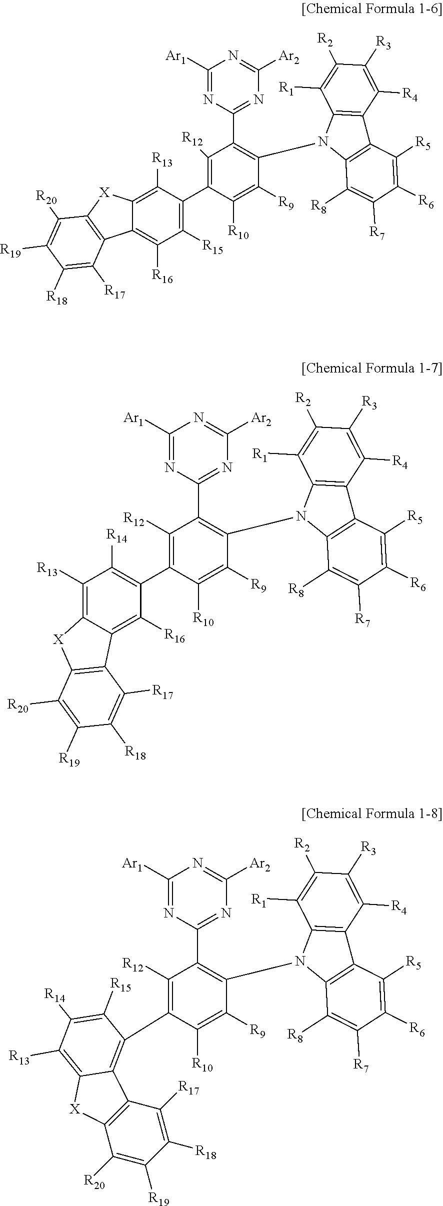
wherein, in Chemical Formulas 1-1 to 1-8,
Ar₁, Ar₂, X, and R₁ to R₂₀ are as defined in claim 1.

3. The compound as claimed in claim 1, wherein:
R₁ to R₈ are each independently deuterium; or phenyl substituted with at least one deuterium.

4. The compound as claimed in claim 1, wherein:
Ar₁ and Ar₂ are each independently phenyl; phenyl substituted with at least one deuterium; biphenylyl; or biphenylyl substituted with at least one deuterium.

5. The compound as claimed in claim 1, wherein:
all the rest of R₉ to R₁ not bonding with Chemical Formula 2 is hydrogen.

6. The compound as claimed in claim 1, wherein:
R₁₂ is hydrogen.

7. The compound as claimed in claim 1, wherein:
the rest of R₁₃ to R₁₆ not bonding with Chemical Formula 1 and R₁₇ to R₂₀ are each independently hydrogen; deuterium; phenyl; or phenyl substituted with at least one deuterium.

8. The compound as claimed in claim 1, wherein:
all of the rest of R₁₃ to R₁₆ not bonding with Chemical Formula 1 and R₁₇ to R₂₀ are hydrogen; or all are deuterium; or one of the rest of R₁₃ to R₁₆ not bonding with Chemical Formula 1 and R₁₇ to R₂₀ is phenyl, and all the rest are hydrogen; or one of the rest of R₁₃ to R₁₆ not bonding with Chemical Formula 1 and R₁₇ to R₂₀ is phenyl substituted with 5 deuteriums, and all the rest are deuterium.

9. The compound as claimed in claim 1, wherein:
the compound represented by Chemical Formula 1 is any one compound selected from anon the following compounds:

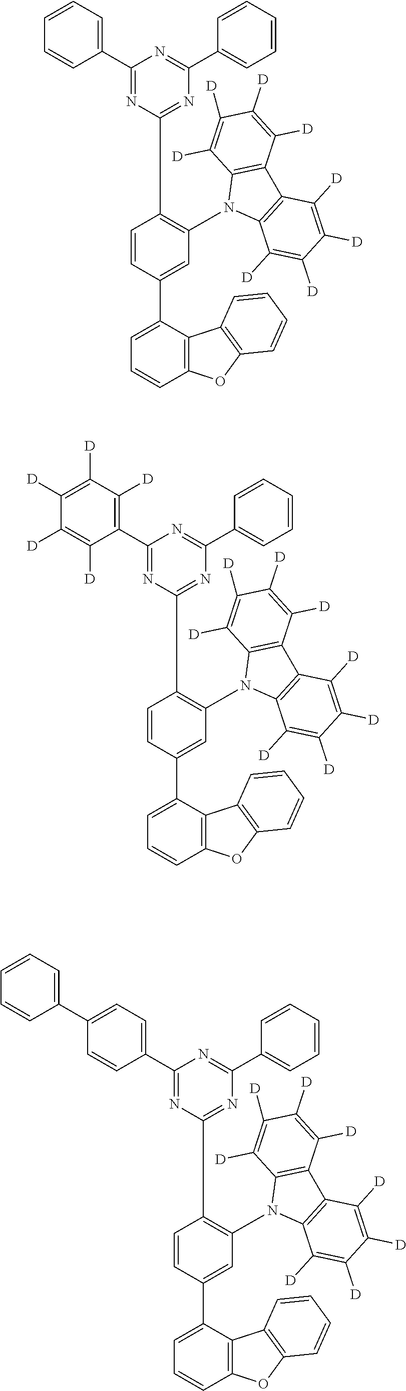 [SMILES: [2H]c1c([2H])c([2H])c2c(c1[2H])c1c([2H])c([2H])c([2H])c([2H])c1n2-c1cc(-c2cccc3oc4ccccc4c23)ccc1-c1nc(-c2ccccc2)nc(-c2ccccc2)n1 ; [2H]c1c([2H])c([2H])c(-c2nc(-c3ccccc3)nc(-c3ccc(-c4cccc5oc6ccccc6c45)cc3-n3c4c([2H])c([2H])c([2H])c([2H])c4c4c([2H])c([2H])c([2H])c([2H])c43)n2)c([2H])c1[2H] ; [2H]c1c([2H])c([2H])c2c(c1[2H])c1c([2H])c([2H])c([2H])c([2H])c1n2-c1cc(-c2cccc3oc4ccccc4c23)ccc1-c1nc(-c2ccccc2)nc(-c2ccc(-c3ccccc3)cc2)n1]

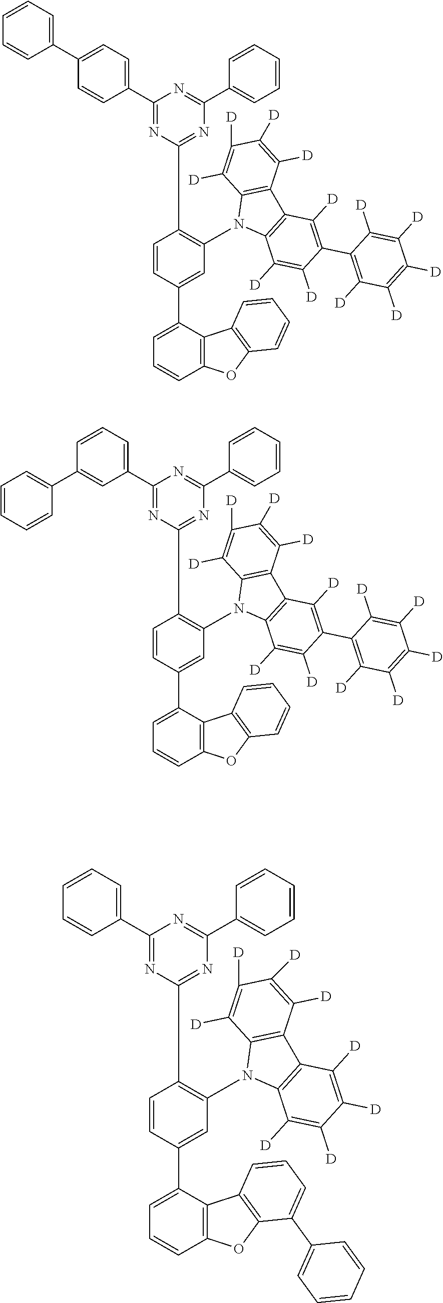 [SMILES: [2H]c1c([2H])c([2H])c(-c2c([2H])c([2H])c3c(c2[2H])c2c([2H])c([2H])c([2H])c([2H])c2n3-c2cc(-c3cccc4oc5ccccc5c34)ccc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2cc(-c3cccc4oc5ccccc5c34)ccc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1cc(-c2cccc3oc4c(-c5ccccc5)cccc4c23)ccc1-c1nc(-c2ccccc2)nc(-c2ccccc2)n1]

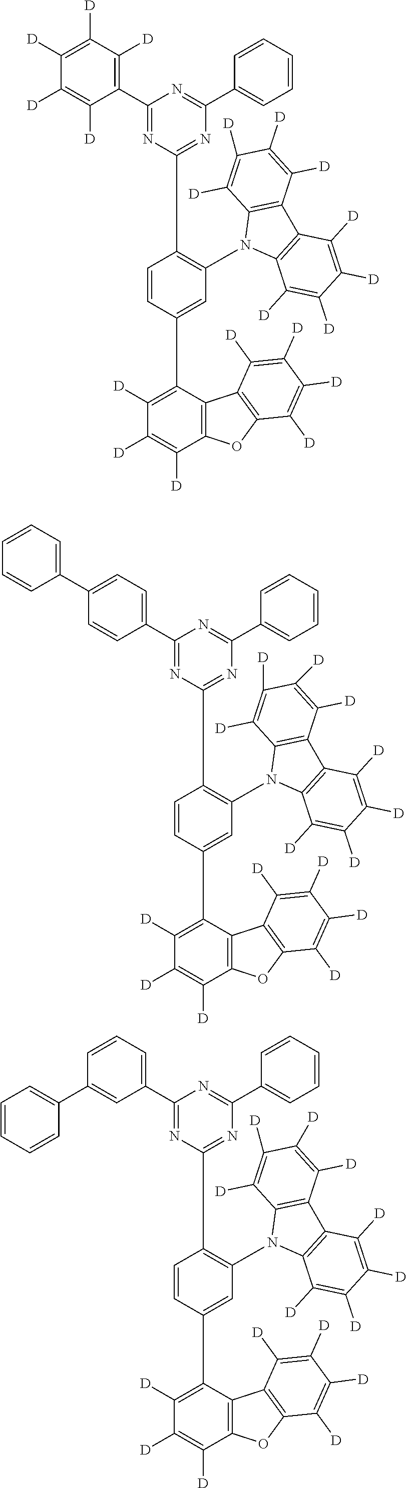

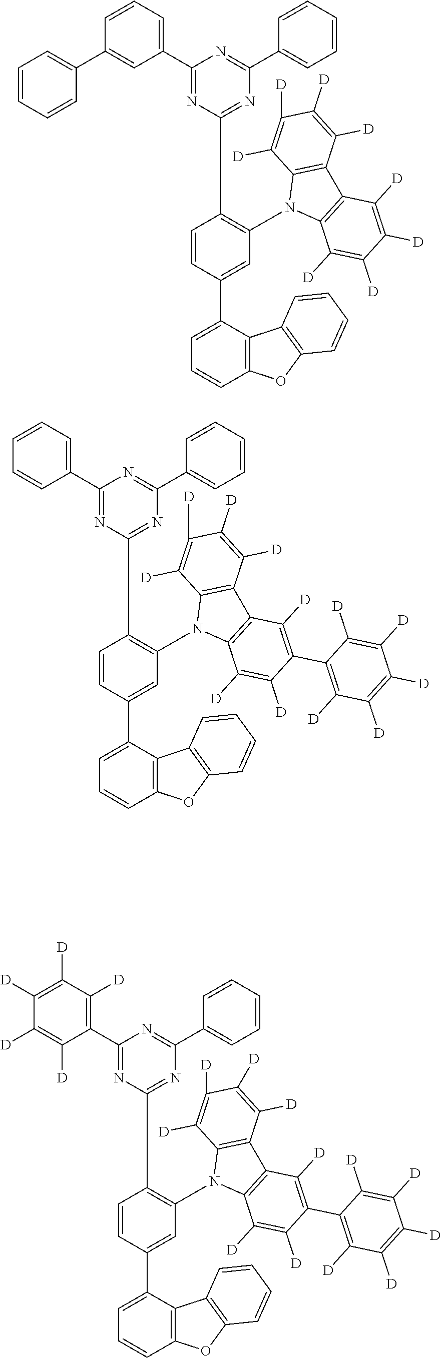

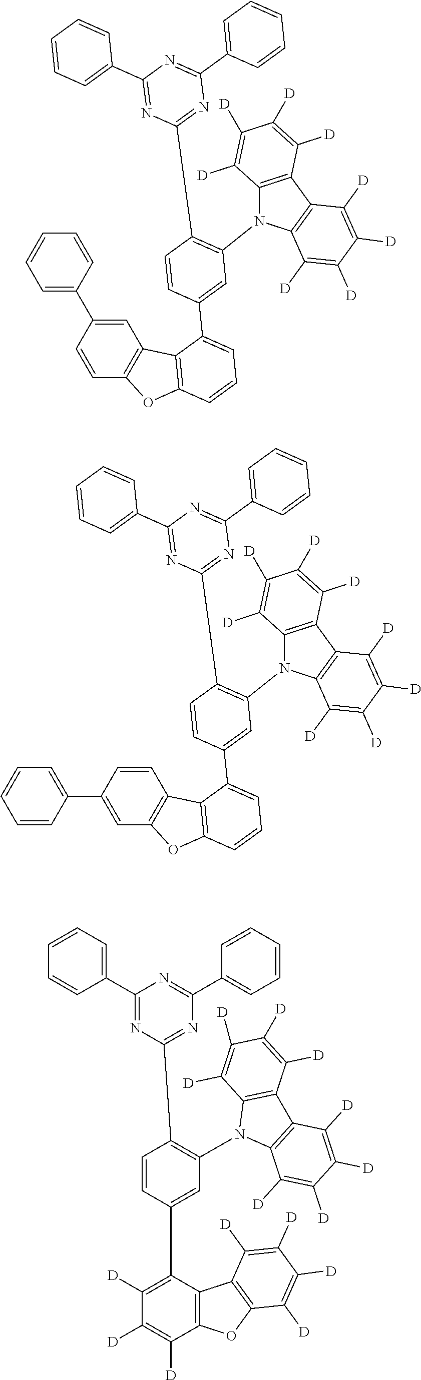 [SMILES: [2H]c1c([2H])c([2H])c2c(c1[2H])c1c([2H])c([2H])c([2H])c([2H])c1n2-c1cc(-c2cccc3oc4ccc(-c5ccccc5)cc4c23)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cc(-c2cccc3oc4cc(-c5ccccc5)ccc4c23)ccc1-c1nc(-c2ccccc2)nc(-c2ccccc2)n1 ; [2H]c1c([2H])c([2H])c2c(oc3c([2H])c([2H])c([2H])c(-c4ccc(-c5nc(-c6ccccc6)nc(-c6ccccc6)n5)c(-n5c6c([2H])c([2H])c([2H])c([2H])c6c6c([2H])c([2H])c([2H])c([2H])c65)c4)c32)c1[2H]]

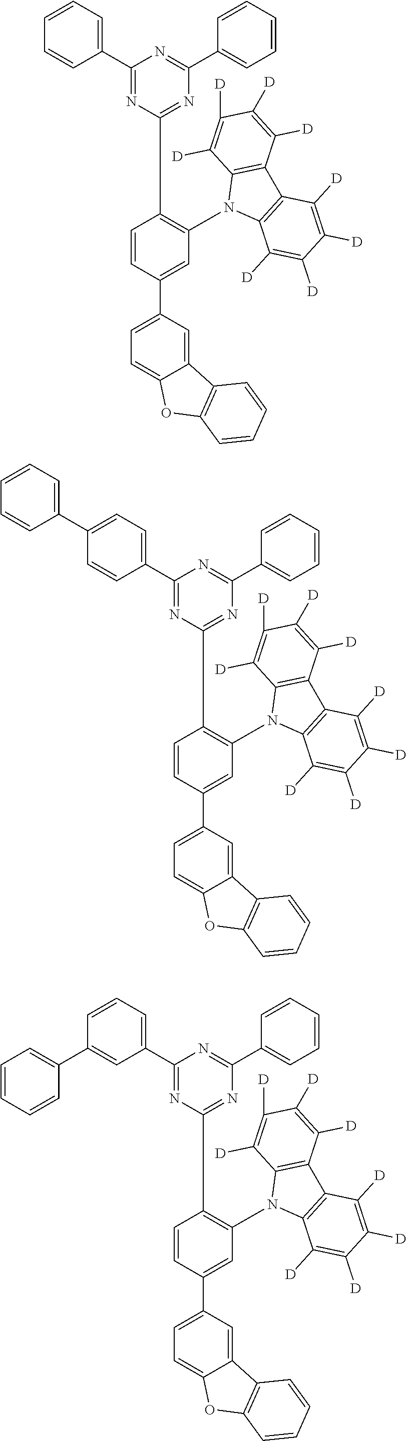

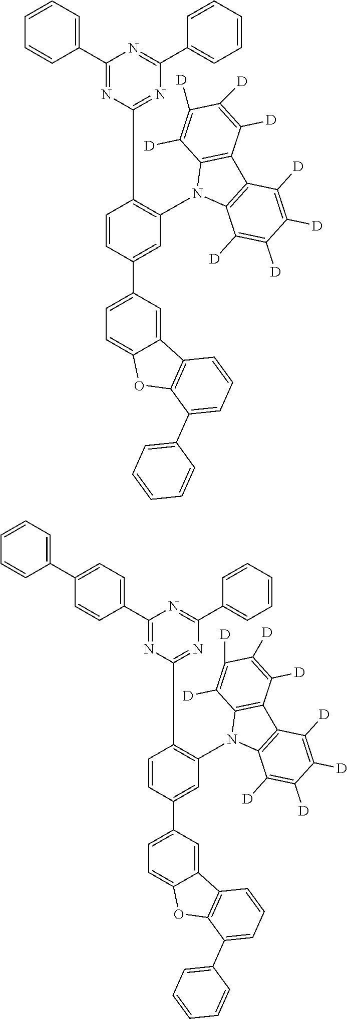 [SMILES: [2H]c1c([2H])c([2H])c2c(c1[2H])c1c([2H])c([2H])c([2H])c([2H])c1n2-c1cc(-c2ccc3oc4c(-c5ccccc5)cccc4c3c2)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cc(-c2ccc3oc4c(-c5ccccc5)cccc4c3c2)ccc1-c1nc(-c2ccccc2)nc(-c2ccc(-c3ccccc3)cc2)n1]

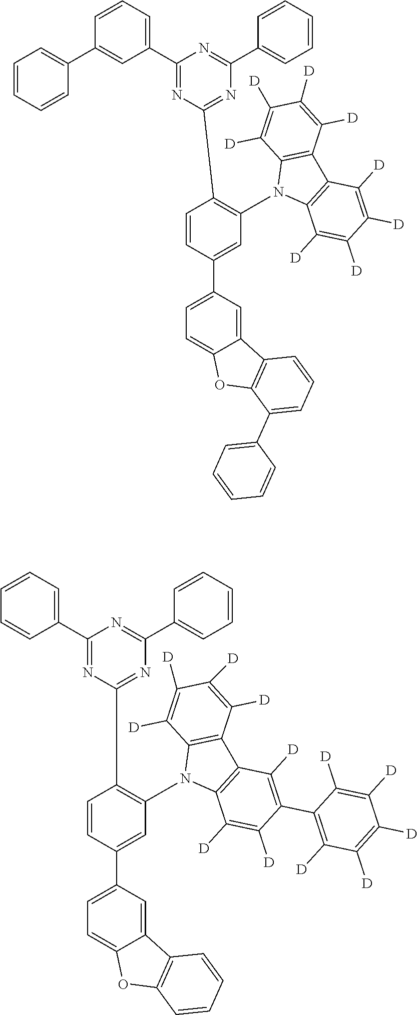 [SMILES: [2H]c1c([2H])c([2H])c2c(c1[2H])c1c([2H])c([2H])c([2H])c([2H])c1n2-c1cc(-c2ccc3oc4c(-c5ccccc5)cccc4c3c2)ccc1-c1nc(-c2ccccc2)nc(-c2cccc(-c3ccccc3)c2)n1 ; [2H]c1c([2H])c([2H])c(-c2c([2H])c([2H])c3c(c2[2H])c2c([2H])c([2H])c([2H])c([2H])c2n3-c2cc(-c3ccc4oc5ccccc5c4c3)ccc2-c2nc(-c3ccccc3)nc(-c3ccccc3)n2)c([2H])c1[2H]]

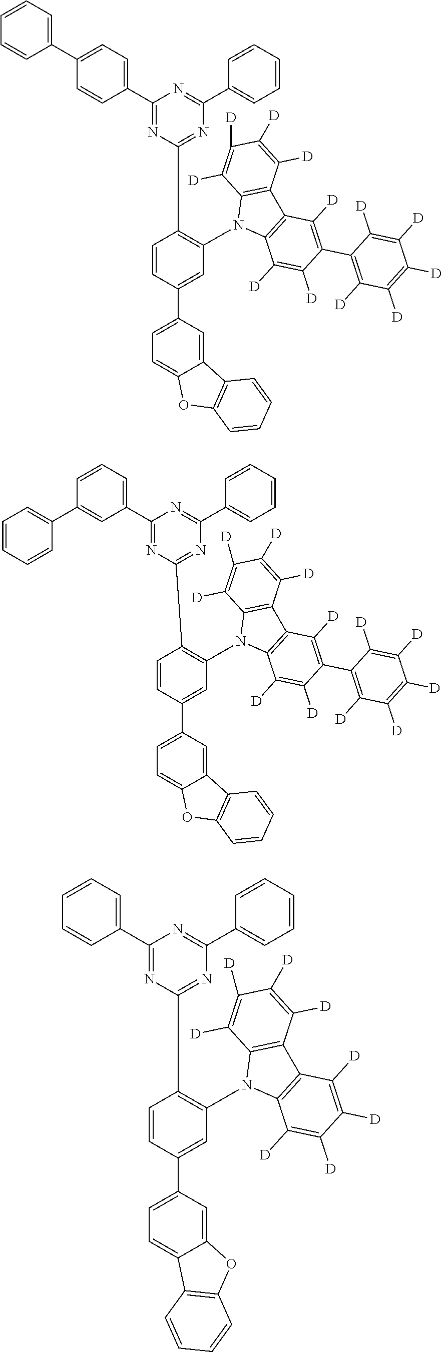

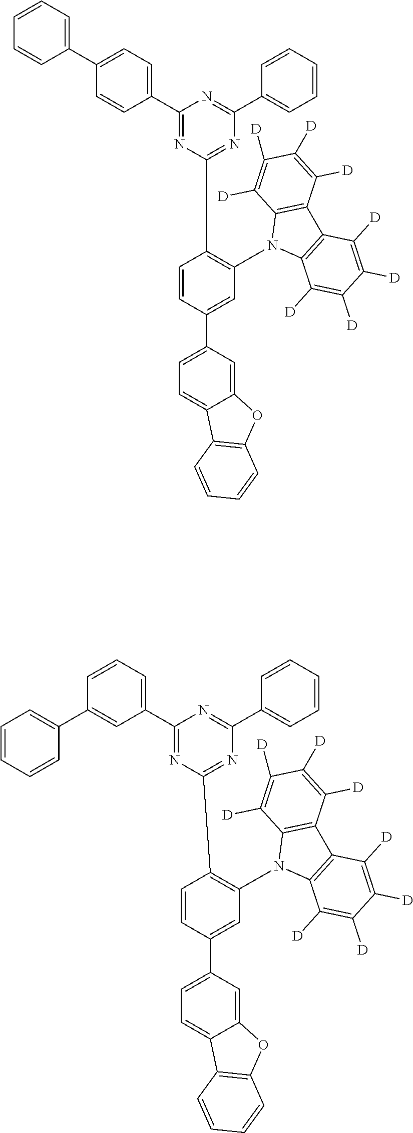 [SMILES: [2H]c1c([2H])c([2H])c2c(c1[2H])c1c([2H])c([2H])c([2H])c([2H])c1n2-c1cc(-c2ccc3c(c2)oc2ccccc23)ccc1-c1nc(-c2ccccc2)nc(-c2ccc(-c3ccccc3)cc2)n1 ; [2H]c1c([2H])c([2H])c2c(c1[2H])c1c([2H])c([2H])c([2H])c([2H])c1n2-c1cc(-c2ccc3c(c2)oc2ccccc23)ccc1-c1nc(-c2ccccc2)nc(-c2cccc(-c3ccccc3)c2)n1]

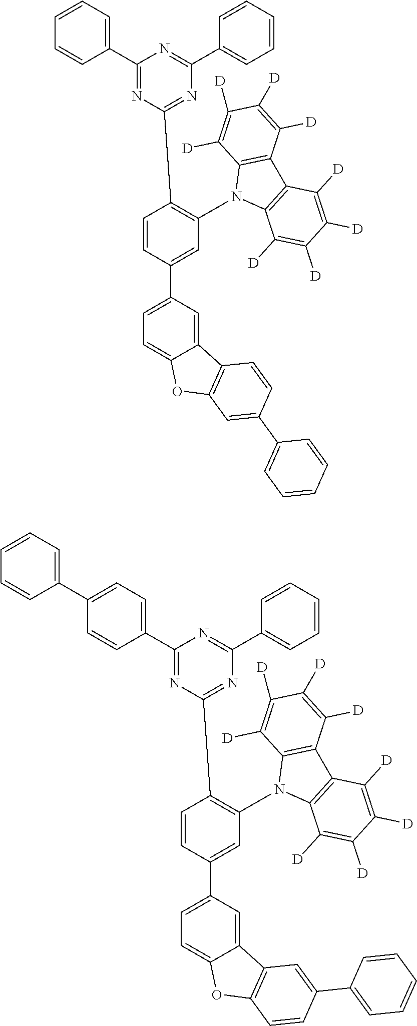 [SMILES: [2H]c1c([2H])c([2H])c2c(c1[2H])c1c([2H])c([2H])c([2H])c([2H])c1n2-c1cc(-c2ccc3oc4cc(-c5ccccc5)ccc4c3c2)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cc(-c2ccc3oc4ccc(-c5ccccc5)cc4c3c2)ccc1-c1nc(-c2ccccc2)nc(-c2ccc(-c3ccccc3)cc2)n1]

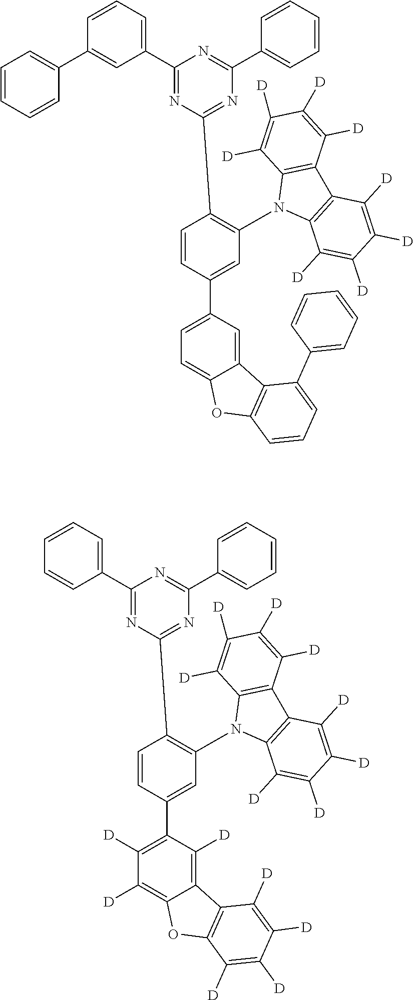 [SMILES: [2H]c1c([2H])c([2H])c2c(c1[2H])c1c([2H])c([2H])c([2H])c([2H])c1n2-c1cc(-c2ccc3oc4cccc(-c5ccccc5)c4c3c2)ccc1-c1nc(-c2ccccc2)nc(-c2cccc(-c3ccccc3)c2)n1 ; [2H]c1c([2H])c([2H])c2c(oc3c([2H])c([2H])c(-c4ccc(-c5nc(-c6ccccc6)nc(-c6ccccc6)n5)c(-n5c6c([2H])c([2H])c([2H])c([2H])c6c6c([2H])c([2H])c([2H])c([2H])c65)c4)c([2H])c32)c1[2H]]

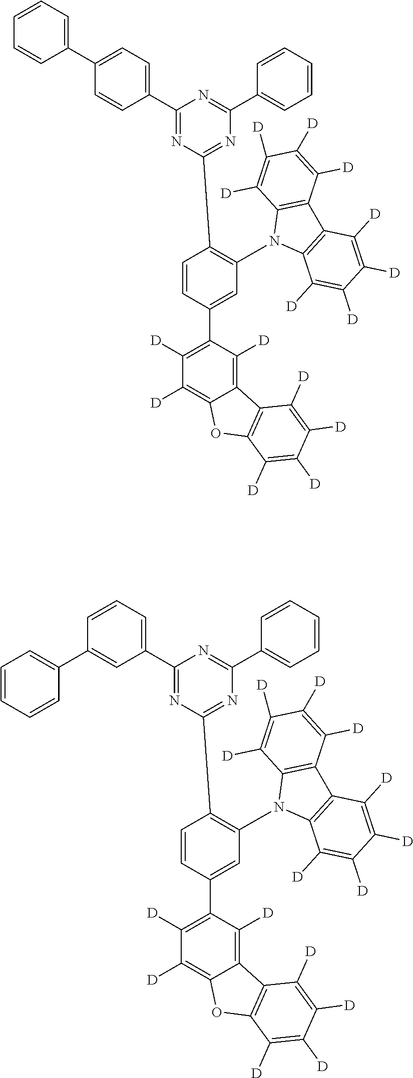 [SMILES: [2H]c1c([2H])c([2H])c2c(oc3c([2H])c([2H])c(-c4ccc(-c5nc(-c6ccccc6)nc(-c6ccc(-c7ccccc7)cc6)n5)c(-n5c6c([2H])c([2H])c([2H])c([2H])c6c6c([2H])c([2H])c([2H])c([2H])c65)c4)c([2H])c32)c1[2H] ; [2H]c1c([2H])c([2H])c2c(oc3c([2H])c([2H])c(-c4ccc(-c5nc(-c6ccccc6)nc(-c6cccc(-c7ccccc7)c6)n5)c(-n5c6c([2H])c([2H])c([2H])c([2H])c6c6c([2H])c([2H])c([2H])c([2H])c65)c4)c([2H])c32)c1[2H]]

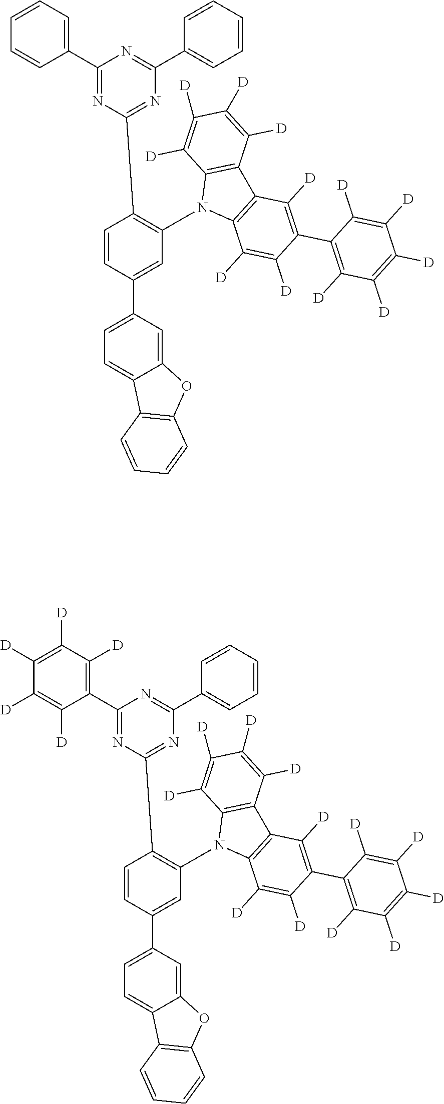 [SMILES: [2H]c1c([2H])c([2H])c(-c2c([2H])c([2H])c3c(c2[2H])c2c([2H])c([2H])c([2H])c([2H])c2n3-c2cc(-c3ccc4c(c3)oc3ccccc34)ccc2-c2nc(-c3ccccc3)nc(-c3ccccc3)n2)c([2H])c1[2H] ; [2H]c1c([2H])c([2H])c(-c2nc(-c3ccccc3)nc(-c3ccc(-c4ccc5c(c4)oc4ccccc45)cc3-n3c4c([2H])c([2H])c([2H])c([2H])c4c4c([2H])c(-c5c([2H])c([2H])c([2H])c([2H])c5[2H])c([2H])c([2H])c43)n2)c([2H])c1[2H]]

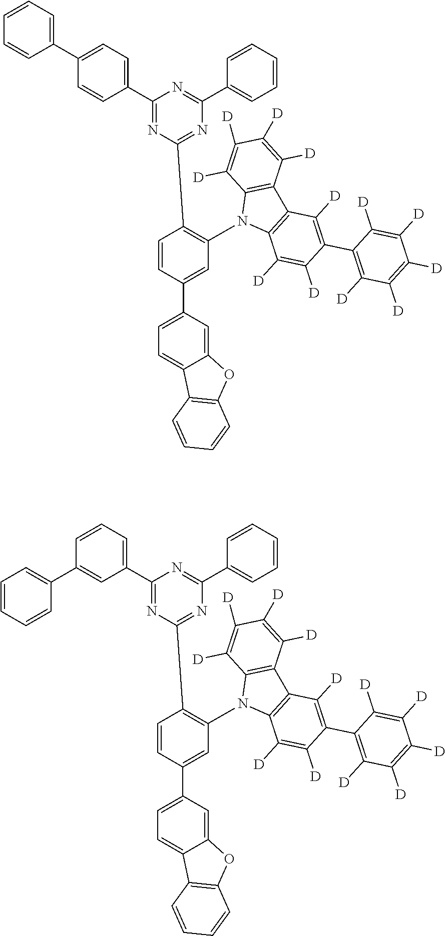 [SMILES: [2H]c1c([2H])c([2H])c(-c2c([2H])c([2H])c3c(c2[2H])c2c([2H])c([2H])c([2H])c([2H])c2n3-c2cc(-c3ccc4c(c3)oc3ccccc34)ccc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2cc(-c3ccc4c(c3)oc3ccccc34)ccc2-c2nc(-c3ccccc3)nc(-c3cccc(-c4ccccc4)c3)n2)c([2H])c1[2H]]

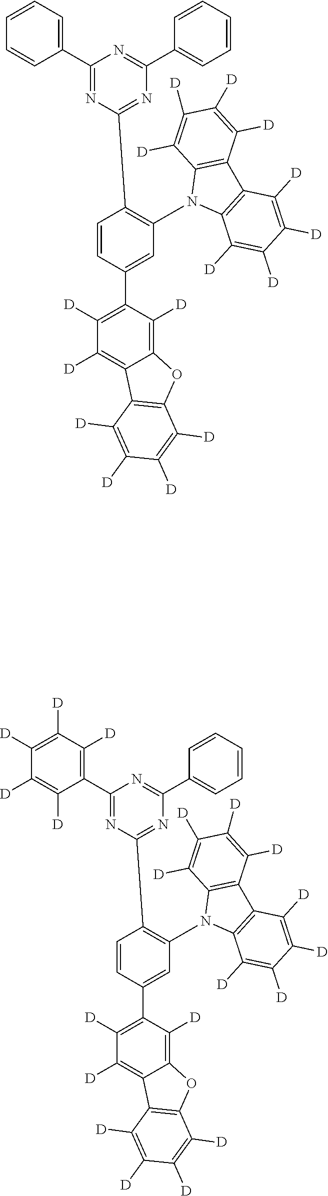 [SMILES: [2H]c1c([2H])c([2H])c2c(oc3c([2H])c(-c4ccc(-c5nc(-c6ccccc6)nc(-c6ccccc6)n5)c(-n5c6c([2H])c([2H])c([2H])c([2H])c6c6c([2H])c([2H])c([2H])c([2H])c65)c4)c([2H])c([2H])c32)c1[2H] ; [2H]c1c([2H])c([2H])c(-c2nc(-c3ccccc3)nc(-c3ccc(-c4c([2H])c([2H])c5c(oc6c([2H])c([2H])c([2H])c([2H])c65)c4[2H])cc3-n3c4c([2H])c([2H])c([2H])c([2H])c4c4c([2H])c([2H])c([2H])c([2H])c43)n2)c([2H])c1[2H]]

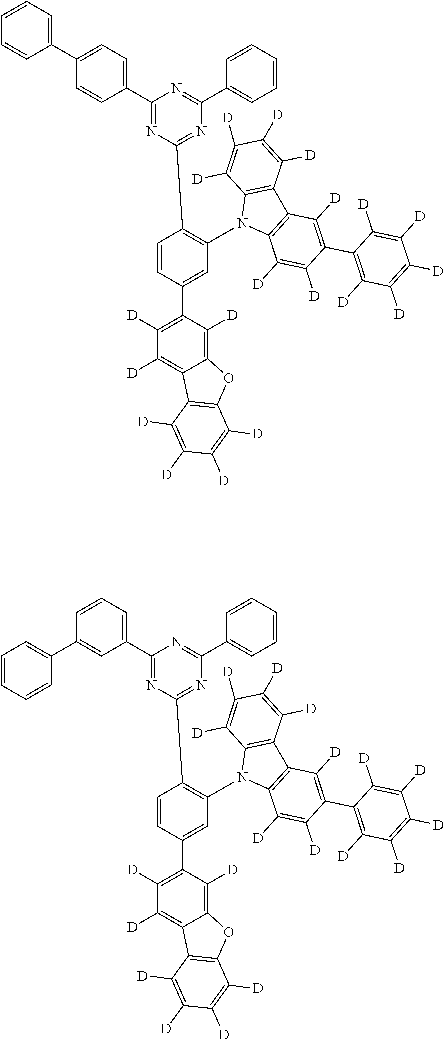 [SMILES: [2H]c1c([2H])c([2H])c(-c2c([2H])c([2H])c3c(c2[2H])c2c([2H])c([2H])c([2H])c([2H])c2n3-c2cc(-c3c([2H])c([2H])c4c(oc5c([2H])c([2H])c([2H])c([2H])c54)c3[2H])ccc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2cc(-c3c([2H])c([2H])c4c(oc5c([2H])c([2H])c([2H])c([2H])c54)c3[2H])ccc2-c2nc(-c3ccccc3)nc(-c3cccc(-c4ccccc4)c3)n2)c([2H])c1[2H]]

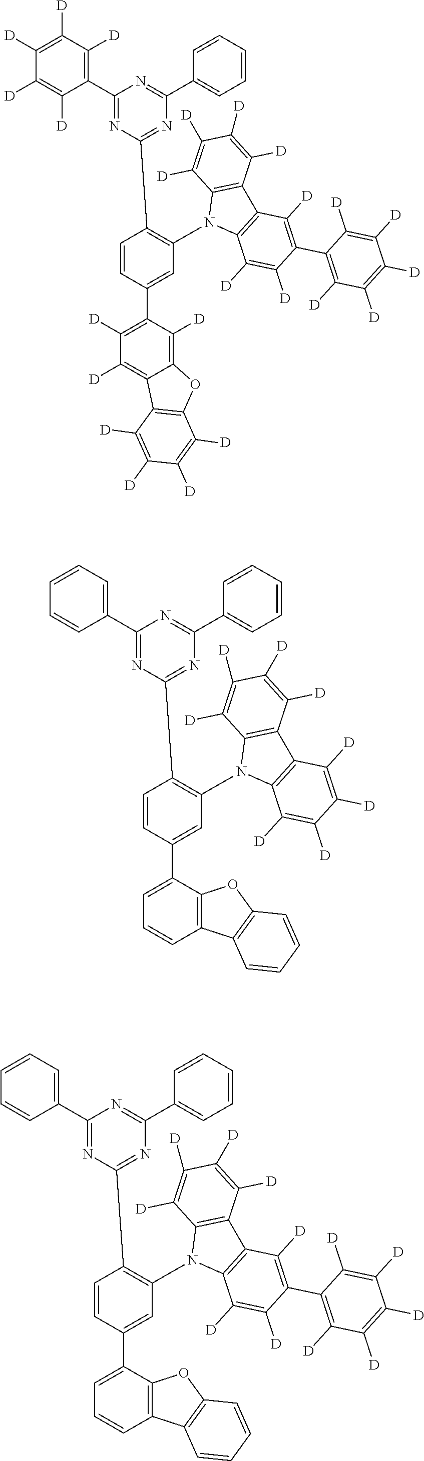

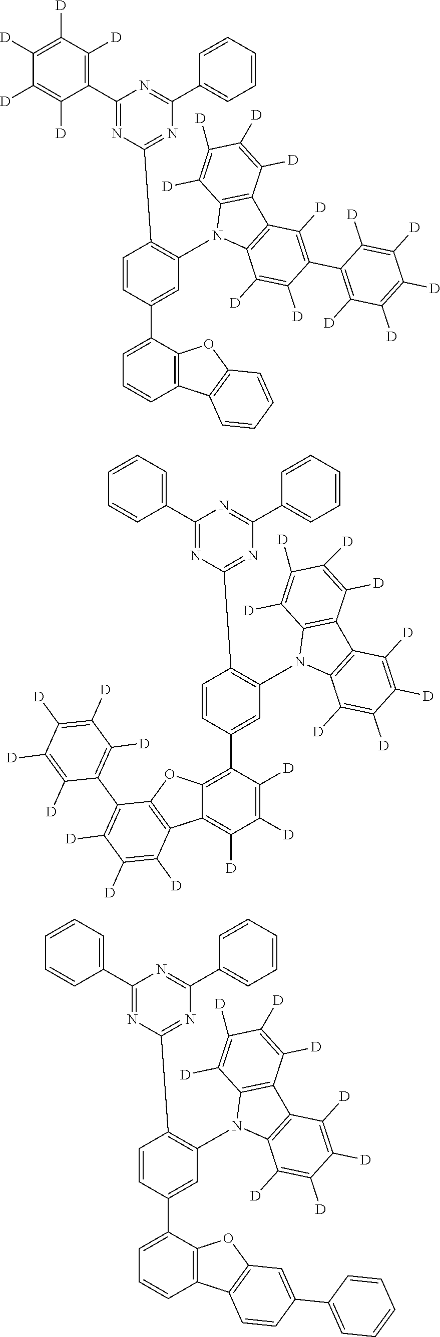

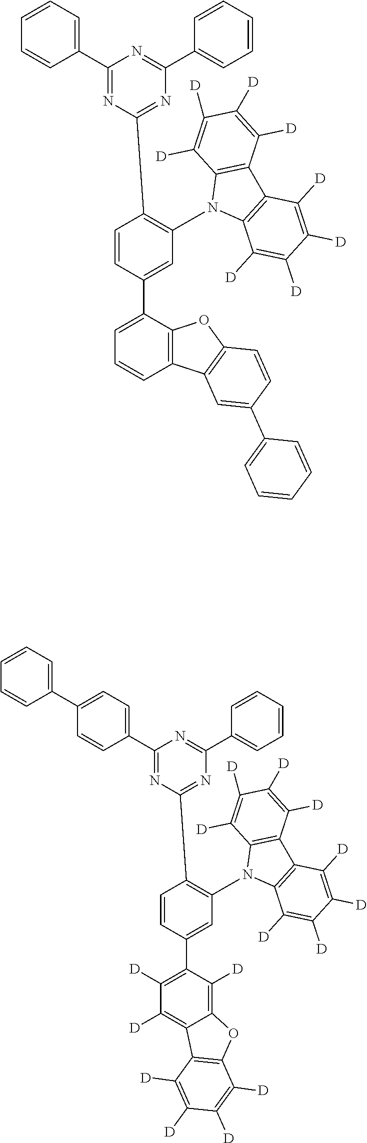 [SMILES: [2H]c1c([2H])c([2H])c2c(c1[2H])c1c([2H])c([2H])c([2H])c([2H])c1n2-c1cc(-c2cccc3c2oc2ccc(-c4ccccc4)cc23)ccc1-c1nc(-c2ccccc2)nc(-c2ccccc2)n1 ; [2H]c1c([2H])c([2H])c2c(oc3c([2H])c(-c4ccc(-c5nc(-c6ccccc6)nc(-c6ccc(-c7ccccc7)cc6)n5)c(-n5c6c([2H])c([2H])c([2H])c([2H])c6c6c([2H])c([2H])c([2H])c([2H])c65)c4)c([2H])c([2H])c32)c1[2H]]

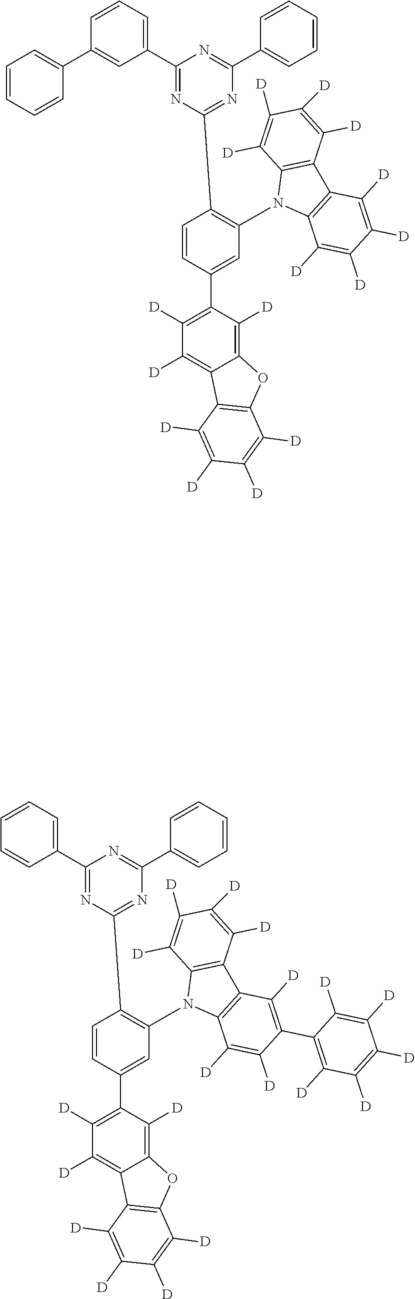 [SMILES: [2H]c1c([2H])c([2H])c2c(oc3c([2H])c(-c4ccc(-c5nc(-c6ccccc6)nc(-c6cccc(-c7ccccc7)c6)n5)c(-n5c6c([2H])c([2H])c([2H])c([2H])c6c6c([2H])c([2H])c([2H])c([2H])c65)c4)c([2H])c([2H])c32)c1[2H] ; [2H]c1c([2H])c([2H])c(-c2c([2H])c([2H])c3c(c2[2H])c2c([2H])c([2H])c([2H])c([2H])c2n3-c2cc(-c3c([2H])c([2H])c4c(oc5c([2H])c([2H])c([2H])c([2H])c54)c3[2H])ccc2-c2nc(-c3ccccc3)nc(-c3ccccc3)n2)c([2H])c1[2H]]

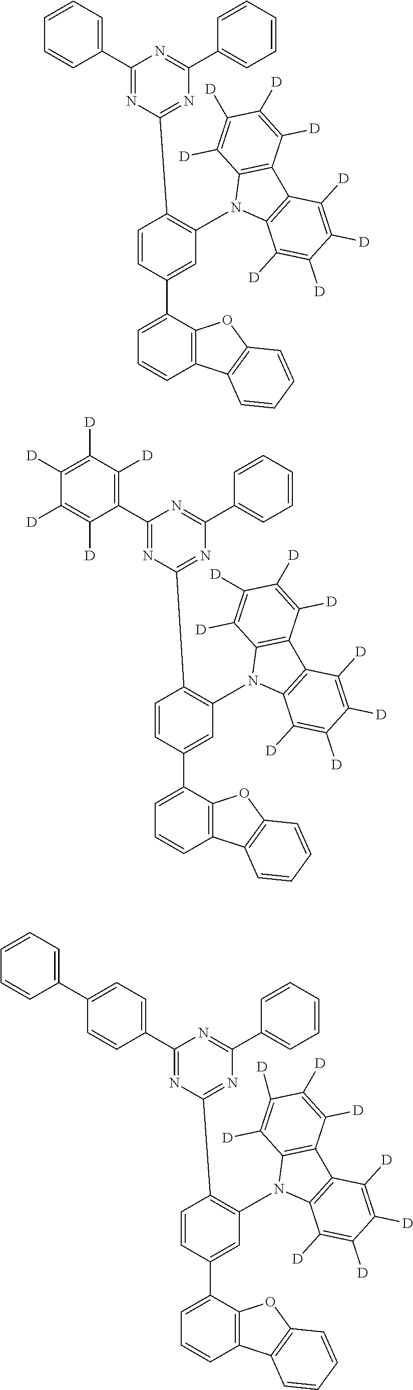 [SMILES: [2H]c1c([2H])c([2H])c2c(c1[2H])c1c([2H])c([2H])c([2H])c([2H])c1n2-c1cc(-c2cccc3c2oc2ccccc23)ccc1-c1nc(-c2ccccc2)nc(-c2ccccc2)n1 ; [2H]c1c([2H])c([2H])c(-c2nc(-c3ccccc3)nc(-c3ccc(-c4cccc5c4oc4ccccc45)cc3-n3c4c([2H])c([2H])c([2H])c([2H])c4c4c([2H])c([2H])c([2H])c([2H])c43)n2)c([2H])c1[2H] ; [2H]c1c([2H])c([2H])c2c(c1[2H])c1c([2H])c([2H])c([2H])c([2H])c1n2-c1cc(-c2cccc3c2oc2ccccc23)ccc1-c1nc(-c2ccccc2)nc(-c2ccc(-c3ccccc3)cc2)n1]

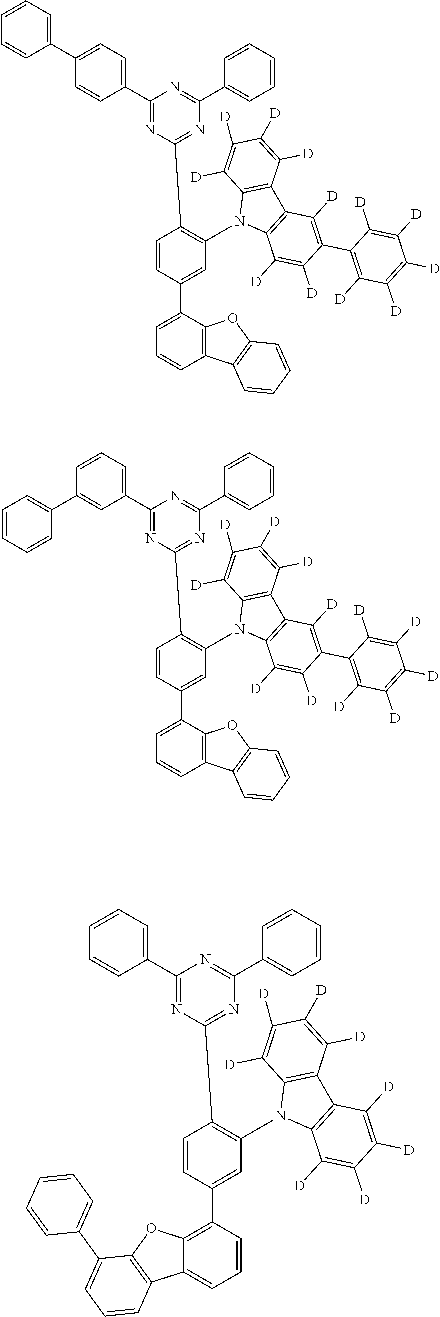 [SMILES: [2H]c1c([2H])c([2H])c(-c2c([2H])c([2H])c3c(c2[2H])c2c([2H])c([2H])c([2H])c([2H])c2n3-c2cc(-c3cccc4c3oc3ccccc34)ccc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2cc(-c3cccc4c3oc3ccccc34)ccc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1cc(-c2cccc3c2oc2c(-c4ccccc4)cccc23)ccc1-c1nc(-c2ccccc2)nc(-c2ccccc2)n1]

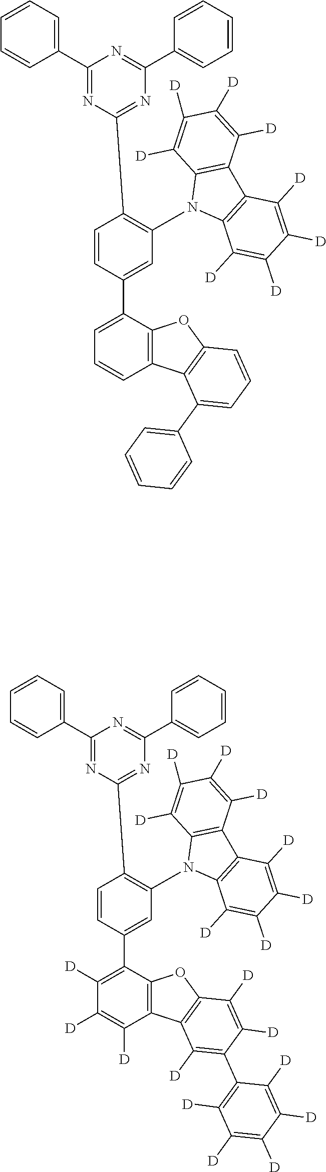 [SMILES: [2H]c1c([2H])c([2H])c2c(c1[2H])c1c([2H])c([2H])c([2H])c([2H])c1n2-c1cc(-c2cccc3c2oc2cccc(-c4ccccc4)c23)ccc1-c1nc(-c2ccccc2)nc(-c2ccccc2)n1 ; [2H]c1c([2H])c([2H])c(-c2c([2H])c([2H])c3oc4c(-c5ccc(-c6nc(-c7ccccc7)nc(-c7ccccc7)n6)c(-n6c7c([2H])c([2H])c([2H])c([2H])c7c7c([2H])c([2H])c([2H])c([2H])c76)c5)c([2H])c([2H])c([2H])c4c3c2[2H])c([2H])c1[2H]]

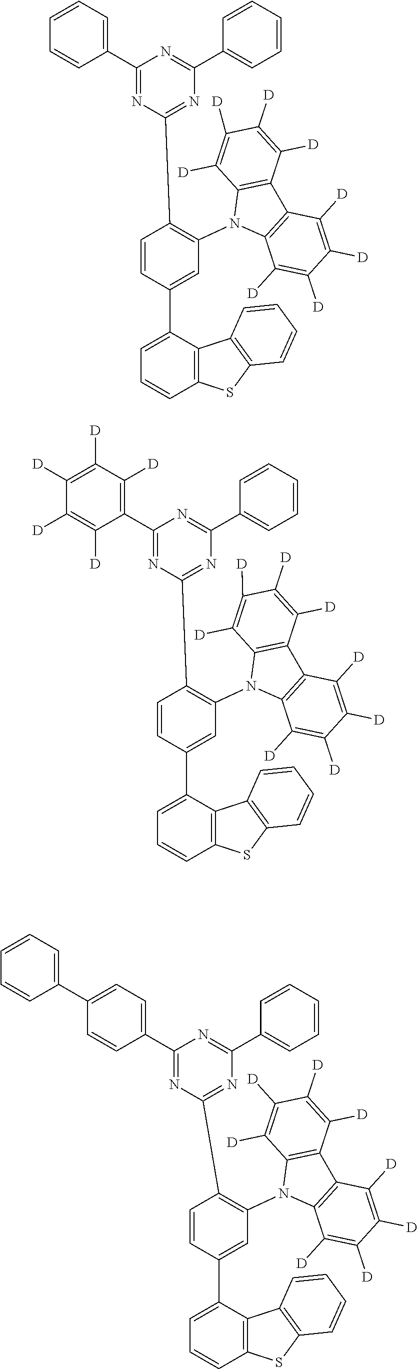 [SMILES: [2H]c1c([2H])c([2H])c2c(c1[2H])c1c([2H])c([2H])c([2H])c([2H])c1n2-c1cc(-c2cccc3sc4ccccc4c23)ccc1-c1nc(-c2ccccc2)nc(-c2ccccc2)n1 ; [2H]c1c([2H])c([2H])c(-c2nc(-c3ccccc3)nc(-c3ccc(-c4cccc5sc6ccccc6c45)cc3-n3c4c([2H])c([2H])c([2H])c([2H])c4c4c([2H])c([2H])c([2H])c([2H])c43)n2)c([2H])c1[2H] ; [2H]c1c([2H])c([2H])c2c(c1[2H])c1c([2H])c([2H])c([2H])c([2H])c1n2-c1cc(-c2cccc3sc4ccccc4c23)ccc1-c1nc(-c2ccccc2)nc(-c2ccc(-c3ccccc3)cc2)n1]

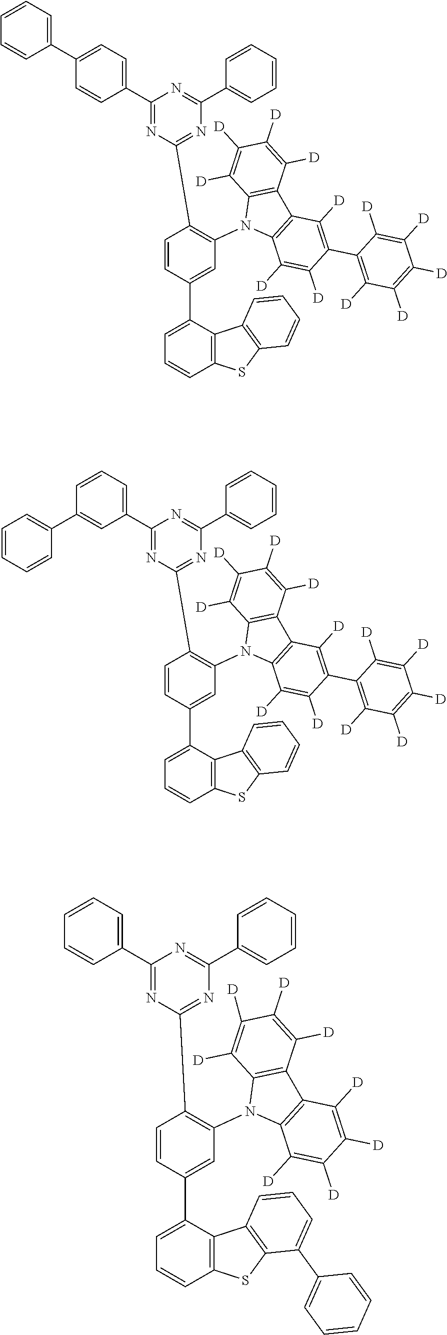 [SMILES: [2H]c1c([2H])c([2H])c(-c2c([2H])c([2H])c3c(c2[2H])c2c([2H])c([2H])c([2H])c([2H])c2n3-c2cc(-c3cccc4sc5ccccc5c34)ccc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2cc(-c3cccc4sc5ccccc5c34)ccc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1cc(-c2cccc3sc4c(-c5ccccc5)cccc4c23)ccc1-c1nc(-c2ccccc2)nc(-c2ccccc2)n1]

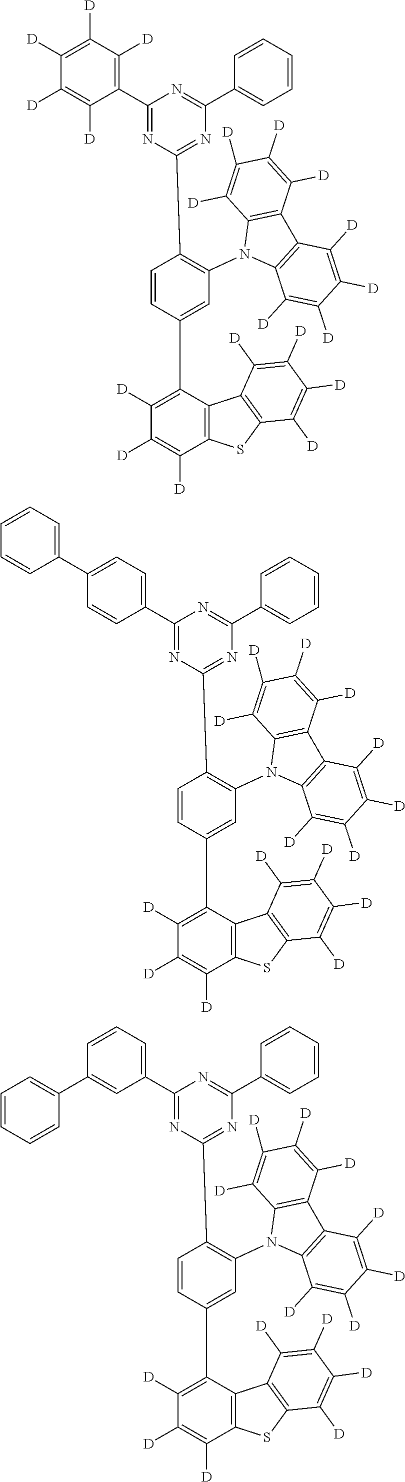

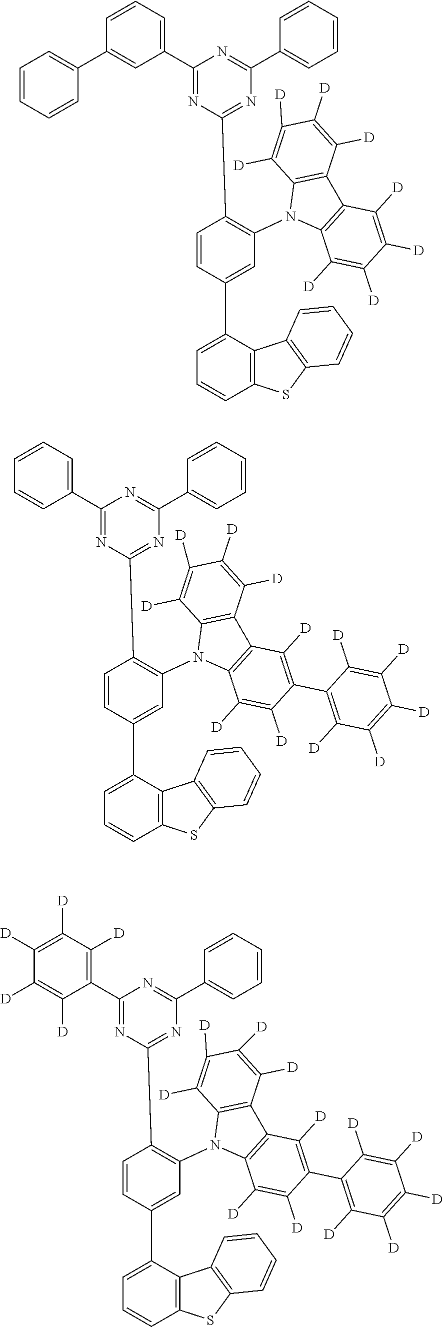

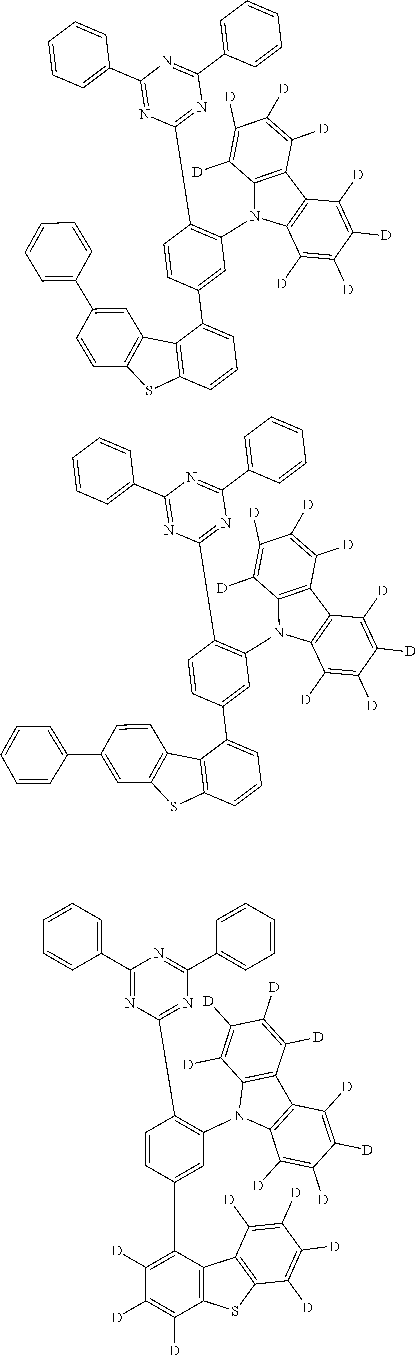 [SMILES: [2H]c1c([2H])c([2H])c2c(c1[2H])c1c([2H])c([2H])c([2H])c([2H])c1n2-c1cc(-c2cccc3sc4ccc(-c5ccccc5)cc4c23)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cc(-c2cccc3sc4cc(-c5ccccc5)ccc4c23)ccc1-c1nc(-c2ccccc2)nc(-c2ccccc2)n1 ; [2H]c1c([2H])c([2H])c2c(sc3c([2H])c([2H])c([2H])c(-c4ccc(-c5nc(-c6ccccc6)nc(-c6ccccc6)n5)c(-n5c6c([2H])c([2H])c([2H])c([2H])c6c6c([2H])c([2H])c([2H])c([2H])c65)c4)c32)c1[2H]]

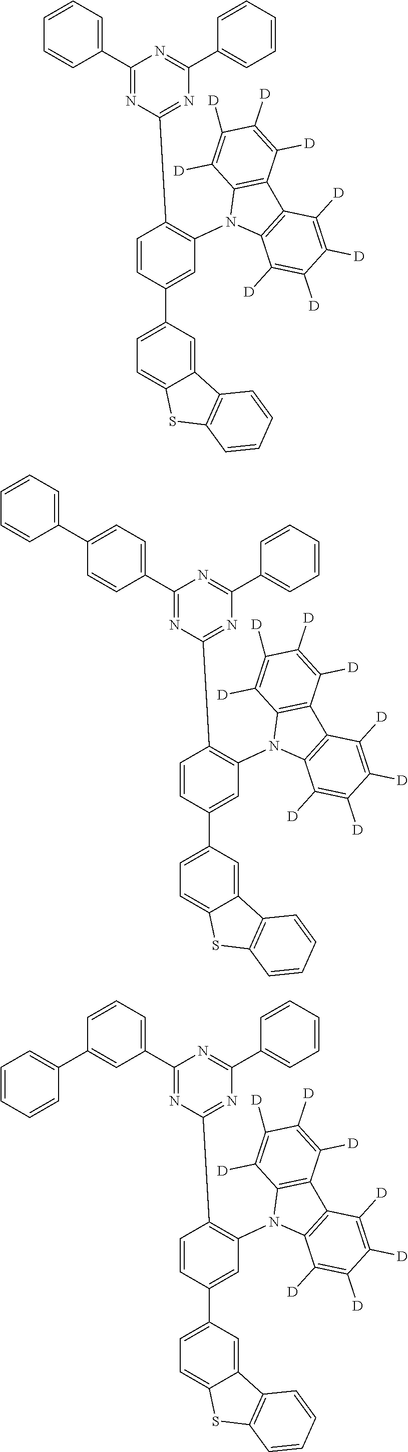

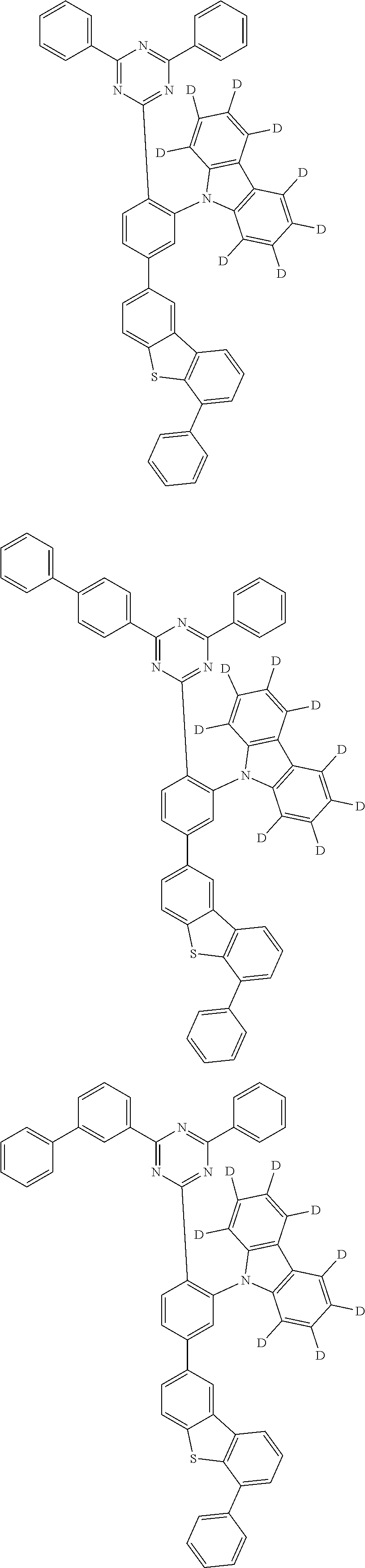

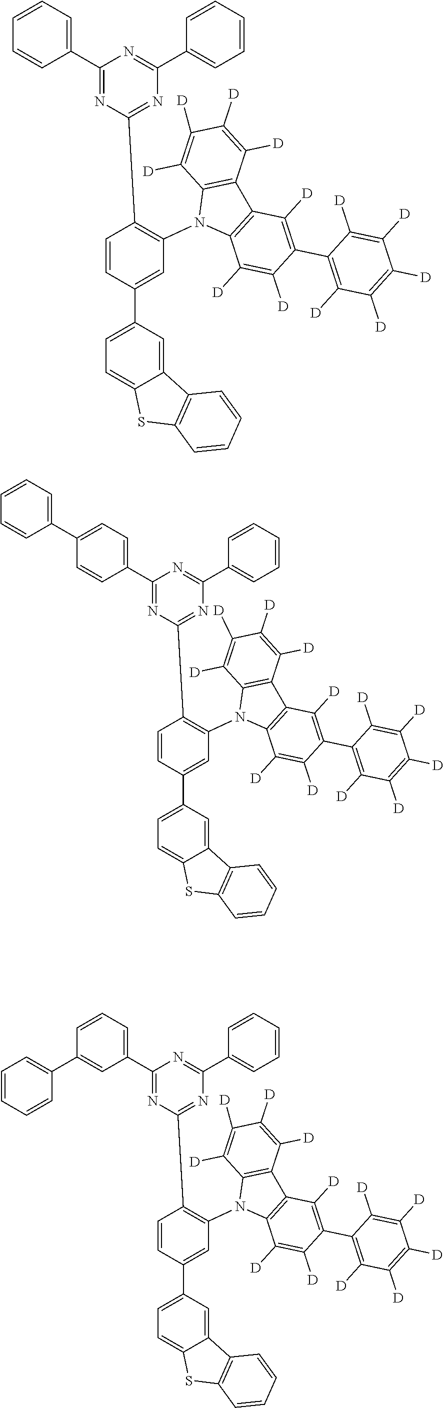

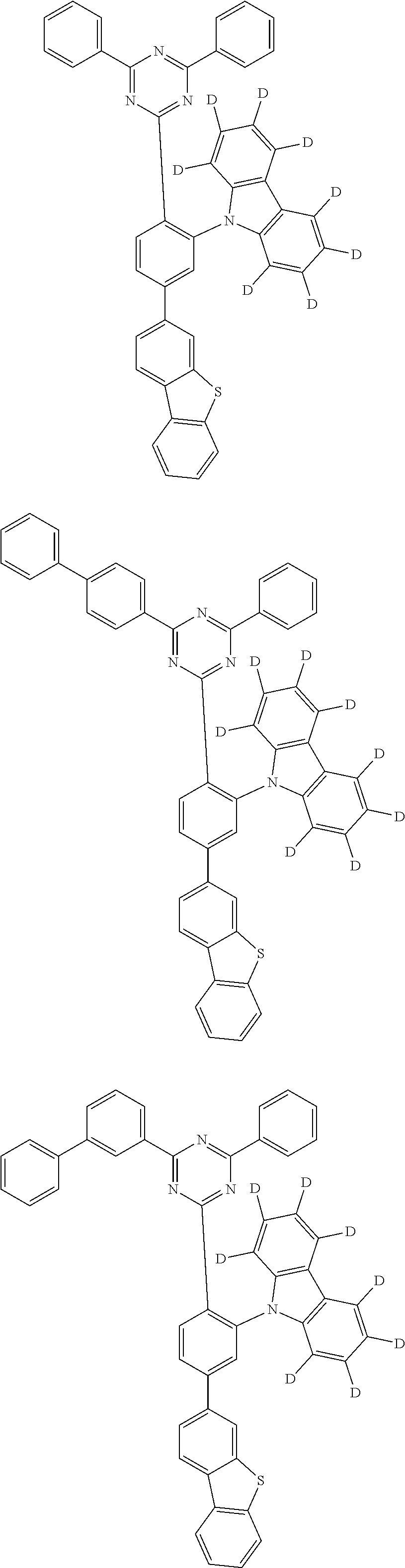 [SMILES: [2H]c1c([2H])c([2H])c2c(c1[2H])c1c([2H])c([2H])c([2H])c([2H])c1n2-c1cc(-c2ccc3c(c2)sc2ccccc23)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cc(-c2ccc3c(c2)sc2ccccc23)ccc1-c1nc(-c2ccccc2)nc(-c2ccc(-c3ccccc3)cc2)n1 ; [2H]c1c([2H])c([2H])c2c(c1[2H])c1c([2H])c([2H])c([2H])c([2H])c1n2-c1cc(-c2ccc3c(c2)sc2ccccc23)ccc1-c1nc(-c2ccccc2)nc(-c2cccc(-c3ccccc3)c2)n1]

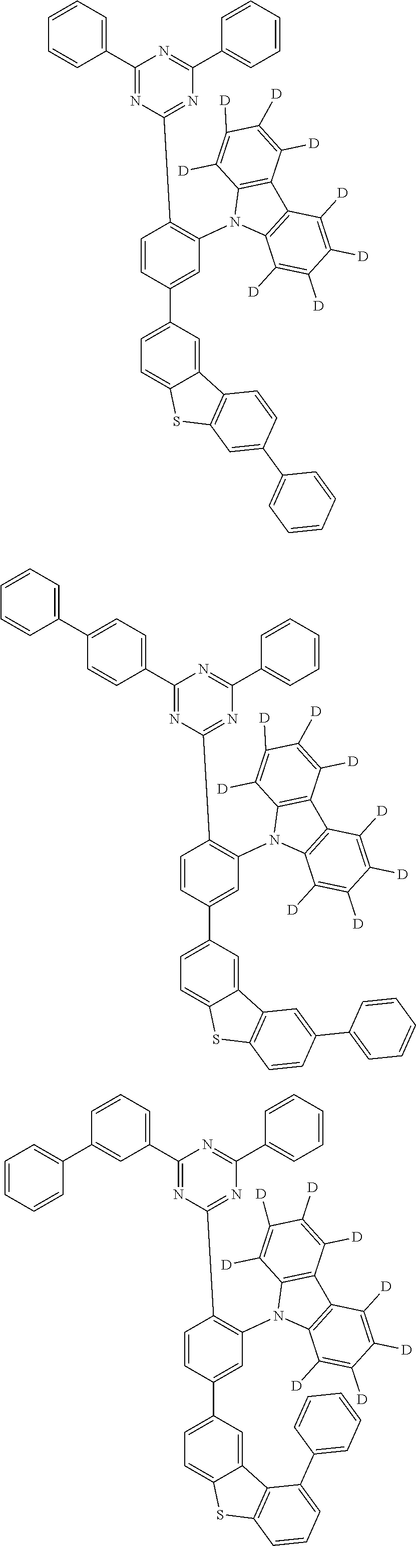

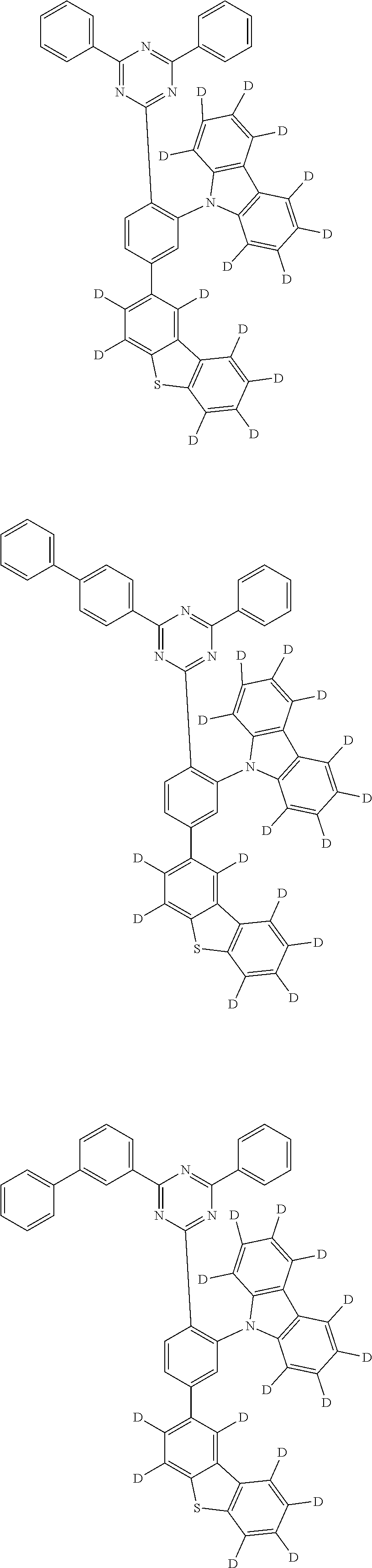

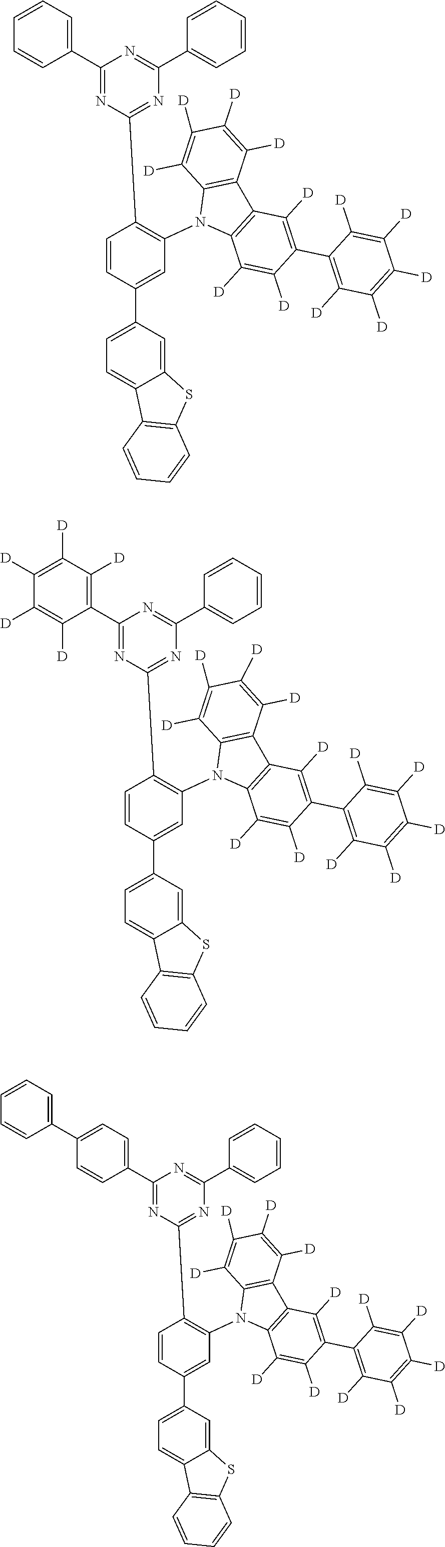

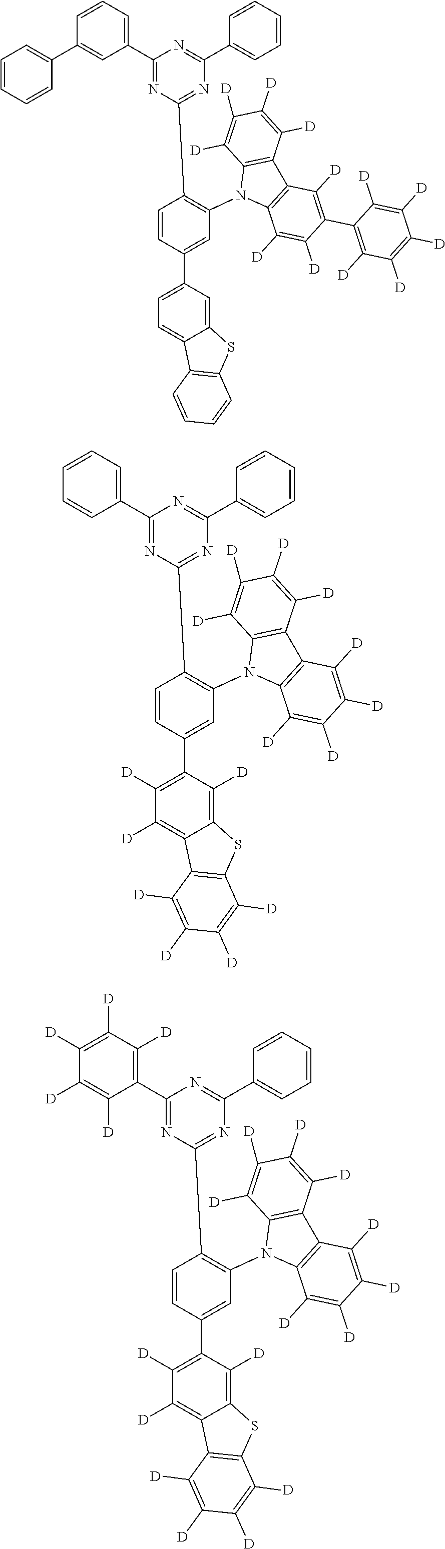

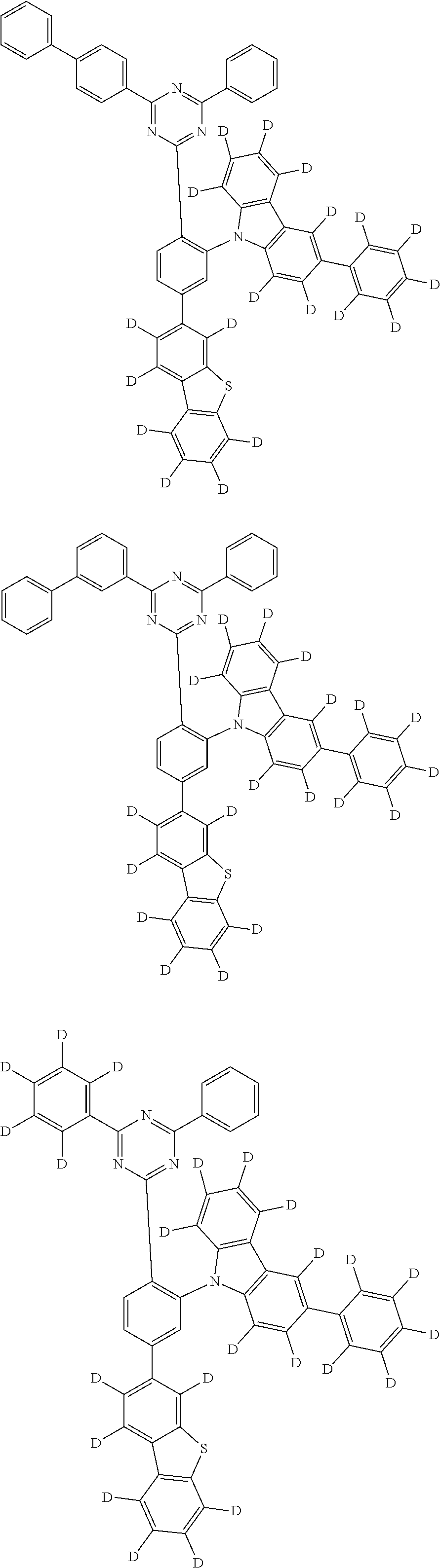

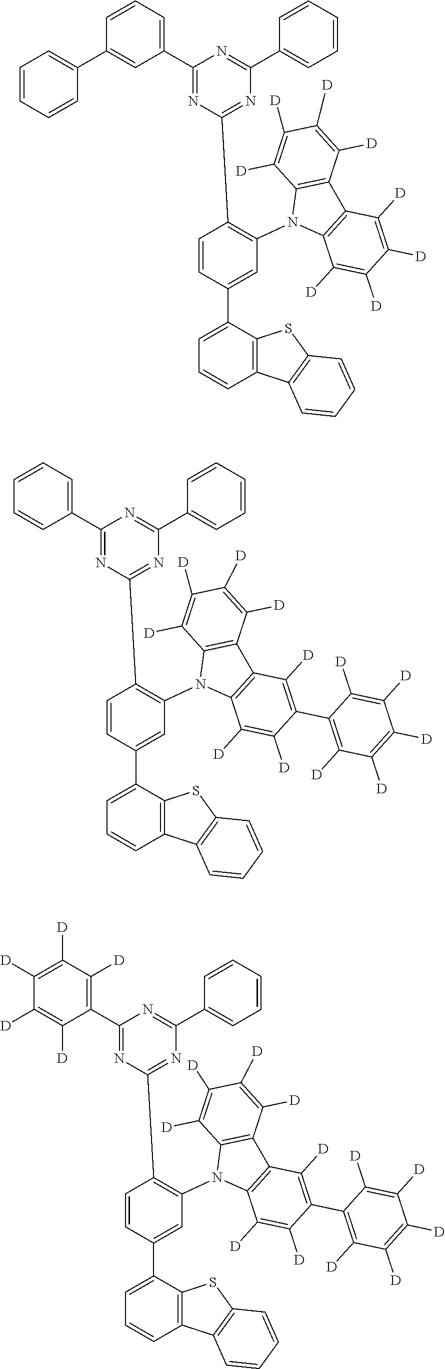 [SMILES: [2H]c1c([2H])c([2H])c2c(c1[2H])c1c([2H])c([2H])c([2H])c([2H])c1n2-c1cc(-c2cccc3c2sc2ccccc23)ccc1-c1nc(-c2ccccc2)nc(-c2cccc(-c3ccccc3)c2)n1 ; [2H]c1c([2H])c([2H])c(-c2c([2H])c([2H])c3c(c2[2H])c2c([2H])c([2H])c([2H])c([2H])c2n3-c2cc(-c3cccc4c3sc3ccccc34)ccc2-c2nc(-c3ccccc3)nc(-c3ccccc3)n2)c([2H])c1[2H] ; [2H]c1c([2H])c([2H])c(-c2nc(-c3ccccc3)nc(-c3ccc(-c4cccc5c4sc4ccccc45)cc3-n3c4c([2H])c([2H])c([2H])c([2H])c4c4c([2H])c(-c5c([2H])c([2H])c([2H])c([2H])c5[2H])c([2H])c([2H])c43)n2)c([2H])c1[2H]]

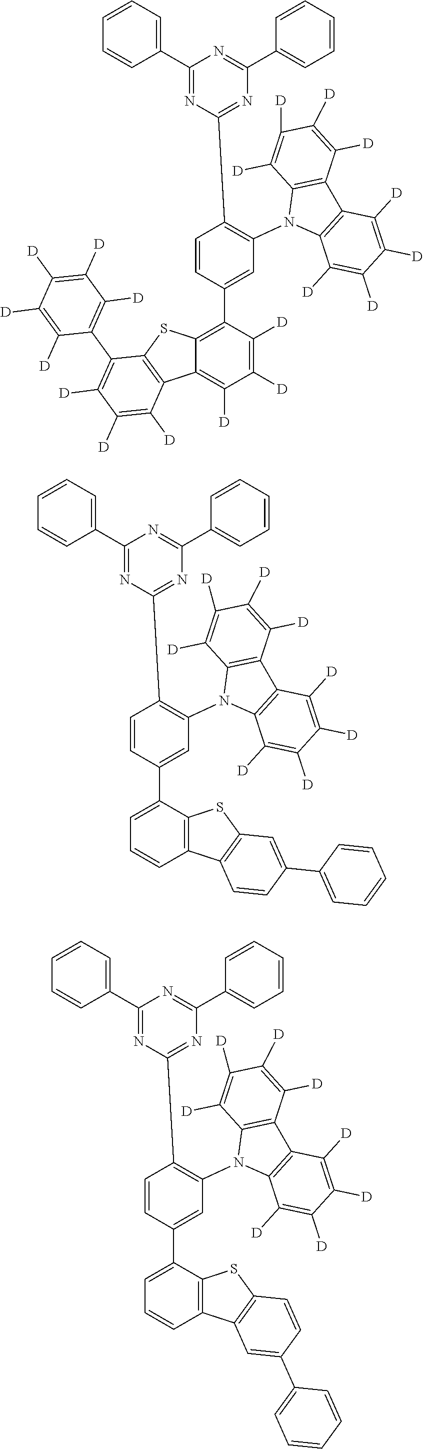 [SMILES: [2H]c1c([2H])c([2H])c(-c2c([2H])c([2H])c([2H])c3c2sc2c(-c4ccc(-c5nc(-c6ccccc6)nc(-c6ccccc6)n5)c(-n5c6c([2H])c([2H])c([2H])c([2H])c6c6c([2H])c([2H])c([2H])c([2H])c65)c4)c([2H])c([2H])c([2H])c23)c([2H])c1[2H] ; [2H]c1c([2H])c([2H])c2c(c1[2H])c1c([2H])c([2H])c([2H])c([2H])c1n2-c1cc(-c2cccc3c2sc2cc(-c4ccccc4)ccc23)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cc(-c2cccc3c2sc2ccc(-c4ccccc4)cc23)ccc1-c1nc(-c2ccccc2)nc(-c2ccccc2)n1]

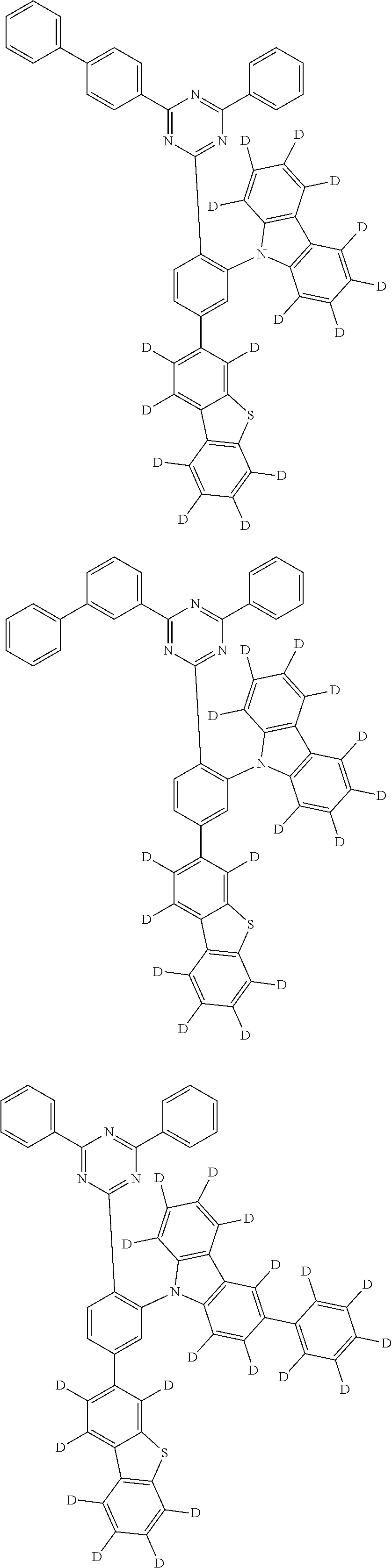

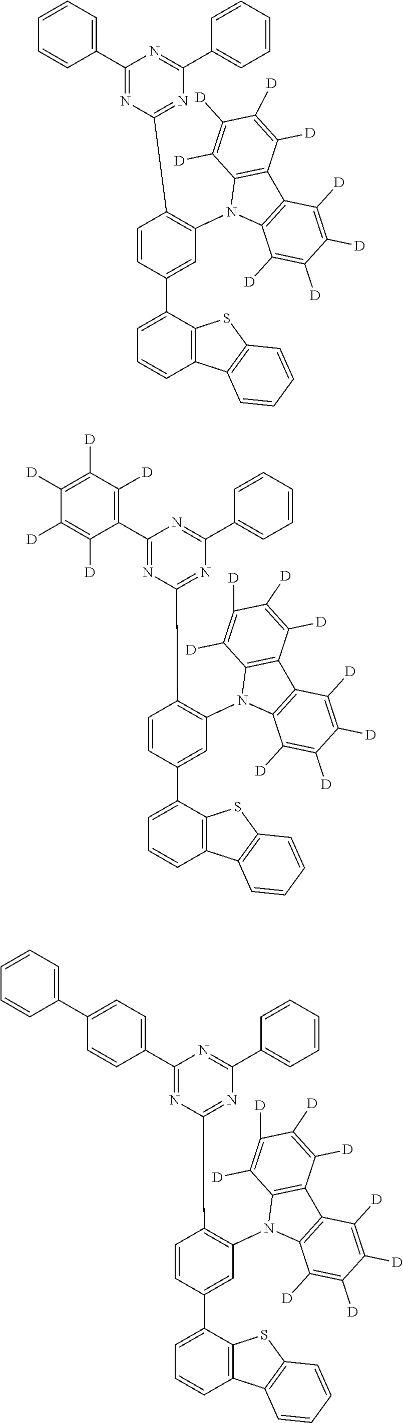 [SMILES: [2H]c1c([2H])c([2H])c2c(c1[2H])c1c([2H])c([2H])c([2H])c([2H])c1n2-c1cc(-c2cccc3c2sc2ccccc23)ccc1-c1nc(-c2ccccc2)nc(-c2ccccc2)n1 ; [2H]c1c([2H])c([2H])c(-c2nc(-c3ccccc3)nc(-c3ccc(-c4cccc5c4sc4ccccc45)cc3-n3c4c([2H])c([2H])c([2H])c([2H])c4c4c([2H])c([2H])c([2H])c([2H])c43)n2)c([2H])c1[2H] ; [2H]c1c([2H])c([2H])c2c(c1[2H])c1c([2H])c([2H])c([2H])c([2H])c1n2-c1cc(-c2cccc3c2sc2ccccc23)ccc1-c1nc(-c2ccccc2)nc(-c2ccc(-c3ccccc3)cc2)n1]

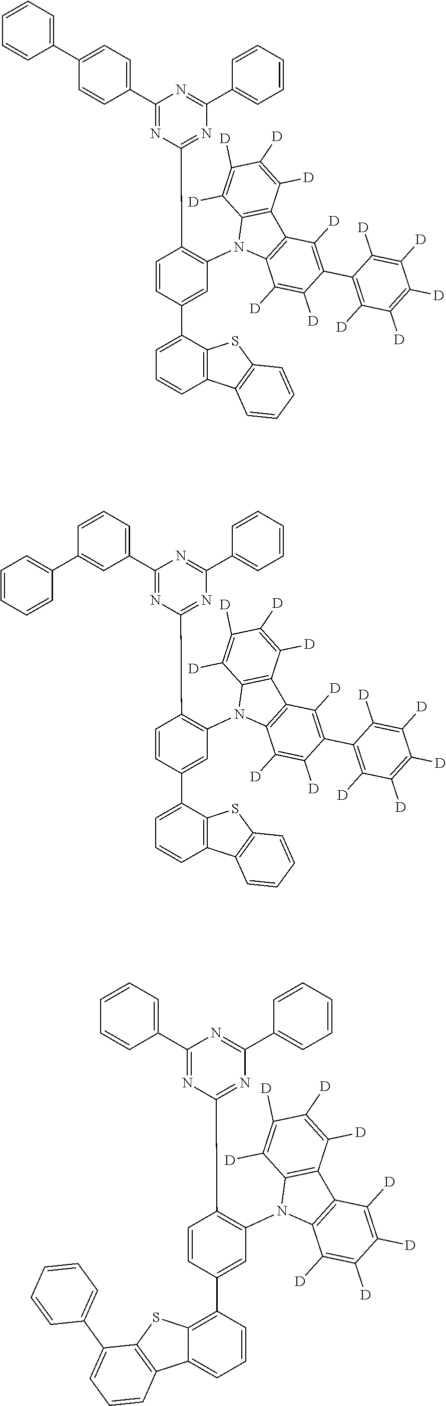 [SMILES: [2H]c1c([2H])c([2H])c(-c2c([2H])c([2H])c3c(c2[2H])c2c([2H])c([2H])c([2H])c([2H])c2n3-c2cc(-c3cccc4c3sc3ccccc34)ccc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2cc(-c3cccc4c3sc3ccccc34)ccc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1cc(-c2cccc3c2sc2c(-c4ccccc4)cccc23)ccc1-c1nc(-c2ccccc2)nc(-c2ccccc2)n1]

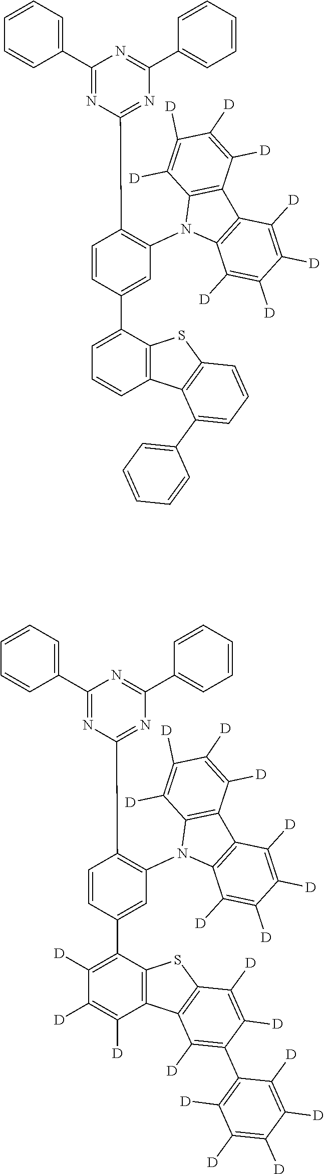 [SMILES: [2H]c1c([2H])c([2H])c2c(c1[2H])c1c([2H])c([2H])c([2H])c([2H])c1n2-c1cc(-c2cccc3c2sc2cccc(-c4ccccc4)c23)ccc1-c1nc(-c2ccccc2)nc(-c2ccccc2)n1 ; [2H]c1c([2H])c([2H])c(-c2c([2H])c([2H])c3sc4c(-c5ccc(-c6nc(-c7ccccc7)nc(-c7ccccc7)n6)c(-n6c7c([2H])c([2H])c([2H])c([2H])c7c7c([2H])c([2H])c([2H])c([2H])c76)c5)c([2H])c([2H])c([2H])c4c3c2[2H])c([2H])c1[2H]]

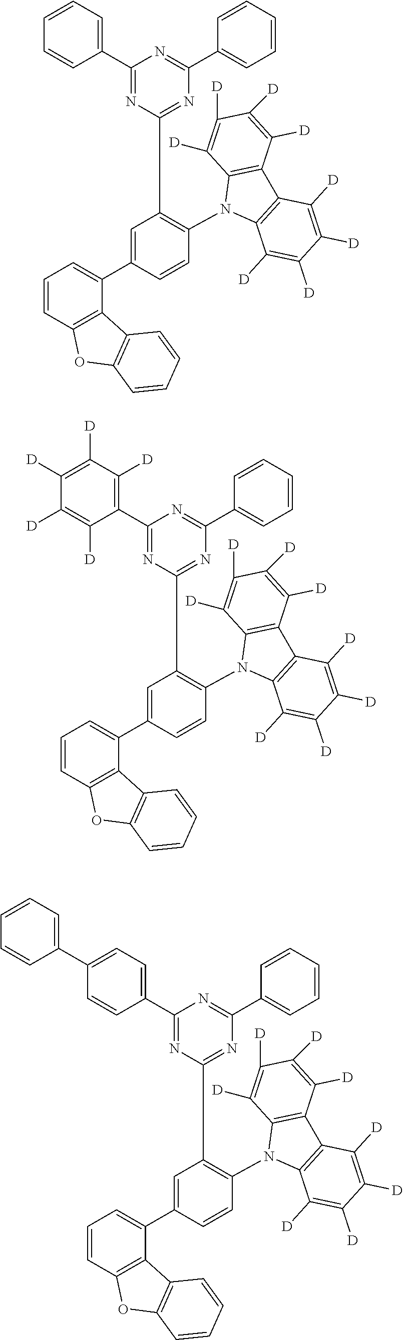 [SMILES: [2H]c1c([2H])c([2H])c2c(c1[2H])c1c([2H])c([2H])c([2H])c([2H])c1n2-c1ccc(-c2cccc3oc4ccccc4c23)cc1-c1nc(-c2ccccc2)nc(-c2ccccc2)n1 ; [2H]c1c([2H])c([2H])c(-c2nc(-c3ccccc3)nc(-c3cc(-c4cccc5oc6ccccc6c45)ccc3-n3c4c([2H])c([2H])c([2H])c([2H])c4c4c([2H])c([2H])c([2H])c([2H])c43)n2)c([2H])c1[2H] ; [2H]c1c([2H])c([2H])c2c(c1[2H])c1c([2H])c([2H])c([2H])c([2H])c1n2-c1ccc(-c2cccc3oc4ccccc4c23)cc1-c1nc(-c2ccccc2)nc(-c2ccc(-c3ccccc3)cc2)n1]

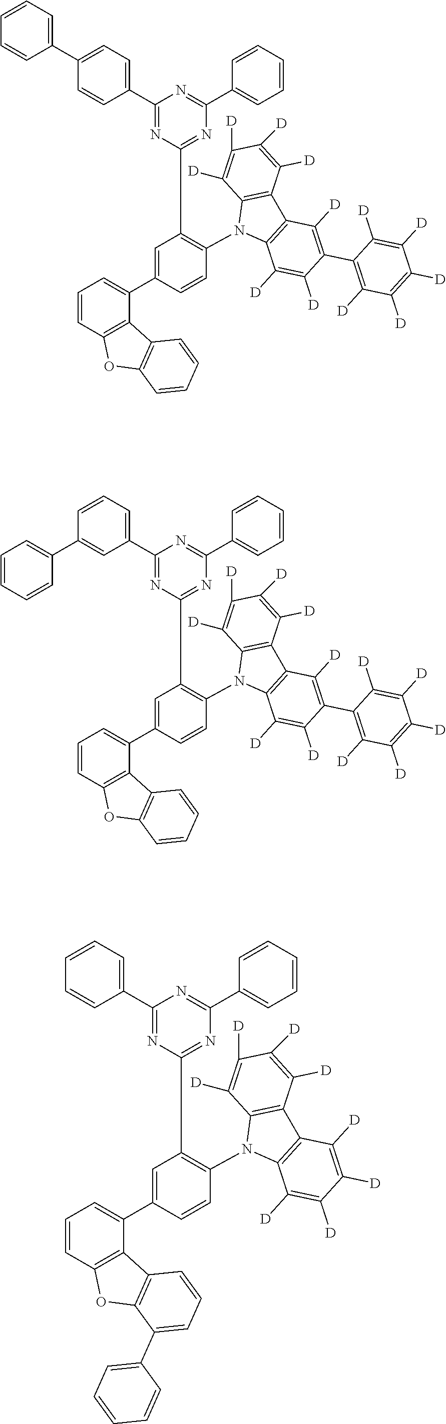 [SMILES: [2H]c1c([2H])c([2H])c(-c2c([2H])c([2H])c3c(c2[2H])c2c([2H])c([2H])c([2H])c([2H])c2n3-c2ccc(-c3cccc4oc5ccccc5c34)cc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2ccc(-c3cccc4oc5ccccc5c34)cc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1ccc(-c2cccc3oc4c(-c5ccccc5)cccc4c23)cc1-c1nc(-c2ccccc2)nc(-c2ccccc2)n1]

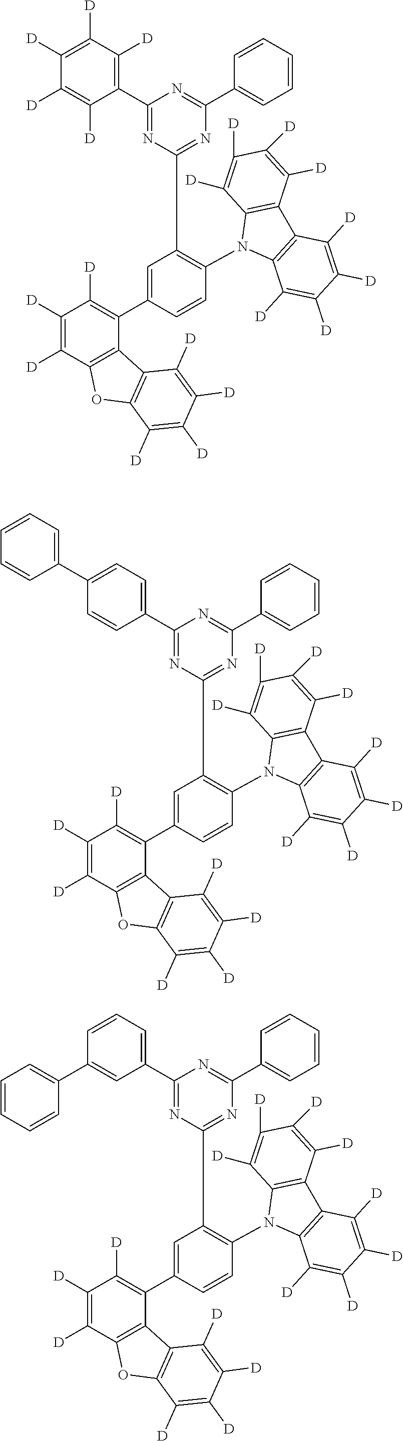

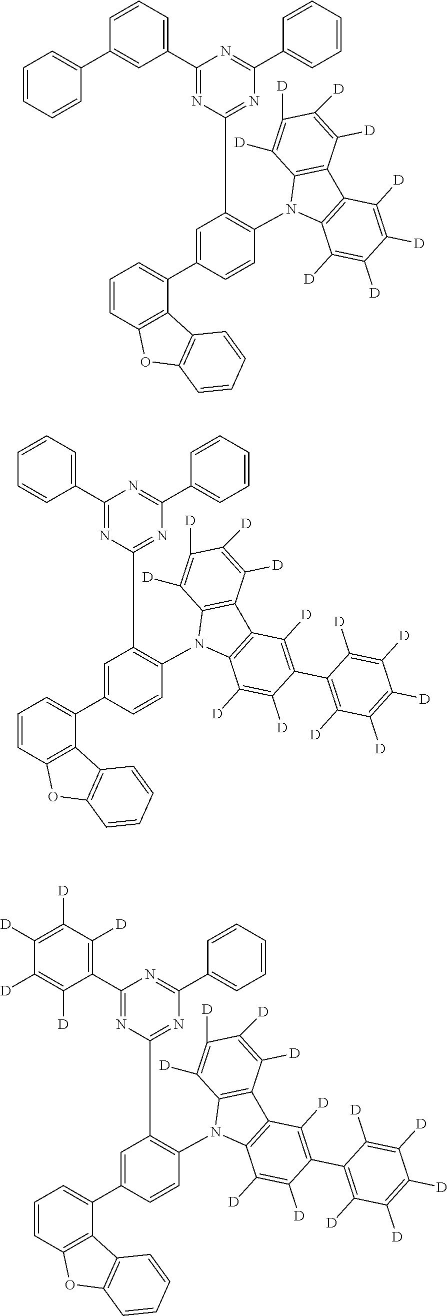 [SMILES: [2H]c1c([2H])c([2H])c2c(c1[2H])c1c([2H])c([2H])c([2H])c([2H])c1n2-c1ccc(-c2cccc3oc4ccccc4c23)cc1-c1nc(-c2ccccc2)nc(-c2cccc(-c3ccccc3)c2)n1 ; [2H]c1c([2H])c([2H])c(-c2c([2H])c([2H])c3c(c2[2H])c2c([2H])c([2H])c([2H])c([2H])c2n3-c2ccc(-c3cccc4oc5ccccc5c34)cc2-c2nc(-c3ccccc3)nc(-c3ccccc3)n2)c([2H])c1[2H] ; [2H]c1c([2H])c([2H])c(-c2nc(-c3ccccc3)nc(-c3cc(-c4cccc5oc6ccccc6c45)ccc3-n3c4c([2H])c([2H])c([2H])c([2H])c4c4c([2H])c(-c5c([2H])c([2H])c([2H])c([2H])c5[2H])c([2H])c([2H])c43)n2)c([2H])c1[2H]]

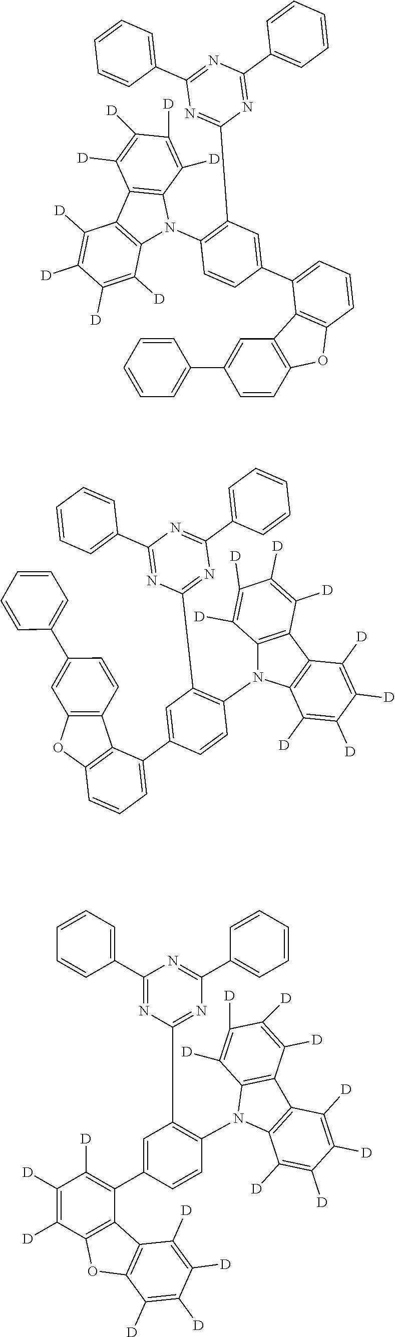

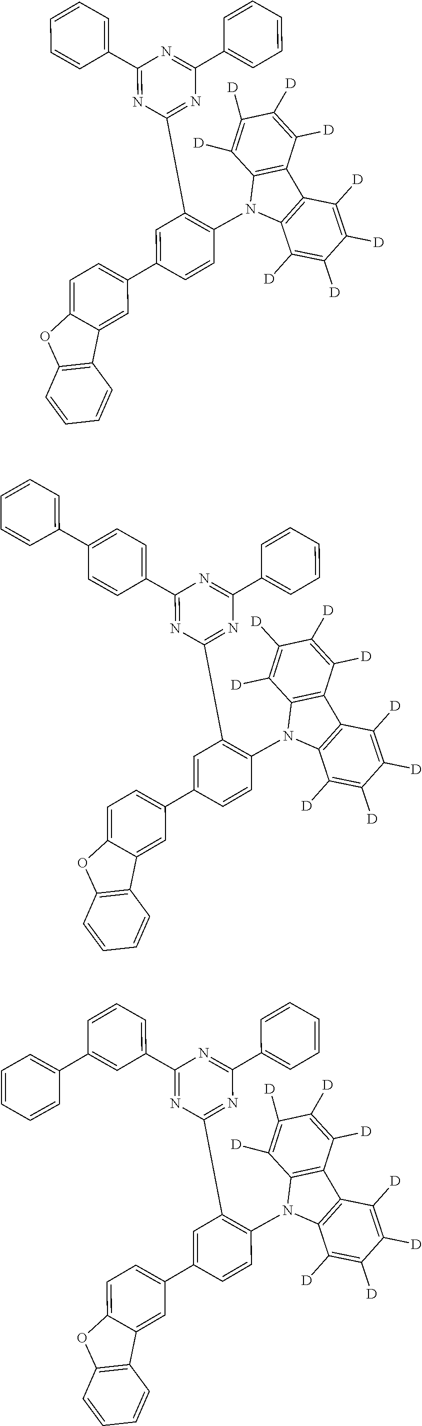

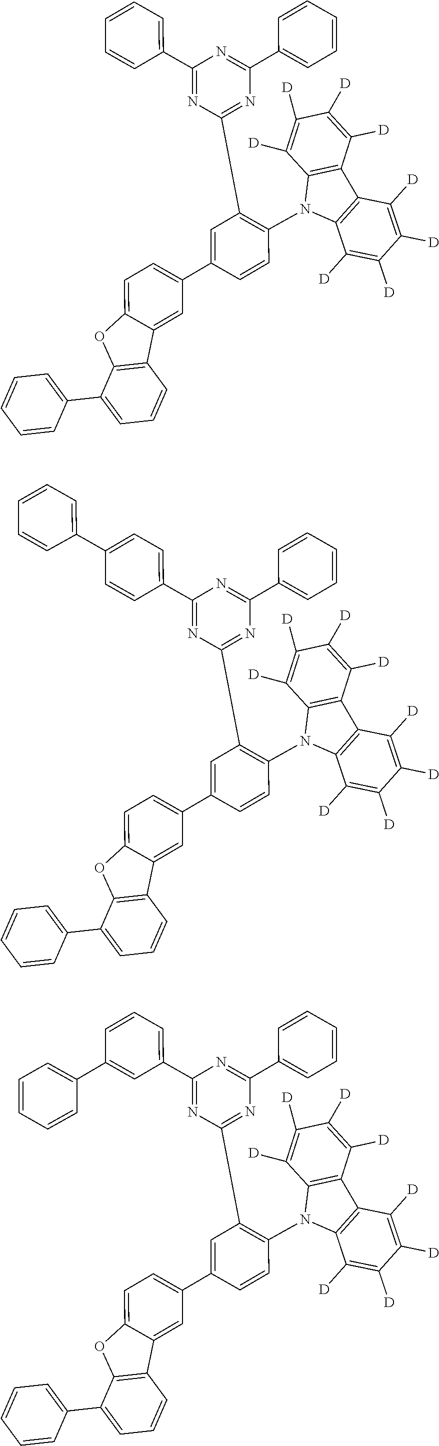

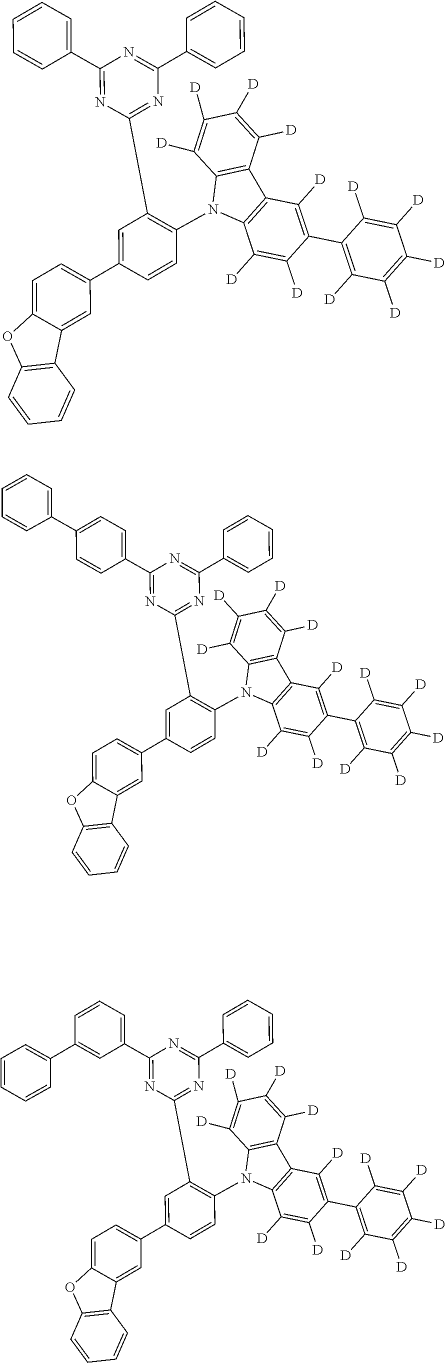

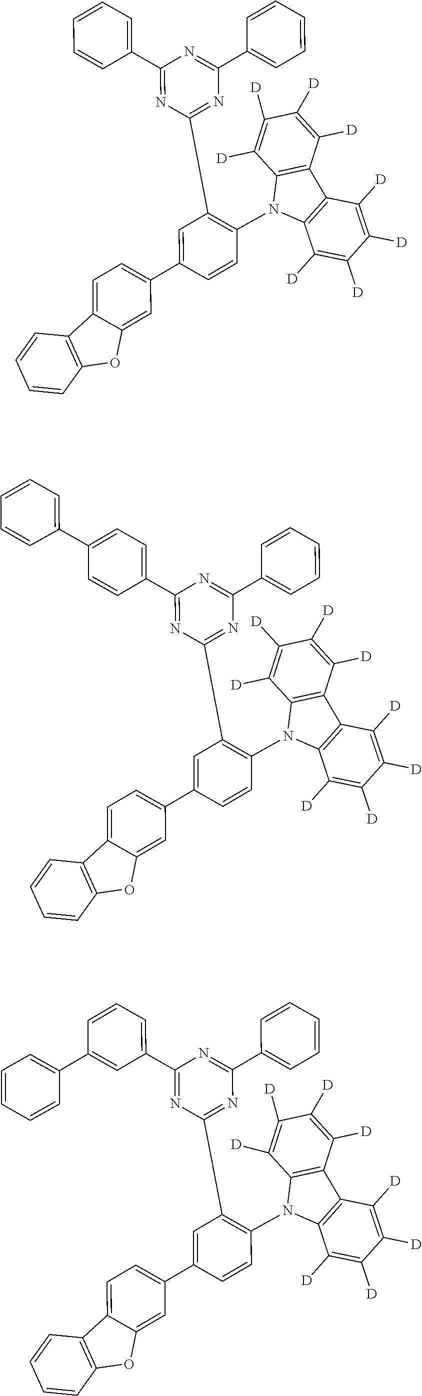

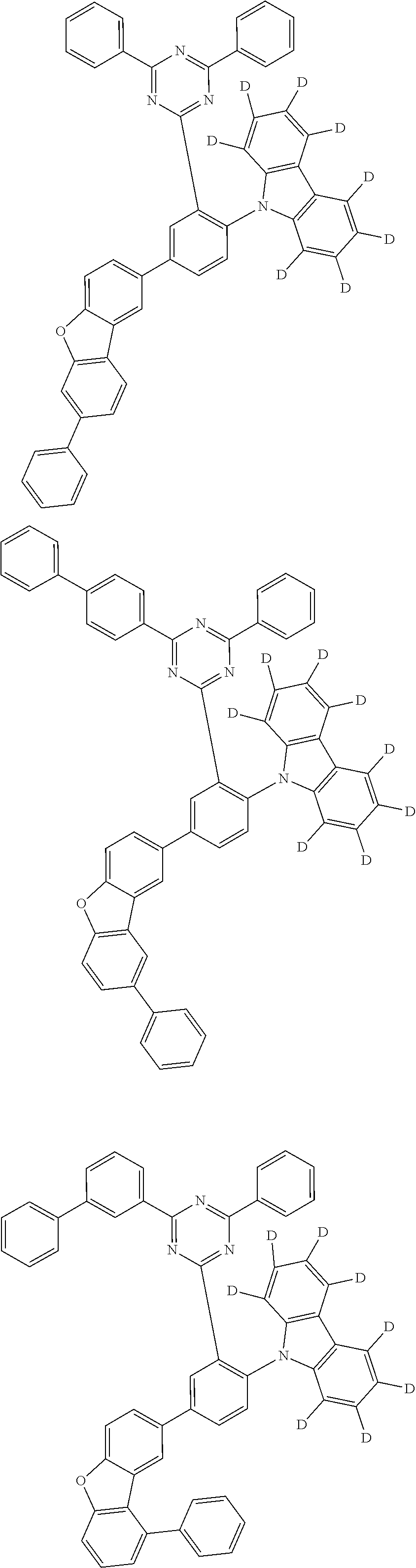

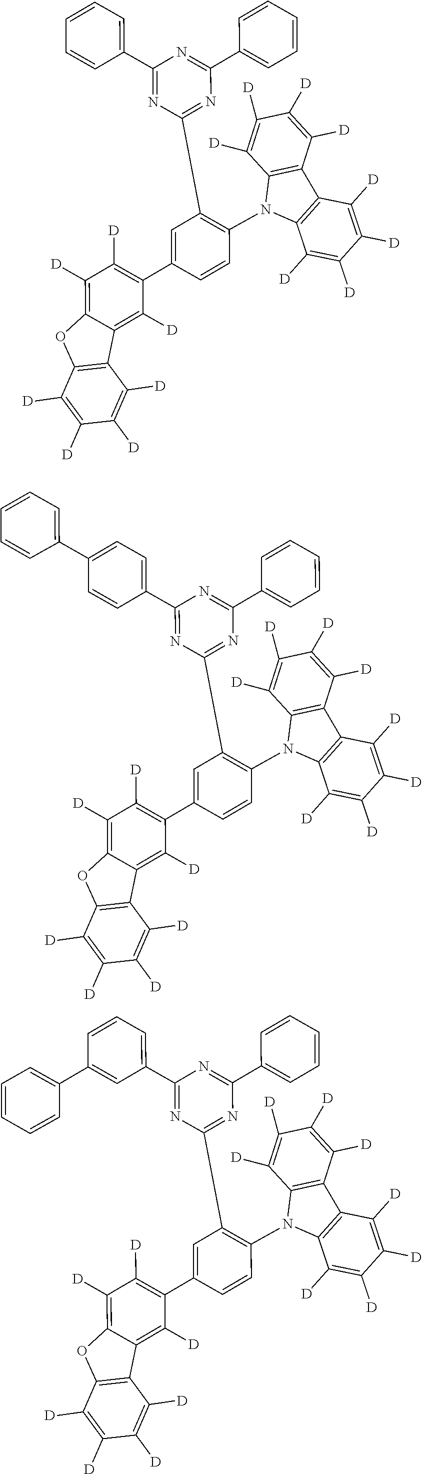

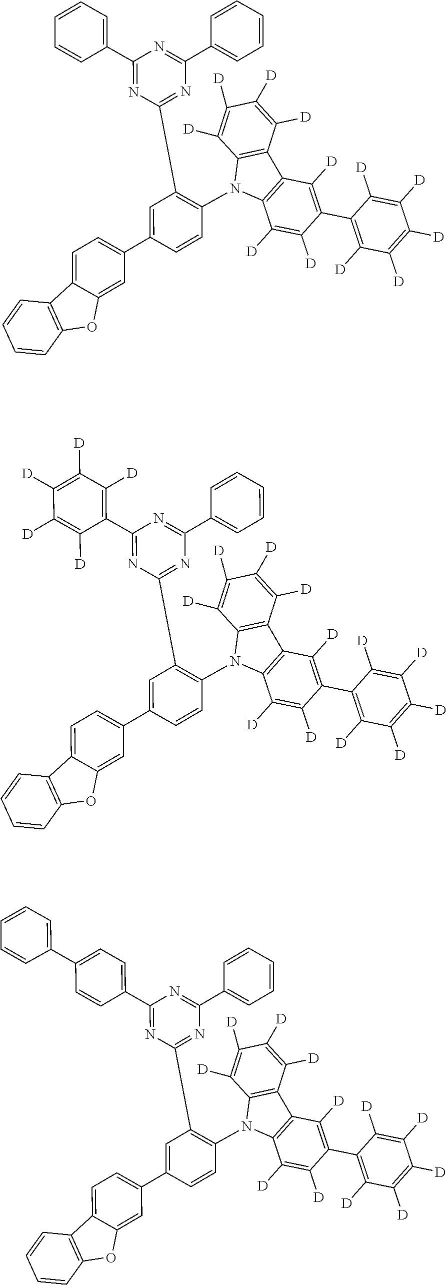

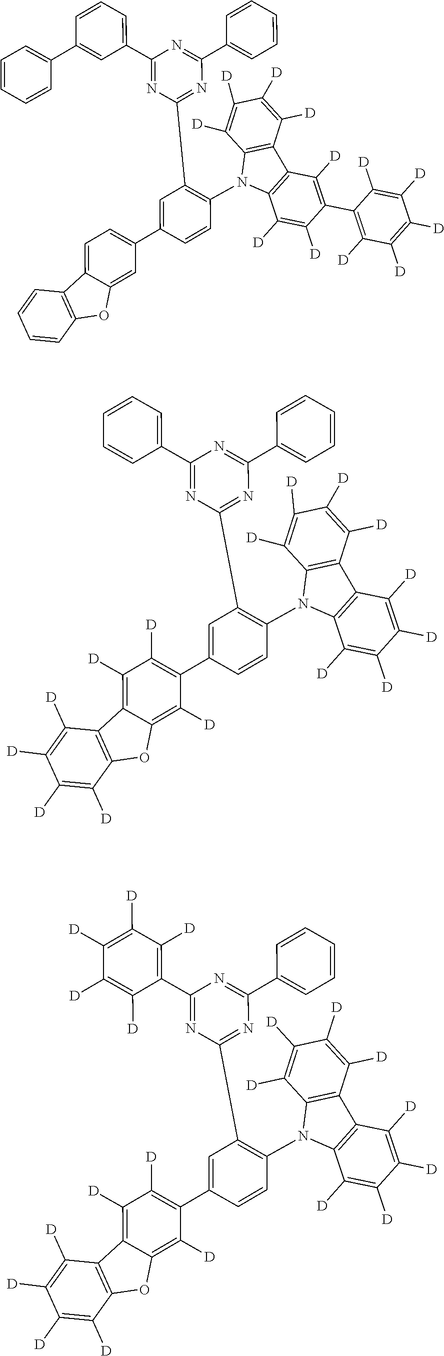

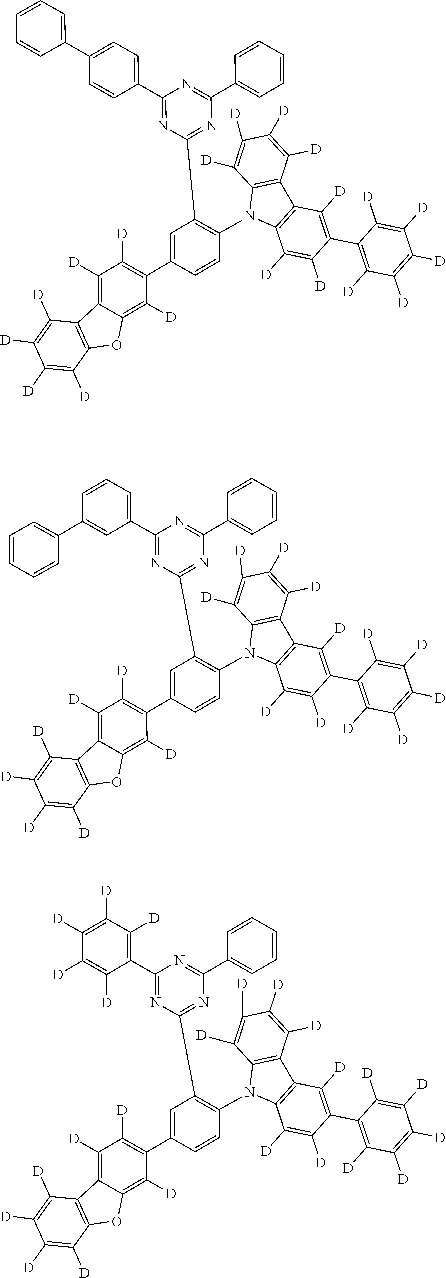

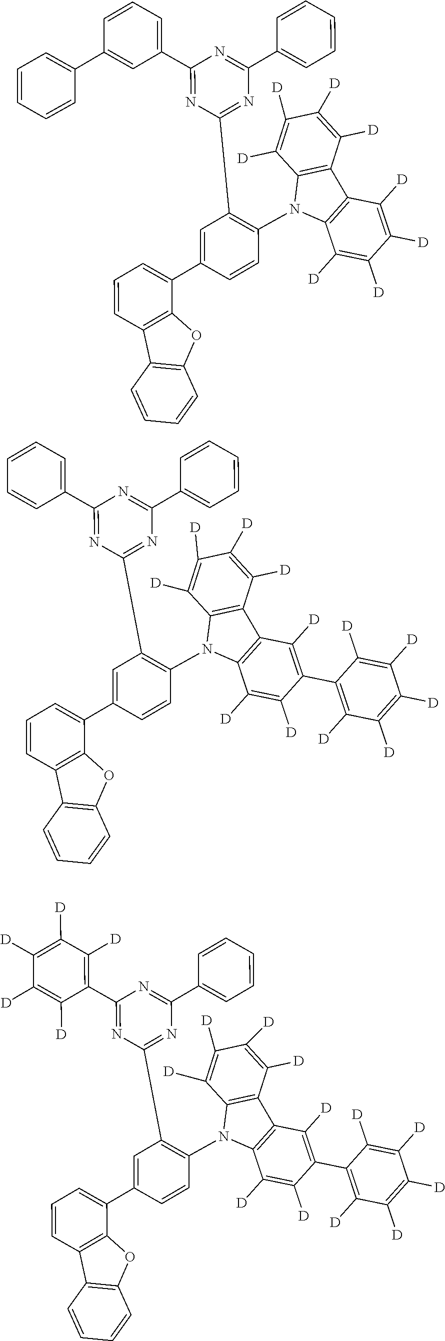

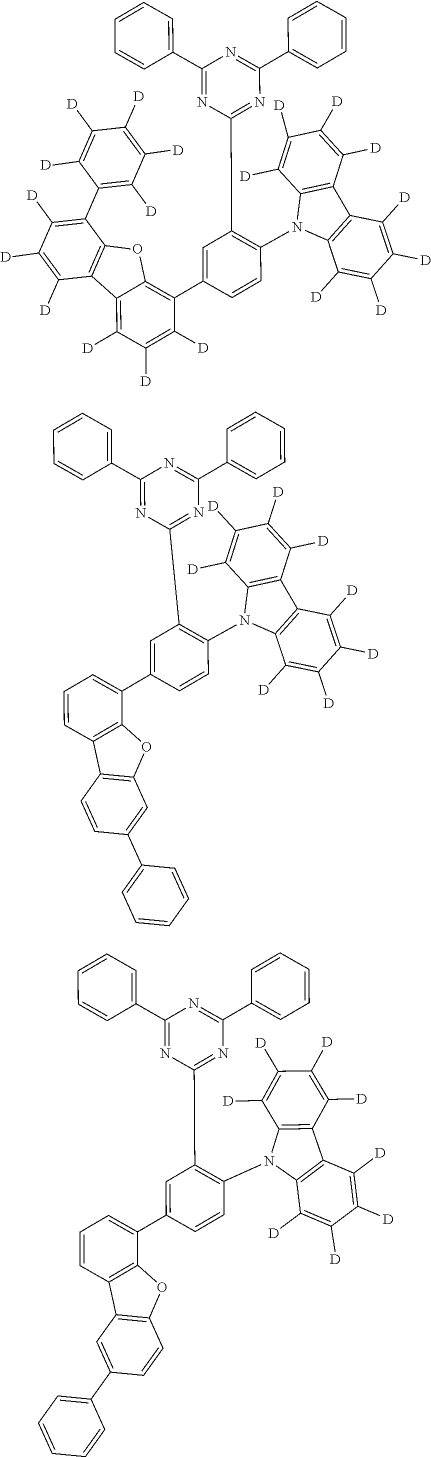

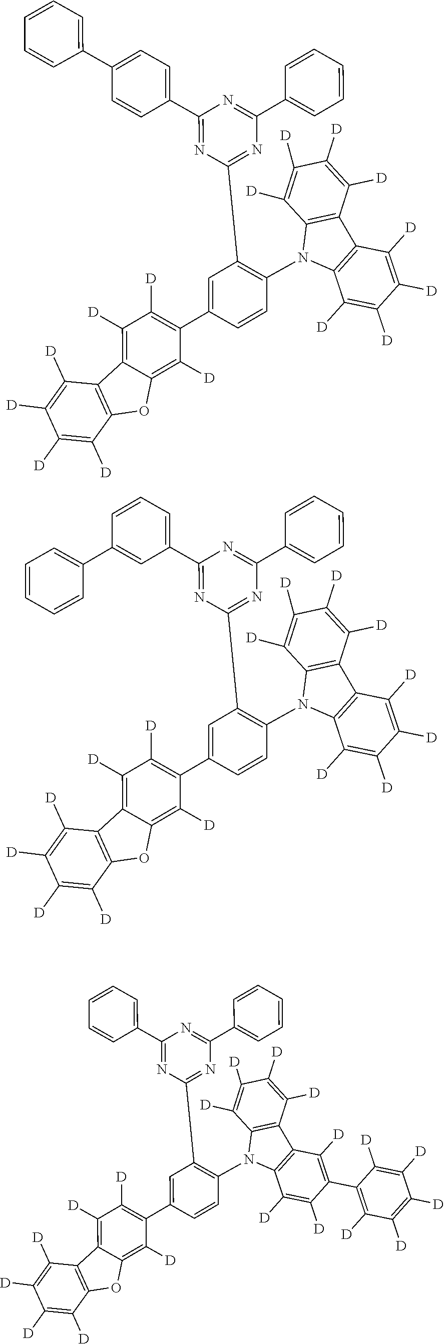

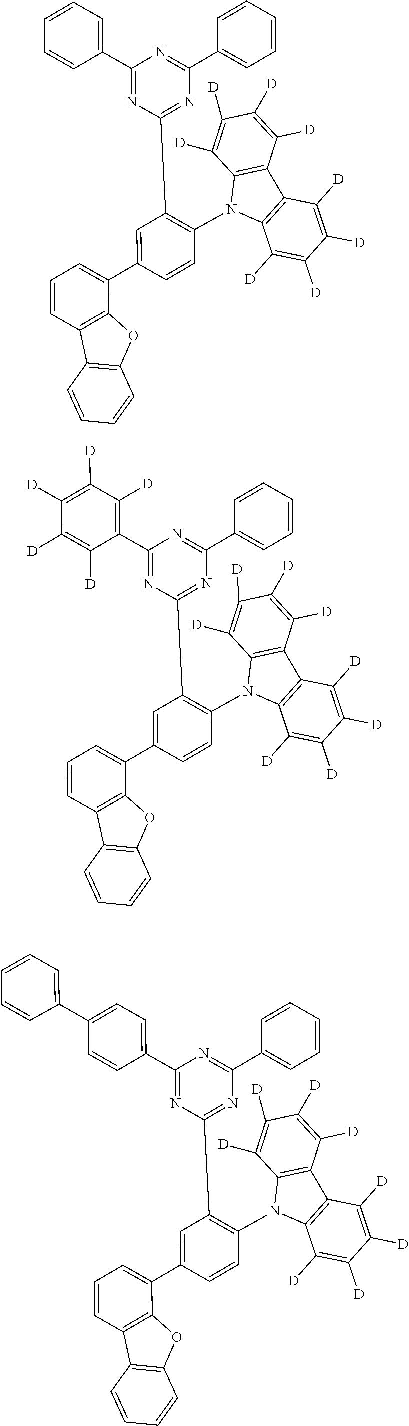

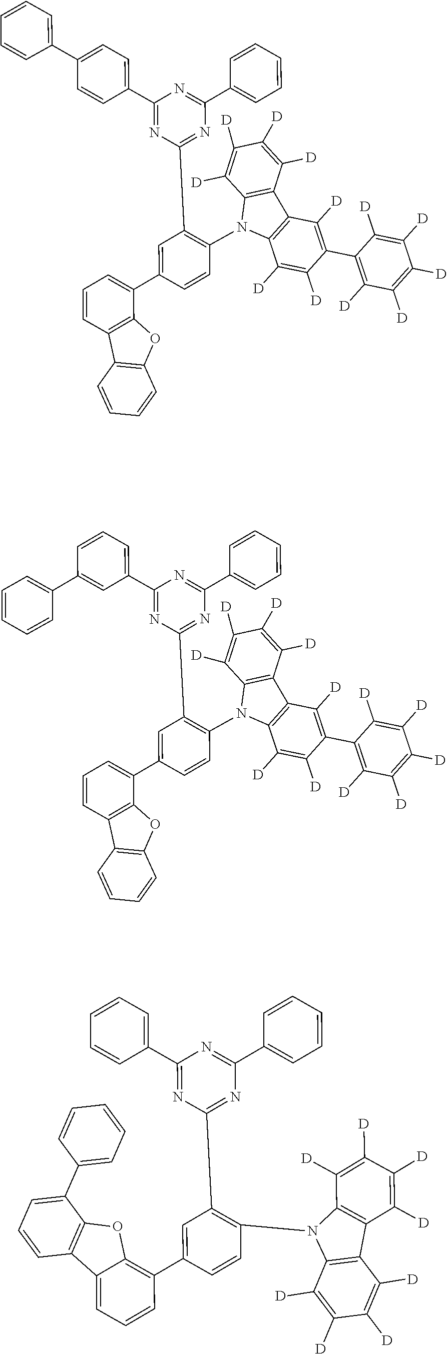 [SMILES: [2H]c1c([2H])c([2H])c(-c2c([2H])c([2H])c3c(c2[2H])c2c([2H])c([2H])c([2H])c([2H])c2n3-c2ccc(-c3cccc4c3oc3ccccc34)cc2-c2nc(-c3ccccc3)nc(-c3ccc(-c4ccccc4)cc3)n2)c([2H])c1[2H] ; [2H]c1c([2H])c([2H])c(-c2c([2H])c([2H])c3c(c2[2H])c2c([2H])c([2H])c([2H])c([2H])c2n3-c2ccc(-c3cccc4c3oc3ccccc34)cc2-c2nc(-c3ccccc3)nc(-c3cccc(-c4ccccc4)c3)n2)c([2H])c1[2H] ; [2H]c1c([2H])c([2H])c2c(c1[2H])c1c([2H])c([2H])c([2H])c([2H])c1n2-c1ccc(-c2cccc3c2oc2c(-c4ccccc4)cccc23)cc1-c1nc(-c2ccccc2)nc(-c2ccccc2)n1]

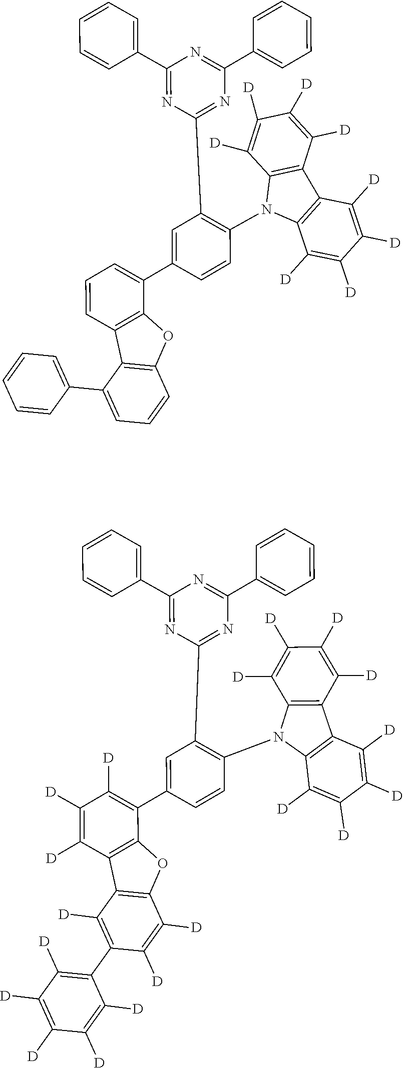 [SMILES: [2H]c1c([2H])c([2H])c2c(c1[2H])c1c([2H])c([2H])c([2H])c([2H])c1n2-c1ccc(-c2cccc3c2oc2cccc(-c4ccccc4)c23)cc1-c1nc(-c2ccccc2)nc(-c2ccccc2)n1 ; [2H]c1c([2H])c([2H])c(-c2c([2H])c([2H])c3oc4c(-c5ccc(-n6c7c([2H])c([2H])c([2H])c([2H])c7c7c([2H])c([2H])c([2H])c([2H])c76)c(-c6nc(-c7ccccc7)nc(-c7ccccc7)n6)c5)c([2H])c([2H])c([2H])c4c3c2[2H])c([2H])c1[2H]]

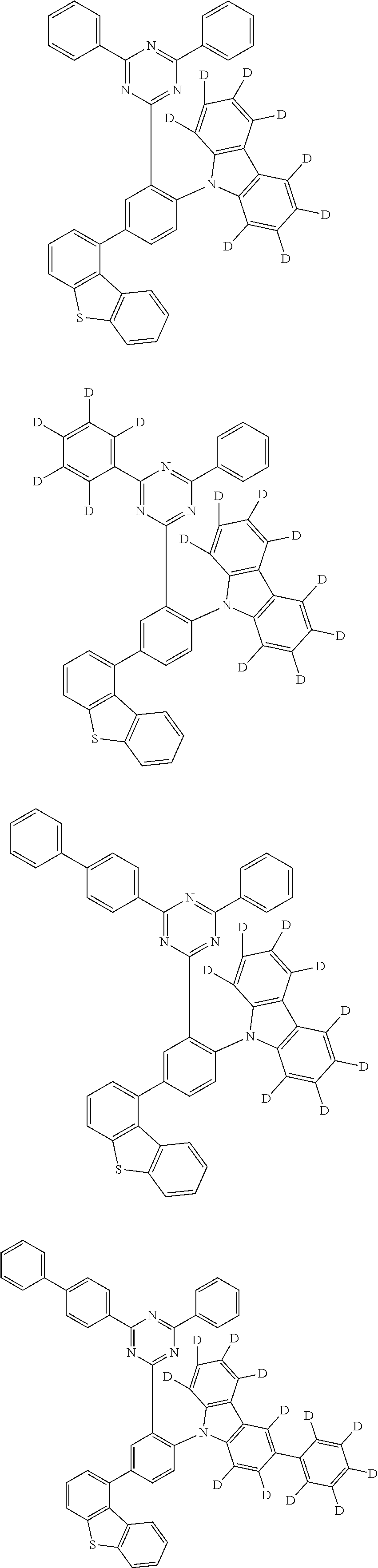

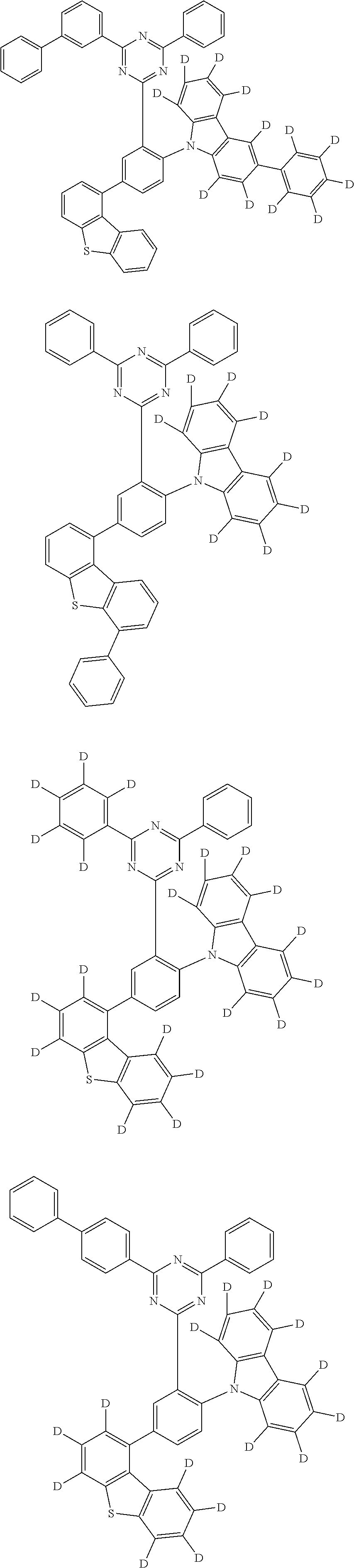

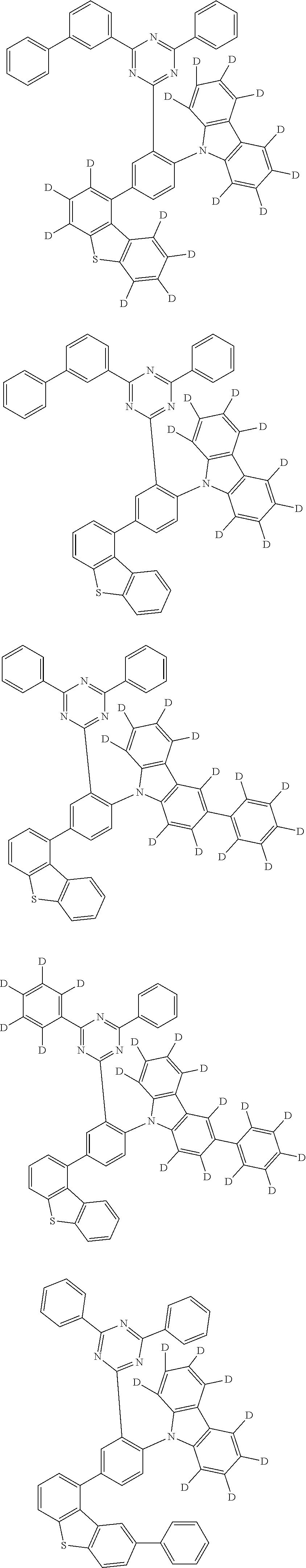

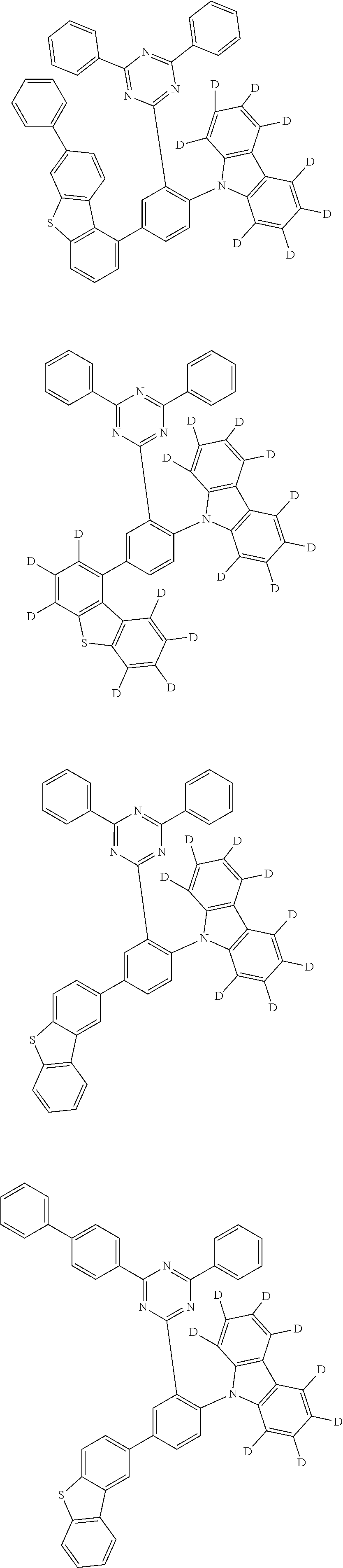

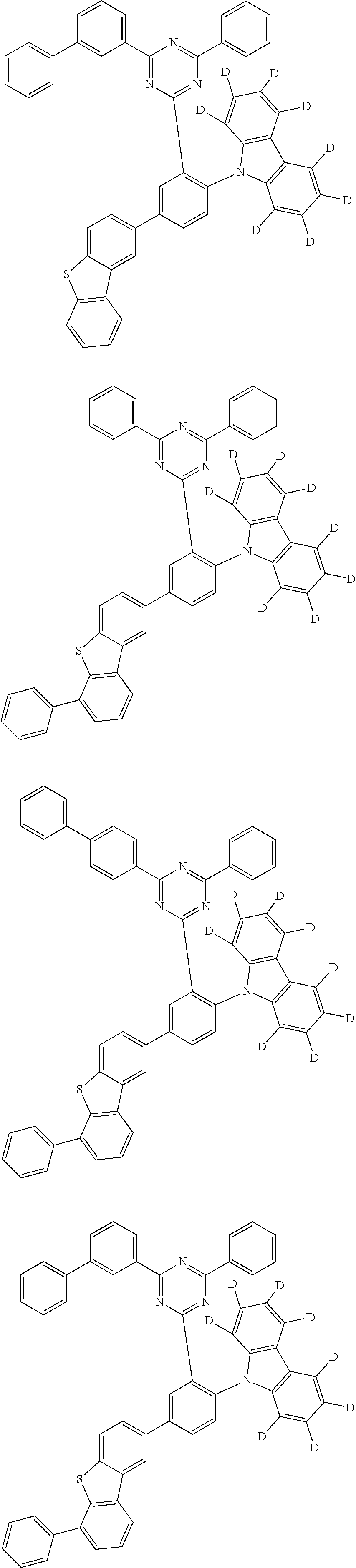

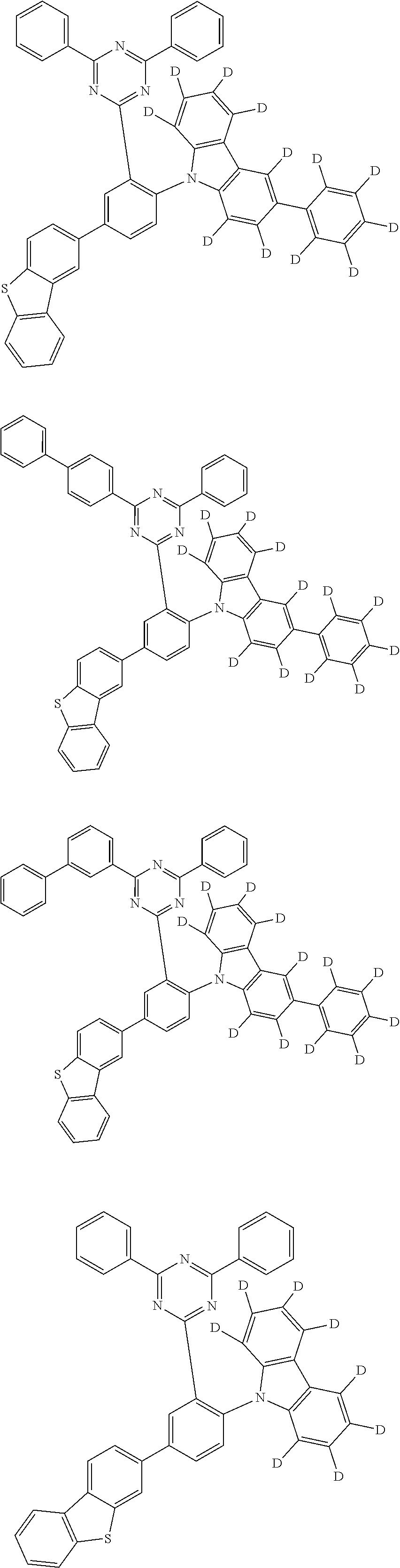

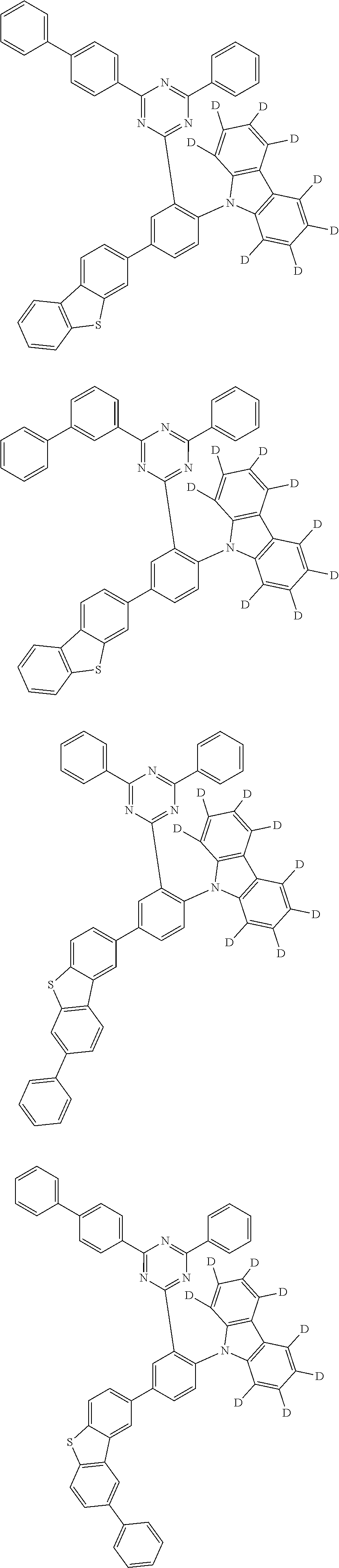

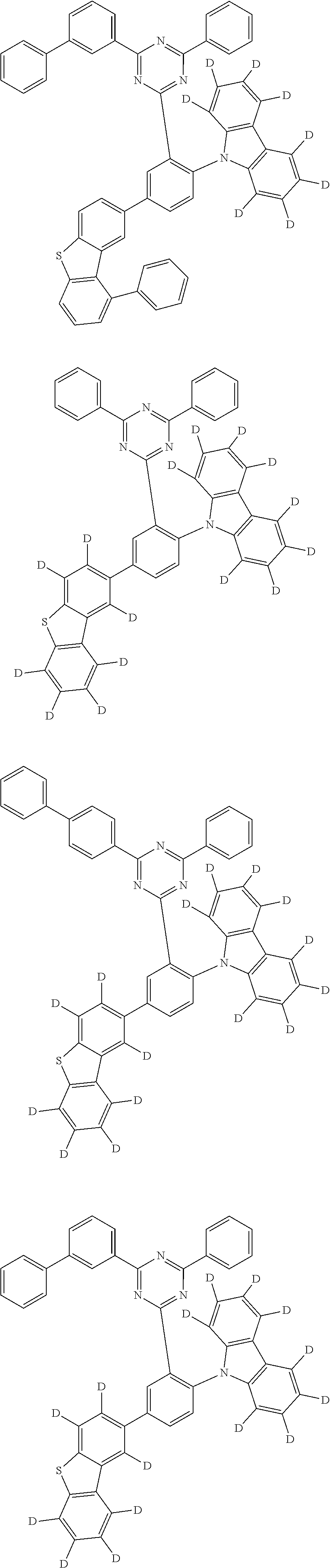

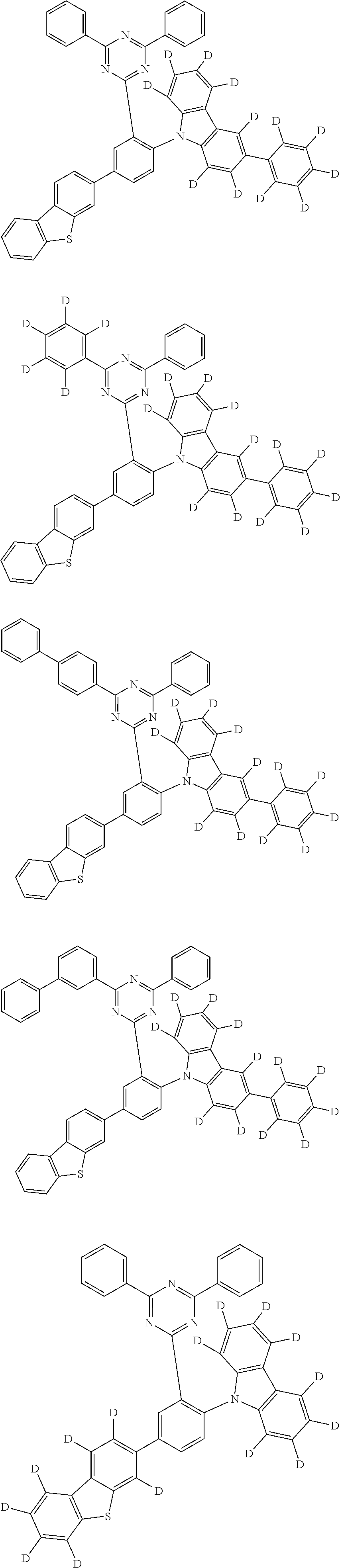

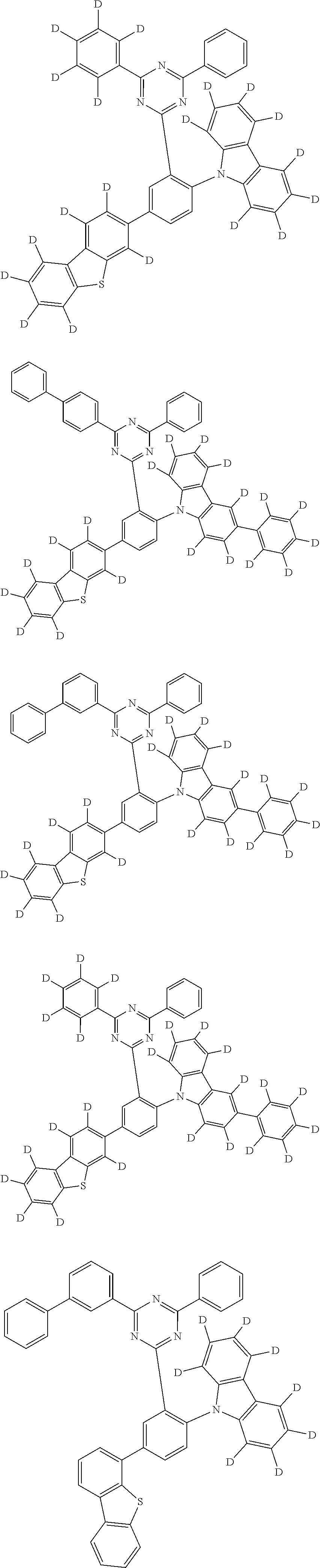

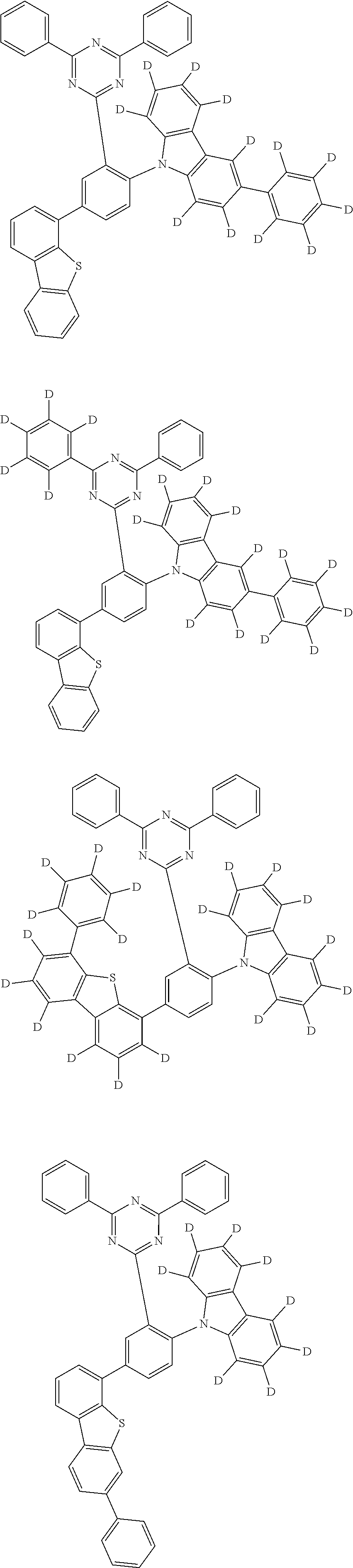

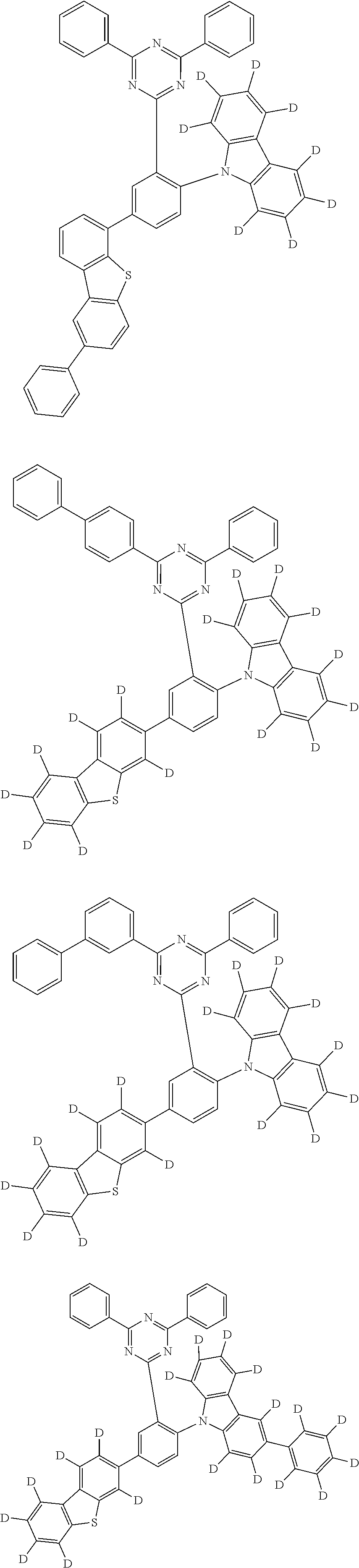

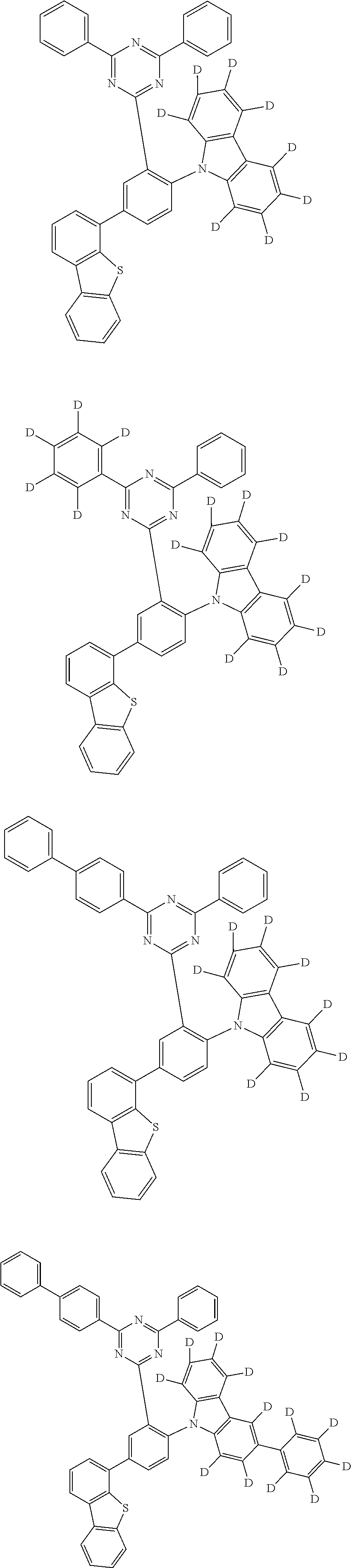

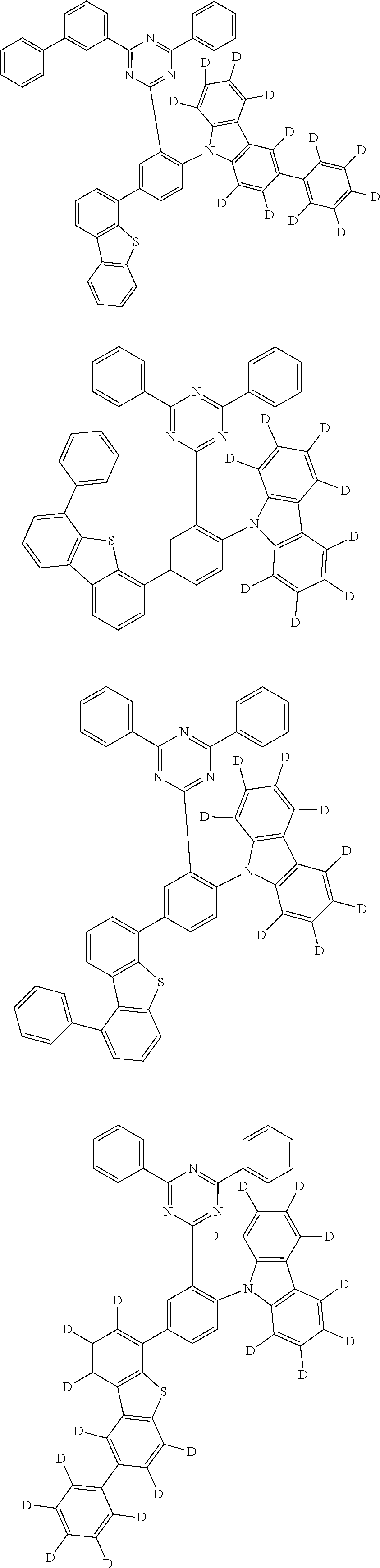

10. An organic light emitting device comprising: a first electrode;
a second electrode that is provided opposite to the first electrode; and one or more organic material layers that are provided between the first electrode and the second electrode, wherein at least one layer of the one or more organic material layers comprises the compound of Chemical Formula 1 of claim 1.

11. An organic light emitting device as claimed in claim 10, wherein:
the organic material layer comprising the compound is a light emitting layer.
